(19) United States
(12) Patent Application Publication
(10) Pub. No.: US 2026/0297552 A1
(43) Pub. Date: Oct. 1, 2026

(54) EVOLVED ADENINE DEAMINASES AND RNA-GUIDED NUCLEASE FUSION PROTEINS WITH INTERNAL INSERTION SITES AND METHODS OF USE

(71) Applicant: Life Edit Therapeutics, Inc., Durham, NC (US)

(72) Inventors: Michael Coyle, Chapel Hill, NC (US); Alexandra Briner Crawley, Cary, NC (US); Tedd D. Elich, Durham, NC (US); Salem Faham, Durham, NC (US); Drew Kelso, Holly Springs, NC (US); Adrian Pickar Oliver, Rougemont, NC (US); Lucas Ferreira Ribeiro, Raleigh, NC (US); Ryan Rickels, Durham, NC (US); Sarah Compton, Durham, NC (US)

(73) Assignee: Life Edit Therapeutics, Inc., Durham, NC (US)

(21) Appl. No.: 19/126,929

(22) PCT Filed: Nov. 6, 2023
(86) PCT No.: PCT/IB2023/061192

§ 371 Date: May 2, 2025

Related U.S. Application Data
(60) Provisional application No. 63/382,344, filed on Nov. 4, 2022.
(60) Provisional application No. 63/485,642, filed on Feb. 17, 2023.

(51) Int. Cl. C12N 9/78; A61K 38/00; C12N 9/22; C12N 15/113

(52) U.S. Cl. CPC C12N 9/78; C12N 9/226; C12N 15/113; A61K 38/00; C07K 2319/85; C12N 2310/20; C12Y 305/04002

(57) ABSTRACT

Compositions and methods comprising deaminases for targeted editing of nucleic acids are provided. Also provided are compositions and methods for localizing a heterologous polypeptide to a target DNA molecule and for targeted editing of nucleic acids are provided. Fusion proteins comprising an RNA-guided nuclease (RGN) and at least one heterologous polypeptide inserted therein and fusion proteins comprising a DNA-binding polypeptide and a deaminase are provided. The heterologous polypeptide can be a prime editing polypeptide or a base-editing polypeptide. Compositions also include nucleic acid molecules encoding the deaminases or the fusion proteins. Vectors and host cells comprising the nucleic acid molecules encoding the deaminases or fusion proteins are also provided.

### CROSS-REFERENCE TO RELATED APPLICATIONS

This application is a national stage filing under 35 U.S.C. 371 of PCT/IB2023/061192, filed Nov. 6, 2023, which was published by the International Bureau in English on May 10, 2024, which claims priority to US Provisional Patent Application Nos. 63/382,344, filed Nov. 4, 2022 and 63/485,642, filed Feb. 17, 2023, each of which is fully incorporated by reference herein.

REFERENCE TO A SEQUENCE LISTING SUBMITTED ELECTRONICALLY AS AN XML FILE The instant application contains a Sequence Listing which has been submitted in xml format via USPTO Patent Center and is hereby incorporated by reference in its entirety. Said xml copy, created on Nov. 6, 2023, is named L103438_1330WO_Seq_List.xml, and is 1,181,580 bytes in size.

### FIELD OF THE INVENTION

The present invention relates to the field of molecular biology and gene editing.

### BACKGROUND OF THE INVENTION

Targeted genome editing or modification is rapidly becoming an important tool for basic and applied research. Initial methods involved engineering nucleases such as meganucleases, zinc finger fusion proteins or TALENs, requiring the generation of chimeric nucleases with engineered, programmable, sequence-specific DNA-binding domains specific for each particular target sequence. RNA-guided nucleases (RGNs), such as the Clustered Regularly Interspaced Short Palindromic Repeats (CRISPR)-associated (Cas) proteins of the CRISPR-Cas bacterial system, allow for the targeting of specific sequences by complexing the nucleases with guide RNA that specifically hybridizes with a particular target sequence. Producing target-specific guide RNAs is less costly and more efficient than generating chimeric nucleases for each target sequence. Such RNA-guided nucleases can be used to edit genomes through the introduction of a sequence-specific, double-stranded break that is repaired via error-prone non-homologous end-joining (NHEJ) to introduce a mutation at a specific genomic location.

Additionally, RGNs are useful for targeted DNA editing approaches. Targeted editing of nucleic acid sequences, for example targeted cleavage, to allow for introduction of a specific modification into genomic DNA, enables a highly nuanced approach to studying gene function and gene expression. RGNs may also be used to generate chimeric proteins which use the RNA-guided activity of the RGN in combination with a DNA modifying enzyme, such as a deaminase, for targeted base editing. Targeted editing may be deployed for targeting genetic diseases in humans or for introducing agronomically beneficial mutations in the genomes of crop plants. The development of genome editing tools provides new approaches to gene editing-based mammalian therapeutics and agrobiotechnology.

### BRIEF SUMMARY OF THE INVENTION

Compositions and methods for localizing a heterologous polynucleotide to a target DNA molecule and modifying a target DNA molecule are provided. Provided compositions comprise deaminase polypeptides and fusion proteins comprising an RNA-guided nuclease (RGN) and at least one heterologous polypeptide, such as a deaminase, inserted therein. The heterologous polypeptide can be a prime editing polypeptide or a base-editing polypeptide. Fusion proteins comprising base editing polypeptides are also referred to herein as a base editor. Thus, in some embodiments, the base editor comprises a nucleic acid molecule-binding polypeptide (e.g., DNA-binding polypeptide) and a deaminase polypeptide. In some embodiments, the base editor comprises an RGN with a deaminase inserted therein. In some embodiments, the base editor comprising an RGN with a deaminase polypeptide inserted therein exhibits improved editing activity and/or a shifted editing window when compared to a parental base editor. In some embodiments, the parental base editor comprises the deaminase fused to the amino terminus of the RGN. Also provided are nucleic acid molecules encoding the disclosed deaminase polypeptides and fusion proteins, vectors comprising the nucleic acid molecules, and cells comprising the deaminase polypeptides or fusion proteins, nucleic acid molecules, or vectors.

Systems comprising the disclosed fusion proteins (or a nucleic acid molecule encoding the same) and one or more guide RNAs (or one or more nucleic acid molecules encoding the same) are provided for localizing a heterologous polypeptide or deaminase to a target DNA molecule, along with cells comprising the systems. Methods for localizing a heterologous polypeptide or deaminase and/or modifying a target DNA molecule comprise delivering a disclosed system to the target DNA molecule or a cell comprising the target DNA molecule.

Pharmaceutical compositions comprising the presently disclosed deaminases and fusion proteins, nucleic acid molecules encoding the same (or vectors or cells comprising the same), or systems (or cells comprising the same) are provided. Methods for treating subjects having or at risk of developing a disease, disorder, or condition comprise administering to the subject the presently disclosed fusion proteins, nucleic acid molecules encoding the same (or vectors or cells comprising the same), systems (or cells comprising the same), or pharmaceutical compositions. In some embodiments, the disease, disorder, or condition is associated with a causal mutation and treating comprises correcting the causal mutation.

### BRIEF DESCRIPTION OF THE FIGURES

FIG. 1 depicts the rational design approach to improve editing efficiency at bases outside the canonical editing window of the parental adenine base editor LPG50148-nAPG07433.1. FIG. 1A provides schematic cartoons of the linear base editor showing the parental adenine base editor (ABE) as an end-to-end fusion between LPG50148 and nAPG07433.1, and the internal base editor wherein LPG50148 is inserted within nAPG07433.1. FIG. 1B shows a structural model of APG07433.1 with the insertion points (blue spheres). LPG50148 (green) was inserted after the positions shown. The G910, V872, and E900 insertion sites are within the wedge domain; the T642 and R670 insertion sites are within the HNH domain; and the D737, A802, S775, S772, K778, and R30 insertion sites are within the RuvCIII domain.

FIG. 2 shows the editing efficiency and editing window of nAPG07433.1 inlaid base editors (IBEs) LPG20002-LPG20006 and the HNH replacement LPG20001. FIG. 2A provides the total editing efficiency of the first five designed nAPG07433.1 IBEs that were tested with genomic targets for the total percent of A to G substitutions. FIG. 2B shows the editing windows of LPG20001-LPG20006. The heatmap provides the percent editing for each position. The rows represent five individual guides (top to bottom: SGN001681, SGN001062, SGN000968, SGN000754, and SGN001064). Positions 1-25 denote the location of the first through 25^{th} target adenines within the protospacer with position 1 being the first adenine 5′ of the PAM.

FIG. 3 shows the editing efficiency and editing window of nAPG07433.1 IBEs LPG20007-LPG20011. The heatmap provides the percent editing for each position. The rows represent four individual guides (top to bottom: SGN001062, SGN000968, SGN000754, and SGN001064).

FIG. 4 describes nAPG07433.1 IBEs 2 and 8. FIG. 4A provides a structural representation of the insertion points for nAPG07433.1 IBEs 2 (S772) and 8 (T642). FIG. 4B depicts the schematic domain organization of LPG20002 and LPG20008 compared to the parental ABE. FIG. 4C shows the average editing efficiency of LPG20002 and LPG20008 for three target sites (SGN001947, SGN001064, and SGN000909) compared to the parental ABE.

FIG. 5 shows the evaluation of nAPG07433.1 IBE2 with deleted linkers. FIG. 5A depicts the insertion of truncated LPG50148 into nAPG07433.1 and the deletion of the N-terminal linker, C-terminal linker, or both linkers to create nAPG07433.1 IBE2 variants. FIG. 5B shows the total editing efficiency (percentage of A to G substitutions) for all LPG20002 variants tested with four genomic targets. FIG. 5C provides a heatmap showing the percent editing for each position. The rows represent four individual target sites (top to bottom: SGN001062, SGN001064, SGN000754, and SGN000968).

FIG. 6 shows the therapeutic potential of engineered IBEs. FIG. 6A provides the results from the disruption of the splice donor in exon 1 in the transthyretin (TTR) gene as a strategy for gene knockout. Plasmids were delivered into HEK cells via nucleofection. FIG. 6B shows the use of LPG20002 variants to reduce the P2-microglobulin (B2M) surface presentation. mRNA was delivered via nucleofection into T cells.

FIG. 7 shows editing activity of nAPG05586 IBEs. FIG. 7A provides the average editing rate across the editing window (adenines 5-24) for nAPG05586 IBEs with four guide RNAs following delivery of plasmids encoding IBEs and guide RNAs into HEK293T cells via nucleofection. FIG. 7B provides the editing rates at every adenine position within the editing window for each of the nAPG05586 IBEs.

FIG. 8 shows editing activity of nAPG01604 IBEs. FIG. 8A provides the average editing rate across the editing window (adenines 5-19) for nAPG01604 IBEs with four guide RNAs following delivery of plasmids encoding IBEs and guide RNAs into HEK293T cells via nucleofection. FIG. 8B provides the editing rates at every adenine position within the editing window for each of the nAPG01604 IBEs.

FIG. 9 shows editing activity of nLPG10145 IBEs. FIG. 9A provides the average editing rate across the editing window (adenines 4-25) for LPG10145 IBEs with four guide RNAs following delivery of plasmids encoding IBEs and guide RNAs into HEK293T cells via nucleofection. FIG. 9B provides the editing rates at every adenine position within the editing window for each of the nLPG10145 IBEs.

FIGS. 10A and 10B depict the first step of the directed evolution strategy for identifying adenine base editor variants that have greater activity than the parental ABE (LPG50148 fused to dAPG07433.1).

FIG. 10A shows the end-to-end fusion proteins of an LPG50148 adenosine deaminase variant and a nuclease inactive version of the APG07433.1 RNA-guided nuclease that are expressed in E. coli and convert stop codons to sense codons in the Kanamycin resistance gene to confer resistance. FIG. 10B demonstrates how the evolved ABEs were enriched for those that had greater editing activity than the parental ABE. E. coli harboring the target plasmid were transformed with a library of mutant LPG50148 fused to dAPG07433.1. To grow in high concentrations of kanamycin, cells are required to repair the antibiotic resistance gene (KanR*) with an editing efficiency higher than the parental ABE. The selections were performed individually against three sequence contexts: TAT, CAC, or TAG. After the enrichment steps, variants with up to an eight-fold improvement were identified.

FIG. 11 shows the complete directed evolution strategy for identifying adenine base editor variants with greater activity than the parental ABE. ABE variants with single point mutations that exhibit up to 8-fold greater activity as compared to the parental ABE were selected in bacteria and then evaluated in a first round of human cell screening in human HEK293T cells. Single point mutants identified in selections performed against TAT or CAC sequence contexts were evaluated at eight endogenous genomic sites, whereas single point mutants identified in selections performed against TAG sequence contexts were evaluated at four endogenous genomic sites. Single mutations showing the highest average fold-change were combined to create the top hits: 20 (LPG50148 with L35N, V81S, N156R, and L162W mutations, set forth as SEQ ID NO: 319), 60 (LPG50148 with I75W and F155W mutations, set forth as SEQ ID NO: 359), 67 (LPG50148 with V81S, F155W, and A160D mutations, set forth as SEQ ID NO: 366), and 68 (LPG50148 with V81S, C145A, and F155W mutations, set forth as SEQ ID NO: 367) for TAT; 106 (LPG50148 with D76G and V81S mutations, set forth as SEQ ID NO: 405) and 107 (LPG50148 with V81S and M117L mutations, set forth as SEQ ID NO: 406) for CAC; and 88 (LPG50148 with I40L and V105M mutations set forth as SEQ ID NO: 387) and 103 (LPG50148 with V81S, C145M, and A160W mutations, set forth as SEQ ID NO: 402) for TAG.

FIG. 12 demonstrates higher editing efficiency of evolved ABE variants LPG50148.2.20, LPG50148.2.60, LPG50148.2.67, LPG50148.2.68, and LPG50148.2.69 as compared to the parental ABE when the ABE is delivered as an mRNA.

FIG. 13 provides results of robustness testing of evolved ABE variants LPG50148.2.20, LPG50148.2.60, LPG50148.2.67, LPG50148.2.68, LPG50148.2.103, LPG50148.2.106, and LPG50148.2.107. FIG. 13A shows the percentage of guides (from 48 genomic sites) with total A-T substitution above 20% editing. FIG. 13B shows the average A to G substitution for all genomic sites. There were three significant effects: variant LPG50148.2.20 (p<0.001), LPG50148.2.103 (p<0.001), and LPG50148.2.67 (p=0.01).

FIG. 14 demonstrates a shift in the optimum editing window for three of the evolved ABE variants. The graphs show the parental ABE and three evolved ABE variants LPG50148.2.20, LPG50148.2.67, and LPG50148.2.103 mean A-T substitution by position showing editor activity windows. Numbers indicate the position within the protospacer upstream of the PAM sequence. The optimum window is ≥30% of maximum editing.

FIG. 15 shows A to G editing within different sequence contexts for multiple evolved ABE variants (LPG50148.2.20, LPG50148.2.60, LPG50148.2.67, LPG50148.2.68, LPG50148.2.103, LPG50148.2.106, and LPG50148.2.107) and the parental ABE. The percent editing reflects the median values for each context across forty-eight genomic sites.

FIG. 16 provides the percent A to G conversion at position 11 in the target sequence of SGN008393 using LPG20047 in primary mouse hepatocytes.

FIG. 17 shows the engineering of a third generation deaminase based on the second generation deaminases provided higher levels of A to G editing, when fused to the N-terminus of nAPG07433.1, against multiple targets.

FIG. 18 shows improved performance of a third generation base editor LPG50324 fused to nAPG07433.1.

FIG. 19 shows improved editing utilizing the LPG50310 deaminase relative to the LPG50265 deaminase in HEK293T cells with plasmid delivery when fused to nAPG07433.1.

### DETAILED DESCRIPTION

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

This disclosure provides deaminases and fusion proteins that comprise a nucleic acid molecule-binding polypeptide, such as a DNA-binding polypeptide, and a deaminase polypeptide. In certain embodiments, the DNA-binding polypeptide is a sequence-specific DNA-binding polypeptide, in that the DNA-binding polypeptide binds to a target sequence at a greater frequency than binding to a randomized background sequence. In some embodiments, the DNA-binding polypeptide is or is derived from a meganuclease, zinc finger fusion protein, or TALEN. In some embodiments, the fusion protein comprises an RNA-guided DNA-binding polypeptide and a deaminase polypeptide. In some embodiments, the RNA-guided DNA-binding polypeptide is an RNA-guided nuclease (RGN), such as a CRISPR-Cas (e.g., Cas9) polypeptide that binds to a guide RNA (also referred to as gRNA), which, in turn, binds a target nucleic acid sequence via strand hybridization.

This disclosure also provides fusion proteins (and nucleic acid molecules encoding the same) that comprise an RGN and at least one heterologous polypeptide that is inserted within the RGN. The heterologous polypeptide can be inserted within surface positions of the RGN (e.g., immediately following an amino acid residue that is on the surface of the RGN). The presently disclosed fusion proteins can comprise an RGN with a base-editing polypeptide or a prime editing polypeptide, some of which exhibit improved editing activity and/or shifted editing windows in comparison to an end-to-end fusion protein.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

The term “fusion protein” as used herein refers to a hybrid polypeptide which comprises protein domains from at least two different proteins. A fusion protein may comprise more than one different domain, for example, an RGN and a deaminase. Fusion proteins of the invention comprise a presently disclosed deaminase and a nucleic acid molecule-binding polypeptide or a heterologous protein (e.g., deaminase) that is inserted within the amino acid sequence of an RGN, which is some instances, can disrupt a domain within the RGN protein. In some embodiments, a fusion protein is in a complex with, or is in association with, a nucleic acid, e.g., RNA.

The heterologous polypeptide inserted into an RGN according to the present invention can be a base-editing polypeptide, for example a deaminase polypeptide or active variant or fragment thereof, that directly chemically modifies (e.g., deaminates) a nucleobase, resulting in conversion from one nucleobase to another. The deamination of a nucleobase by a deaminase can lead to a point mutation at the respective residue, which is referred to herein as “nucleic acid editing”, or “base editing”. Fusion proteins comprising an RNA-guided nuclease (RGN) polypeptide and a deaminase can thus be used for the targeted editing of nucleic acid sequences.

The presently disclosed fusion proteins can comprise an RGN fused to a prime editing polypeptide. Prime editing is a versatile and precise genome editing method that directly writes new genetic information into a specified DNA site using a nucleic acid programmable DNA binding protein working in association with a polymerase (described in, e.g., U.S. Pat. No. 11,447,770B1; WO2021072328; WO2021226558; WO2020156575; WO2021042047; U.S. Ser. No. 11/193,123; each incorporated by reference in its entirety herein). The prime editing system uses an RGN that is a nickase, and the system is programmed with a prime editing (PE) guide RNA (“PEgRNA”).

The presently disclosed fusion proteins are useful for targeted editing of DNA in vitro, e.g., for the generation of genetically modified cells. These genetically modified cells may be plant cells or animal cells. Such fusion proteins may also be useful for the introduction of targeted mutations, e.g., for the correction of genetic defects in mammalian cells ex vivo, e.g., in cells obtained from a subject that are subsequently re-introduced into the same or another subject; and for the introduction of targeted mutations, e.g., the correction of genetic defects or the introduction of deactivating mutations in disease-associated genes in a mammalian subject. Such fusion proteins may also be useful for the introduction of targeted mutations in plant cells, e.g., for the introduction of beneficial or agronomically valuable traits or alleles.

Any of the fusion proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

### II. Nucleic Acid Molecule-Binding Polypeptides

Some aspects of this disclosure provide fusion proteins that comprise a nucleic acid molecule-binding polypeptide and a deaminase polypeptide. While binding to and targeted editing of RNA molecules is contemplated by the present invention, in some embodiments, the nucleic acid molecule-binding polypeptide of the fusion protein is a DNA-binding polypeptide. Such fusion proteins are useful for targeted editing of DNA in vitro, ex vivo, or in vivo. These fusion proteins are active in mammalian cells and are useful for targeted editing of DNA molecules.

In some embodiments, the presently disclosed fusion proteins comprise a DNA-binding polypeptide. As used herein, the term “DNA-binding polypeptide” refers to any polypeptide which is capable of binding to DNA. In certain embodiments, the DNA-binding polypeptide portion of the presently disclosed fusion proteins binds to double-stranded DNA. In some embodiments, the DNA-binding polypeptide binds to DNA in a sequence-specific manner. As used herein, the terms “sequence-specific” or “sequence-specific manner” refer to the selective interaction with a specific nucleotide sequence.

Two polynucleotide sequences can be considered to be substantially complementary when the two sequences hybridize to each other under stringent conditions. Likewise, a DNA-binding polypeptide is considered to bind to a particular target sequence in a sequence-specific manner if the DNA-binding polypeptide binds to its sequence under stringent conditions. By “stringent conditions” or “stringent hybridization conditions” is intended conditions under which the two polynucleotide sequences (or the polypeptide binds to its specific target sequence) will bind to each other to a detectably greater degree than to other sequences (e.g., at least 2-fold over background). Stringent conditions are sequence-dependent and will be different in different circumstances. Typically, stringent conditions will be those in which the salt concentration is less than 1.5 M Na ion, typically about 0.01 to 1.0 M Na ion concentration (or other salts) at pH 7.0 to 8.3, and the temperature is at least 30° C. for short sequences (e.g., 10 to 50 nucleotides) and at least 60° C. for long sequences (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The Tm is the temperature (under defined ionic strength and pH) at which 50% of a complementary target sequence hybridizes to a perfectly matched sequence. For DNA-DNA hybrids, the Tm can be approximated from the equation of Meinkoth and Wahl (1984) Anal. Biochem. 138:267-284: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

In certain embodiments, the sequence-specific DNA-binding polypeptide is an RNA-guided, DNA-binding polypeptide (RGDBP). As used herein, the terms “RNA-guided, DNA-binding polypeptide” and “RGDBP” refer to polypeptides capable of binding to DNA through the hybridization of an associated RNA molecule with the target DNA sequence.

In some embodiments, the DNA-binding polypeptide of the fusion protein is a nuclease, such as a sequence-specific nuclease. As used herein, the term “nuclease” refers to an enzyme that catalyzes the cleavage of phosphodiester bonds between nucleotides in a nucleic acid molecule. In some embodiments, the DNA-binding polypeptide is an endonuclease, which is capable of cleaving phosphodiester bonds between nucleotides within a nucleic acid molecule, whereas in certain embodiments, the DNA-binding polypeptide is an exonuclease that is capable of cleaving the nucleotides at either end (5′ or 3′) of a nucleic acid molecule. In some embodiments, the sequence-specific nuclease is selected from the group consisting of a meganuclease, a zinc finger nuclease, a TAL-effector DNA binding domain-nuclease fusion protein (TALEN), and an RNA-guided nuclease (RGN) or variants thereof wherein the nuclease activity has been reduced or inhibited.

As used herein, the term “meganuclease” or “homing endonuclease” refers to endonucleases that bind a recognition site within double-stranded DNA that is 12 to 40 bp in length. Non-limiting examples of meganucleases are those that belong to the LAGLIDADG family that comprise the conserved amino acid motif LAGLIDADG (SEQ ID NO: 700). The term “meganuclease” can refer to a dimeric or single-chain meganuclease.

As used herein, the term “zinc finger nuclease” or “ZFN” refers to a chimeric protein comprising a zinc finger DNA-binding domain and a nuclease domain.

As used herein, the term “TAL-effector DNA binding domain-nuclease fusion protein” or “TALEN” refers to a chimeric protein comprising a TAL effector DNA-binding domain and a nuclease domain.

In certain embodiments, the DNA-binding polypeptide is one which is capable of generating a single-stranded region within a double-stranded DNA molecule. An example of a single-stranded region is the single-stranded loop comprised within an R-loop, which is a three-stranded nucleic acid structure comprising a region of single-stranded DNA that is formed within a double-stranded DNA molecule that results from the hybridization of the complementary strand to a single-stranded RNA or DNA molecule. An adenine within or adjacent to the single-stranded region of the R loop can be deaminated by an adenine deaminase that has activity on single-stranded nucleic acids (e.g., ssDNA). In some of these embodiments, the DNA-binding polypeptide that is capable of generating an R-loop within a double-stranded DNA molecule is an RNA-guided DNA-binding polypeptide or a RGN nuclease. As used herein, the term “RNA-guided nuclease” or “RGN” refers to an RNA-guided, DNA-binding polypeptide that has nuclease activity. RGNs are considered “RNA-guided” because guide RNAs form a complex with the RNA-guided nucleases to direct the RNA-guided nuclease to bind to a target sequence and in some embodiments, introduce a single-stranded or double-stranded break at the target sequence.

Although an RGN can be capable of cleaving the target sequence upon binding, the term RGN also encompasses nuclease-dead RGNs that are capable of binding to, but not cleaving, a target sequence. The terms “cleave” or “cleavage” refer to the hydrolysis of at least one phosphodiester bond within the backbone of one or both strands of a double-stranded target sequence (e.g., target DNA sequence) that can result in either single-stranded or double-stranded breaks within the target DNA sequence. Cleavage of a target sequence by an RGN can result in a single- or double-stranded break. RGNs only capable of cleaving a single strand of a double-stranded target nucleic acid molecule are referred to herein as nickases. Such RGNs have a single functioning nuclease domain. RGN nickases can be naturally occurring nickases or can be RGN proteins that naturally cleave both strands of a double-stranded nucleic acid molecule that have been mutated within one or more nuclease domains such that the nuclease activity of these mutated domains is reduced or eliminated, to become a nickase.

RNA-guided nucleases (RGNs) allow for the targeted manipulation of a single site within a genome and are useful in the context of gene targeting for therapeutic and research applications. In a variety of organisms, including mammals, RNA-guided nucleases have been used for genome engineering by stimulating either non-homologous end joining or homologous recombination. RGNs include CRISPR-Cas proteins, which are RNA-guided nucleases directed to the target sequence by a guide RNA (gRNA) as part of a Clustered Regularly Interspaced Short Palindromic Repeats (CRISPR) RNA-guided nuclease system, or active variants or fragments thereof.

Some aspects of this disclosure provide fusion proteins that comprise an RNA-guided DNA-binding polypeptide and a deaminase polypeptide, such as an adenine deaminase polypeptide. In some embodiments, the RNA-guided DNA-binding polypeptide is an RNA-guided nuclease (RGN). In further embodiments, the RNA-guided nuclease is a naturally occurring CRISPR-Cas protein or an active variant or fragment thereof. CRISPR-Cas systems are classified into Class 1 or Class 2 systems. Class 2 systems comprise a single effector nuclease and include Types II, V, and VI. The Class 1 and 2 systems are subdivided into types (Types I, II, III, IV, V, VI), with some types further divided into subtypes (e.g., Type II-A, Type II-B, Type II-C, Type V-A, Type V-B).

In certain embodiments, the RGN is a naturally occurring Type II CRISPR-Cas protein or an active variant or fragment thereof. As used herein, the term “Type II CRISPR-Cas protein,” “Type II CRISPR-Cas effector protein,” or “Type II RNA-guided nuclease” refers to an RGN that requires a trans-activating RNA (tracrRNA) and comprises two nuclease domains (i.e., RuvC and HNH), each of which is responsible for cleaving a single strand of a double-stranded DNA molecule. In some embodiments, the present invention provides a fusion protein comprising a presently disclosed deaminase fused to a Cas9 protein, such as Streptococcus pyogenes Cas9 (SpCas9), the sequence of which is set forth as SEQ ID NO: 415, or a SpCas9 nickase, which are described in U.S. Pat. Nos. 10,000,772 and 8,697,359, each of which is herein incorporated by reference in its entirety. In some embodiments, the present invention provides a fusion protein comprising a presently disclosed deaminase fused to Streptococcus thermophilus Cas9 (StCas9), the sequence of which is set forth as SEQ ID NO: 701, or a StCas9 nickase, and are disclosed in U.S. Pat. No. 10,113,167, which is herein incorporated by reference in its entirety. In some embodiments, the present invention provides a fusion protein comprising a presently disclosed deaminase fused to Streptococcus aureus Cas9 (SaCas9), which is set forth as SEQ ID NO: 702, or a SaCas9 nickase, which are disclosed in U.S. Pat. No. 9,752,132, which is herein incorporated by reference in its entirety.

In some embodiments, the CRISPR-Cas protein is a naturally-occurring Type V CRISPR-Cas protein or an active variant or fragment thereof. As used herein, the term “Type V CRISPR-Cas protein,” “Type V CRISPR-Cas effector protein,” or “Type V RNA-guided nuclease” refers to an RGN that cleaves dsDNA and comprises a single RuvC nuclease domain or a split-RuvC nuclease domain and lacks an HNH domain (Zetsche et al 2015, Cell doi:10.1016/j.cell.2015.09.038; Shmakov et al 2017, Nat Rev Microbiol doi:10.1038/nrmicro.2016.184; Yan et al 2018, Science doi:10.1126/science.aav7271; Harrington et al 2018, Science doi:10.1126/science.aav4294). In some embodiments, a presently disclosed fusion protein comprises a Cas12 (e.g., Cas12a). It is to be noted that Cas12a is also referred to as Cpf1, and does not require a tracrRNA, although other Type V CRISPR-Cas proteins, such as Cas12b, do require a tracrRNA. Most Type V effectors can also target ssDNA (single-stranded DNA), often without a PAM requirement (Zetsche et al 2015; Yan et al 2018; Harrington et al 2018). The terms “Type V CRISPR-Cas protein” and “Type V RGN” encompasses the unique RGNs comprising split RuvC nuclease domains, such as those disclosed in WO 2021/138247, the contents of each of which are incorporated herein by reference in its entirety. In some embodiments, the present invention provides a fusion protein comprising a presently disclosed deaminase fused to Francisella novicida Cas12a (FnCas12a), the sequence of which is set forth as SEQ ID NO: 703 and is disclosed in U.S. Pat. No. 9,790,490, which is herein incorporated by reference in its entirety, or any of the nuclease-inactivating mutants of FnCas12a disclosed within U.S. Pat. No. 9,790,490.

In some embodiments, the CRISPR-Cas protein is a naturally-occurring Type VI CRISPR-Cas protein or an active variant or fragment thereof. As used herein, the term “Type VI CRISPR-Cas protein,” “Type VI CRISPR-Cas effector protein,” or “Type VI RGN” refers to a CRISPR-Cas effector protein that does not require a tracrRNA and comprises two HEPN domains that cleave RNA. In some embodiments, the present invention provides a fusion protein comprising a presently disclosed deaminase fused to a Cas13.

In some embodiments, the presently disclosed fusion proteins comprise an RGN, or a nickase or nuclease-dead variant thereof is one that was disclosed in International Appl. Publ. Nos. WO 2019/236566, WO 2020/139783, WO 2021/030344, WO 2021/138247, WO 2021/231437, or WO 2021/217002, or International Appl. No. PCT/IB2023/058160 filed Aug. 12, 2023, each of which is incorporated by reference herein in its entirety.

In some embodiments, the presently disclosed fusion proteins comprise an RGN, or a nickase or nuclease-dead variant thereof, listed in Table 1, and/or set forth as SEQ ID NOs: 1-4, 49-162, 435, 575, 576, 698, and 699. The guide RNA sequences (crRNA repeat and tracrRNA sequences) that can be used with each RGN of Table 1 are also provided, as well as the consensus PAM sequence. In certain embodiments, the fusion protein comprises an active variant of an RGN (one able to bind to a nucleic acid molecule in an RNA-guided manner) listed in Table 1 and/or set forth as SEQ ID NOs: 1-4, 49-162, 435, 575, 576, 698, and 699having between 80% and 99% or more sequence identity to any one of the amino acid sequences listed in Table 1 and/or set forth as SEQ ID NOs: 1-4, 49-162, 435, 575, 576, 698, and 699, including but not limited to about or more than about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In some embodiments, the fusion protein comprises an RGN having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an RGN amino acid sequence disclosed in Table 1 and/or set forth as SEQ ID NOs: 1-4, 49-162, 435, 575, 576, 698, and 699. In other embodiments, the fusion protein comprises a fragment of an RGN listed in Table 1 such as one that differs by as few as 1-15 amino acid residues, as few as 1-10, such as 6-10, as few as 5, as few as 4, as few as 3, as few as 2, or as few as 1 amino acid residue. In specific embodiments, the RGN comprises an N-terminal or a C-terminal truncation, which can comprise at least a deletion of 1, 2, 3, 4, 5, 10, 15, 20, 25, 30, 35, 40, 45, 50, 55, 60 amino acids or more from either the N or C terminus of the polypeptide. In some embodiments, the RGN comprises an internal deletion which can comprise at least a deletion of 1, 2, 3, 4, 5, 6, 7, 8, 9, 10, 11, 12, 13, 14, 15, 16, 17, 18, 19, 20, 25, 30, 35, 40, 45, 50, 55, 60 amino acids or more.

TABLE 1
Non-limiting examples of RNA-guided nucleases

| | | crRNA | | |
|---|---|---|---|---|
| | RGN SEQ | repeat | | |
| RGN Name | ID NO: | sequence | tracrRNA | PAM |
| APG05083.1 | 60 | 432 | 433 | 434 |
| APG07433.1 | 1 | 416 | 417 | 418 |
| APG07513.1 | 435 | 436 | 437 | 438 |
| APG08290.1 | 57 | 439 | 440 | 441 |
| APG05459.1 | 58 | 442 | 443 | 444 |
| APG04583.1 | 61 | 445 | 446 | 447 |
| APG01688.1 | 62 | 448 | 449 | 450 |
| APG00969 | 63 | 451 | 452 | 453 |
| APG03128 | 64 | 454 | 455 | 456 |
| APG09748 | 140 | 457 | 458 | 459 |
| APG00771 | 65 | 460 | 461 | 462 |
| APG02789 | 141 | 463 | 464 | 465 |
| APG09106 | 142 | 466 | 467 | 468 |
| APG05733.1 | 66 | 469 | 470 | 471 |
| APG06207.1 | 67 | 472 | 473 | 474 |
| APG01647.1 | 68 | 475 | 476 | 477 |
| APG08032.1 | 69 | 478 | 479 | 480 |
| APG05712.1 | 70 | 481 | 482 | 483 |
| APG01658.1 | 71 | 484 | 485 | 486 |
| APG06498.1 | 72 | 487 | 488 | 489 |
| APG09106.1 | 142 | 466 | 467 | 468 |
| APG09882.1 | 73 | 493 | 494 | 495 |
| APG02675.1 | 74 | 496 | 497 | 498 |
| APG01405.1 | 75 | 499 | 500 | 501 |
| APG06250.1 | 76 | 502 | 503 | 504 |
| APG06877.1 | 77 | 505 | 506 | 507 |
| APG09053.1 | 78 | 508 | 509 | 510 |
| APG04293.1 | 79 | 511 | 512 | 513 |
| APG01308.1 | 80 | 514 | 515 | 516 |
| APG06646.1 | 81 | 517 | 518 | 519 |
| APG09624 | 144 | 520 | 521 | ND |
| APG05405 | 146 | 522 | 523 | ND |
| APG06622 | 82 | 524 | 525 | 526 |
| APG02787 | 83 | 527 | 528 | 529 |
| APG06248 | 84 | 530 | 531 | 532 |
| APG06007 | 85 | 533 | 534 | 535 |
| APG02874 | 86 | 536 | 537 | 538 |
| APG03850 | 87 | 539 | 540 | 541 |
| APG07553 | 88 | 542 | 543 | 544 |
| APG03031 | 89 | 545 | 546 | 547 |
| APG09208 | 90 | 548 | 549 | 550 |
| APG05586 | 2 | 419 | 420 | 421 |
| APG08770 | 91 | 551 | 552 | 553 |
| APG08167 | 92 | 554 | 555 | 556 |
| APG01604 | 3 | 422 | 423 | 424 |
| APG03021 | 93 | 557 | 558 | 559 |
| APG06015 | 94 | 560 | 561 | 562 |
| APG09344 | 95 | 563 | 564 | 565 |
| APG07991 | 96 | 566 | 567 | 568 |
| APG01868 | 97 | 569 | 570 | 571 |
| APG02998 | 98 | 572 | 573 | 574 |
| APG09298 | 99 | 419 | 420 | 421 |
| APG06251 | 100 | 419 | 420 | 421 |
| APG03066 | 101 | 419 | 420 | 421 |
| APG01560 | 102 | 419 | 420 | 421 |
| APG02777 | 103 | 419 | 420 | 421 |
| APG05761 | 104 | 419 | 420 | 421 |
| APG02479 | 105 | 419 | 420 | 421 |
| APG08385 | 106 | 419 | 420 | 421 |
| APG09217 | 107 | 419 | 420 | 421 |
| APG06657 | 108 | 419 | 420 | 421 |
| APG05586 | 171 | 419 | 420 | 421 |
| APG07433.1 | 575 | 416 | 417 | 418 |
| deletion | | | | |
| variant | | | | |
| APG08290.1 | 576 | 439 | 440 | 441 |
| deletion | | | | |
| variant | | | | |
| LPG10134 | 109 | 163 | 175 | NGG |
| LPG10136 | 110 | 164 | 176 | NNNNCC |
| LPG10138 | 111 | 165 | 177 | NNNNC |
| LPG10139 | 112 | 166 | 178 | NNNNC |
| LPG10141 | 113 | 167 | 179 | NGGNR |
| LPG10145 | 4 | 168 | 180 | NNGG |
| LPG10146 | 114 | 169 | 181 | NNRNNAY |
| LPG10147 | 115 | 170 | 182 | NNAAAT |
| LPG10150 | 116 | 171 | 183 | NNRRWC |
| LPG10155 | 117 | 172 | 184 | NNAAAT |
| LPG10159 | 118 | 173 | 185 | NRAAR |
| LPG10160 | 119 | 174 | 186 | NAAT |
| LPG10165 | 120 | 187 | 208 | nnnnGRRA |
| LPG10166 | 121 | 188 | 209 | nnnAAC, |
| | | | | nnnAAA, |
| | | | | nnnCAC, |
| | | | | nnnnCAC |
| LPG10167 | 122 | 189 | 210 | nnnnCC |
| LPG10168 | 123 | 190 | 211 | nnnnCC |
| LPG10169 | 124 | 191 | 212 | nnGAA |
| LPG10171 | 125 | 192 | 213 | nnnnGnnA |
| LPG10186 | 126 | 193 | 214 | nVG |
| LPG10190 | 127 | 194 | 215 | nnnnCCD, |
| | | | | nnnnCTG |
| LPG10191 | 128 | 195 | 216 | nnAHAAY |
| LPG10194 | 129 | 196 | 217 | VGGNR |
| LPG10195 | 130 | 197 | 218 | NNRA |
| LPG10196 | 131 | 198 | 219 | NNNYTT |
| LPG10197 | 132 | 199 | 220 | nnAAA |
| LPG10198 | 133 | 200 | 221 | nnnnCMAA |
| LPG10200 | 134 | 201 | 222 | nGR |
| LPG10203 | 135 | 202 | 223 | nnnnGGG |
| LPG10204 | 136 | 203 | 224 | nnnnGTG, |
| | | | | nnnnGCGA |
| LPG10205 | 137 | 204 or 205 | 225 or 226 | nnnnGTG, |
| | | | | nnnnGCGA |
| LPG10207 | 138 | 206 | 227 | nnnnCC |
| LPG10208 | 139 | 207 | 228 | nnnnGTG, |
| | | | | nnnnGCGA |

* ND: not determined

The RGN component of the presently disclosed fusion proteins can be APG07433.1 (disclosed in International Patent Publication No. WO 2019/236566, which is incorporated herein by reference in its entirety; and set forth herein as SEQ ID NO: 1), APG05586 (disclosed in International Patent Publication No. WO 2021/217002, which is incorporated herein by reference in its entirety; and set forth herein as SEQ ID NO: 2), APGO1604 (disclosed in International Patent Publication No. WO 2021/217002, which is incorporated herein by reference in its entirety; and set forth herein as SEQ ID NO: 3), LPG10145 (disclosed in International Patent Publication No. WO 2023/139557, which is incorporated herein by reference in its entirety; and set forth herein as SEQ ID NO: 4), LPG10196 (disclosed in International Appl. No. PCT/I1B2023/058160 filed Aug. 12, 2023, which is incorporated herein by reference in its entirety; and set forth herein as SEQ ID NO: 131); or an active fragment or variant thereof (i.e., one able to bind to a nucleic acid molecule in an RNA-guided manner), such as a nickase variant thereof.

The RGN can have between 80% and 99% or more sequence identity to any one of SEQ ID NOs: 1, 2, 3, 4, and 131 (that retains RGN activity), including but not limited to at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more. In certain embodiments, the fusion protein comprises an RGN having 80%, 81%, 82%, 83%, 84%, 85%, 86%, 87%, 88%, 89%, 90%, 91%, 92%, 93%, 94%, 95%, 96%, 97%, 98%, 99%, or more sequence identity to any one of SEQ ID NOs: 1, 2, 3, 4, and 131 (that retains RGN activity). The fusion protein can comprise an active fragment of an RGN set forth as any one of SEQ ID NOs: 1, 2, 3, 4, and 131, such as one that differs from any one of SEQ ID NOs: 1, 2, 3, 4, and 131 by as few as 1-15 amino acid residues, as few as 1-10, such as 6-10, as few as 5, as few as 4, as few as 3, as few as 2, or as few as 1 amino acid residue. In certain embodiments, the active RGN fragment comprises an N-terminal or a C-terminal truncation, which can comprise at least a deletion of 1, 2, 3, 4, 5, 10, 15, 20, 25, 30, 35, 40, 45, 50, 55, 60 amino acids or more from either the N or C terminus of the polypeptide set forth in any one of SEQ ID NOs: 1, 2, 3, 4, and 131. In some embodiments, the active RGN variant or fragment comprises an internal deletion which can comprise at least a deletion of 1, 2, 3, 4, 5, 6, 7, 8, 9, 10, 11, 12, 13, 14, 15, 16, 17, 18, 19, 20, 25, 30, 35, 40, 45, 50, 55, 60 amino acids or more of any one of SEQ ID NOs: 1, 2, 3, 4, and 131. An active fragment of an RGN can comprise at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth in any one of SEQ ID NOs: 1, 2, 3, 4, and 131.

In some embodiments, the RGN of the fusion protein is an RGN nickase which comprises a mutation (e.g., a D10A mutation, wherein amino acid numbering is based on the Streptococcus pyogenes Cas9 sequence set forth as SEQ ID NO: 415) which renders the RGN capable of cleaving only the non-base edited, target strand (the strand which comprises the PAM and is base paired to a gRNA) of a nucleic acid duplex. A nickase comprising a D10A mutation, or an equivalent mutation, has an inactivated RuvC nuclease domain and cleaves the targeted strand. D10A nickases are not able to cleave the non-targeted strand of the DNA, i.e., the strand where base editing is desired. In these embodiments, the RGN nicks the target strand, while the complementary, non-target strand is modified by the deaminase. Cellular DNA-repair machinery may repair the nicked, target strand using the modified non-target strand as a template, thereby introducing a mutation in the DNA.

Thus, in some embodiments, the nickase comprises an inactive RuvC domain. RuvC domains have an RNase H fold structure (see, e.g., Nishimasu et al. (2014) Cell 156(5):935-949, which is incorporated by reference in its entirety). RuvC domains of RGNs are often split RuvC domains, comprising two or more non-adjacent regions within the linear amino acid sequence. For example, the RuvC domain of Streptococcus pyogenes Cas9 comprises amino acid residues 1-59, 718-769 and 909-1098 of SEQ ID NO: 415. A non-limiting example of a mutation within a RuvC domain that inactivates its nuclease activity is the D10A mutation that mutates the first aspartic acid residue in the split RuvC nuclease domain. nAPG07433.1 (set forth as SEQ ID NO: 49) is a nickase variant (with an inactivated RuvC domain) of APG07433.1, which is set forth as SEQ ID NO: 1, and is described in WO 2019/236566 (incorporated by reference in its entirety herein). nAPG05586 (set forth as SEQ ID NO: 50) is a nickase variant (with an inactivated RuvC domain) of APG05586 (set forth as SEQ ID NO: 2). nAPG01604 (set forth as SEQ ID NO: 51) is a nickase variant (with an inactivated RuvC domain) of APG01604 (set forth as SEQ ID NO: 3). nLPG10145 (set forth as SEQ ID NO: 52) is a nickase variant (with an inactivated RuvC domain) of LPG10145 (set forth as SEQ ID NO: 4). nLPG10196 (set forth as SEQ ID NO: 698) is a nickase variant (with an inactivated RuvC domain) of LPG10196 (set forth as SEQ ID NO: 131).

In some embodiments, the RGN of the fusion protein is an RGN nickase comprising a mutation (e.g., a H840A mutation, wherein amino acid numbering is based on the Streptococcus pyogenes Cas9 sequence set forth as SEQ ID NO: 415), which renders the RGN capable of cleaving only the non-target strand (the strand which does not comprise the PAM and is not base paired to a gRNA) of a nucleic acid duplex. In some of these embodiments, the nickase comprises an inactive HNH nuclease domain. The HNH nuclease domain of RGNs have a $$a-metal fold (see, e.g., Nishimasu et al. 2014). The HNH nuclease domain of the Streptococcus pyogenes Cas9, for example, comprises amino acid residues 775-908 of SEQ ID NO: 415. A non-limiting example of a mutation within a HNH domain that inactivates its nuclease activity is the H840A mutation that mutates the first histidine of the HNH nuclease domain. The RGN with an inactivated HNH domain acts on the non-target strand. nAPG07433.1 (set forth as SEQ ID NO: 56) is a nickase variant (with an inactivated HNH domain) of APG07433.1, which is set forth as SEQ ID NO: 1, and is described in WO 2019/236566 (incorporated by reference in its entirety herein). nAPG05586 (set forth as SEQ ID NO: 53) is a nickase variant (with an inactivated HNH domain) of APG05586 (set forth as SEQ ID NO: 2). nAPG01604 (set forth as SEQ ID NO: 54) is a nickase variant (with an inactivated HNH domain) of APG01604 (set forth as SEQ ID NO: 3). nLPG10145 (set forth as SEQ ID NO: 55) is a nickase variant (with an inactivated HNH domain) of LPG10145 (set forth as SEQ ID NO: 4). nLPG10196 (set forth as SEQ ID NO: 699) is a nickase variant (with an inactivated HNH domain) of LPG10196 (set forth as SEQ ID NO: 131).

Methods for inactivating a RuvC and/or HNH domain of a RGN are known in the art and generally comprise mutating the first aspartic acid within a split RuvC domain and/or the first histidine of the HNH domain. Typically, the aspartic acid residue or histidine residue is mutated to an alanine. Other amino acid residues within the RuvC domain that can be mutated to inactivate nuclease activity of the domain include Glu762, His983, and Asp986 (typically to an alanine), wherein amino acid numbering is based on the Streptococcus pyogenes Cas9 sequence set forth as SEQ ID NO: 415. Other amino acid residues within the HNH domain that can be mutated include D839 and N863 (typically to an alanine), wherein amino acid numbering is based on the Streptococcus pyogenes Cas9 sequence set forth as SEQ ID NO: 415.

Unless otherwise noted, when using the nomenclature “n” followed by the name of a nuclease, this refers to a nickase in which the RuvC domain has been inactivated, (e.g., with a D10A mutation).

In some embodiments, the fusion protein comprises an RGN nickase retaining nickase activity comprises an amino acid sequence having about 60% to about 99.5% identity to any one of SEQ ID NOs: 49-56, 698, and 699. In some embodiments the RGN nickase retaining nickase activity comprises an amino acid sequence that has at least 60%, at least 65%, at least 70%, at least 75%, at least 80%, at least 85%, at least 90%, at least 95%, at least 96%, at least 97%, at least 98%, at least 99%, or at least 99.5% identity to any one of SEQ ID NOs: 49-56, 698, and 699.

In some embodiments, the fusion protein comprises an RGN nickase comprising an amino acid sequence having between 80% and 99% or more sequence identity to any one of SEQ ID NOs: 49-56, 698, and 699, including but not limited to at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more.

In some embodiments, the RGN of the fusion protein is nuclease dead. As used herein, an RGN protein that has been mutated to become nuclease-inactive or “dead” can be referred to as an RNA-guided, DNA-binding polypeptide or a nuclease-inactive RGN or nuclease-dead RGN. Methods for generating a nuclease inactive RGN are known in the art and generally comprise mutating the sole nuclease domain or all of the nuclease domains of an RGN to render the nuclease domain(s) inactive. In those embodiments where the RGN only comprises a single nuclease domain (e.g., RuvC domain), the nuclease inactive variant will have at least one mutation within the RuvC domain that results in inactivation of the RuvC nuclease domain. In those embodiments wherein the RGN comprises more than one nuclease domain, such as a RuvC and an HNH domain, at least one mutation within each of the RuvC and the HNH domain renders both nuclease domains inactive.

One exemplary suitable nuclease inactive RGN is the D10A/H840A Cas9 mutant (see, e.g., Qi et al., Cell. 2013; 152(5): 1173-83, the entire contents of which are incorporated herein by reference).

Additionally, suitable nuclease-inactive variants of other known RNA guided nucleases (RGNs) can be determined.

Other additional exemplary suitable nuclease inactive RGN variants include, but are not limited to, D10A/D839A/H840A, and D10A/D839A/H840A/N863A mutant domains (See, e.g., Mali et al., Nature Biotechnology. 2013; 31(9): 833-838, the entire contents of which are incorporated herein by reference).

Additional suitable RGN proteins mutated to be nickases or inactive nucleases will be apparent to those of skill in the art based on this disclosure and knowledge in the field (such as for example the RGNs disclosed in PCT Publication No. WO 2019/236566, which is herein incorporated by reference in its entirety) and are within the scope of this disclosure.

Any method known in the art for introducing mutations into an amino acid sequence, such as PCR-mediated mutagenesis and site-directed mutagenesis, can be used for generating nickases or nuclease-dead RGNs. See, e.g., U.S. Publ. No. 2014/0068797 and U.S. Pat. No. 9,790,490; each of which is incorporated herein by reference in its entirety.

The fusion proteins of the invention comprising an RGN utilize guide RNAs that bind to the RGN component of the fusion protein and guide the fusion protein to a target sequence. The term “guide RNA” refers to a nucleotide sequence having sufficient complementarity with a target nucleotide sequence to hybridize with the target sequence and direct sequence-specific binding of an associated RGN to the target nucleotide sequence. More specifically, when the target nucleotide sequence is double-stranded as is the case with DNA, the target nucleotide sequence is comprised of a target strand (which comprises the PAM sequence) and the non-target strand. In these embodiments, the guide RNA has sufficient complementarity with the non-target strand of a double-stranded target sequence (e.g., target DNA sequence) such that the guide RNA hybridizes with the non-target strand and directs sequence-specific binding of an associated RNA-guided nuclease (RGN) to the target sequence (e.g., target DNA sequence). Therefore, in some embodiments, a guide RNA includes a spacer that is identical to the sequence of the target strand except that uracil (U) replaces thymidine (T) in the guide RNA.

The guide RNA is one or more RNA molecules (generally, one or two), that can bind to the RGN and guide the RGN to bind to a particular target nucleotide sequence, and in those instances wherein the RGN has nickase or nuclease activity, also cleave the target nucleotide sequence. A guide RNA comprises a CRISPR RNA (crRNA) and in some embodiments, a trans-activating CRISPR RNA (tracrRNA). In some embodiments, a portion of the guideRNA comprises DNA nucleotides. In certain embodiments, the guideRNA comprises artificial, non-naturally-occurring nucleotide analogs or one or more nucleotides are chemically modified, including the modifications described in International Appl. No. PCT/IB2023/058418, filed Aug. 25, 2023, which is herein incorporated by reference in its entirety.

A CRISPR RNA comprises a spacer sequence and a CRISPR repeat sequence. The “spacer sequence” is the nucleotide sequence that directly hybridizes with the non-target strand of a target sequence of interest. The spacer sequence is engineered to be fully or partially complementary with the non-target strand of a target sequence of interest. In various embodiments, the spacer sequence comprises from about 8 nucleotides to about 30 nucleotides, or more. For example, the spacer sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the spacer sequence is 8, 9, 10, 11, 12, 13, 14, 15, 16, 17, 18, 19, 20, 21, 22, 23, 24, 25, 26, 27, 28, 29, 30, or more nucleotides in length. In some embodiments, the spacer sequence is about 10 to about 26 nucleotides in length, or about 12 to about 30 nucleotides in length. In some embodiments, the spacer sequence is about 30 nucleotides in length. In some embodiments, the spacer sequence is 30 nucleotides in length. In some embodiments, the degree of complementarity between a spacer sequence and its corresponding target sequence, when optimally aligned using a suitable alignment algorithm, is between 50% and 99% or more, including but not limited to about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In some embodiments, the degree of complementarity between a spacer sequence and its corresponding target sequence, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more. In some embodiments, the spacer sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24).

The CRISPR RNA repeat sequence comprises a nucleotide sequence that forms a structure, either on its own or in concert with a hybridized tracrRNA, that is recognized by the RGN molecule. In various embodiments, the CRISPR RNA repeat sequence comprises from about 8 nucleotides to about 30 nucleotides, or more. In some embodiments, the CRISPR RNA repeat sequence comprises from 8 nucleotides to 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the CRISPR repeat sequence is 8, 9, 10, 11, 12, 13, 14, 15, 16, 17, 18, 19, 20, 21, 22, 23, 24, 25, 26, 27, 28, 29, 30, or more nucleotides in length. In some embodiments, the degree of complementarity between a CRISPR repeat sequence and its corresponding tracrRNA sequence, when optimally aligned using a suitable alignment algorithm, is between 50% and 99%, or more, including but not limited to about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In some embodiments, the degree of complementarity between a CRISPR repeat sequence and its corresponding tracrRNA sequence, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, the guide RNA further comprises a tracrRNA molecule. A trans-activating CRISPR RNA or tracrRNA molecule comprises a nucleotide sequence comprising a region that has sufficient complementarity to hybridize to a CRISPR repeat sequence of a crRNA, which is referred to herein as the anti-repeat region. In some embodiments, the tracrRNA molecule further comprises a region with secondary structure (e.g., stem-loop) or forms secondary structure upon hybridizing with its corresponding crRNA. In some embodiments, the region of the tracrRNA that is fully or partially complementary to a CRISPR repeat sequence is at the 5′ end of the molecule and the 3′ end of the tracrRNA comprises secondary structure. This region of secondary structure generally comprises several hairpin structures, including the nexus hairpin, which is found adjacent to the anti-repeat sequence. There are often terminal hairpins at the 3′ end of the tracrRNA that can vary in structure and number, but often comprise a GC-rich Rho-independent transcriptional terminator hairpin followed by a string of Us at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In various embodiments, the anti-repeat region of the tracrRNA that is fully or partially complementary to the CRISPR repeat sequence comprises from about 6 nucleotides to about 30 nucleotides, or more. For example, the region of base pairing between the tracrRNA anti-repeat sequence and the CRISPR repeat sequence can be about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the region of base pairing between the tracrRNA anti-repeat sequence and the CRISPR repeat sequence is 6, 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the anti-repeat region of the tracrRNA that is fully or partially complementary to a CRISPR repeat sequence is about 10 nucleotides in length. In some embodiments, the anti-repeat region of the tracrRNA that is fully or partially complementary to a CRISPR repeat sequence is nucleotides in length. In some embodiments, the degree of complementarity between a CRISPR repeat sequence and its corresponding tracrRNA anti-repeat sequence, when optimally aligned using a suitable alignment algorithm, is between 50% and 99% or more, including but not limited to about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In some embodiments, the degree of complementarity between a CRISPR repeat sequence and its corresponding tracrRNA anti-repeat sequence, when optimally aligned using a suitable alignment algorithm, is at least 50%, at least 60%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more.

In various embodiments, the entire tracrRNA comprises from about 60 nucleotides to more than about 210 nucleotides. In some embodiments, the entire tracrRNA comprises from 60 nucleotides to more than 210 nucleotides. For example, the tracrRNA can be about 60, about 65, about 70, about 75, about 80, about 85, about 90, about 95, about 100, about 105, about 110, about 115, about 120, about 125, about 130, about 135, about 140, about 150, about 160, about 170, about 180, about 190, about 200, about 210 or more nucleotides in length. In some embodiments, the tracrRNA is 60, 65, 70, 75, 80, 85, 90, 95, 100, 105, 110, 115, 120, 125, 130, 135, 140, 150, 160, 170, 180, 190, 200, 210 or more nucleotides in length. In some embodiments, the tracrRNA is about 100 to about 200 nucleotides in length, including about 95, about 96, about 97, about 98, about 99, about 100, about 105, about 106, about 107, about 108, about 109, and about 100 nucleotides in length. In some embodiments, the tracrRNA is 100 to 110 nucleotides in length, including 95, 96, 97, 98, 99, 100, 105, 106, 107, 108, 109, and 110 nucleotides in length.

Guide RNAs form a complex with an RNA-guided, DNA-binding polypeptide or an RNA-guided nuclease, or a fusion protein comprising the same, to direct the RNA-guided nuclease, or fusion protein comprising the same, to bind to a target sequence. If the guide RNA complexes with an RGN, the bound RGN introduces a single-stranded or double-stranded break at the target sequence. After the target sequence has been cleaved, the break can be repaired such that the DNA sequence of the target sequence is modified during the repair process. Provided herein are methods for using mutant variants of RNA-guided nucleases, which are either nuclease inactive or nickases, which are linked to base-editing polypepitdes (e.g., deaminases) or primed editing polypeptides to modify a target sequence in the DNA of host cells. The mutant variants of RNA-guided nucleases in which the nuclease activity is inactivated or significantly reduced may be referred to as RNA-guided, DNA-binding polypeptides (RGDBP), as the polypeptides are capable of binding to, but not necessarily cleaving, a target sequence. RNA-guided nucleases only capable of cleaving a single strand of a double-stranded nucleic acid molecule are referred to herein as nickases.

A target nucleotide sequence is bound by an RNA-guided, DNA-binding polypeptide (e.g., RGN) and hybridizes with the guide RNA associated with the RGDBP (e.g., RGN). The target sequence can then be subsequently cleaved if the RGDBP possesses nuclease activity (i.e., is an RGN), which encompasses activity as a nickase.

The guide RNA can be a single guide RNA or a dual-guide RNA system. A single guide RNA comprises the crRNA and optionally tracrRNA on a single molecule of RNA, whereas a dual-guide RNA system comprises a crRNA and a tracrRNA present on two distinct RNA molecules, hybridized to one another through at least a portion of the CRISPR repeat sequence of the crRNA and at least a portion of the tracrRNA, which may be fully or partially complementary to the CRISPR repeat sequence of the crRNA. In some of those embodiments wherein the guide RNA is a single guide RNA, the crRNA and optionally tracrRNA are separated by a linker nucleotide sequence.

Suitable crRNA repeat, tracrRNA, and guide RNA sequences for the RGN component of the presently disclosed fusion proteins are disclosed in International Patent Publication Nos. WO 2019/236566, WO 2021/030344, WO 2020/139783, WO 2021/217002, WO 2021/138247, WO 2021/231437, WO 2023/139557, and PCT International Appl. No. PCT/IB2023/058160 filed Aug. 12, 2023, each of which is incorporated by reference in its entirety, and/or are provided herein in Table 1.

In general, the linker nucleotide sequence between a crRNA and a tracrRNA is one that does not include complementary bases in order to avoid the formation of secondary structure within or comprising nucleotides of the linker nucleotide sequence. In some embodiments, the linker nucleotide sequence between the crRNA and tracrRNA is at least 3, at least 4, at least 5, at least 6, at least 7, at least 8, at least 9, at least 10, at least 11, at least 12, or more nucleotides in length. In some embodiments, the linker nucleotide sequence between the crRNA and tracrRNA is 3, 4, 5, 6, 7, 8, 9, 10, 11, 12, or more nucleotides in length. In some embodiments, the linker nucleotide sequence of a single guide RNA is at least 4 nucleotides in length. In some embodiments, the linker nucleotide sequence of a single guide RNA is 4 nucleotides in length.

In certain embodiments, the guide RNA can be introduced into a target cell, organelle, or embryo as an RNA molecule. The guide RNA can be transcribed in vitro or chemically synthesized. In some embodiments, a nucleotide sequence encoding the guide RNA is introduced into the cell, organelle, or embryo. In some embodiments, the nucleotide sequence encoding the guide RNA is operably linked to a promoter (e.g., an RNA polymerase III promoter). The promoter can be a native promoter or heterologous to the guide RNA-encoding nucleotide sequence. In some embodiments, the promoter is selected from any one of the promoters disclosed in International Appl. No. PCT/US2022/032940, filed Jun. 10, 2022, which is herein incorporated by reference in its entirety.

In various embodiments, the guide RNA can be introduced into a target cell, organelle, or embryo as a ribonucleoprotein complex, as described herein, wherein the guide RNA is bound to an RNA-guided nuclease polypeptide.

The guide RNA directs an associated RGDBP (e.g., RGN) to a particular target nucleotide sequence of interest through hybridization of the guide RNA to the target nucleotide sequence. A target nucleotide sequence can comprise DNA, RNA, or a combination of both and can be single-stranded or double-stranded. A target nucleotide sequence can be genomic DNA (i.e., chromosomal DNA), plasmid DNA, or an RNA molecule (e.g., messenger RNA, ribosomal RNA, transfer RNA, micro RNA, small interfering RNA). The target nucleotide sequence can be bound (and in some embodiments, cleaved) by an RNA-guided, DNA-binding polypeptide in vitro or in a cell. The chromosomal sequence targeted by the RGDBP (e.g., RGN) can be a nuclear, plastid or mitochondrial chromosomal sequence. In some embodiments, the target nucleotide sequence is unique in the target genome.

In some embodiments, the target nucleotide sequence is adjacent to a protospacer adjacent motif (PAM). A PAM is generally within about 1 to about 10 nucleotides from the target nucleotide sequence, including about 1, about 2, about 3, about 4, about 5, about 6, about 7, about 8, about 9, or about 10 nucleotides from the target nucleotide sequence. In some embodiments, a PAM is within 1 to 10 nucleotides from the target nucleotide sequence, including 1, 2, 3, 4, 5, 6, 7, 8, 9, or 10 nucleotides from the target nucleotide sequence. Unless otherwise stated, the PAM is immediately adjacent to the target nucleotide sequence, either at its 5′ or 3′ end. In some embodiments, the PAM is 3′ of the target sequence. Generally, the PAM is a consensus sequence of about 2-6 nucleotides, but in some embodiments, is 1, 2, 3, 4, 5, 6, 7, 8, 9, or more nucleotides in length.

Suitable PAM sequences for the RGN component of the presently disclosed fusion proteins are disclosed in International Patent Publication Nos. WO 2019/236566, WO 2021/030344, WO 2020/139783, WO 2021/217002, WO 2021/138247, WO 2021/231437, WO 2023/139557, and PCT International Appl. No. PCT/IB2023/058160 filed Aug. 12, 2023, each of which is incorporated by reference in its entirety, and/or are provided herein in Table 1.

The PAM restricts which sequences a given RGDBP (e.g., RGN) can target, as its PAM needs to be proximal to the target nucleotide sequence. Upon recognizing its corresponding PAM sequence, the RGN can cleave the target nucleotide sequence at a specific cleavage site. As used herein, a cleavage site is made up of the two particular nucleotides within a target nucleotide sequence between which the nucleotide sequence is cleaved by an RGN. The cleavage site can comprise the 1^{st} and 2^{nd}, 2^{nd} and 3^{rd}, 3^{rd} and 4^{th}, 4^{th} and 5^{th}, 5^{th} and 6 ^{th}, 7^{th} and 8^{th}, or 8^{th} and 9^{th} nucleotides from the PAM in either the 5′ or 3′ direction. As RGNs can cleave a target nucleotide sequence resulting in staggered ends, in some embodiments, the cleavage site is defined based on the distance of the two nucleotides from the PAM on the positive (+) strand of the polynucleotide and the distance of the two nucleotides from the PAM on the negative (−) strand of the polynucleotide.

RGDBPs and RGNs can be used to deliver a fused polypeptide, polynucleotide, or small molecule payload to a particular genomic location.

In those embodiments wherein a fusion protein comprises a meganuclease as the DNA-binding component, a target sequence can comprise a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. In those embodiments wherein the DNA-binding polypeptide comprises a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). In those embodiments wherein the DNA-binding polypeptide component of a fusion protein comprises a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

### III. Deaminases

Some aspects of the invention provide adenine deaminases which were produced through the directed evolution and optimization of a previously disclosed adenine deaminase, LPG50148 (set forth herein as SEQ ID NO: 5 without its starting methionine), which was disclosed in International Appl. No.

WO 2022/056254, which is herein incorporated by reference in its entirety. These evolved adenine deaminases are set forth as SEQ ID NOs: 6, 300-414, 596-598, and 720-723 (again, without the starting methionine).

The term “deaminase” refers to an enzyme that catalyzes a deamination reaction. The deaminases of the invention are nucleobase deaminases and the terms “deaminase” and “nucleobase deaminase” are used interchangeably herein. The deaminase may be a naturally-occurring deaminase enzyme or an active fragment or variant thereof. A deaminase may be active on single-stranded nucleic acids, such as ssDNA or ssRNA, or on double-stranded nucleic acids, such as dsDNA or dsRNA. In some embodiments, the deaminase is only capable of deaminating ssDNA and does not act on dsDNA.

The deaminases of the invention may be used for the editing of DNA or RNA molecules and are useful as deaminases alone or as components of fusion proteins. In some embodiments, the deaminases may be used for editing of ssDNA or ssRNA molecules. Deamination of adenine, adenosine, or deoxyadenosine yields inosine, which is treated as guanine by polymerases.

To date there are no known naturally occurring adenine deaminases that deaminate adenine in DNA. Several methods have been employed to evolve and optimize adenine deaminase acting on tRNA (ADAT) proteins to be active on DNA molecules in mammalian cells (Gaudelli et al, 2017; Koblan, L. W. et al, 2018, Nat Biotechnol 36, 843-846; Richter, M. F. et al, 2020, Nat Biotechnol, doi:10.1038/s41587-020-0562-8, each of which are incorporated by reference in their entirety herein).

In some embodiments, the presently disclosed adenine deaminases are used in combination with a cytosine deaminase that catalyzes the hydrolytic deamination of a cytosine, cytidine, or deoxycytidine to uracil, such as those disclosed in International Appl. Publ. Nos. WO 2020/189783 and WO 2022/204093 and U.S. Appl. Publ. No. 2022/0145296, each of which is herein incorporated by reference in its entirety.

The presently disclosed deaminases or active variants or fragments thereof may be introduced into the cell as part of a deaminase-DNA-binding polypeptide fusion, and/or may be co-expressed with a DNA-binding polypeptide-deaminase fusion, to increase the efficiency of introducing the desired A>N (wherein N is C, T, or G) mutation, such as an A>G mutation, in a target DNA molecule.

The presently disclosed deaminases comprise an amino acid sequence having about 50% to about 100% identity to any of SEQ ID NOs: 5, 6, 300-414, 596-598, and 720-723. In some embodiments, a deaminase has about 50% to about 100% identity to any of SEQ ID NOs: 5, 6, 300-414, 596-598, and 720-723 and has at least one of the amino acid residues set forth in any one of Tables 2, 4, 6, and 19. In some embodiments, the deaminase has an amino acid sequence having at least 50%, at least 55%, at least 60%, at least 65%, at least 70%, at least 75%, at least 80%, at least 85%, at least 90%, at least 95%, at least 96%, at least 97%, at least 98%, at least 99%, or 100% identity to any of SEQ ID NOs: 5, 6, 300-414, 596-598, and 720-723 and has at least one of the amino acid residues set forth in any one of Tables 2, 4, 6, and 19. In some embodiments, the deaminases comprise an amino acid sequence selected from SEQ ID NOs: 303, 319, 366, 375, 406, 596, 597, and 720. Non-limiting examples of such fusion proteins are described in the Examples section herein.

The deaminase can have at least 50%, at least 55%, at least 60%, at least 65%, at least 70%, at least 75%, at least 80%, at least 85%, at least 90%, at least 95%, at least 96%, at least 97%, at least 98%, at least 99%, or more sequence identity to SEQ ID NO: 5, wherein the deaminase has at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.

In some embodiments, the deaminase has:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.

In some embodiments, the deaminase comprises an amino acid sequence that has an N at a position corresponding to position 35 of SEQ ID NO: 5; an S at a position corresponding to position 81 of SEQ ID NO: 5; an R at a position corresponding to position 156 of SEQ ID NO: 5; and a W at a position corresponding to position 162 of SEQ ID NO: 5. In some embodiments, the deaminase comprises an amino acid sequence that has a W at a position corresponding to position 75 of SEQ ID NO: 5; an S at a position corresponding to position 81 of SEQ ID NO: 5; a W at a position corresponding to position 155 of SEQ ID NO: 5; and a D at a position corresponding to position 160 of SEQ ID NO: 5. In some embodiments, the deaminase comprises an amino acid sequence that has an S at a position corresponding to position 81 of SEQ ID NO: 5; and an L at a position corresponding to position 117 of SEQ ID NO: 5. In some embodiments, the deaminase comprises an amino acid sequence that has an A at a position corresponding to position 68 of SEQ ID NO: 5; and an S at a position corresponding to position 81 of SEQ ID NO: 5. In some embodiments, the deaminase comprises an amino acid sequence that has a Y at a position corresponding to position 75 of SEQ ID NO: 5; an S at a position corresponding to position 81 of SEQ ID NO: 5; an L at a position corresponding to position 117 of SEQ ID NO: 5; a G at a position corresponding to position 121 of SEQ ID NO: 5; a L at a position corresponding to position 145 of SEQ ID NO: 5; an F at a position corresponding to position 155 of SEQ ID NO: 5; and a D at a position corresponding to position 160 of SEQ ID NO: 5. In some embodiments, the deaminase comprises an amino acid sequence that has an S at a position corresponding to position 81 of SEQ ID NO: 5; a W at a position corresponding to position 155 of SEQ ID NO: 5; and a D at a position corresponding to position 160 of SEQ ID NO: 5. In some embodiments, the deaminase comprises an amino acid sequence that has an S at a position corresponding to position 81 of SEQ ID NO: 5; an L at a position corresponding to position 109 of SEQ ID NO: 5; an E at a position corresponding to position 153 of SEQ ID NO: 5; a W at a position corresponding to position 155 of SEQ ID NO: 5; and a D at a position corresponding to position 160 of SEQ ID NO: 5.

In some embodiments, the deaminase comprises the amino acid sequence of SEQ ID NO: 303. In some embodiments, the deaminase comprises the amino acid sequence of SEQ ID NO: 319. In some embodiments, the deaminase comprises the amino acid sequence of SEQ ID NO: 365. In some embodiments, the deaminase comprises the amino acid sequence of SEQ ID NO: 375. In some embodiments, the deaminase comprises the amino acid sequence of SEQ ID NO: 406. In some embodiments, the deaminase comprises the amino acid sequence of SEQ ID NO: 596. In some embodiments, the deaminase comprises the amino acid sequence of SEQ ID NO: 597. In some embodiments, the deaminase comprises the amino acid sequence of SEQ ID NO: 720.

In some embodiments, the deaminase consists of the amino acid sequence of SEQ ID NO: 303. In some embodiments, the deaminase consists of the amino acid sequence of SEQ ID NO: 319. In some embodiments, the deaminase consists of the amino acid sequence of SEQ ID NO: 365. In some embodiments, the deaminase consists of the amino acid sequence of SEQ ID NO: 375. In some embodiments, the deaminase consists of the amino acid sequence of SEQ ID NO: 406. In some embodiments, the deaminase consists of the amino acid sequence of SEQ ID NO: 596. In some embodiments, the deaminase consists of the amino acid sequence of SEQ ID NO: 597. In some embodiments, the deaminase consists of the amino acid sequence of SEQ ID NO: 720.

The deaminases of the invention can have improved deaminase activity when compared to the parental LPG50148 deaminase. Improved deaminase activity can be measured by any method known in the art to measure the deamination of a nucleobase (e.g., adenine) alone or when fused to a DNA binding polypeptide (e.g., RGN). The deaminases disclosed herein can have 1.1-fold to 10-fold, 1.1-fold to 20-fold, 1.1-fold to 30-fold, or more greater efficiency in deaminating a nucleobase than the parental LPG50148 deaminase, including but not limited to about 1.1-fold, about 1.5-fold, about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 11-fold, about 12-fold, about 13-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, and about 30-fold.

### IV. Heterologous Polypeptide

In some embodiments, the presently disclosed fusion proteins comprise a heterologous polypeptide inserted within an RGN. A heterologous polypeptide is any polypeptide that is not naturally bound to an RGN protein. In some embodiments, the heterologous polypeptide comprises a protein domain with biological activity. The heterologous polypeptide can comprise a detectable label, a selectable marker, or a purification tag. In some embodiments, the heterologous polypeptide is a base-editing polypeptide (e.g., deaminase) or a prime-editing polypeptide.

### A. Prime Editing Polypeptides

The heterologous polypeptide of the presently disclosed fusion proteins can be a prime-editing polypeptide, such as a reverse transcriptase.

Prime editing is a versatile and precise genome editing method that directly writes new genetic information into a specified DNA site using a nucleic acid programmable DNA binding protein working in association with a polymerase (described in, e.g., U.S. Pat. No. 11,447,770B1; WO2021072328; WO2021226558; WO2020156575; WO2021042047; U.S. Ser. No. 11/193,123; each incorporated by reference in its entirety herein).

The prime editing system uses an RGN that is a nickase (generally one wherein the HNH domain has been inactivated), and the system is programmed with a prime editing (PE) guide RNA (“PEgRNA”). The PEgRNA is a guide RNA that both specifies the target sequence and provides the template for polymerization of the replacement strand containing the edit by way of an extension engineered onto the guide RNA (e.g., at the 5′ or 3′ end, or at an internal portion of the guide RNA). The RGN nickase/prime editing polypeptide fusion is guided to the target sequence by the PEgRNA and nicks the target strand upstream of sequence to be edited and upstream of the PAM, creating a 3′ flap on the target strand. The pegRNA includes a primer binding site (PBS) that is complementary to the 3′ flap of the target strand. In some embodiments, a PBS is at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In certain embodiments, the pegRNA comprises a PBS that is at least 5 (e.g., at least 5, 6, 7, 8, 9, 10, 11, 12, 13, 14, 15, 16, 17, 28, 19, or 20) nucleotides in length. In some embodiments, the pegRNA may comprise a PBS that is at least 8 nucleotides in length. Hybridrization of the PBS and 3′ flap of the target strand allows polymerization of the replacement strand containing the edit using the extension of the PEgRNA as template. The extension of the PEgRNA can be formed from RNA or DNA. In the case of an RNA extension, the polymerase of the prime editor can be an RNA-dependent DNA polymerase (such as a reverse transcriptase). In the case of a DNA extension, the polymerase of the prime editor may be a DNA-dependent DNA polymerase.

The replacement strand containing the desired edit (e.g., a single nucleobase substitution) shares the same sequence as the target strand of the target sequence to be edited (with the exception that it includes the desired edit). Through DNA repair and/or replication machinery, the target strand of the target sequence is replaced by the newly synthesized replacement strand containing the desired edit. In some cases, prime editing may be thought of as a “search-and-replace” genome editing technology since the prime editors not only search and locate the desired target sequence to be edited, but at the same time, encode a replacement strand containing a desired edit which is installed in place of the corresponding target strand of the target sequence. Thus, in some embodiments, a guide RNA useful in the presently disclosed compositions and methods comprises an extension comprising an edit template for prime editing. In some embodiments, a prime editing polypeptide that can be fused to an RGN includes a DNA polymerase (e.g., an RNA-dependent DNA polymerase). In certain embodiments, the DNA polymerase is a reverse transcriptase. In certain embodiments, the RGN is a nickase, such as one wherein the HNH domain has been inactivated.

### B. Base-Editing Polypeptide

The heterologous polypeptide of the presently disclosed fusion proteins can be a base-editing polypeptide, such as a deaminase.

In some embodiments, the deaminase component of the presently disclosed fusion proteins (wherein the deaminase is inserted into an RGN) is any one of SEQ ID NOs: 5, 6, 229-414, 596, 597, and 720-723, including those deaminases disclosed herein (SEQ ID NOs: 300-414, 596-598, and 720-723), or an active fragment or variant thereof. The deaminase component can have between 80% and 99% or more sequence identity to any one of SEQ ID NOs: 5, 6, 229-414, 596, 597, and 720-723 (that retains deaminase activity), including but not limited to at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more. The fusion protein can comprise an active fragment of a deaminase set forth as any one of SEQ ID NOs: 5, 6, 229-414, 596, 597, and 720-723, such as one that differs from any one of SEQ ID NOs: 5, 6, 229-414, 596, 597, and 720-723 by as few as 1-15 amino acid residues, as few as 1-10, such as 6-10, as few as 5, as few as 4, as few as 3, as few as 2, or as few as 1 amino acid residue. In some embodiments, the deaminase that is inserted into an RGN has about 50% to about 100% identity to any of SEQ ID NOs: 5, 6, 300-414, 596-598, and 720-723 and has at least one of the amino acid residues set forth in any one of Tables 2, 4, 6, and 19. In some embodiments, the deaminase has an amino acid sequence having at least 50%, at least 55%, at least 60%, at least 65%, at least 70%, at least 75%, at least 80%, at least 85%, at least 90%, at least 95%, at least 96%, at least 97%, at least 98%, at least 99%, or 100% identity to any of SEQ ID NOs: 5, 6, 300-414, 596-598, and 720-723 and has at least one of the amino acid residues set forth in any one of Tables 2, 4, 6, and 19

In certain embodiments, the active deaminase fragment comprises an N-terminal or a C-terminal truncation, which can comprise at least a deletion of 1, 2, 3, 4, 5, 10, 15, 20, 25, 30, 35, 40, 45, 50, 55, 60 amino acids or more from either the N or C terminus of the polypeptide set forth in any one of SEQ ID NOs: 5, 6, 229-414, 596, 597, and 720-723. The fusion protein can comprise a deaminase that lacks the first and last amino acid residues in comparison to a parental deaminase from which the deaminase is derived, wherein deaminase activity is retained. The fusion protein can comprise a deaminase that lacks the first and last amino acid residues of any one of SEQ ID NOs: 5, 6, 229-414, 596, 597, and 720-723, wherein deaminase activity is retained. In some embodiments, the active deaminase variant comprises an internal deletion which can comprise at least a deletion of 1, 2, 3, 4, 5, 6, 7, 8, 9, 10, 11, 12, 13, 14, 15, 16, 17, 18, 19, 20, 25, 30, 35, 40, 45, 50, 55, 60 amino acids or more of any one of SEQ ID NOs: 5, 6, 229-414, 596, 597, and 720-723. An active fragment of a deaminase can comprise at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth in any one of SEQ ID NOs: 5, 6, 229-414, 596, 597, and 720-723.

In some embodiments, the deaminase component of the presently disclosed fusion proteins (wherein the deaminase is inserted into an RGN) is any one of SEQ ID Nos: 303, 319, 365, 375, 406, 597, and 720 or an active variant or fragment thereof.

“Base editors” are fusion proteins comprising an RGN operably linked to a deaminase such that the fusion protein is capable of deaminating a nucleobase of (or adjacent to) a target nucleic acid sequence when bound to a guide RNA. The RGN of base editors generally comprise an RGN nickase (generally one with an inactivated RuvC domain) or a dead RGN that has no nuclease activity. Base editor fusion proteins wherein a deaminase has been inserted within an RGN are referred to herein as “inlaid base editors”.

The presently disclosed fusion proteins can comprise an adenine deaminase, such as those disclosed herein (e.g., 5, 6, 248-414, 596, 597, and 720-723). A base editor comprising an RGN and an adenine deaminase is referred to herein as an “A-based editor”, “adenine base editor”, or an “ABE” and can be used for the targeted editing of nucleic acid sequences. To date there are no known naturally occurring adenine deaminases that deaminate adenine in DNA. Several methods have been employed to evolve and optimize adenine deaminase acting on tRNA (ADAT) proteins to be active on DNA molecules in mammalian cells (Gaudelli et al, 2017; Koblan, L. W. et al, 2018, Nat Biotechnol 36, 843-846; Richter, M. F. et al, 2020, Nat Biotechnol, doi:10.1038/s41587-020-0562-8, each of which are incorporated by reference in their entirety herein).

Non-limiting examples of adenine deaminases that can be used in the present invention include, but are not limited to those disclosed in PCT International Publ. No. WO 2022/056254, and SEQ ID NOs: 5, 6, 248-414, 596, 597, and 720-723 or active variants or fragments thereof.

Adenine base editors (ABEs) comprise an RGN and an adenine deaminase. ABEs function through the deamination of adenine into inosine on a DNA target molecule (Gaudelli, N. M. et al. 2017). Inosine is recognized as a guanine by polymerases and allows for the incorporation of a cytosine on the complementary DNA strand across from the inosine. After a round of replication post-deamination, there is a resulting A:T to G:C base pair change in the genome. In some embodiments, the adenine deaminases or active variants or fragments thereof of the fusion protein introduce A>N mutations in a DNA molecule, wherein N is C, G, or T. In further embodiments, they introduce A>G mutations in a DNA molecule.

In some embodiments, the presently disclosed fusion proteins comprising an RGN with a deaminase inserted therein can comprise a cytosine deaminase that catalyzes the hydrolytic deamination of cytosine, cytidine, or deoxycytidine to uracil. Cytosine deaminases may work on either DNA or RNA, and typically operate on single-stranded nucleic acid molecules. In some embodiments, the cytosine deaminase is an apolipoprotein B mRNA-editing complex (APOBEC) family deaminase. In some embodiments, the cytosine deaminase is an APOBEC1 family deaminase. In some embodiments, the cytosine deaminase is an activation-induced cytidine deaminase (AID). In some embodiments, the cytosine deaminase is an ACF1/ASE deaminase.

A fusion protein comprising an RGN and a cytosine deaminase is referred to herein as a “C-base editor”, “cytosine base editor” or “CBE”. CBEs can convert a cytosine to uracil, which can be subsequently converted to thymine through DNA replication or repair. In some embodiments, a CBE converts a cytosine to a guanine or a cytosine to an adenine. Without being bound by any theory or mechanism of action, it is believed that conversion of a cytosine to a guanine or adenine by a cytosine base editor is due to the deamination of the cytosine into a uracil and the subsequent activity of a uracil DNA glycosylase during base excision repair of the uracil residue. In some embodiments, the cytosine deaminases or active variants or fragments thereof of the fusion protein introduce C>N mutations in a DNA molecule, wherein N is A, G, or T. In further embodiments, they introduce C>G or C>A mutations in a DNA molecule.

Non-limiting examples of cytosine deaminases that can be used in the present invention include, but are not limited to those disclosed in PCT International Publ. Nos. WO 2020/139783, and WO 2022/204093 and SEQ ID NOs: 229-247 or active variants or fragments thereof.

The mutation rate of adenines or cytosines within or adjacent to the target sequence to which the RGN-deaminase fusion protein binds can be measured using any method known in the art, including polymerase chain reaction (PCR), restriction fragment length polymorphism (RFLP), or DNA sequencing.

### V. Fusion Proteins

The invention provides various types of fusion proteins. A person of ordinary skill in the art will appreciate that anytime a protein is fused to another protein, the N-terminal methionine (Met) of the C-terminal protein may optionally be omitted from the sequence. Therefore, as used herein, reference to a given amino acid sequence being fused to another amino acid sequence explicitly includes such sequences with or optionally without a N-terminal Met regardless of whether such sequence comprises an N-terminal Met in the sequence listing or not.

Some aspects of the invention involve fusion proteins comprising a heterologous polypeptide inserted within an RGN. The heterologous polypeptide can be inserted into the RGN at its surface (i.e., inserted between surface amino acid residues or amino acid residues within a surface loop).

The heterologous polypeptide can be inserted within a linker domain 2, a wedge (WED) domain, a RuvC domain, an HNH domain, a Rec-2 domain, or a PAM-interacting (PI) domain. In some embodiments, the RuvC domain is the RuvCIII domain. A Rec or recognition lobe mediates nucleic acid binding through multiple Rec domains (e.g., Rec1-3) by sensing nucleic acids, regulates the HNH conformational transition, and locks the catalytic HNH domain at the cleavage site. A wedge domain is responsible for the recognition of guide RNA scaffolds. Non-limiting examples of domains within an RGN include: RuvC-I from amino acid residues 1-54; BH from amino acid residues 55-83; REC1 from amino acid residues 84-244; REC2 from amino acid residues 245-462; RuvC-II from amino acid residues 463-521; L1 from amino acid residues 522-552; HNH from amino acid residues 553-672; L2 from amino acid residues 673-685; RuvC-III from amino acid residues 686-833; WED from amino acid residues 834-938; and PI from amino acid residues 939-1071; all in reference to APG07433.1, which is set forth as SEQ ID NO: 1. APG05586 (set forth as SEQ ID NO: 2) has the following domains: RuvC-I from amino acid residues 1-33; BH from amino acid residues 34-71; REC1 from amino acid residues 72-232; REC2 from amino acid residues 233-468; RuvC-II from amino acid residues 469-517; L1 from amino acid residues 518-552; HNH from amino acid residues 553-672; L2 from amino acid residues 673-687; RuvC-III from amino acid residues 688-837; WED from amino acid residues 838-998; and PI from amino acid residues 999-1150. LPG10145 (set forth as SEQ ID NO: 4) has the following domains: RuvC-I from amino acid residues 1-42; BH from amino acid residues 43-79; REC1 from amino acid residues 80-236; REC2 from amino acid residues 237-476; RuvC-II from amino acid residues 477-524; L1 from amino acid residues 525-560; HNH from amino acid residues 561-676; L2 from amino acid residues 677-690; RuvC-III from amino acid residues 691-828; WED from amino acid residues 829-976; and PI from amino acid residues 977-1130. APG01604 (set forth as SEQ ID NO: 3) has the following domains: RuvC-I from amino acid residues 1-40; BH from amino acid residues 41-74; REC1 from amino acid residues 75-223; REC2 from amino acid residues 224-430; RuvC-II from amino acid residues 431-483; L1 from amino acid residues 484-516; HNH from amino acid residues 517-631; L2 from amino acid residues 632-651; RuvC-III from amino acid residues 652-775; WED from amino acid residues 776-909; and PI from amino acid residues 910-1052.

A PAM-interacting domain is the domain that binds to the PAM sequence. The general domains of RGN proteins can be determined via structural comparison to RGN proteins with defined domains.

In those embodiments wherein the fusion protein comprises an RGN having at least 90% sequence identity to SEQ ID NO: 1 or 49, the heterologous polypeptide (e.g., deaminase) can be inserted within the RGN immediately after the amino acid position selected from the group consisting of:
i) amino acid position corresponding to position 30 of SEQ ID NO: 1;
ii) amino acid position corresponding to position 642 of SEQ ID NO: 1;
iii) amino acid position corresponding to position 670 of SEQ ID NO: 1;
iv) amino acid position corresponding to position 737 of SEQ ID NO: 1;
v) amino acid position corresponding to position 772 of SEQ ID NO: 1;
vi) amino acid position corresponding to position 775 of SEQ ID NO: 1;
vii) amino acid position corresponding to position 778 of SEQ ID NO: 1; and
viii) amino acid position corresponding to position 802 of SEQ ID NO: 1.

In those embodiments wherein the fusion protein comprises an RGN having at least 90% sequence identity to SEQ ID NO: 2 or 50, the heterologous polypeptide can be inserted within the RGN immediately after the amino acid position selected from the group consisting of:
i) amino acid position corresponding to position 678 of SEQ ID NO: 2;
ii) amino acid position corresponding to position 736 of SEQ ID NO: 2;
iii) amino acid position corresponding to position 778 of SEQ ID NO: 2;
iv) amino acid position corresponding to position 788 of SEQ ID NO: 2; and
v) amino acid position corresponding to position 922 of SEQ ID NO: 2.

In those embodiments wherein the fusion protein comprises an RGN having at least 90% sequence identity to SEQ ID NO: 3 or 51, the heterologous polypeptide can be inserted within the RGN immediately after the amino acid position selected from the group consisting of:
i) amino acid position corresponding to position 725 of SEQ ID NO: 3;
ii) amino acid position corresponding to position 739 of SEQ ID NO: 3; and
iii) amino acid position corresponding to position 744 of SEQ ID NO: 3.

In those embodiments wherein the fusion protein comprises an RGN having at least 90% sequence identity to SEQ ID NO: 4 or 52, the heterologous polypeptide can be inserted within the RGN immediately after the amino acid position selected from the group consisting of:
i) amino acid position corresponding to position 347 of SEQ ID NO: 4;
ii) amino acid position corresponding to position 524 of SEQ ID NO: 4;
iii) amino acid position corresponding to position 666 of SEQ ID NO: 4;
iv) amino acid position corresponding to position 680 of SEQ ID NO: 4;
v) amino acid position corresponding to position 740 of SEQ ID NO: 4;
vi) amino acid position corresponding to position 785 of SEQ ID NO: 4;
vii) amino acid position corresponding to position 910 of SEQ ID NO: 4; and
viii) amino acid position corresponding to position 1077 of SEQ ID NO: 4.

In those embodiments wherein the fusion protein comprises an RGN having at least 90% sequence identity to SEQ ID NO: 131 or 698, the heterologous polypeptide can be inserted within the RGN immediately after the amino acid position selected from the group consisting of:
i) amino acid position corresponding to position 766 of SEQ ID NO: 131; and
ii) amino acid position corresponding to position 806 of SEQ ID NO: 131.

The fusion proteins of the invention can comprise base editor fusion proteins, wherein the heterologous polypeptide is a deaminase and the deaminase is inserted within an RGN, such as an RGN nickase or nuclease-inactive RGN. In some embodiments, the RGN component of the base editor fusion protein comprises an RGN nickase or nuclease-inactive RGN having about 60% to about 99.5% identity to any one of SEQ ID NOs: 49-52 and 698. In some embodiments the RGN nickase or nuclease-inactive RGN comprises an amino acid sequence that has at least 60%, at least 65%, at least 70%, at least 75%, at least 80%, at least 85%, at least 90%, at least 95%, at least 96%, at least 97%, at least 98%, at least 99%, or at least 99.5% identity to any one of SEQ ID NOs: 49-52 and 698.

In some embodiments, the base editor fusion protein comprises an RGN nickase or nuclease-inactive RGN comprising an amino acid sequence having between 80% and 99% or more sequence identity to any one of SEQ ID NOs: 49-52 and 698, including but not limited to at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more.

The fusion proteins of the invention can comprise base editor fusion proteins (wherein the heterologous polypeptide is a deaminase and the deaminase is inserted within an RGN, such as an RGN nickase or nuclease-inactive RGN) wherein the base editor fusion protein has improved editing activity when compared to a parental base editor comprising the deaminase fused to the amino terminus of the RGN. Improved editing activity can be measured by any method known in the art. The base editor fusion proteins disclosed herein can have 1.1-fold to 10-fold, 1.1-fold to 20-fold, 1.1-fold to 30-fold, or more greater efficiency in editing than the parental end-to-end fusion protein, including but not limited to about 1.1-fold, about 1.5-fold, about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 11-fold, about 12-fold, about 13-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, and about 30-fold.

The binding of a base editor fusion protein to a target sequence results in modification of a nucleotide adjacent to the target sequence. The nucleobase adjacent to the target sequence that is modified by the deaminase may be 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs from the 5′ or 3′ end of the target sequence. The full range of nucleotides that can be edited using a base editor fusion protein (e.g., RGN fused to a deaminase), usually expressed as the distance of the nucleotides from the PAM sequence (e.g., nucleotides at positions 8 and 22 upstream, i.e., 5′, from the PAM sequence), is referred to herein as the “editing window” of a particular base editor fusion protein. The editing window may be, for example, 1-100 base pairs 5′ or 3′ of the PAM sequence, including but not limited to 5-50, 5-25, 8-22, 10-20, or 10-21 base pairs 5′ or 3′ of the PAM sequence.

Non-limiting examples of inlaid base editor fusion proteins include:
a) LPG50274/LPG50148.2.76 (set forth as SEQ ID NO: 375) or an active variant or fragment thereof inserted within nAPG05586 (set forth as SEQ ID NO: 50) or an active variant or fragment thereof after an amino acid position corresponding to position 922 of SEQ ID NO: 2, such as the sequence set forth as SEQ ID NO: 599;
b) LPG50274/LPG50148.2.76 (set forth as SEQ ID NO: 375) or an active variant or fragment thereof inserted within nLPG10145 (set forth as SEQ ID NO: 52) or an active variant or fragment thereof after an amino acid position corresponding to position 910 of SEQ ID NO: 4, such as the sequence set forth as SEQ ID NO: 600;
c) LPG50221/LPG50148.2.20 (set forth as SEQ ID NO: 319) or an active variant or fragment thereof inserted within nAPG05586 (set forth as SEQ ID NO: 50) or an active variant or fragment thereof after an amino acid position corresponding to position 922 of SEQ ID NO: 2, such as the sequence set forth as SEQ ID NO: 602;
d) LPG50221/LPG50148.2.20 (set forth as SEQ ID NO: 319) or an active variant or fragment thereof inserted within nLPG10145 (set forth as SEQ ID NO: 52) or an active variant or fragment thereof after an amino acid position corresponding to position 910 of SEQ ID NO: 4, such as the sequence set forth as SEQ ID NO: 603;
e) LPG50274/LPG50148.2.76 (set forth as SEQ ID NO: 375) or an active variant or fragment thereof inserted within nAPG07433.1 (set forth as SEQ ID NO: 49) or an active variant or fragment thereof after an amino acid position corresponding to position 772 of SEQ ID NO: 1, such as the sequence set forth as SEQ ID NO: 606;
f) LPG50274/LPG50148.2.76 (set forth as SEQ ID NO: 375) or an active variant or fragment thereof inserted within nAPG05586 (set forth as SEQ ID NO: 50) or an active variant or fragment thereof after an amino acid position corresponding to position 678 of SEQ ID NO: 2, such as the sequence set forth as SEQ ID NO: 610;
g) LPG50221/LPG50148.2.20 (set forth as SEQ ID NO: 319) or an active variant or fragment thereof inserted within nAPG07433.1 (set forth as SEQ ID NO: 49) or an active variant or fragment thereof after an amino acid position corresponding to position 772 of SEQ ID NO: 1, such as the sequence set forth as SEQ ID NO: 612;
h) LPG50324 (set forth as SEQ ID NO: 597) or an active variant or fragment thereof inserted within nAPG05586 (set forth as SEQ ID NO: 50) or an active variant or fragment thereof after an amino acid position corresponding to position 922 of SEQ ID NO: 2, such as the sequence set forth as SEQ ID NO: 614;
i) LPG50221/LPG50148.2.20 (set forth as SEQ ID NO: 319) or an active variant or fragment thereof inserted within nAPG05586 (set forth as SEQ ID NO: 50) or an active variant or fragment thereof after an amino acid position corresponding to position 678 of SEQ ID NO: 2, such as the sequence set forth as SEQ ID NO: 615;
j) LPG50319/LPG50148.2.107 (set forth as SEQ ID NO: 406) or an active variant or fragment thereof inserted within nAPG05586 (set forth as SEQ ID NO: 50) or an active variant or fragment thereof after an amino acid position corresponding to position 678 of SEQ ID NO: 2, such as the sequence set forth as SEQ ID NO: 617;
k) LPG50324 (set forth as SEQ ID NO: 597) or an active variant or fragment thereof inserted within nAPG05586 (set forth as SEQ ID NO: 50) or an active variant or fragment thereof after an amino acid position corresponding to position 678 of SEQ ID NO: 2, such as the sequence set forth as SEQ ID NO: 619;
l) LPG50320 (set forth as SEQ ID NO: 596) or an active variant or fragment thereof inserted within nAPG05586 (set forth as SEQ ID NO: 50) or an active variant or fragment thereof after an amino acid position corresponding to position 678 of SEQ ID NO: 2, such as the sequence set forth as SEQ ID NO: 620;
m) LPG50319/LPG50148.2.107 (set forth as SEQ ID NO: 406) or an active variant or fragment thereof inserted within nLPG10145 (set forth as SEQ ID NO: 52) or an active variant or fragment thereof after an amino acid position corresponding to position 910 of SEQ ID NO: 4, such as the sequence set forth as SEQ ID NO: 621;
n) LPG50324 (set forth as SEQ ID NO: 597) or an active variant or fragment thereof inserted within nLPG10145 (set forth as SEQ ID NO: 52) or an active variant or fragment thereof after an amino acid position corresponding to position 910 of SEQ ID NO: 4, such as the sequence set forth as SEQ ID NO: 622;
o) LPG50319/LPG50148.2.107 (set forth as SEQ ID NO: 406) or an active variant or fragment thereof inserted within nAPG05586 (set forth as SEQ ID NO: 50) or an active variant or fragment thereof after an amino acid position corresponding to position 922 of SEQ ID NO: 2, such as the sequence set forth as SEQ ID NO: 623;
p) LPG50320 (set forth as SEQ ID NO: 596) or an active variant or fragment thereof inserted within nLPG10145 (set forth as SEQ ID NO: 52) or an active variant or fragment thereof after an amino acid position corresponding to position 910 of SEQ ID NO: 4, such as the sequence set forth as SEQ ID NO: 624;
q) LPG50320 (set forth as SEQ ID NO: 596) or an active variant or fragment thereof inserted within nAPG05586 (set forth as SEQ ID NO: 50) or an active variant or fragment thereof after an amino acid position corresponding to position 922 of SEQ ID NO: 2, such as the sequence set forth as SEQ ID NO: 626;
r) LPG50319/LPG50148.2.107 (set forth as SEQ ID NO: 406) or an active variant or fragment thereof inserted within nAPG07433.1 (set forth as SEQ ID NO: 49) or an active variant or fragment thereof after an amino acid position corresponding to position 772 of SEQ ID NO: 1, such as the sequence set forth as SEQ ID NO: 630;
s) LPG50320 (set forth as SEQ ID NO: 596) or an active variant or fragment thereof inserted within nAPG07433.1 (set forth as SEQ ID NO: 49) or an active variant or fragment thereof after an amino acid position corresponding to position 772 of SEQ ID NO: 1, such as the sequence set forth as SEQ ID NO: 631; and
t) LPG50324 (set forth as SEQ ID NO: 597) or an active variant or fragment thereof inserted within nAPG07433.1 (set forth as SEQ ID NO: 49) or an active variant or fragment thereof after an amino acid position corresponding to position 772 of SEQ ID NO: 1, such as the sequence set forth as SEQ ID NO: 632.

The fusion proteins of the invention can comprise base editor fusion proteins (wherein the heterologous polypeptide is a deaminase and the deaminase is inserted within an RGN, such as an RGN nickase or nuclease-inactive RGN) wherein the base editor fusion protein has a shifted editing window when compared to a parental base editor comprising the deaminase fused to the amino terminus of the RGN. The editing window can be shifted to be narrower or broader than the parental base editor. A shifted editing window is one that is broader or narrower (for example, by 1, 2, 3, 4, 5, 6, 7, 8, 9, 10, 11, 12, 13, 14, 15, 16, 17, 18, 19, 20 or more nucleotides) when compared to the parental end-to-end fusion protein, and/or the editing window moves in the 5′ or 3′ direction of the target molecule when compared to the parental end-to-end fusion protein, for example 1, 2, 3, 4, 5, 6, 7, 8, 9, 10, 11, 12, 13, 14, 15, 16, 17, 18, 19, 20, or more nucleotides in the 5′ or 3′ direction.

In some embodiments, the fusion protein comprises one or more peptide linkers between the heterologous polypeptide and RGN. A person of ordinary skill in the art will appreciate that anytime a protein is fused to another protein, a linker may optionally be added to the sequence. Therefore, as used herein, reference to a given fusion protein explicitly includes such sequences with or optionally without a linker regardless of whether such sequence is listed as having a linker or not. The linker between a deaminase and RGN can determine the editing window of the fusion protein. Various linker lengths and flexibilities can be employed, ranging from very flexible linkers of the form (GGGGS)ₙ and (G)ₙ to more rigid linkers of the form (EAAAK)ₙ and (XP)ₙ, to achieve the optimal length and rigidity for deaminase activity for the specific applications. The term “peptide linker,” as used herein, refers to a peptide linking two polypeptides. In some embodiments, a linker joins an RNA guided nuclease and a deaminase. In some embodiments, a linker joins a dead or inactive RGN and a deaminase. In some embodiments, the linker is 3-100 amino acids in length, for example, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, a shorter linker is preferred to decrease the overall size or length of the fusion protein or its coding sequence. Non-limiting examples of peptide linkers between an RGN and heterologous polypeptide inserted therein include GGS, SGG, and GSSG (set forth as SEQ ID NO: 642). Linkers can be at the N-terminus, C-terminus, or both of the inserted heterologous polypeptide.

In some embodiments, the RGN and heterologous polypeptide inserted therein are directly fused to one another with no linker sequences in between.

Another aspect of the invention involves fusion proteins comprising a DNA-binding polypeptide (e.g., nuclease-inactive or a nickase RGN) operably linked to a deaminase of the invention (e.g., SEQ ID NOs: 6, 300-414, 596-598, and 720-723 or active fragments or variants thereof). In some embodiments, a DNA-binding polypeptide (e.g., nuclease inactive RGN or nickase RGN) fused to a deaminase of the invention can be targeted to a particular location of a nucleic acid molecule (i.e., target nucleic acid molecule), which in some embodiments is a particular genomic locus, to alter the expression of a desired sequence. In some embodiments, the binding of a fusion protein to a target sequence results in deamination of a nucleobase, resulting in conversion from one nucleobase to another. In some embodiments, the binding of this fusion protein to a target sequence results in deamination of a nucleobase adjacent to the target sequence.

Some aspects of this disclosure provide fusion proteins comprising (i) a DNA-binding polypeptide (e.g., a nuclease-inactive or nickase RGN polypeptide); (ii) a deaminase polypeptide; and optionally (iii) a second deaminase. The second deaminase may be the same deaminase as the first or may be a different deaminase. In some embodiments, both the first and the second deaminase are adenine deaminases of the invention.

The instant disclosure provides fusion proteins of various configurations. In some embodiments, the fusion protein is an end-to-end fusion wherein the deaminase polypeptide is fused to the N-terminus of the DNA-binding polypeptide (e.g., RGN polypeptide) or the deaminase polypeptide is fused to the C-terminus of the DNA-binding polypeptide (e.g., RGN polypeptide).

Non-limiting examples of an end-to-end base editor fusion protein include those comprising the deaminase or an active variant or fragment thereof fused to the N-terminus of the nickase or an active variant or fragment thereof of any one of the end-to-end fusion proteins used in the examples herein, including but not limited to:
a) LPG50221/LPG50148.2.20 (set forth as SEQ ID NO: 319) or an active variant or fragment thereof fused to the N-terminus of nAPG07433.1 (set forth as SEQ ID NO: 49) or an active variant or fragment thereof, such as the sequence set forth as SEQ ID NO: 578 or 582;
b) LPG50274/LPG50148.2.76 (set forth as SEQ ID NO: 375) or an active variant or fragment thereof fused to the N-terminus of nAPG07433.1 (set forth as SEQ ID NO: 49) or an active variant or fragment thereof, such as the sequence set forth as SEQ ID NO: 594 or 577;
c) LPG50319/LPG50148.2.107 (set forth as SEQ ID NO: 406) or an active variant or fragment thereof fused to the N-terminus of nAPG07433.1 (set forth as SEQ ID NO: 49) or an active variant or fragment thereof, such as the sequence set forth as SEQ ID NO: 588 or 616;
d) LPG50320 (set forth as SEQ ID NO: 596) or an active variant or fragment thereof fused to the N-terminus of nAPG07433.1 (set forth as SEQ ID NO: 49) or an active variant or fragment thereof, such as the sequence set forth as SEQ ID NO: 593 or 604; and
e) LPG50324 (set forth as SEQ ID NO: 597) or an active variant or fragment thereof fused to the N-terminus of nAPG07433.1 (set forth as SEQ ID NO: 49) or an active variant or fragment thereof, such as the sequence set forth as SEQ ID NO: 605.

In some embodiments, the end-to-end deaminase and DNA-binding polypeptide (e.g., RNA-guided, DNA-binding polypeptide) are fused to each other via a linker. The term “linker,” as used herein, refers to a chemical group or a molecule linking two molecules or moieties, e.g., a binding domain and a cleavage domain of a nuclease. In some embodiments, a linker joins an RNA guided nuclease and a deaminase. In some embodiments, a linker joins a dead or inactive RGN and a deaminase. In further embodiments, a linker joins two deaminases. In some embodiments, a linker joins an RNA guided nuclease and a USP. In some embodiments, a linker joins a deaminase and a USP. In certain embodiments, a linker joins an RNA guided nuclease-deaminase fusion with a USP. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 3-100 amino acids in length, for example, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, a shorter linker is preferred to decrease the overall size or length of the fusion protein or its coding sequence.

In some embodiments, the linker between a deaminase and DNA-binding polypeptide in an end-to-end fusion comprises any one of SEQ ID NOs: 704, 705, and 707-712.Additional suitable linker motifs and linker configurations will be apparent to those of skill in the art. In some embodiments, suitable linker motifs and configurations include those described in Chen et al., 2013 (Adv Drug Deliv Rev. 65(10):1357-69, the entire contents of which are incorporated herein by reference). Additional suitable linker sequences will be apparent to those of skill in the art. In some embodiments, the linker sequence comprises the amino acid sequence set forth as SEQ ID NO: 233 or 234.

In some embodiments, the general architecture of exemplary fusion proteins provided herein comprises the structure: [NH₂]-[deaminase]-[DBP]-[COOH]; [NH₂]-[DBP]-[deaminase]-[COOH]; [NH₂]-[DBP]-[deaminase]-[deaminase]-[COOH]; [NH₂]-[deaminase]-[DBP]-[deaminase]-[COOH]; or [NH₂]-[deaminase]-[deaminase]-[DBP]-[COOH], wherein DBP is a DNA-binding polypeptide, NH₂ is the N-terminus of the fusion protein and COOH is the C-terminus of the fusion protein. In some embodiments, the fusion protein comprises more than two deaminase polypeptides.

In certain embodiments, the general architecture of exemplary fusion proteins provided herein comprises the structure: [NH₂]-[deaminase]-[RGN]-[COOH]; [NH₂]-[RGN]-[deaminase]-[COOH]; [NH₂]-[RGN]-[deaminase]-[deaminase]-[COOH]; [NH₂]-[deaminase]-[RGN]-[deaminase]-[COOH]; or [NH₂]-[deaminase]-[deaminase]-[RGN]-[COOH], wherein NH₂ is the N-terminus of the fusion protein and COOH is the C-terminus of the fusion protein. In some embodiments, the fusion protein comprises more than two deaminase polypeptides.

In some embodiments, the fusion protein comprises the structure: [NH₂]-[deaminase]-[nuclease-inactive RGN]-[COOH]; [NH₂]-[deaminase]-[deaminase]-[nuclease-inactive RGN]-[COOH]; [NH₂]-[nuclease-inactive RGN]-[deaminase]-[COOH]; [NH₂]-[deaminase]-[nuclease-inactive RGN]-[deaminase]-[COOH]; or [NH₂]-[nuclease-inactive RGN]-[deaminase]-[deaminase]-[COOH]. It should be understood that “nuclease-inactive RGN” represents any RGN, including any CRISPR-Cas protein, which has been mutated to be nuclease-inactive. In some embodiments, the fusion protein comprises more than two deaminase polypeptides.

In some embodiments, the fusion protein comprises the structure: [NH₂]-[deaminase]-[RGN nickase]-[COOH]; [NH₂]-[deaminase]-[deaminase]-[RGN nickase]-[COOH]; [NH₂]-[RGN nickase]-[deaminase]-[COGH]; [NH₂]-[deaminase]-[RGN nickase]-[deaminase]-[COOH]; or [NH₂]-[RGN nickase]-[deaminase]-[deaminase]-[COOH]. It should be understood that “RGN nickase” represents any RGN, including any CRISPR-Cas protein, which has been mutated to be active as a nickase.

In some embodiments, fusion proteins as provided herein comprise the full-length sequence of a deaminase. In some embodiments, however, fusion proteins as provided herein do not comprise a full-length sequence of a deaminase, but only a fragment thereof.

In some embodiments, a fusion protein of the invention comprises a DNA-binding polypeptide (e.g., an RGN) and a deaminase, wherein the deaminase has an amino acid sequence having about 50% to about 100% identity to any of SEQ ID NOs: 6, 300-414, 596-598, and 720-723. In some embodiments, a fusion protein of the invention comprises a DNA-binding polypeptide (e.g., an RGN) and a deaminase, wherein the deaminase has an amino acid sequence having about 50% to about 100% identity to any of SEQ ID NOs: 6, 300-414, 596-598, and 720-723 and has at least one of the amino acid residues set forth in any one of Tables 2, 4, 6, and 19. In some embodiments, a fusion protein of the invention comprises a DNA-binding polypeptide (e.g., an RGN) and a deaminase, wherein the deaminase has an amino acid sequence having at least 50%, at least 55%, at least 60%, at least 65%, at least 70%, at least 75%, at least 80%, at least 85%, at least 90%, at least 95%, at least 96%, at least 97%, at least 98%, at least 99%, or 100% identity to any of SEQ ID NOs: 6, 300-414, 596-598, and 720-723 and has at least one of the amino acid residues set forth in any one of Tables 2, 4, 6, and 19. Examples of such fusion proteins are described in the Examples section herein.

In some embodiments, the fusion protein comprises one deaminase polypeptide. In some embodiments, the fusion protein comprises at least two deaminase polypeptides, operably linked either directly or via a peptide linker. In some embodiments, the fusion protein comprises one deaminase polypeptide, and a second deaminase polypeptide is co-expressed with the fusion protein.

In some embodiments, the “-” used in the general architecture above indicates the presence of an optional linker sequence. In some embodiments, the fusion proteins provided herein do not comprise a linker sequence. In some embodiments, at least one of the optional linker sequences are present.

Other exemplary features that may be present on the presently disclosed deaminases or fusion proteins are localization sequences, such as nuclear localization sequences, cytoplasmic localization sequences, export sequences, such as nuclear export sequences, or other localization sequences, as well as sequence tags that are useful for solubilization, purification or detection of the fusion proteins. Suitable localization signal sequences and sequences of protein tags that are provided herein, and include, but are not limited to, biotin carboxylase carrier protein (BCCP) tags, myc-tags, calmodulin-tags, FLAG-tags (e.g., 3XFLAG-tag), hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), streptags, biotin ligase tags, FlAsH tags, V5 tags, and SBP-tags. Additional suitable sequences will be apparent to those of skill in the art.

The presently disclosed compositions and methods can utilize deaminases or fusion proteins comprising at least one nuclear localization signal (NLS) to enhance transport of the deaminase or fusion protein to the nucleus of a cell. Nuclear localization signals are known in the art and generally comprise a stretch of basic amino acids (see, e.g., Lange et al., J. Biol. Chem. (2007) 282:5101-5105). In some embodiments, the deaminase or fusion protein comprises 2, 3, 4, 5, 6 or more nuclear localization signals. The nuclear localization signal(s) can be a heterologous NLS. Non-limiting examples of nuclear localization signals useful for the presently disclosed RGNs are the nuclear localization signals of SV40 Large T-antigen, nucleoplasmin, c-Myc (see, e.g., Ray et al. (2015) Bioconjug Chem 26(6):1004-7), POLD1 DNA polymerase delta 1, active adenosine deaminase that acts on RNA (ADAR2), Interacts with RNA polymerase II 1 (Iwr1) (Czeko E., et al., Mol Cell 2011, which is incorporated by reference in its entirety), and OpT (U.S. Publ. No. 2020/0109382, which is incorporated by reference in its entirety). In embodiments, the RGN comprises the NLS sequence set forth as any one of SEQ ID NOs: 430, 431, and 713-718. The deaminase or fusion protein can comprise one or more NLS sequences at its N-terminus, C-terminus, or both the N-terminus and C-terminus. For example, the deaminase or fusion protein can comprise two NLS sequences at the N-terminal region and four NLS sequences at the C-terminal region. In some embodiments, the deaminase or fusion protein comprises a SV40 NLS (such as the sequence set forth as SEQ ID NO: 430) at the N-terminus and a nucleoplasmin NLS (such as the sequence set forth as SEQ ID NO: 431) at its C-terminus. In some embodiments, the deaminase or fusion protein comprises a c-Myc promoter (such as the sequence set forth as SEQ ID NO: 718) at both its N-terminus and its C-terminus.

When an NLS is attached at the N-terminus, C-terminus, or both, of a deaminase or fusion protein, an NLS linker protein can be present to separate the deaminase or fusion protein from the NLS. Such an NLS linker protein can be 1, 2, 3, 4, 5, 6, 7, 8, 9, 10, 11, 12, 13, 14, 15, or more amino acids in length. In some embodiments, the NLS linker protein is at least 1, at least 2, at least 3, at least 4, at least 5, at least 6, at least 7, or at least 8 amino acids in length. In some embodiments, the NLS linker protein between an NLS and a deaminase or fusion protein has the sequence set forth as SEQ ID NO: 706, 719, or 728. In some embodiments, the deaminase or fusion protein comprises a c-Myc promoter (such as the sequence set forth as SEQ ID NO: 718) at its N-terminus, separated from the deaminase or fusion protein by an NLS linker protein having the sequence set forth as SEQ ID NO: 728, and a c-Myc promoter (such as the sequence set forth as SEQ ID NO: 718) at its C-terminus, separated from the deaminase or fusion protein by an NLS linker protein having the sequence set forth as SEQ ID NO: 728.

In some embodiments, the presently disclosed compositions and methods utilize deaminases or fusion proteins comprising at least one cell-penetrating domain that facilitates cellular uptake of the deaminase or fusion protein. Cell-penetrating domains are known in the art and generally comprise stretches of positively charged amino acid residues (i.e., polycationic cell-penetrating domains), alternating polar amino acid residues and non-polar amino acid residues (i.e., amphipathic cell-penetrating domains), or hydrophobic amino acid residues (i.e., hydrophobic cell-penetrating domains) (see, e.g., Milletti F. (2012) Drug Discov Today 17:850-860). A non-limiting example of a cell-penetrating domain is the trans-activating transcriptional activator (TAT) from the human immunodeficiency virus 1.

The nuclear localization signal and/or cell-penetrating domain can be located at the N-terminus, the C-terminus, and/or in an internal location of the deaminase or fusion protein.

VI. Nucleotides Encoding Deaminases, Fusion Proteins and/or gRNA

The present disclosure provides polynucleotides encoding the presently disclosed deaminases, fusion proteins or a gRNA. In some embodiments, the polynucleotide encodes for a fusion protein which comprises a deaminase and a DNA-binding polypeptide, for example, a meganuclease, a zinc finger fusion protein, or a TALEN. The present disclosure further provides polynucleotides encoding for fusion proteins which comprise a deaminase and an RNA-guided, DNA-binding polypeptide (RGDBP). Such RNA-guided, DNA-binding polypeptides may be an RGN or RGN variant. The protein variant may be nuclease-inactive or a nickase. The RGN may be a CRISPR-Cas protein or active variant or fragment thereof. Examples of CRISPR-Cas nucleases are well-known in the art, and similar corresponding mutations can create mutant variants which are also nickases or are nuclease inactive.

An embodiment of the invention provides a polynucleotide encoding a fusion protein which comprises an RGDBP (e.g., RGN) and a deaminase described herein. In some embodiments, a second polynucleotide encodes the guide RNA required by the RGDBP for targeting to the nucleotide sequence of interest. In some embodiments, the guide RNA and the fusion protein are encoded by the same polynucleotide.

In some embodiments, the guide RNA and the fusion protein are encoded by the same polynucleotide. In other embodiments, the guide RNA and the fusion protein are encoded by two separate polynucleotides.

The use of the term “polynucleotide” is not intended to limit the present disclosure to polynucleotides comprising DNA, though such DNA polynucleotides are contemplated. Those of ordinary skill in the art will recognize that polynucleotides can comprise ribonucleotides (RNA) (e.g., mRNA) and combinations of ribonucleotides and deoxyribonucleotides. Such deoxyribonucleotides and ribonucleotides include both naturally occurring molecules and synthetic analogues. The polynucleotides disclosed herein also encompass all forms of sequences including, but not limited to, single-stranded forms, double-stranded forms, stem-and-loop structures, circular forms (e.g., including circular RNA), and the like.

An embodiment of the invention is a nucleic acid molecule comprising a sequence encoding a deaminase having about 50% to about 100% identity to any of SEQ ID NOs: 5, 6, 300-414, 596-598, and 720-723. An embodiment of the invention is a nucleic acid molecule comprising a sequence encoding a deaminase having about 50% to about 100% identity to any of SEQ ID NOs: 5, 6, 300-414, 596-598, and 720-723and having at least one of the amino acid residues set forth in any one of Tables 2, 4, 6, and 19. An embodiment of the invention is a nucleic acid molecule comprising a sequence encoding a deaminase having at least 50%, at least 55%, at least 60%, at least 65%, at least 70%, at least 75%, at least 80%, at least 85%, at least 90%, at least 95%, at least 96%, at least 97%, at least 98%, at least 99% or 100% identity to any of SEQ ID NOs: 5, 6, 300-414, 596-598, and 720-723and having at least one of the amino acid residues set forth in any one of Tables 2, 4, 6, and 19, wherein the nucleic acid molecule encodes a deaminase having deaminase activity. The nucleic acid molecule may further comprise a heterologous promoter or terminator. The nucleic acid molecule may encode a fusion protein, where the encoded deaminase is operably linked to a DNA-binding polypeptide, and optionally a second deaminase. In some embodiments, the nucleic acid molecule encodes a fusion protein, where the encoded deaminase is operably linked to an RGN, and optionally a second deaminase.

In some embodiments, a nucleic acid molecule comprising a polynucleotide which encodes a deaminase or fusion protein of the invention is codon optimized for expression in an organism of interest. A “codon-optimized” coding sequence is a polynucleotide coding sequence having its frequency of codon usage designed to mimic the frequency of preferred codon usage or transcription conditions of a particular host cell. Expression in the particular host cell or organism is enhanced as a result of the alteration of one or more codons at the nucleic acid level such that the translated amino acid sequence is not changed. Nucleic acid molecules can be codon optimized, either wholly or in part. Codon tables and other references providing preference information for a wide range of organisms are available in the art (see, e.g., Campbell and Gowri (1990) Plant Physiol. 92:1-11 for a discussion of plant-preferred codon usage). Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

In some embodiments, polynucleotides encoding the deaminases, fusion proteins and/or gRNAs described herein are provided in expression cassettes for in vitro expression or expression in a cell, organelle, embryo, or organism of interest. The cassette may include 5′ and 3′ regulatory sequences operably linked to a polynucleotide encoding a deaminase, fusion protein and/or gRNA provided herein that allows for expression of the polynucleotide. The cassette may additionally contain at least one additional gene or genetic element to be cotransformed into the organism. Where additional genes or elements are included, the components are operably linked. The term “operably linked” is intended to mean a functional linkage between two or more elements. For example, an operable linkage between a promoter and a coding region of interest (e.g., a region coding for a deaminase, RGN and/or gRNA) is a functional link that allows for expression of the coding region of interest. Operably linked elements may be contiguous or non-contiguous. When used to refer to the joining of two protein coding regions, by operably linked is intended that the coding regions are in the same reading frame. In some embodiments, the additional gene(s) or element(s) are provided on multiple expression cassettes. For example, the nucleotide sequence encoding a presently disclosed deaminase or fusion protein can be present on one expression cassette, whereas the nucleotide sequence encoding a gRNA can be on a separate expression cassette. Another example may have the nucleotide sequence encoding a presently disclosed deaminase alone on a first expression cassette, a second expression cassette encoding a fusion protein comprising a deaminase, and a nucleotide sequence encoding a gRNA on a third expression cassette. Such an expression cassette is provided with a plurality of restriction sites and/or recombination sites for insertion of the polynucleotides to be under the transcriptional regulation of the regulatory regions. Expression cassettes which comprise a selectable marker gene may also be present.

The expression cassette may include in the 5′-3′ direction of transcription, a transcriptional (and, in some embodiments, translational) initiation region (i.e., a promoter), a deaminase-encoding polynucleotide of the invention, fusion protein-encoding polynucleotide of the invention, and a transcriptional (and in some embodiments, translational) termination region (i.e., termination region) functional in the organism of interest. Promoters useful in the invention are capable of directing or driving expression of a coding sequence in a host cell. The regulatory regions (e.g., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. As used herein, a chimeric gene comprises a coding sequence operably linked to a transcription initiation region that is heterologous to the coding sequence.

Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, inducible, growth stage-specific, cell type-specific, tissue-preferred, tissue-specific, or other promoters for expression in the organism of interest. See, for example, promoters set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters also include CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); and MAS (Velten et al. (1984) EMBO J. 3:2723-2730).

Examples of inducible promoters are the Adh1 promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the Axig1 promoter which is auxin induced and tapetum specific but also active in callus (PCT US01/22169), the steroid-responsive promoters (see, for example, the ERE promoter which is estrogen induced, and the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

In some embodiments, tissue-specific or tissue-preferred promoters are utilized to target expression of an expression construct within a particular tissue. In certain embodiments, the tissue-specific or tissue-preferred promoters are active in plant tissue. Examples of promoters under developmental control in plants include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues.

In some embodiments, the nucleic acid molecules encoding a deaminase or fusion protein described herein comprise a cell type-specific promoter. A “cell type specific” promoter is a promoter that primarily drives expression in certain cell types in one or more organs. Some examples of plant cells in which cell type specific promoters functional in plants may be primarily active include, for example, BETL cells, vascular cells in roots, leaves, stalk cells, and stem cells. The nucleic acid molecules can also include cell type preferred promoters. A “cell type preferred” promoter is a promoter that primarily drives expression mostly, but not necessarily entirely or solely in certain cell types in one or more organs. Some examples of plant cells in which cell type preferred promoters functional in plants may be preferentially active include, for example, BETL cells, vascular cells in roots, leaves, stalk cells, and stem cells.

In some embodiments, the nucleic acid sequence encoding the deaminase, the fusion protein and/or gRNA is operably linked to a promoter sequence that is recognized by a phage RNA polymerase for example, for in vitro mRNA synthesis. In such embodiments, the in vitro-transcribed RNA can be purified for use in the methods described herein. For example, the promoter sequence can be a T7, T3, or SP6 promoter sequence or a variation of a T7, T3, or SP6 promoter sequence. In such embodiments, the expressed protein and/or RNAs can be purified for use in the methods of genome modification described herein.

In certain embodiments, the polynucleotide encoding the deaminase, fusion protein and/or gRNA is linked to a polyadenylation signal (e.g., SV40 polyA signal and other signals functional in plants) and/or at least one transcriptional termination sequence. In some embodiments, the sequence encoding the deaminase or fusion protein is linked to sequence(s) encoding at least one nuclear localization signal, at least one cell-penetrating domain, and/or at least one signal peptide capable of trafficking proteins to particular subcellular locations, as described elsewhere herein.

In some embodiments, the polynucleotide encoding the deaminase, fusion protein and/or gRNA is present in a vector or multiple vectors. A “vector” refers to a polynucleotide composition for transferring, delivering, or introducing a nucleic acid into a host cell. Suitable vectors include plasmid vectors, phagemids, cosmids, artificial/mini-chromosomes, transposons, and viral vectors (e.g., lentiviral vectors, adeno-associated viral vectors, baculoviral vector). In some embodiments, the vector comprises additional expression control sequences (e.g., enhancer sequences, Kozak sequences, polyadenylation sequences, transcriptional termination sequences), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

In some embodiments, the vector comprises a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D).

In some embodiments, the expression cassette or vector comprising the sequence encoding a fusion protein further comprises a sequence encoding a gRNA. In some embodiments, the sequence(s) encoding the gRNA are operably linked to at least one transcriptional control sequence for expression of the gRNA in the organism or host cell of interest. For example, the polynucleotide encoding the gRNA can be operably linked to a promoter sequence that is recognized by RNA polymerase III (Pol III). Examples of suitable Pol III promoters include, but are not limited to, mammalian U6, U3, H1, and 7SL RNA promoters, rice U6 and U3 promoters, and the promoters disclosed in PCT International Appl. No. PCT/US2022/032940 filed Jun. 10, 2022, which is herein incorporated by reference in its entirety.

As indicated, expression constructs comprising nucleotide sequences encoding the deaminases, fusion proteins and/or gRNAs can be used to transform organisms of interest. Methods for transformation involve introducing a nucleotide construct into an organism of interest. By “introducing” is intended to introduce the nucleotide construct to the host cell in such a manner that the construct gains access to the interior of the host cell. The methods of the invention do not require a particular method for introducing a nucleotide construct to a host organism, only that the nucleotide construct gains access to the interior of at least one cell of the host organism. In some embodiments, an mRNA encoding a deaminase or a fusion protein is introduced into a host cell. In some embodiments wherein the fusion protein comprises an RGN, an mRNA encoding the fusion protein is introduced into a cell and a gRNA is introduced into the cell. The host cell can be a eukaryotic or prokaryotic cell. In some embodiments, the eukaryotic host cell is a plant cell, a mammalian cell, or an insect cell. Methods for introducing nucleotide constructs into plants and other host cells are known in the art including, but not limited to, stable transformation methods, transient transformation methods, and virus-mediated methods.

The methods result in a transformed organism, such as a plant, including whole plants, as well as plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic organisms” or “transformed organisms” or “stably transformed” organisms or cells or tissues refers to organisms that have incorporated or integrated a polynucleotide encoding a deaminase or fusion protein of the invention. It is recognized that other exogenous or endogenous nucleic acid sequences or DNA fragments may also be incorporated into the host cell. Agrobacterium- and biolistic-mediated transformation remain the two predominantly employed approaches for transformation of plant cells. However, transformation of a host cell may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, and viral mediated, liposome mediated and the like. Viral-mediated introduction of a polynucleotide encoding a deaminase, fusion protein, and/or gRNA includes retroviral, lentiviral, adenoviral, and adeno-associated viral mediated introduction and expression, as well as the use of Caulimoviruses (e.g., cauliflower mosaic virus), Geminiviruses (e.g., bean golden yellow mosaic virus or maize streak virus), and RNA plant viruses (e.g., tobacco mosaic virus).

Transformation protocols as well as protocols for introducing polypeptides or polynucleotide sequences into plants may vary depending on the type of host cell (e.g., monocot or dicot plant cell) targeted for transformation. Methods for transformation are known in the art and include those set forth in U.S. Pat. Nos. 8,575,425; 7,692,068; 8,802,934; 7,541,517; each of which is herein incorporated by reference. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G.W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; Jones et al. (2005) Plant Methods 1:5;

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The cells that have been transformed may be grown into a transgenic organism, such as a plant, in accordance with conventional ways. See, for example, McCormick et al. (1986) Plant Cell Reports 5:81-84. These plants may then be grown, and either pollinated with the same transformed strain or different strains, and the resulting hybrid having the deaminase or fusion protein polynucleotide identified. Two or more generations may be grown to ensure that the deaminase or fusion protein polynucleotide is stably maintained and inherited and then seeds harvested to ensure the presence of the deaminase or fusion protein polynucleotide. In this manner, the present invention provides transformed seed (also referred to as “transgenic seed”) having a nucleotide construct of the invention, for example, an expression cassette of the invention, stably incorporated into their genome.

In some embodiments, cells that have been transformed are introduced into an organism. These cells could have originated from the organism, wherein the cells are transformed in an ex vivo approach.

The sequences provided herein may be used for transformation of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. Further provided is a processed plant product or byproduct that retains the sequences disclosed herein, including for example, soymeal.

In some embodiments, the polynucleotides encoding the deaminases, fusion proteins and/or gRNAs are used to transform any eukaryotic species, including but not limited to animals (e.g., mammals, insects, fish, birds, and reptiles), fungi, amoeba, algae, and yeast. In some embodiments, the polynucleotides encoding the deaminases, fusion proteins and/or gRNAs are used to transform any prokaryotic species, including but not limited to, archaea and bacteria (e.g., Bacillus spp., Klebsiella spp. Streptomyces spp., Rhizobium spp., Escherichia spp., Pseudomonas spp., Salmonella spp., Shigella spp., Vibrio spp., Yersinia spp., Mycoplasma spp., Agrobacterium spp., and Lactobacillus spp.).

In some embodiments, conventional viral and non-viral based gene transfer methods are used to introduce nucleic acids in mammalian cells or target tissues. Such methods can be used to administer nucleic acids encoding a deaminase or fusion protein of the invention and optionally a gRNA to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Non-limiting examples include vectors utilizing Caulimoviruses (e.g., cauliflower mosaic virus), Geminiviruses (e.g., bean golden yellow mosaic virus or maize steak virus), and RNA plant viruses (e.g., tobacco mosaic virus). For a review of gene therapy procedures, see Anderson, Science 256: 808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

Methods of non-viral delivery of nucleic acids include lipofection, Agrobacterium-mediated transformation, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired gene into the target cell to provide permanent transgene expression. Widely used retroviral vectors include those based upon murine leukemia virus (MuLV), gibbon ape leukemia virus (GaLV), Simian Immuno deficiency virus (SIV), human 55mmune deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Katin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Packaging cells are typically used to form virus particles that are capable of infecting a host cell. Such cells include 293 cells, which package adenovirus, and ψJ2 cells or PA317 cells, which package retrovirus.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

Ideally, the coding sequence of a fusion protein of the invention and a corresponding guide RNA for targeting the fusion protein may all be packaged into a single AAV vector. The generally accepted size limit for AAV vectors is 4.7 kb, although larger sizes may be contemplated at the expense of reduced packing efficiency. To ensure that the expression cassettes for both the fusion protein and its corresponding guide RNA could fit into an AAV vector, active deletion variants of RGNs may be used. In addition to shortening the amino acid sequence and therefore the coding sequence of the RGN and/or the deaminase of the fusion protein, the peptide linker which links the RGN and the deaminase may also be shortened or removed. Finally, the genetic elements, such as the promoters, enhancers, and/or terminators, may also be engineered via deletion analysis to determine the minimal size required for each to be functional. The present invention also teaches methods of using said fusion proteins for targeted base editing through in vivo AAV vector delivery.

In some embodiments, a host cell is transiently or non-transiently transfected with one or more vectors described herein. In some embodiments, a cell is transfected as it naturally occurs in a subject. In some embodiments, a cell that is transfected is taken from a subject.

In some embodiments, a cell that is transfected is a eukaryotic cell. In some embodiments, the eukaryotic cell is an animal cell (e.g., mammals, insects, fish, birds, and reptiles). In some embodiments, a cell that is transfected is a human cell. In some embodiments, a cell that is transfected is a cell of hematopoietic origin, such as an immune cell (i.e., a cell of the innate or adaptive immune system) including but not limited to a B cell, a T cell, a natural killer (NK) cell, a pluripotent stem cell, an induced pluripotent stem cell, a chimeric antigen receptor T (CAR-T) cell, a monocyte, a macrophage, and a dendritic cell. The cell that is transfected can be an allogenic cell (e.g., an allogenic T-cell) or an autologous cell (e.g., an autologous T-cell).

In some embodiments, the cell is derived from cells taken from a subject, such as a cell line. In some embodiments, the cell or cell line is prokaryotic. In some embodiments, the cell or cell line is eukaryotic. In further embodiments, the cell or cell line is derived from insect, avian, plant, or fungal species. In some embodiments, the cell or cell line may be mammalian, such as for example human, monkey, mouse, cow, swine, goat, hamster, rat, cat, or dog. A wide variety of cell lines for tissue culture are known in the art. Examples of cell lines include, but are not limited to, C8161, CCRF-CEM, MOLT, mIMCD-3, NHDF, HeLaS3, Huhl, Huh4, Huh7, HUVEC, HASMC, HEKn, HEKa, MiaPaCell, Panel, PC-3, TFl, CTLL-2, CIR, Rat6, CVI, RPTE, AlO, T24, 182, A375, ARH-77, Calul, SW480, SW620, SKOV3, SK-UT, CaCo2, P388D1, SEM-K2, WEHI-231, HB56, T1B55, lurkat, 145.01, LRMB, Bel-1, BC-3, IC21, DLD2, Raw264.7, NRK, NRK-52E, MRC5, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-Ll, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10Tl/2, C6/36, Cal-27, CHO, CHO-7, CHO-IR, CHO-Kl, CHO-K2, CHO-T, CHO Dhfr−/−, COR-L23, COR-L23/CPR, COR-L235010, CORL23/R23, COS-7, COV-434, CML Tl, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, lurkat, lY cells, K562 cells, Ku812, KCL22, KGl, KYOl, LNCap, Ma-Mel 1-48, MC-38, MCF-7, MCF-10A, MDA-MB-231, MDA-MB-468, MDA-MB-435, MDCKII, MDCKII, MOR/0.2R, MONO-MAC 6, MTD-IA, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-IA/PNT 2, RenCa, RIN-5F, RMA/RMAS, Saos-2 cells, Sf-9, SkBr3, T2, T-47D, T84, THP1 cell line, U373, U87, U937, VcaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, a cell transfected with one or more vectors described herein is used to establish a new cell line comprising one or more vector-derived sequences. In some embodiments, a cell transiently transfected with a fusion protein of the invention and optionally a gRNA, or with a ribonucleoprotein complex of the invention, and modified through the activity of a fusion protein or ribonucleoprotein complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal or transgenic plant. In some embodiments, the transgenic animal is an insect. In further embodiments, the insect is an insect pest, such as a mosquito or tick. In some embodiments, the insect is a plant pest, such as a corn rootworm or a fall armyworm. In some embodiments, the transgenic animal is a bird, such as a chicken, turkey, goose, or duck. In some embodiments, the transgenic animal is a mammal, such as a human, mouse, rat, hamster, monkey, ape, rabbit, swine, cow, horse, goat, sheep, cat, or dog.

### VII. Variants and Fragments of Polypeptides and Polynucleotides

The present disclosure provides deaminases and fusion proteins comprising the same, and fusion proteins comprising RGNs and a heterologous polypeptide (e.g., deaminases) which are active on DNA molecules. RGNs can comprise any one of SEQ ID NOs: 1-4, 49-162, 435, 575, 576, 698, and 699, active variants or fragments thereof, and polynucleotides encoding the same. Deaminases can comprise any one of SEQ ID NOs: 6, 229-414, 596, 597, and 720-723, active variants or fragments thereof, and polynucleotides encoding the same.

While the activity of a variant or fragment may be altered compared to the polynucleotide or polypeptide of interest, the variant and fragment should retain the functionality of the polynucleotide or polypeptide of interest. For example, a variant or fragment may have increased activity, decreased activity, different spectrum of activity or any other alteration in activity when compared to the polynucleotide or polypeptide of interest.

Fragments and variants of deaminases of the invention which have adenine deaminase activity will retain said activity if they are part of a fusion protein further comprising a DNA-binding polypeptide or a fragment thereof.

Fragments and variants of fusion proteins of the invention, such as fusion protein fragments and variants that comprise a fragment or variant of a deaminase or fusion protein fragments and variants that comprise a fragment or variant of a DNA-binding polypeptide (e.g., RGN), which have base editing activity will retain said activity

The term “fragment” refers to a portion of a polynucleotide or polypeptide sequence of the invention. “Fragments” or “biologically active portions” include polynucleotides comprising a sufficient number of contiguous nucleotides to retain the biological activity. “Fragments” or “biologically active portions” include polypeptides comprising a sufficient number of contiguous amino acid residues to retain the biological activity. Fragments of the RGNs or deaminases disclosed herein include those that are shorter than the full-length sequences due to the use of an alternate downstream start site. In some embodiments, a biologically active portion of a deaminase or RGN is a polypeptide that comprises, for example, 10, 20, 30, 40, 50, 60, 70, 80, 90, 100, 110, 120, 130, 140, 150, 160, or more contiguous amino acid residues of any of SEQ ID NOs: 1-4, 49-162, 435, 575, 576, 698, and 699, or a variant thereof. In some embodiments, a biologically active portion of a deaminase or RGN is a polypeptide that comprises, for example, 10, 20, 30, 40, 50, 60, 70, 80, 90, 100, 110, 120, 130, 140, 150, 160, or more contiguous amino acid residues of any of SEQ ID NOs: 5, 6, 229-414, 596, 597, and 720-723, or a variant thereof. Such biologically active portions can be prepared by recombinant techniques and evaluated for activity.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Variants of a particular polynucleotide disclosed herein will have about 40% to about 99% or more sequence identity to that particular polynucleotide as determined by sequence alignment programs and parameters described elsewhere herein. Generally, variants of a particular polynucleotide disclosed herein will have at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more sequence identity to that particular polynucleotide as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is about 40% to about 99% or more sequence identity, and in some embodiments, is at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more sequence identity.

In some embodiments, the presently disclosed polynucleotides encode a deaminase comprising an amino acid sequence having at least about 40% to about 99% or greater identity to an amino acid sequence of any of SEQ ID NOs: 5, 6, 300-414, 596-598, and 720-723, wherein the deaminase comprises at least one of the amino acid residues set forth in any one of Tables 2, 4, 6, and 19. In some embodiments, the presently disclosed polynucleotides encode a deaminase comprising an amino acid sequence having at least 40%, at least 45%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater identity to an amino acid sequence of any of SEQ ID NOs: 5, 6, 300-414, 596-598, and 720-723, wherein the deaminase comprises at least one of the amino acid residues set forth in any one of Tables 2, 4, 6, and 19.

A biologically active variant of an adenine deaminase of the invention may differ by as few as 1-15 amino acid residues, as few as 1-10, such as 6-10, as few as 5, as few as 4, as few as 3, as few as 2, or as few as 1 amino acid residue. In specific embodiments, the polypeptides comprise an N-terminal or a C-terminal truncation, which can comprise at least a deletion of 5, 10, 15, 20, 25, 30, 35, 40, 45, 50, 55, 60 amino acids or more from either the N or C terminus of the polypeptide. In some embodiments, the polypeptides comprise an internal deletion which can comprise at least a deletion of 1, 2, 3, 4, 5, 6, 7, 8, 9, 10, 11, 12, 13, 14, 15, 16, 17, 18, 19, 20, 25, 30, 35, 40, 45, 50, 55, 60 amino acids or more.

It is recognized that modifications may be made to the RGNs and deaminases provided herein creating variant proteins and polynucleotides. Changes designed by man may be introduced through the application of site-directed mutagenesis techniques. In some embodiments, native, as yet-unknown or as yet unidentified polynucleotides and/or polypeptides structurally and/or functionally-related to the sequences disclosed herein may also be identified that fall within the scope of the present invention. Conservative amino acid substitutions may be made in nonconserved regions that do not alter the function of the polypeptide as an adenine deaminase.

Variant polynucleotides and proteins also encompass sequences and proteins derived from a mutagenic and recombinogenic procedure such as DNA shuffling. With such a procedure, one or more different RGNs or deaminases is manipulated to create a new deaminase possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the sequences provided herein and other subsequently identified genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458. A “shuffled” nucleic acid is a nucleic acid produced by a shuffling procedure such as any shuffling procedure set forth herein. Shuffled nucleic acids are produced by recombining (physically or virtually) two or more nucleic acids (or character strings), for example in an artificial, and optionally recursive, fashion. Generally, one or more screening steps are used in shuffling processes to identify nucleic acids of interest; this screening step can be performed before or after any recombination step. In some (but not all) shuffling embodiments, it is desirable to perform multiple rounds of recombination prior to selection to increase the diversity of the pool to be screened. The overall process of recombination and selection are optionally repeated recursively. Depending on context, shuffling can refer to an overall process of recombination and selection, or, alternately, can simply refer to the recombinational portions of the overall process.

As used herein, “sequence identity” or “identity” in the context of two polynucleotides or polypeptide sequences makes reference to the residues in the two sequences that are the same when aligned for maximum correspondence over a specified comparison window. It is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Protein sequences that differ by such conservative substitutions are said to have “sequence similarity” or “similarity”. Means for measuring sequence similarity are well known to those of skill in the art. Typically, this involves scoring a conservative substitution as a partial rather than a full mismatch. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acid positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

### VIII. Antibodies

Antibodies to the deaminases, fusion proteins, or ribonucleoproteins of the present invention, including deaminases having the amino acid sequence set forth as any one of SEQ ID NOs: 6, 300-414, 596-598, and 720-723, or active variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265). These antibodies can be used in kits for the detection and isolation of deaminases or fusion proteins or ribonucleoproteins described herein. Thus, this disclosure provides kits comprising antibodies that specifically bind to the polypeptides or ribonucleoproteins described herein, including, for example, polypeptides comprising a sequence of at least 85% identity to any of SEQ ID NOs: 5, 6, 300-414, 596-598, and 720-723, wherein the deaminase comprises at least one of the amino acid residues set forth in any one of Tables 2, 4, 6, and 19.

IX. Systems and Ribonucleoprotein Complexes for Binding and/or Modifying a Target Sequence of Interest and Methods of Making the Same

The present disclosure provides a system which targets to a nucleic acid sequence and modifies a target nucleic acid sequence. In some embodiments, the system targets a heterologous polypeptide to a nucleic acid sequence and can modify a target nucleic acid sequence. In those embodiments wherein the heterologous polypeptide comprises a base-editing polypeptide (e.g., a deaminase) or a prime-editing polypeptide, the base-editing polypeptide (e.g., deaminase) or prime-editing polypeptide fused to the RGN (e.g., RGN nickase or nuclease-inactive RGN) is responsible for modifying the targeted nucleic acid sequence. The guide RNA hybridizes to the target sequence of interest and also forms a complex with the RGN component of the fusion protein, thereby directing the fusion protein to bind to the target sequence.

In some embodiments, an RNA-guided, DNA-binding polypeptide, such as an RGN (e.g., RGN nickase or nuclease-active RGN), and the gRNA are responsible for targeting the ribonucleoprotein complex to a nucleic acid sequence of interest and the deaminase polypeptide fused to the RGDBP is responsible for modifying the targeted nucleic acid sequence. In those embodiments wherein the deaminase is an adenine deaminase, the base editor modifies A>N. In some embodiments, the adenine deaminase converts A>G. The guide RNA hybridizes to the target sequence of interest and also forms a complex with the RNA-guided, DNA-binding polypeptide, thereby directing the RNA-guided, DNA-binding polypeptide to bind to the target sequence. The RNA-guided, DNA-binding polypeptide is part of a fusion protein that also comprises a deaminase, such as one described herein. In some embodiments, the RNA-guided, DNA-binding polypeptide is an RGN, such as a Cas9. Other examples of RNA-guided, DNA-binding polypeptides include RGNs such as those described in International Patent Application Publication Nos. WO 2019/236566 and WO 2020/139783, each of which is incorporated by reference in its entirety. In some embodiments, the RNA-guided, DNA-binding polypeptide is a Type II CRISPR-Cas polypeptide, or an active variant or fragment thereof. In some embodiments, the RNA-guided, DNA-binding polypeptide is a Type V CRISPR-Cas polypeptide, or an active variant or fragment thereof. In some embodiments, the RNA-guided, DNA-binding polypeptide is a Type VI CRISPR-Cas polypeptide. In some embodiments, the DNA-binding polypeptide of the fusion protein does not require an RNA guide, such as a zinc finger nuclease, TALEN, or meganuclease polypeptide. In some embodiments, the nuclease activity of a DNA-binding polypeptide has been partially or completely inactivated. In further embodiments, the RNA-guided, DNA-binding polypeptide comprises an amino acid sequence of an RGN, such as for example APG07433.1 (SEQ ID NO: 1), or an active variant or fragment thereof such as nickase nAPG07433.1 (SEQ ID NO: 49).

In some embodiments, the system for binding and modifying a target sequence of interest provided herein is a ribonucleoprotein complex, which is at least one molecule of an RNA bound to at least one protein (i.e., the fusion protein). The ribonucleoprotein complexes provided herein comprise at least one guide RNA as the RNA component and a fusion protein comprising an RNA-guided, DNA-binding polypeptide (e.g., RGN) and a heterologous polypeptide (e.g., deaminase) inserted therein as the protein component. In some embodiments, the ribonucleoprotein complex is purified from a cell or organism that has been transformed with polynucleotides that encode the fusion protein and a guide RNA and cultured under conditions to allow for the expression of the fusion protein and guide RNA.

Methods are provided for making a deaminase, a fusion protein, or a fusion protein ribonucleoprotein complex. Such methods comprise culturing a cell comprising a nucleotide sequence encoding a deaminase, a fusion protein, and in some embodiments a nucleotide sequence encoding a guide RNA, under conditions in which the deaminase or fusion protein (and in some embodiments, the guide RNA) is expressed. The deaminase, fusion protein, or fusion ribonucleoprotein can then be purified from a lysate of the cultured cells.

Methods for purifying a deaminase, a fusion protein, or fusion ribonucleoprotein complex from a lysate of a biological sample are known in the art (e.g., size exclusion and/or affinity chromatography, 2D-PAGE, HPLC, reversed-phase chromatography, immunoprecipitation). In particular methods, the deaminase or fusion protein is recombinantly produced and comprises a purification tag to aid in its purification, including but not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6xHis, biotin carboxyl carrier protein (BCCP), and calmodulin. Generally, the tagged deaminase, fusion protein, or fusion ribonucleoprotein complex is purified using immunoprecipitation or other similar methods known in the art.

An “isolated” or “purified” polypeptide, or biologically active portion thereof, is substantially or essentially free from components that normally accompany or interact with the polypeptide as found in its naturally occurring environment. Thus, an isolated or purified polypeptide is substantially free of other cellular material, or culture medium when produced by recombinant techniques, or substantially free of chemical precursors or other chemicals when chemically synthesized. A protein that is substantially free of cellular material includes preparations of protein having less than 30%, less than 20%, less than 10%, less than 5%, or less than 1% (by dry weight) of contaminating protein. When the protein of the invention or biologically active portion thereof is recombinantly produced, optimally culture medium represents less than 30%, less than 20%, less than 10%, less than 5%, or less than 1% (by dry weight) of chemical precursors or non-protein-of-interest chemicals.

Particular methods provided herein for binding and/or cleaving a target sequence of interest involve the use of an in vitro assembled ribonucleoprotein complex. In vitro assembly of a ribonucleoprotein complex can be performed using any method known in the art in which a fusion protein comprising an RGDBP (e.g., RGN) is contacted with a guide RNA under conditions to allow for binding of the RGDBP (e.g., RGN) fusion protein to the guide RNA. As used herein, “contact”, “contacting”, “contacted,” refer to placing the components of a desired reaction together under conditions suitable for carrying out the desired reaction. The RGDBP (e.g., RGN) fusion protein can be purified from a biological sample, cell lysate, or culture medium, produced via in vitro translation, or chemically synthesized. The guide RNA can be purified from a biological sample, cell lysate, or culture medium, transcribed in vitro, or chemically synthesized. The RGDBP (e.g., RGN) fusion protein and guide RNA can be brought into contact in solution (e.g., buffered saline solution) to allow for in vitro assembly of the ribonucleoprotein complex.

### X. Methods for Localizing a Heterologous Polypeptide to a Target DNA Molecule or Modifying a Target DNA Molecule

The present disclosure provides methods for localizing a heterologous polypeptide (e.g., deaminase) to a target nucleic acid molecule (e.g., target DNA molecule) of interest or modifying the target nucleic acid molecule. The methods include delivering a presently disclosed fusion protein or a polynucleotide encoding the same to a target sequence or a cell, organelle, or embryo comprising a target sequence. In certain embodiments, the methods include delivering a system comprising at least one guide RNA or a polynucleotide encoding the same, and at least one presently disclosed fusion protein to the target sequence or a cell, organelle, or embryo comprising the target sequence.

In some embodiments, the methods comprise contacting a DNA molecule with (a) a fusion protein; and (b) a gRNA targeting the fusion protein of (a) to a target nucleotide sequence of the DNA molecule; wherein the DNA molecule is contacted with the fusion protein and the gRNA in an amount effective and under conditions suitable for the binding of the RGBDP (e.g., RGN) component to the target sequence.

The target DNA molecule can comprise a sequence associated with a disease or disorder, and wherein the editing of at least one nucleobase within the causal mutation can result in a sequence that is not associated with a disease or disorder. In some embodiments, the disease or disorder affects animals. In further embodiments, the disease or disorder affects mammals, such as humans, cows, horses, dogs, cats, goats, sheep, swine, monkeys, rats, mice, or hamsters. In some embodiments, the target DNA sequence resides in an allele of a crop plant, wherein the particular allele of the trait of interest results in a plant of lesser agronomic value. The editing of at least one nucleobase within this region results in an allele that improves the trait and increases the agronomic value of the plant.

After delivery of a polynucleotide encoding a guide RNA and/or a fusion protein, the cell or embryo can then be cultured under conditions in which the guide RNA and/or fusion protein are expressed. In various embodiments, the method comprises contacting a target sequence with a ribonucleoprotein complex comprising a gRNA and a fusion protein. In certain embodiments, the method comprises introducing into a cell, organelle, or embryo comprising a target sequence a ribonucleoprotein complex of the invention. The ribonucleoprotein complex of the invention can be one that has been purified from a biological sample, recombinantly produced and subsequently purified, or in vitro-assembled as described herein. In those embodiments wherein the ribonucleoprotein complex that is contacted with the target sequence or a cell organelle, or embryo has been assembled in vitro, the method can further comprise the in vitro assembly of the complex prior to contact with the target sequence, cell, organelle, or embryo.

A purified or in vitro assembled ribonucleoprotein complex of the invention can be introduced into a cell, organelle, or embryo using any method known in the art, including, but not limited to electroporation. In some embodiments, a fusion protein (or polynucleotide encoding the same) and a polynucleotide encoding or comprising the guide RNA is introduced into a cell, organelle, or embryo using any method known in the art (e.g., electroporation).

Upon delivery to or contact with the target sequence or cell, organelle, or embryo comprising the target sequence, the guide RNA directs the fusion protein to bind to the target sequence in a sequence-specific manner. The target sequence can subsequently be modified in those instances wherein the fusion protein comprises a base-editing polypeptide (e.g., deaminase) or a prime editing polypeptide.

In some embodiments wherein the fusion protein is a base editor (i.e., comprises a base-editing polypeptide such as a deaminase), the binding of the fusion protein to a target sequence results in modification of a nucleotide adjacent to the target sequence. The nucleobase adjacent to the target sequence that is modified by the deaminase may be 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs from the 5′ or 3′ end of the target sequence. The full range of nucleotides that can be edited using a base editor fusion protein (e.g., RGN fused to a deaminase), usually expressed as the distance of the nucleotides from the PAM sequence (e.g., nucleotides at positions 8 and 22 upstream, i.e., 5′, from the PAM sequence), is referred to herein as the “editing window” of a particular base editor fusion protein. The editing window may be, for example, 1-100 base pairs 5′ or 3′ of the PAM sequence, including but not limited to 5-50, 5-25, 8-22, 10-20, or 10-21 base pairs 5′ or 3′ of the PAM sequence. A fusion protein comprising an adenine deaminase, such as those disclosed herein, and an RNA-guided, DNA-binding polypeptide (e.g., RGN) can introduce targeted A>N mutations in the targeted DNA molecule. In some embodiments, the fusion protein introduces targeted A>G mutations in the targeted DNA molecule.

Methods to measure binding of the fusion protein to a target sequence are known in the art and include chromatin immunoprecipitation assays, gel mobility shift assays, DNA pull-down assays, reporter assays, microplate capture and detection assays. Likewise, methods to measure cleavage or modification of a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. In some embodiments, the nicking triggered exponential amplification reaction (NTEXPAR) assay is used (see, e.g., Zhang et al. (2016) Chem. Sci. 7:4951-4957). In vivo cleavage can be evaluated using the Surveyor assay (Guschin et al. (2010) Methods Mol Biol 649:247-256).

Methods to measure base editing activity of a base editor fusion protein or prime editor fusion protein are known in the art and include those described herein and any form of sequence analysis (e.g., PCR, sequencing, or gel electrophoresis with or without the attachment of an appropriate label) of a target nucleic acid molecule after contact with a base editor or prime editor. Base editor fusion proteins comprising the deaminases disclosed herein can exhibit improved editing activity (i.e., improved editing efficiency or reduced RNA editing), a larger editing window, or both when compared to a similar base editor fusion protein comprising the parental LPG50148 deaminase. The efficiency of base editing of a first base editor fusion protein comprising the deaminases of the invention (e.g., SEQ ID NOs: 5, 6, 300-414, 596-598, and 720-723, or an active variant or fragment thereof comprising at least one of the amino acid residues set forth in any one of Tables 2, 4, 6, and 19) can have 1.1-fold to 10-fold, 1.1-fold to 20-fold, 1.1-fold to 30-fold, or more greater editing efficiency than a second base editor fusion protein comprising the parental LPG50148 deaminase and the same DNA binding polypeptide (e.g., RGN) as the first base editor fusion protein, including but not limited to about 1.1-fold, about 1.5-fold, about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 11-fold, about 12-fold, about 13-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, and about 30-fold. Base editor fusion proteins comprising the deaminases of the invention may exhibit reduced RNA editing when compared to a similar base editor fusion protein comprising the parental LPG50148 deaminase. A first base editor fusion protein comprising a deaminase of the invention can exhibit a RNA editing rate of about 1% to about 99% of a second base editor fusion protein comprising the parental LPG50148 deaminase and the same DNA binding polypeptide (e.g., RGN) as the first base editor fusion protein, including but not limited to about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%.

The editing window of a first base editor fusion protein comprising the deaminases of the invention (e.g., SEQ ID NOs: 5, 6, 300-414, 596-598, and 720-723, or an active variant or fragment thereof comprising at least one of the amino acid residues set forth in any one of Tables 2, 4, 6, and 19) may be longer by at least one nucleotide on either side or both sides of the editing window of a second base editor fusion protein comprising the parental LPG50148 deaminase and the same DNA-binding protein (e.g., RGN) as the first base editor fusion protein, including 1, 2, 3, 4, 5, 6, 7, 8, 9, 10, 11, 12, 13, 14, 15, 16, 17, 18, 19, 20, or more nucleotides longer on either side or both sides of the parental base editor editing window.

In some embodiments, the methods involve the use of a fusion protein, wherein the RGDBP (e.g., RGN) is complexed with more than one guide RNA. The more than one guide RNA can target different regions of a single gene or can target multiple genes. This multiple targeting enables the deaminase or prime editing polypeptide of the fusion protein to modify nucleic acids, thereby introducing multiple mutations in the target nucleic acid molecule (e.g., genome) of interest.

In those embodiments wherein the method involves the use of an RNA-guided nuclease (RGN), such as a nickase RGN (i.e., is only able to cleave a single strand of a double-stranded polynucleotide, for example nAPG07433.1 (SEQ ID NO: 49), the method can comprise introducing two different RGNs or RGN variants that target identical or overlapping target sequences and cleave different strands of the polynucleotide. For example, an RGN nickase that only cleaves the positive (+) strand of a double-stranded polynucleotide can be introduced along with a second RGN nickase that only cleaves the negative (−) strand of a double-stranded polynucleotide. In some embodiments, two different fusion proteins are provided, where each fusion protein comprises a different RGN with a different PAM recognition sequence, so that a greater diversity of nucleotide sequences may be targeted for mutation.

One of ordinary skill in the art will appreciate that any of the presently disclosed methods can be used to target a single target sequence or multiple target sequences. Thus, methods comprise the use of a fusion protein comprising a single RGDBP (e.g., RGN) in combination with multiple, distinct guide RNAs, which can target multiple, distinct sequences within a single gene and/or multiple genes. Also encompassed herein are methods wherein multiple, distinct guide RNAs are introduced in combination with multiple, distinct RGN fusion proteins. These guide RNAs and guide RNA/fusion protein systems can target multiple, distinct sequences within a single gene and/or multiple genes.

In some embodiments, a fusion protein comprising an RNA-guided, DNA-binding polypeptide (e.g., RGN) and a heterologous polypeptide (e.g., deaminase) may be used for generating mutations in a targeted gene or targeted region of a gene of interest. In some embodiments, a fusion protein of the invention may be used for saturation mutagenesis of a targeted gene or region of a targeted gene of interest followed by high-throughput forward genetic screening to identify novel mutations and/or phenotypes. In some embodiments, a fusion protein described herein may be used for generating mutations in a targeted genomic location, which may or may not comprise coding DNA sequence. Libraries of cell lines generated by the targeted mutagenesis described above may also be useful for study of gene function or gene expression.

### XI. Target Polynucleotides

In one aspect, the invention provides for methods of modifying a target polynucleotide in a eukaryotic cell, which may be in vivo, ex vivo or in vitro. In some embodiments, the method comprises sampling a cell or population of cells from a human or non-human animal or plant (including microalgae) and modifying the cell or cells. Culturing may occur at any stage ex vivo. The cell or cells may even be re-introduced into the human, non-human animal or plant (including micro-algae).

Using natural variability, plant breeders combine most useful genes for desirable qualities, such as yield, quality, uniformity, hardiness, and resistance against pests. These desirable qualities also include growth, day length preferences, temperature requirements, initiation date of floral or reproductive development, fatty acid content, insect resistance, disease resistance, nematode resistance, fungal resistance, herbicide resistance, tolerance to various environmental factors including drought, heat, wet, cold, wind, and adverse soil conditions including high salinity. The sources of these useful genes include native or foreign varieties, heirloom varieties, wild plant relatives, and induced mutations, e.g., treating plant material with mutagenic agents. Using the present invention, plant breeders are provided with a new tool to induce mutations. Accordingly, one skilled in the art can employ the present invention to induce the rise of useful genes, with more precision than previous mutagenic agents and hence accelerate and improve plant breeding programs.

The target polynucleotide of a fusion protein of the invention can be any polynucleotide endogenous or exogenous to the eukaryotic cell. For example, the target polynucleotide can be a polynucleotide residing in the nucleus of the eukaryotic cell. In some embodiments, the target polynucleotide is a sequence coding a gene product (e.g., a protein) or a non-coding sequence (e.g., a regulatory polynucleotide or a junk DNA). In some embodiments, the target sequence for a fusion protein of the invention is associated with a PAM (protospacer adjacent motif); that is, a short sequence recognized by the RNA-guided DNA-binding polypeptide (e.g., RGN). The precise sequence and length requirements for the PAM differ depending on the RNA-guided DNA-binding polypeptide used, but PAMs are typically 2-5 base pair sequences adjacent the protospacer (that is, the target sequence).

The target polynucleotide of a fusion protein of the invention may include a number of disease-associated genes and polynucleotides as well as signaling biochemical pathway-associated genes and polynucleotides. Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissue compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease (e.g., a causal mutation). The transcribed or translated products may be known or unknown, and further may be at a normal or abnormal level.

Non-limiting examples of disease-associated genes that can be targeted using the presently disclosed methods and compositions are available from McKusick-Nathans Institute of Genetic Medicine, Johns Hopkins University (Baltimore, Md.) and National Center for Biotechnology Information, National Library of Medicine (Bethesda, Md.), available on the World Wide Web.

In some embodiments, the methods comprise contacting a DNA molecule comprising a target DNA sequence with a fusion protein of the invention, wherein the DNA molecule is contacted with the fusion protein in an amount effective and under conditions suitable for the modification of the target DNA molecule. In certain embodiments, the methods comprise contacting a DNA molecule comprising a target DNA sequence with (a) an RGN fusion protein of the invention comprising a base-editing polypeptide; and (b) a gRNA targeting the fusion protein of (a) to a target nucleotide sequence of the DNA strand; wherein the DNA molecule is contacted with the fusion protein and the gRNA in an amount effective and under conditions suitable for the deamination of at least one nucleobase. In some embodiments, the target DNA sequence comprises a sequence associated with a disease or disorder, and wherein the deamination of the nucleobase results in a sequence that is not associated with a disease or disorder. In some embodiments, the target DNA sequence resides in an allele of a crop plant, wherein the particular allele of the trait of interest results in a plant of lesser agronomic value. The deamination of the nucleobase results in an allele that improves the trait and increases the agronomic value of the plant.

In some embodiments, the target DNA sequence comprises a point mutation associated with a disease or disorder, and wherein the deamination of the mutant base results in a sequence that is not associated with a disease or disorder. In some embodiments, the deamination corrects a point mutation in the sequence associated with the disease or disorder.

In some embodiments, the sequence associated with the disease or disorder encodes a protein, and the deamination or prime editing introduces a stop codon into the sequence associated with the disease or disorder, resulting in a truncation of the encoded protein. In some embodiments, the contacting is performed in vivo in a subject susceptible to having, having, or diagnosed with the disease or disorder. In some embodiments, the disease or disorder is a disease associated with a point mutation, or a single-base mutation, in the genome. In some embodiments, the disease is a genetic disease, a cancer, a metabolic disease, or a lysosomal storage disease.

### XII. Pharmaceutical Compositions and Methods of Treatment

Methods of treating a disease in a subject in need thereof are provided herein. The methods comprise administering to a subject in need thereof a presently disclosed fusion protein or a polynucleotide encoding the same, a gRNA or a polynucleotide encoding the same, a presently disclosed fusion protein system, or a cell modified by or comprising any one of these compositions.

In some embodiments, the treatment comprises in vivo gene editing by administering to a subject in need thereof a presently disclosed fusion protein, gRNA, or a presently disclosed fusion protein system or polynucleotide(s) encoding the same. In some embodiments, the treatment comprises ex vivo gene editing wherein cells are genetically modified ex vivo with a presently disclosed fusion protein, gRNA, or a presently disclosed fusion protein system or polynucleotide(s) encoding the same and then the modified cells are administered to a subject. In some embodiments, the genetically modified cells originate from the subject that is then administered the modified cells, and the transplanted cells are referred to herein as autologous. In some embodiments, the genetically modified cells originate from a different subject (i.e., donor) within the same species as the subject that is administered the modified cells (i.e., recipient), and the transplanted cells are referred to herein as allogeneic. In some examples described herein, the cells can be expanded in culture prior to administration to a subject in need thereof.

In some embodiments, the disease to be treated with the presently disclosed compositions is one that can be treated with immunotherapy, such as with a chimeric antigen receptor (CAR) T cell. Such diseases include but are not limited to cancer.

In some embodiments, the modification (e.g., deamination) of a target sequence results in the correction of a genetic defect or in the correction of a point mutation that leads to a loss of function in a gene product. In some embodiments, the genetic defect is associated with a disease or disorder, e.g., a lysosomal storage disorder or a metabolic disease, such as, for example, type I diabetes. Thus, in some embodiments, the disease to be treated with the presently disclosed compositions is associated with a sequence (i.e., the sequence is causal for the disease or disorder or causal for symptoms associated with the disease or disorder) that is mutated in order to treat the disease or disorder or the reduction of symptoms associated with the disease or disorder.

In some embodiments, the disease to be treated with the presently disclosed compositions is associated with a causal mutation. As used herein, a “causal mutation” refers to a particular nucleotide, nucleotides, or nucleotide sequence in the genome that contributes to the severity or presence of a disease or disorder in a subject. The correction of the causal mutation leads to the improvement of at least one symptom resulting from a disease or disorder. In some embodiments, the correction of the causal mutation leads to the improvement of at least one symptom resulting from a disease or disorder. In some embodiments, the causal mutation is adjacent to a PAM site recognized by the RGDBP (e.g., RGN) of a fusion protein disclosed herein. The causal mutation can be corrected with the fusion protein. Non-limiting examples of disease-associated genes and mutations are available from McKusick-Nathans Institute of Genetic Medicine, Johns Hopkins University (Baltimore, Md.) and National Center for Biotechnology Information, National Library of Medicine (Bethesda, Md.), available on the World Wide Web.

In some embodiments, the methods provided herein are used to introduce a deactivating point mutation into a gene or allele that encodes a gene product that is associated with a disease or disorder. For example, in some embodiments, methods are provided herein that employ a fusion protein to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein. In some embodiments, the purpose of the methods provided herein is to restore the function of a dysfunctional gene via genome editing. The fusion proteins provided herein can be validated for gene editing-based human therapeutics in vitro, e.g., by correcting a disease associated mutation in human cell culture. It will be understood by the skilled artisan that the fusion proteins provided herein, e.g., the fusion proteins comprising an RNA-guided, DNA-binding polypeptide and an adenine deaminase polypeptide can be used to correct any single point A>G mutation. Deamination of the mutant A to G leads to a correction of the mutation.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, and the delivery system in which it is carried.

The term “administering” refers to the placement of an active ingredient into a subject, by a method or route that results in at least partial localization of the introduced active ingredient at a desired site, such as a site of injury or repair, such that a desired effect(s) is produced. In those embodiments wherein cells are administered, the cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the lifetime of the patient, i.e., long-term engraftment. For example, in some aspects described herein, an effective amount of photoreceptor cells or retinal progenitor cells is administered via a systemic route of administration, such as an intraperitoneal or intravenous route.

In some embodiments, the administering comprises administering by viral delivery. In some embodiments, the administering comprises administering by electroporation. In some embodiments, the administering comprises administering by nanoparticle delivery. In some embodiments, the administering comprises administering by liposome delivery. In some embodiments, the administering comprises administering by lipid nanoparticle (LNP) delivery. In some embodiments, the LNP or lipid components thereof is described in WO2022173531, WO2022150485, or U.S. Provisional Appl. No. 63/492,537 filed Mar. 28, 2023, the contents of each of which is herein incorporated by reference in its entirety. Any effective route of administration can be used to administer an effective amount of a pharmaceutical composition described herein. In some embodiments, the administering comprises administering by a method selected from the group consisting of: intravenously, subcutaneously, intramuscularly, orally, rectally, by aerosol, parenterally, ophthalmicly, pulmonarily, transdermally, vaginally, otically, nasally, and by topical administration, or any combination thereof. In some embodiments, for the delivery of cells, administration by injection or infusion is used.

As used herein, the term “subject” refers to any individual for whom diagnosis, treatment or therapy is desired. In some embodiments, the subject is an animal. In some embodiments, the subject is a mammal. In some embodiments, the subject is a human being. The human being can be an adult, adolescent, child, or infant.

The efficacy of a treatment can be determined by the skilled clinician. However, a treatment is considered an “effective treatment,” if any one or all of the signs or symptoms of a disease or disorder are altered in a beneficial manner (e.g., decreased by at least 10%), or other clinically accepted symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art. Treatment includes: (1) inhibiting the disease, e.g., arresting, or slowing the progression of symptoms; or (2) relieving the disease, e.g., causing regression of symptoms; and (3) preventing or reducing the likelihood of the development of symptoms.

Pharmaceutical compositions comprising the presently disclosed fusion proteins or polynucleotides encoding the same, the presently disclosed systems, or cells comprising any of the fusion proteins, polynucleotides encoding the same, or the systems, and a pharmaceutically acceptable carrier are provided.

As used herein, a “pharmaceutically acceptable carrier” refers to a material that does not cause significant irritation to an organism and does not abrogate the activity and properties of the active ingredient (e.g., a deaminase or fusion protein or nucleic acid molecule encoding the same). Carriers must be of sufficiently high purity and of sufficiently low toxicity to render them suitable for administration to a subject being treated. The carrier can be inert, or it can possess pharmaceutical benefits. In some embodiments, a pharmaceutically acceptable carrier comprises one or more compatible solid or liquid filler, diluents or encapsulating substances which are suitable for administration to a human or other vertebrate animal. In some embodiments, the pharmaceutical composition comprises a pharmaceutically acceptable carrier that is non-naturally occurring. In some embodiments, the pharmaceutically acceptable carrier and the active ingredient are not found together in nature and are thus, heterologous.

Pharmaceutical compositions used in the presently disclosed methods can be formulated with suitable carriers, excipients, and other agents that provide suitable transfer, delivery, tolerance, and the like. A multitude of appropriate formulations are known to those skilled in the art. See, e.g., Remington, The Science and Practice of Pharmacy (21^{st} ed. 2005). Non-limiting examples include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. Administered intravenously, particular carriers are physiological saline or phosphate buffered saline (PBS). Pharmaceutical compositions for oral or parenteral use may be prepared into dosage forms in a unit dose suited to fit a dose of the active ingredients. Such dosage forms in a unit dose include, for example, tablets, pills, capsules, injections (ampoules), suppositories, etc. These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

In some embodiments wherein cells comprising or modified with the presently disclosed fusion proteins, systems, or polynucleotides encoding the same are administered to a subject, the cells are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the cells described herein using routine experimentation.

A cell composition can also be emulsified or presented as a liposome composition, provided that the emulsification procedure does not adversely affect cell viability. The cells and any other active ingredient can be mixed with excipients that are pharmaceutically acceptable and compatible with the active ingredient, and in amounts suitable for use in the therapeutic methods described herein.

Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

### XIII. Cells Comprising a Polynucleotide Genetic Modification

Provided herein are cells and organisms comprising a target nucleic acid molecule of interest that has been modified using a process mediated by a fusion protein, optionally with a gRNA, as described herein.

In some embodiments, the fusion protein comprises a deaminase polypeptide comprising an amino acid sequence of any of SEQ ID NOs: 5, 6, 300-414, 596-598, and 720-723, or an active variant or fragment thereof comprising at least one of the amino acid residues set forth in any one of Tables 2, 4, 6, and 19. In some embodiments, the fusion protein comprises a deaminase comprising an amino acid sequence having about 50% to about 99% identity or more to any of SEQ ID NOs: 5, 6, 300-414, 596-598, and 720-723. In some embodiments, the fusion protein comprises a deaminase comprising an amino acid sequence having at least 50%, at least 55%, at least 60%, at least 65%, at least 70%, at least 75%, at least 80%, at least 85%, at least 90%, at least 95%, at least 96%, at least 97%, at least 98%, or at least 99% identity to any of SEQ ID NOs: 5, 6, 300-414, 596-598, and 720-723. In some embodiments, the fusion protein comprises a deaminase and a DNA-binding polypeptide (e.g., an RNA-guided, DNA-binding polypeptide). In further embodiments, the fusion protein comprises a deaminase and an RGN or a variant thereof, such as for example APG07433.1 (SEQ ID NO: 1) or its nickase variant nAPG07433.1 (SEQ ID NO: 49). In some embodiments, the fusion protein comprises a deaminase and a Cas9 or a variant thereof, such as for example dCas9 or nickase Cas9. In some embodiments, the fusion protein comprises a nuclease-inactive or nickase variant of a Type II CRISPR-Cas polypeptide. In some embodiments, the fusion protein comprises a nuclease-inactive or nickase variant of a Type V CRISPR-Cas polypeptide. In some embodiments, the fusion protein comprises a nuclease-inactive or nickase variant of a Type VI CRISPR-Cas polypeptide.

The modified cells can be eukaryotic (e.g., mammalian, plant, insect, avian cell) or prokaryotic. Also provided are organelles and embryos comprising at least one nucleotide sequence that has been modified by a process utilizing a fusion protein as described herein. The genetically modified cells, organisms, organelles, and embryos can be heterozygous or homozygous for the modified nucleotide sequence. The mutation(s) introduced by the fusion protein can result in altered expression (up-regulation or down-regulation), inactivation, or the expression of an altered protein product or an integrated sequence. In those instances wherein the mutation(s) results in either the inactivation of a gene or the expression of a non-functional protein product, the genetically modified cell, organism, organelle, or embryo is referred to as a “knock out”. The knock out phenotype can be the result of a deletion mutation (i.e., deletion of at least one nucleotide), an insertion mutation (i.e., insertion of at least one nucleotide), or a nonsense mutation (i.e., substitution of at least one nucleotide such that a stop codon is introduced).

In some embodiments, the mutation(s) introduced by the fusion protein results in the production of a variant protein product. The expressed variant protein product can have at least one amino acid substitution and/or the addition or deletion of at least one amino acid. The variant protein product can exhibit modified characteristics or activities when compared to the wild-type protein, including but not limited to altered enzymatic activity or substrate specificity.

In some embodiments, the mutation(s) introduced by the fusion protein result in an altered expression pattern of a protein. As a non-limiting example, mutation(s) in the regulatory regions controlling the expression of a protein product can result in the overexpression or downregulation of the protein product or an altered tissue or temporal expression pattern.

The cells that have been modified can be grown into an organism, such as a plant, in accordance with conventional ways. See, for example, McCormick et al. (1986) Plant Cell Reports 5:81-84. These plants may then be grown, and either pollinated with the same modified strain or different strains, and the resulting hybrid having the genetic modification. The present invention provides genetically modified seed. Progeny, variants, and mutants of the regenerated plants are also included within the scope of the invention, provided that these parts comprise the genetic modification. Further provided is a processed plant product or byproduct that retains the genetic modification, including for example, soymeal.

The methods provided herein may be used for modification of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

The methods provided herein can also be used to genetically modify any prokaryotic species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

The methods provided herein can be used to genetically modify any eukaryotic species or cells therefrom, including but not limited to animals (e.g., mammals, insects, fish, birds, and reptiles), fungi, amoeba, algae, and yeast. In some embodiments, the cell that is modified by the presently disclosed methods include cells of hematopoietic origin, such as immune cells (i.e., a cell of the innate or adaptive immune system) including but not limited to B cells, T cells, natural killer (NK) cells, pluripotent stem cells, induced pluripotent stem cells, chimeric antigen receptor T (CAR-T) cells, monocytes, macrophages, and dendritic cells.

Cells that have been modified may be introduced into an organism. These cells could have originated from the same organism (e.g., person) in the case of autologous cellular transplants, wherein the cells are modified in an ex vivo approach. In some embodiments, the cells originated from another organism within the same species (e.g., another person) in the case of allogeneic cellular transplants.

### XIV. Kits

Some aspects of this disclosure provide kits comprising a deaminase or fusion protein of the invention. In certain embodiments, the disclosure provides kits comprising a fusion protein and guide RNAs. In addition, in some embodiments, the kit comprises suitable reagents, buffers, and/or instructions for using the fusion protein, e.g., for in vitro or in vivo DNA or RNA editing. In some embodiments, the kit comprises instructions regarding the design and use of suitable gRNAs for targeted editing of a nucleic acid sequence.

The article “a” and “an” are used herein to refer to one or more than one (i.e., to at least one) of the grammatical object of the article. By way of example, “a polypeptide” means one or more polypeptides.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated herein by reference.

Although the foregoing invention has been described in some detail by way of illustration and example for purposes of clarity of understanding, it will be obvious that certain changes and modifications may be practiced within the scope of the appended claims.

Non-limiting embodiments include:
1. A fusion protein comprising an RNA-guided nuclease (RGN) and at least one heterologous polypeptide inserted therein, wherein said RGN comprises an amino acid sequence with at least 90% sequence identity to any one of SEQ ID NOs: 1-4, 49-162, 435, 575, 576, 698, and 699.
2. The fusion protein of embodiment 1, wherein said RGN comprises an amino acid sequence with at least 95% sequence identity to any one of SEQ ID NOs: 1-4, 49-162, 435, 575, 576, 698, and 699.
3. The fusion protein of embodiment 1, wherein said RGN comprises the amino acid sequence of any one of SEQ ID NOs: 1-4, 49-162, 435, 575, 576, 698, and 699.
4. The fusion protein of embodiment 1, wherein said RGN comprises an amino acid sequence with at least 90% sequence identity to any one of SEQ ID NOs: 1-4 and 57-139.
5. The fusion protein of embodiment 1, wherein said RGN comprises an amino acid sequence with at least 95% sequence identity to any one of SEQ ID NOs: 1-4 and 57-139.
6. The fusion protein of embodiment 1, wherein said RGN comprises the amino acid sequence of any one of SEQ ID NOs: 1-4 and 57-139.
7. The fusion protein of any one of embodiments 1-6, wherein said heterologous polypeptide is inserted within a linker domain 2, a wedge domain, a RuvC domain, an HNH domain, a Rec-2 domain, or a PAM-interacting domain of said RGN.
8. The fusion protein of embodiment 7, wherein said RuvC domain is a RuvCIII domain.
9. The fusion protein of any one of claims 1-8, wherein:
a) said RGN comprises an amino acid sequence with at least 90% sequence identity to SEQ ID NO: 1 and said at least one heterologous polypeptide is inserted within said RGN immediately after the amino acid position selected from the group consisting of:
   i) amino acid position corresponding to position 30 of SEQ ID NO: 1;
   ii) amino acid position corresponding to position 642 of SEQ ID NO: 1;
   iii) amino acid position corresponding to position 670 of SEQ ID NO: 1;
   iv) amino acid position corresponding to position 737 of SEQ ID NO: 1;
   v) amino acid position corresponding to position 772 of SEQ ID NO: 1;
   vi) amino acid position corresponding to position 775 of SEQ ID NO: 1;
   vii) amino acid position corresponding to position 778 of SEQ ID NO: 1; and
   viii) amino acid position corresponding to position 802 of SEQ ID NO: 1;
b) said RGN comprises an amino acid sequence with at least 90% sequence identity to SEQ ID NO: 2 and said at least one heterologous polypeptide is inserted within said RGN immediately after the amino acid position selected from the group consisting of:
   i) amino acid position corresponding to position 678 of SEQ ID NO: 2;
   ii) amino acid position corresponding to position 736 of SEQ ID NO: 2;
   iii) amino acid position corresponding to position 778 of SEQ ID NO: 2;
   iv) amino acid position corresponding to position 788 of SEQ ID NO: 2; and
   v) amino acid position corresponding to position 922 of SEQ ID NO: 2;
c) said RGN comprises an amino acid sequence with at least 90% sequence identity to SEQ ID NO: 3 and said at least one heterologous polypeptide is inserted within said RGN immediately after the amino acid position selected from the group consisting of:
   i) amino acid position corresponding to position 725 of SEQ ID NO: 3;
   ii) amino acid position corresponding to position 739 of SEQ ID NO: 3; and
   iii) amino acid position corresponding to position 744 of SEQ ID NO: 3;
d) said RGN comprises an amino acid sequence with at least 90% sequence identity to SEQ ID NO: 4 and said at least one heterologous polypeptide is inserted within said RGN immediately after the amino acid position selected from the group consisting of:
   i) amino acid position corresponding to position 347 of SEQ ID NO: 4;
   ii) amino acid position corresponding to position 524 of SEQ ID NO: 4;
   iii) amino acid position corresponding to position 666 of SEQ ID NO: 4;
   iv) amino acid position corresponding to position 680 of SEQ ID NO: 4;
   v) amino acid position corresponding to position 740 of SEQ ID NO: 4;
   vi) amino acid position corresponding to position 785 of SEQ ID NO: 4;
   vii) amino acid position corresponding to position 910 of SEQ ID NO: 4; and
   viii) amino acid position corresponding to position 1077 of SEQ ID NO: 4; or
e) said RGN comprises an amino acid sequence with at least 90% sequence identity to SEQ ID NO: 131 and said at least one heterologous polypeptide is inserted within said RGN immediately after the amino acid position selected from the group consisting of:
   i) amino acid position corresponding to position 766 of SEQ ID NO: 131; and
   ii) amino acid position corresponding to position 806 of SEQ ID NO: 131.
10. The fusion protein of any one of embodiments 1-9, wherein said heterologous polypeptide comprises a base-editing polypeptide or a prime editing polypeptide.
11. The fusion protein of embodiment 10, wherein said prime editing polypeptide comprises a DNA polymerase.
12. The fusion protein of embodiment 10, wherein said prime editing polypeptide is a reverse transcriptase.
13. The fusion protein of embodiment 10, wherein said base-editing polypeptide comprises a deaminase.
14. The fusion protein of embodiment 13, wherein said deaminase is a cytosine deaminase or an adenine deaminase.
15. The fusion protein of embodiment 13 or 14, wherein said fusion protein is a base editor fusion protein, and wherein said base editor fusion protein has improved editing activity in comparison to a parental base editor comprising said deaminase fused to the amino terminus of said RGN.
16. The fusion protein of embodiments 13-15, wherein said at least one heterologous polypeptide is inserted within an RGN comprises an amino acid sequence with at least 90% sequence identity to SEQ ID NO: 1 immediately after the amino acid position selected from the group consisting of:
a) amino acid position corresponding to position 642 of SEQ ID NO: 1;
b) amino acid position corresponding to position 670 of SEQ ID NO: 1;
c) amino acid position corresponding to position 737 of SEQ ID NO: 1;
d) amino acid position corresponding to position 772 of SEQ ID NO: 1;
e) amino acid position corresponding to position 775 of SEQ ID NO: 1; and
f) amino acid position corresponding to position 778 of SEQ ID NO: 1; and
wherein said fusion protein is a base editor fusion protein, and wherein said base editor fusion protein has an editing window that is shifted in comparison to a parental base editor comprising said deaminase fused to the amino terminus of said RGN.
17. The fusion protein of any one of embodiments 13-16, wherein said deaminase lacks the first and last amino acid residues in comparison to a parental deaminase from which said deaminase is derived.
18. The fusion protein of any one of embodiments 13-17, wherein said deaminase comprises an adenine deaminase and said fusion protein is an adenosine base editor (ABE) fusion protein.
19. The fusion protein of embodiment 18, wherein said adenine deaminase comprises an amino acid sequence with at least 90% sequence identity to any one of SEQ ID NOs: 5, 6, 248-414, 596, 597, and 720-723.
20. The fusion protein of embodiment 18, wherein said adenine deaminase comprises an amino acid sequence with at least 95% sequence identity to any one of SEQ ID NOs: 5, 6, 248-414, 596, 597, and 720-723.
21. The fusion protein of embodiment 18, wherein said adenine deaminase comprises the amino acid sequence of any one of SEQ ID NOs: 5, 6, 248-414, 596, 597, and 720-723.
22. The fusion protein of embodiment 18, wherein said adenine deaminase comprises an amino acid sequence with at least 90% sequence identity to SEQ ID NO: 5 or 6.
23. The fusion protein of embodiment 18, wherein said adenine deaminase comprises an amino acid sequence with at least 95% sequence identity to SEQ ID NO: 5 or 6.
24. The fusion protein of embodiment 18, wherein said adenine deaminase comprises the amino acid sequence of SEQ ID NO: 5 or 6.
25. The fusion protein of embodiment 18, wherein said ABE fusion protein comprises an amino acid sequence with at least 90% sequence identity to any one of SEQ ID NOs: 8-10, 13-21, 27-31, 36-38, 40-41, 43-48, 647, and 650.
26. The fusion protein of embodiment 18, wherein said ABE fusion protein comprises an amino acid sequence with at least 95% sequence identity to any one of SEQ ID NOs: 8-10, 13-21, 27-31, 36-38, 40-41, 43-48, 647, and 650.
27. The fusion protein of embodiment 18, wherein said ABE fusion protein comprises the amino acid sequence set forth in any one of SEQ ID NOs: 8-10, 13-21, 27-31, 36-38, 40-41, 43-48, 647, and 650.
28. The fusion protein of embodiment 18, wherein said ABE fusion protein comprises an amino acid sequence with at least 90% sequence identity to any one of SEQ ID NOs: 8-10 and 14-16, and wherein said ABE fusion protein has an editing window that is shifted in comparison to a parental ABE base editor comprising said adenine deaminase fused to the amino terminus of said RGN.
29. The fusion protein of embodiment 18, wherein said ABE fusion protein has at least 95% sequence identity to any one of SEQ ID NOs: 8-10 and 14-16, and wherein said ABE fusion protein has an editing window that is shifted in comparison to a parental ABE base editor comprising said adenine deaminase fused to the amino terminus of said RGN.
30. The fusion protein of embodiment 18, wherein said ABE fusion protein comprises the amino acid sequence set forth in any one of SEQ ID NOs: 8-10 and 14-16.
31. The fusion protein of any one of embodiments 1-30, wherein said RGN is a nickase or a nuclease-inactive RGN.
32. The fusion protein of embodiment 31, wherein said RGN nickase comprises an amino acid sequence with at least 95% sequence identity to any one of SEQ ID NOs: 49-56, 698, and 699 and retains nickase activity.
33. The fusion protein of embodiment 31, wherein said RGN nickase comprises the amino acid sequence of any one of SEQ ID NOs: 49-56, 698, and 699.
34. The fusion protein of any one of embodiments 1-33, wherein said RGN and said heterologous polypeptide are directly fused to one another with no linker sequences in between.
35. The fusion protein of any of embodiments 1-34, wherein the fusion protein further comprises at least one nuclear localization signal (NLS).
36. A ribonucleoprotein (RNP) complex comprising the fusion protein of any one of embodiments 1-35 and 232-243 and a guide RNA bound to the fusion protein.
37. A nucleic acid molecule comprising a polynucleotide encoding a fusion protein, wherein said fusion protein comprises an RNA-guided nuclease (RGN) and at least one heterologous polypeptide inserted therein, wherein said RGN comprises an amino acid sequence with at least 90% sequence identity to any one of SEQ ID NOs: 1-4, 49-162, 435, 575, 576, 698, and 699.
38. The nucleic acid molecule of embodiment 37, wherein said RGN comprises an amino acid sequence with at least 95% sequence identity to any one of SEQ ID NOs: 1-4, 49-162, 435, 575, 576, 698, and 699.
39. The nucleic acid molecule of embodiment 37, wherein said RGN comprises the amino acid sequence of any one of SEQ ID NOs: 1-4, 49-162, 435, 575, 576, 698, and 699.
40. The nucleic acid molecule of embodiment 37, wherein said RGN comprises an amino acid sequence with at least 90% sequence identity to any one of SEQ ID NOs: 1-4, 57-139.
41. The nucleic acid molecule of embodiment 37, wherein said RGN comprises an amino acid sequence with at least 95% sequence identity to any one of SEQ ID NOs: 1-4, 57-139.
42. The nucleic acid molecule of embodiment 37, wherein said RGN comprises the amino acid sequence of any one of SEQ ID NOs: 1-4, 57-139.
43. The nucleic acid molecule of any one of embodiments 1-6, wherein said heterologous polypeptide is inserted within a linker domain 2, a wedge domain, a RuvC domain, an HNH domain, a Rec-2 domain, or a PAM-interacting domain of said RGN.
44. The nucleic acid molecule of embodiment 7, wherein said RuvC domain is a RuvCIII domain.
45. The nucleic acid molecule of any one of embodiments 37-44, wherein:
a) said RGN comprises an amino acid sequence with at least 90% sequence identity to SEQ ID NO: 1 and said at least one heterologous polypeptide is inserted within said RGN immediately after the amino acid position selected from the group consisting of:
   i) amino acid position corresponding to position 30 of SEQ ID NO: 1;
   ii) amino acid position corresponding to position 642 of SEQ ID NO: 1;
   iii) amino acid position corresponding to position 670 of SEQ ID NO: 1;
   iv) amino acid position corresponding to position 737 of SEQ ID NO: 1;
   v) amino acid position corresponding to position 772 of SEQ ID NO: 1;
   vi) amino acid position corresponding to position 775 of SEQ ID NO: 1;
   vii) amino acid position corresponding to position 778 of SEQ ID NO: 1; and
   viii) amino acid position corresponding to position 802 of SEQ ID NO: 1;
b) said RGN comprises an amino acid sequence with at least 90% sequence identity to SEQ ID NO: 2 and said at least one heterologous polypeptide is inserted within said RGN immediately after the amino acid position selected from the group consisting of:
   i) amino acid position corresponding to position 678 of SEQ ID NO: 2;
   ii) amino acid position corresponding to position 736 of SEQ ID NO: 2;
   iii) amino acid position corresponding to position 778 of SEQ ID NO: 2;
   iv) amino acid position corresponding to position 788 of SEQ ID NO: 2; and
   v) amino acid position corresponding to position 922 of SEQ ID NO: 2;
c) said RGN comprises an amino acid sequence with at least 90% sequence identity to SEQ ID NO: 3 and said at least one heterologous polypeptide is inserted within said RGN immediately after the amino acid position selected from the group consisting of:
   i) amino acid position corresponding to position 725 of SEQ ID NO: 3;
   ii) amino acid position corresponding to position 739 of SEQ ID NO: 3; and
   iii) amino acid position corresponding to position 744 of SEQ ID NO: 3;
d) said RGN comprises an amino acid sequence with at least 90% sequence identity to SEQ ID NO: 4 and said at least one heterologous polypeptide is inserted within said RGN immediately after the amino acid position selected from the group consisting of:
   i) amino acid position corresponding to position 347 of SEQ ID NO: 4;
   ii) amino acid position corresponding to position 524 of SEQ ID NO: 4;
   iii) amino acid position corresponding to position 666 of SEQ ID NO: 4;
   iv) amino acid position corresponding to position 680 of SEQ ID NO: 4;
   v) amino acid position corresponding to position 740 of SEQ ID NO: 4;
   vi) amino acid position corresponding to position 785 of SEQ ID NO: 4;
   vii) amino acid position corresponding to position 910 of SEQ ID NO: 4; and
   viii) amino acid position corresponding to position 1077 of SEQ ID NO: 4; or
e) said RGN comprises an amino acid sequence with at least 90% sequence identity to SEQ ID NO: 131 and said at least one heterologous polypeptide is inserted within said RGN immediately after the amino acid position selected from the group consisting of:
   i) amino acid position corresponding to position 766 of SEQ ID NO: 131; and
   ii) amino acid position corresponding to position 806 of SEQ ID NO: 131.
46. The nucleic acid molecule of embodiment 45, wherein said heterologous polypeptide comprises a base-editing polypeptide or a prime editing polypeptide.
47. The nucleic acid molecule of embodiment 46, wherein said prime editing polypeptide comprises a DNA polymerase.
48. The nucleic acid molecule of embodiment 46, wherein said prime editing polypeptide comprises a reverse transcriptase.
49. The nucleic acid molecule of embodiment 46, wherein said base-editing polypeptide comprises a deaminase.
50. The nucleic acid molecule of embodiment 49, wherein said deaminase is a cytosine deaminase or an adenine deaminase.
51. The nucleic acid molecule of embodiment 49 or 50, wherein said fusion protein is a base editor fusion protein, and wherein said base editor fusion protein has improved editing activity in comparison to a parental base editor comprising said deaminase fused to the amino terminus of said RGN.
52. The nucleic acid molecule of any one of embodiments 49-51, wherein said at least one heterologous polypeptide is inserted within said RGN comprising an amino acid sequence with at least 90% sequence identity to SEQ ID NO: 1 immediately after the amino acid position selected from the group consisting of:
a) amino acid position corresponding to position 642 of SEQ ID NO: 1;
b) amino acid position corresponding to position 670 of SEQ ID NO: 1;
c) amino acid position corresponding to position 737 of SEQ ID NO: 1;
d) amino acid position corresponding to position 772 of SEQ ID NO: 1;
e) amino acid position corresponding to position 775 of SEQ ID NO: 1; and
f) amino acid position corresponding to position 778 of SEQ ID NO: 1; and
wherein said fusion protein is a base editor fusion protein, and wherein said base editor fusion protein has an editing window that is shifted in comparison to a parental base editor comprising said deaminase fused to the amino terminus of said RGN.
53. The nucleic acid molecule of any one of embodiments 49-52, wherein said deaminase lacks the first and last amino acid residues in comparison to a parental deaminase from which said deaminase is derived.
54. The nucleic acid molecule of any one of embodiments 49-53, wherein said deaminase comprises an adenine deaminase and is an adenosine base editor (ABE) fusion protein.
55. The nucleic acid molecule of embodiment 54, wherein said adenine deaminase comprises an amino acid sequence with at least 90% sequence identity to any one of SEQ ID NOs: 5, 6, 248-414, 596, 597, and 720-723.
56. The nucleic acid molecule of embodiment 54, wherein said adenine deaminase comprises an amino acid sequence with at least 95% sequence identity to any one of SEQ ID NOs: 5, 6, 248-414, 596, 597, and 720-723.
57. The nucleic acid molecule of embodiment 54, wherein said adenine deaminase comprises the amino acid sequence of any one of SEQ ID NOs: 5, 6, 248-414, 596, 597, and 720-723.
58. The nucleic acid molecule of embodiment 54, wherein said adenine deaminase comprises an amino acid sequence with at least 90% sequence identity to SEQ ID NO: 5 or 6.
59. The nucleic acid molecule of embodiment 54, wherein said adenine deaminase comprises an amino acid sequence with at least 95% sequence identity to SEQ ID NO: 5 or 6.
60. The nucleic acid molecule of embodiment 54, wherein said adenine deaminase comprises the amino acid sequence of SEQ ID NO: 5 or 6.
61. The nucleic acid molecule of embodiment 54, wherein said fusion protein comprises an amino acid sequence with at least 90% sequence identity to any one of SEQ ID NOs: 8-10, 13-21, 27-31, 36-38, 40-41, 43-48, 647, and 650.
62. The nucleic acid molecule of embodiment 54, wherein said ABE fusion protein comprises an amino acid sequence with at least 95% sequence identity to any one of SEQ ID NOs: 8-10, 13-21, 27-31, 36-38, 40-41, 43-48, 647, and 650.
63. The nucleic acid molecule of embodiment 54, wherein said ABE fusion protein comprises the amino acid sequence set forth in any one of SEQ ID NOs: 8-10, 13-21, 27-31, 36-38, 40-41, 43-48, 647, and 650.
64. The nucleic acid molecule of embodiment 54, wherein said ABE fusion protein comprises an amino acid sequence with at least 90% sequence identity to any one of SEQ ID NOs: 8-10 and 14-16, and wherein said ABE fusion protein has an editing window that is shifted in comparison to a parental ABE base editor comprising said adenine deaminase fused to the amino terminus of said RGN.
65. The nucleic acid molecule of embodiment 54, wherein said ABE fusion protein comprises an amino acid sequence with at least 95% sequence identity to any one of SEQ ID NOs: 8-10 and 14-16, and wherein said ABE fusion protein has an editing window that is shifted in comparison to a parental ABE base editor comprising said adenine deaminase fused to the amino terminus of said RGN.
66. The nucleic acid molecule of embodiment 54, wherein said ABE fusion protein comprises the amino acid sequence set forth in any one of SEQ ID NOs: 8-10 and 14-16.
67. The nucleic acid molecule of any one of embodiments 37-66, wherein said RGN is a nickase or a nuclease-inactive RGN.
68. The nucleic acid molecule of embodiment 67, wherein said RGN nickase comprises an amino acid sequence with at least 95% sequence identity to any one of SEQ ID NOs: 49-56, 698, and 699 and retains nickase activity.
69. The nucleic acid molecule of embodiment 67, wherein said RGN nickase comprises the amino acid sequence of any one of SEQ ID NOs: 49-56, 698, and 699.
70. The nucleic acid molecule of any one of embodiments 37-69, wherein said RGN and said heterologous polypeptide are directly fused to one another with no linker sequences in between.
71. The nucleic acid molecule any of embodiments 37-70, wherein the fusion protein further comprises at least one nuclear localization signal (NLS).
72. The nucleic acid molecule of any one of embodiments 37-71, wherein the nucleic acid molecule is codon optimized for expression in a eukaryotic cell.
73. The nucleic acid molecule of any one of embodiments 37-71, wherein the nucleic acid molecule is codon optimized for expression in a prokaryotic cell.
74. A vector comprising the nucleic acid molecule of any one of embodiments 37-73 and 244-255.
75. The vector of embodiment 74, further comprising at least one nucleotide sequence encoding a guide RNA capable of hybridizing to a non-target strand of a target sequence of said RGN.
76. The vector of embodiment 74, wherein the guide RNA is a single guide RNA (sgRNA).
77. The vector of embodiment 74, wherein the guide RNA is a dual guide RNA.
78. A cell comprising the fusion protein of any of embodiments 1-35 and 232-243 or the RNP complex of embodiment 36.
79. A cell comprising the fusion protein of any one of embodiments 1-35 and 232-243, wherein the cell further comprises a guide RNA.
80. A cell comprising the nucleic acid molecule of any one of embodiments 37-73 and 244-255.
81. A cell comprising the vector of any one of embodiments 74-77.
82. The cell of any one of embodiments 78-81, wherein the cell is a prokaryotic cell.
83. The cell of any one of embodiments 78-81, wherein the cell is a eukaryotic cell.
84. The cell of embodiment 83, wherein the eukaryotic cell is a mammalian cell.
85. The cell of embodiment 84, wherein the mammalian cell is a human cell.
86. The cell of embodiment 85, wherein the human cell is an immune cell.
87. The cell of embodiment 86, wherein the immune cell is a stem cell.
88. The cell of embodiment 87, wherein the stem cell is an induced pluripotent stem cell.
89. The cell of embodiment 83, wherein the eukaryotic cell is an insect or avian cell.
90. The cell of embodiment 83, wherein the eukaryotic cell is a fungal cell.
91. The cell of embodiment 83, wherein the eukaryotic cell is a plant cell.
92. A plant comprising the cell of embodiment 91.
93. A seed comprising the cell of embodiment 91.
94. A pharmaceutical composition comprising a pharmaceutically acceptable carrier and the fusion protein of any one of embodiments 1-35 and 232-243, the RNP complex of embodiment 36, the nucleic acid molecule of any one of embodiments 37-73 and 244-255, the vector of any one of embodiments 74-77, or the cell of any one of embodiments 84-88.
95. A method for making a fusion protein comprising culturing the cell of any one of embodiments 78-91 under conditions in which the fusion protein is expressed.
96. A method for making a fusion protein comprising introducing into a cell the nucleic acid molecule of any of embodiments 37-73 and 237-243 or a vector of any one of embodiments 74-77 and culturing the cell under conditions in which the fusion protein is expressed.
97. The method of embodiment 95 or 96, further comprising purifying said fusion protein.
98. A method for making an RGN fusion ribonucleoprotein complex, comprising introducing into a cell the nucleic acid molecule of any one of embodiments 37-73 and 237-243 and a nucleic acid molecule comprising an expression cassette encoding for a guide RNA, or the vector of any of embodiments 74-77, and culturing the cell under conditions in which the fusion protein and the gRNA are expressed and form an RGN fusion ribonucleoprotein complex.
99. The method of embodiment 98, further comprising purifying said RGN fusion ribonucleoprotein complex.
100. A system for localizing a heterologous polypeptide to a target DNA molecule comprising a target DNA sequence, said system comprising:
a) a fusion protein, or a nucleotide sequence encoding the fusion protein, wherein said fusion protein comprises an RNA-guided nuclease (RGN) and at least one heterologous polypeptide inserted therein; wherein said RGN comprises an amino acid sequence with at least 90% sequence identity to any one of SEQ ID NOs: 1-4, 49-162, 435, 575, 576, 698, and 699; and
b) one or more guide RNAs capable of hybridizing to a non-target strand of said target DNA sequence or one or more nucleotide sequences encoding the one or more guide RNAs; and
wherein the one or more guide RNAs are capable of forming a complex with the fusion protein in order to direct said fusion protein to bind to said target DNA sequence.
101. The system of embodiment 100, wherein said RGN comprises an amino acid sequence with at least 95% sequence identity to any one of SEQ ID NOs: 1-4, 49-162, 435, 575, 576, 698, and 699.
102. The system of embodiment 100, wherein said RGN comprises the amino acid sequence of any one of SEQ ID NOs: 1-4, 49-162, 435, 575, 576, 698, and 699.
103. The system of embodiment 100, wherein said RGN comprises an amino acid sequence with at least 90% sequence identity to any one of SEQ ID NOs: 1-4 and 57-139.
104. The system of embodiment 100, wherein said RGN comprises an amino acid sequence with at least 95% sequence identity to any one of SEQ ID NOs: 1-4 and 57-139.
105. The system of embodiment 100, wherein said RGN comprises the amino acid sequence of any one of SEQ ID NOs: 1-4 and 57-139.
106. The system of any one of embodiments 100-105, wherein said heterologous polypeptide is inserted within a linker domain 2, a wedge domain, a RuvC domain, an HNH domain, a Rec-2 domain, or a PAM-interacting domain of said RGN.
107. The system of embodiment 106, wherein said RuvC domain is a RuvCIII domain.
108. The system of any one of embodiments 100-107, wherein:
   i) said RGN comprises an amino acid sequence with at least 90% sequence identity to SEQ ID NO: 1 and said at least one heterologous polypeptide is inserted within said RGN immediately after the amino acid position selected from the group consisting of:
      A. amino acid position corresponding to position 30 of SEQ ID NO: 1;
      B. amino acid position corresponding to position 642 of SEQ ID NO: 1;
      C. amino acid position corresponding to position 670 of SEQ ID NO: 1;
      D. amino acid position corresponding to position 737 of SEQ ID NO: 1;
      E. amino acid position corresponding to position 772 of SEQ ID NO: 1;
      F. amino acid position corresponding to position 775 of SEQ ID NO: 1;
      G. amino acid position corresponding to position 778 of SEQ ID NO: 1; and
      H. amino acid position corresponding to position 802 of SEQ ID NO: 1;
   ii) said RGN comprises an amino acid sequence with at least 90% sequence identity to SEQ ID NO: 2 and said at least one heterologous polypeptide is inserted within said RGN immediately after the amino acid position selected from the group consisting of:
      A. amino acid position corresponding to position 678 of SEQ ID NO: 2;
      B. amino acid position corresponding to position 736 of SEQ ID NO: 2;
      C. amino acid position corresponding to position 778 of SEQ ID NO: 2;
      D. amino acid position corresponding to position 788 of SEQ ID NO: 2; and
      E. amino acid position corresponding to position 922 of SEQ ID NO: 2;
   iii) said RGN comprises an amino acid sequence with at least 90% sequence identity to SEQ ID NO: 3 and said at least one heterologous polypeptide is inserted within said RGN immediately after the amino acid position selected from the group consisting of:
   A. amino acid position corresponding to position 725 of SEQ ID NO: 3;
   B. amino acid position corresponding to position 739 of SEQ ID NO: 3; and
   C. amino acid position corresponding to position 744 of SEQ ID NO: 3;
   iv) said RGN comprises an amino acid sequence with at least 90% sequence identity to SEQ ID NO: 4 and said at least one heterologous polypeptide is inserted within said RGN immediately after the amino acid position selected from the group consisting of:
      A. amino acid position corresponding to position 347 of SEQ ID NO: 4;
      B. amino acid position corresponding to position 524 of SEQ ID NO: 4;
      C. amino acid position corresponding to position 666 of SEQ ID NO: 4;
      D. amino acid position corresponding to position 680 of SEQ ID NO: 4;
      E. amino acid position corresponding to position 740 of SEQ ID NO: 4;
      F. amino acid position corresponding to position 785 of SEQ ID NO: 4;
      G. amino acid position corresponding to position 910 of SEQ ID NO: 4; and
      H. amino acid position corresponding to position 1077 of SEQ ID NO: 4; or
   v) said RGN comprises an amino acid sequence with at least 90% sequence identity to SEQ ID NO: 131 and said at least one heterologous polypeptide is inserted within said RGN immediately after the amino acid position selected from the group consisting of:
      A. amino acid position corresponding to position 766 of SEQ ID NO: 131; and
      B. amino acid position corresponding to position 806 of SEQ ID NO: 131.
109. The system of any one of embodiments 100-108, wherein said heterologous polypeptide comprises a base-editing polypeptide or a prime editing polypeptide.
110. The system of embodiment 109, wherein said prime editing polypeptide comprises a DNA polymerase.
111. The system of embodiment 109, wherein said prime editing polypeptide comprises a reverse transcriptase.
112. The system of embodiment 109, wherein said base-editing polypeptide comprises a deaminase.
113. The system of embodiment 112, wherein said deaminase is a cytosine deaminase or an adenine deaminase.
114. The system of embodiment 112 or 113, wherein said fusion protein is a base editor fusion protein, and wherein said base editor fusion protein has improved editing activity in comparison to a parental base editor comprising said deaminase fused to the amino terminus of said RGN.
115. The system of any one of embodiments 112-114, wherein said at least one heterologous polypeptide is inserted within said RGN comprising an amino acid sequence with at least 90% sequence identity to SEQ ID NO: 1 immediately after the amino acid position selected from the group consisting of:
a) amino acid position corresponding to position 642 of SEQ ID NO: 1;
b) amino acid position corresponding to position 670 of SEQ ID NO: 1;
c) amino acid position corresponding to position 737 of SEQ ID NO: 1;
d) amino acid position corresponding to position 772 of SEQ ID NO: 1;
e) amino acid position corresponding to position 775 of SEQ ID NO: 1; and
f) amino acid position corresponding to position 778 of SEQ ID NO: 1; and
wherein said fusion protein is a base editor fusion protein, and wherein said base editor fusion protein has an editing window that is shifted in comparison to a parental base editor comprising said deaminase fused to the amino terminus of said RGN.
116. The system of any one of embodiments 112-115, wherein said deaminase lacks the first and last amino acid residues in comparison to a parental deaminase from which said deaminase is derived.
117. The system of any one of embodiments 112-116, wherein said deaminase comprises an adenine deaminase and said fusion protein is an adenosine base editor (ABE) fusion protein.
118. The system of embodiment 117, wherein said adenine deaminase comprises an amino acid sequence with at least 90% sequence identity to any one of SEQ ID NOs: 5, 6, 248-414, 596, 597, and 720-723.
119. The system of embodiment 117, wherein said adenine deaminase comprises an amino acid sequence with at least 95% sequence identity to any one of SEQ ID NOs: 5, 6, 248-414, 596, 597, and 720-723.
120. The system of embodiment 117, wherein said adenine deaminase comprises the amino acid sequence of any one of SEQ ID NOs: 5, 6, 248-414, 596, 597, and 720-723.
121. The system of embodiment 117, wherein said adenine deaminase comprises an amino acid sequence with at least 90% sequence identity to SEQ ID NO: 5 or 6.
122. The system of embodiment 117, wherein said adenine deaminase comprises an amino acid sequence with at least 95% sequence identity to SEQ ID NO: 5 or 6.
123. The system of embodiment 117, wherein said adenine deaminase comprises the amino acid sequence of SEQ ID NO: 5 or 6.
124. The system of embodiment 117, wherein said ABE fusion protein comprises an amino acid sequence with at least 90% sequence identity to any one of SEQ ID NOs: 8-10, 13-21, 27-31, 36-38, 40-41, 43-48, 647, and 650.
125. The system of embodiment 117, wherein said ABE fusion protein comprises an amino acid sequence with at least 95% sequence identity to any one of SEQ ID NOs: 8-10, 13-21, 27-31, 36-38, 40-41, 43-48, 647, and 650.
126. The system of embodiment 117, wherein said ABE fusion protein comprises the amino acid sequence set forth in any one of SEQ ID NOs: 8-10, 13-21, 27-31, 36-38, 40-41, 43-48, 647, and 650.
127. The system of embodiment 117, wherein said ABE fusion protein comprises an amino acid sequence with at least 90% sequence identity to any one of SEQ ID NOs: 8-10 and 14-16, and wherein said ABE fusion protein has an editing window that is shifted in comparison to a parental ABE base editor comprising said adenine deaminase fused to the amino terminus of said RGN.
128. The system of embodiment 117, wherein said ABE fusion protein comprises an amino acid sequence with at least 95% sequence identity to any one of SEQ ID NOs: 8-10 and 14-16, and wherein said ABE fusion protein has an editing window that is shifted in comparison to a parental ABE base editor comprising said adenine deaminase fused to the amino terminus of said RGN.
129. The system of embodiment 117, wherein said ABE fusion protein comprises the amino acid sequence set forth in any one of SEQ ID NOs: 8-10 and 14-16.
130. The system of any one of embodiments 100-129, wherein said RGN is a nickase or a nuclease-inactive RGN.
131. The system of embodiment 130, wherein said RGN nickase comprises an amino acid sequence with at least 95% sequence identity to any one of SEQ ID NOs: 49-56, 698, and 699 and retains nickase activity.
132. The system of embodiment 130, wherein said RGN nickase comprises the amino acid sequence of any one of SEQ ID NOs: 49-56, 698, and 699.
133. The system of any one of embodiments 100-132, wherein said RGN and said heterologous polypeptide are directly fused to one another without linker sequences in between.
134. The system of any of embodiments 100-133, wherein the fusion protein further comprises at least one nuclear localization signal (NLS).
135. The system of any one of embodiments 100-134, wherein the nucleotide sequence encoding the fusion protein is codon optimized for expression in a eukaryotic cell.
136. The system of any one of embodiments 100-135, wherein at least one of said nucleotide sequences encoding the one or more guide RNAs and said nucleotide sequence encoding the fusion protein is operably linked to a promoter heterologous to said nucleotide sequence.
137. The system of any one of embodiments 100-136, wherein the target DNA sequence is a eukaryotic target DNA sequence.
138. The system of any one of embodiments 100-137, wherein the target DNA sequence is located adjacent to a protospacer adjacent motif (PAM) that is recognized by the RGN.
139. The system of any one of embodiments 100-138, wherein nucleotide sequences encoding the one or more guide RNAs and the nucleotide sequence encoding a fusion protein are located on one vector.
140. A cell comprising the system of any one of embodiments 100-139 and 256-267.
141. The cell of embodiment 140, wherein the cell is a eukaryotic cell.
142. The cell of embodiment 141, wherein the eukaryotic cell is a mammalian cell.
143. The cell of embodiment 142, wherein the mammalian cell is a human cell.
144. The cell of embodiment 143, wherein the human cell is an immune cell.
145. The cell of embodiment 144, wherein the immune cell is a stem cell.
146. The cell of embodiment 145, wherein the stem cell is an induced pluripotent stem cell.
147. The cell of embodiment 141, wherein the eukaryotic cell is an insect cell.
148. The cell of embodiment 141, wherein the eukaryotic cell is a plant cell.
149. A plant comprising the cell of embodiment 148.
150. A seed comprising the cell of embodiment 148.
151. The cell of embodiment 140, wherein the cell is a prokaryotic cell.
152. A pharmaceutical composition comprising a pharmaceutically acceptable carrier and the system of any one of embodiments 100-139 and 256-267 or the cell of any one of embodiments 142-146.
153. A method for localizing a heterologous polypeptide to a target DNA molecule comprising a target DNA sequence, said method comprising delivering a system according to any one of embodiments 100-139 and 256-267 to said target DNA molecule or a cell comprising the target DNA molecule.
154. A method for modifying a target DNA molecule comprising a target DNA sequence, said method comprising delivering a system according to any one of embodiments 100-139 and 256-267 to said target DNA molecule or a cell comprising the target DNA molecule.
155. The method of embodiment 154, wherein said deaminase comprises an adenine deaminase and wherein said modified target DNA molecule comprises an A>N mutation of at least one nucleotide within the target DNA molecule, wherein N is C, G, or T.
156. The method of embodiment 155, wherein said modified target DNA molecule comprises an A>G mutation of at least one nucleotide within the target DNA molecule.
157. A method for localizing a heterologous polypeptide to a target DNA molecule comprising a target DNA sequence comprising:
a) assembling a ribonucleotide complex in vitro by combining:
   i) one or more guide RNAs capable of hybridizing to a non-target strand of the target DNA sequence; and
   ii) a fusion protein comprising an RNA-guided nuclease polypeptide (RGN) and at least one heterologous polypeptide inserted therein, wherein said RGN comprises an amino acid sequence with at least 90% sequence identity to any one of SEQ ID NOs: 1-4, 49-162, 435, 575, 576, 698, and 699;
under conditions suitable for formation of the ribonucleotide complex;
and
b) contacting said target DNA molecule or a cell comprising said target DNA molecule with the in vitro-assembled ribonucleotide complex;
wherein the one or more guide RNAs hybridize to a non-target strand of the target DNA sequence, thereby directing said fusion protein to bind to said target DNA sequence.
158. The method of embodiment 157, wherein said RGN comprises an amino acid sequence with at least 95% sequence identity to any one of SEQ ID NOs: 1-4, 49-162, 435, 575, 576, 698, and 699.
159. The method of embodiment 157, wherein said RGN comprises an amino acid sequence with the amino acid sequence of any one of SEQ ID NOs: 1-4, 49-162, 435, 575, 576, 698, and 699.
160. The method of embodiment 157, wherein said RGN comprises at least 90% sequence identity to any one of SEQ ID NOs: 1-4 and 57-139.
161. The method of embodiment 157, wherein said RGN comprises an amino acid sequence with at least 95% sequence identity to any one of SEQ ID NOs: 1-4 and 57-139.
162. The method of embodiment 157, wherein said RGN comprises the amino acid sequence of any one of SEQ ID NOs: 1-4 and 57-139.
163. The method of any one of embodiments 157-162, wherein said heterologous polypeptide is inserted within a linker domain 2, a wedge domain, a RuvC domain, an HNH domain, a Rec-2 domain, or a PAM-interacting domain of said RGN.
164. The method of embodiment 163, wherein said RuvC domain is a RuvCIII domain.
165. The method of any one of embodiments 157-164, wherein:
   a) said RGN comprises an amino acid sequence with at least 90% sequence identity to SEQ ID NO: 1 and said at least one heterologous polypeptide is inserted within said RGN immediately after the amino acid position selected from the group consisting of:
      I) amino acid position corresponding to position 30 of SEQ ID NO: 1;
      II) amino acid position corresponding to position 642 of SEQ ID NO: 1;
      III) amino acid position corresponding to position 670 of SEQ ID NO: 1;
      IV) amino acid position corresponding to position 737 of SEQ ID NO: 1;
      V) amino acid position corresponding to position 772 of SEQ ID NO: 1;
      VI) amino acid position corresponding to position 775 of SEQ ID NO: 1;
      VII) amino acid position corresponding to position 778 of SEQ ID NO: 1; and
      VIII) amino acid position corresponding to position 802 of SEQ ID NO: 1;
   b) said RGN comprises an amino acid sequence with at least 90% sequence identity to SEQ ID NO: 2 and said at least one heterologous polypeptide is inserted within said RGN immediately after the amino acid position selected from the group consisting of:
      I) amino acid position corresponding to position 678 of SEQ ID NO: 2;
      II) amino acid position corresponding to position 736 of SEQ ID NO: 2;
      III) amino acid position corresponding to position 778 of SEQ ID NO: 2;
      IV) amino acid position corresponding to position 788 of SEQ ID NO: 2; and
      V) amino acid position corresponding to position 922 of SEQ ID NO: 2;
   c) said RGN comprises an amino acid sequence with at least 90% sequence identity to SEQ ID NO: 3 and said at least one heterologous polypeptide is inserted within said RGN immediately after the amino acid position selected from the group consisting of:
      I) amino acid position corresponding to position 725 of SEQ ID NO: 3;
      II) amino acid position corresponding to position 739 of SEQ ID NO: 3;
      III) amino acid position corresponding to position 744 of SEQ ID NO: 3;
   d) said RGN comprises an amino acid sequence with at least 90% sequence identity to SEQ ID NO: 4 and said at least one heterologous polypeptide is inserted within said RGN immediately after the amino acid position selected from the group consisting of:
      I) amino acid position corresponding to position 347 of SEQ ID NO: 4;
      II) amino acid position corresponding to position 524 of SEQ ID NO: 4;
      III) amino acid position corresponding to position 666 of SEQ ID NO: 4;
      IV) amino acid position corresponding to position 680 of SEQ ID NO: 4;
      V) amino acid position corresponding to position 740 of SEQ ID NO: 4;
      VI) amino acid position corresponding to position 785 of SEQ ID NO: 4;
      VII) amino acid position corresponding to position 910 of SEQ ID NO: 4; and
      VIII) amino acid position corresponding to position 1077 of SEQ ID NO: 4; or
   e) said RGN comprises an amino acid sequence with at least 90% sequence identity to SEQ ID NO: 131 and said at least one heterologous polypeptide is inserted within said RGN immediately after the amino acid position selected from the group consisting of:
      I) amino acid position corresponding to position 766 of SEQ ID NO: 131; and
      II) amino acid position corresponding to position 806 of SEQ ID NO: 131.
166. The method of any one of embodiments 157-165, wherein said heterologous polypeptide comprises a base editing polypeptide or a prime editing polypeptide and wherein said method further comprises modifying said target DNA molecule to generate a modified target DNA molecule.
167. The method of embodiment 166, wherein the target DNA molecule comprises a causal mutation for a disease or disorder and wherein modification of the target DNA molecule corrects said causal mutation.
168. The method of embodiment 167, wherein the correction of the causal mutation comprises correcting a nonsense mutation.
169. The method of any one of embodiments 166-168, wherein said prime editing polypeptide comprises a DNA polymerase.
170. The method of any one of embodiments 166-168, wherein said prime editing polypeptide comprises a reverse transcriptase.
171. The method of any one of embodiments 166-168, wherein said base-editing polypeptide comprises a deaminase.
172. The method of embodiment 171, wherein said deaminase is a cytosine deaminase or an adenine deaminase.
173. The method of embodiment 171 or 172, wherein said fusion protein is a base editor fusion protein, and wherein said base editor fusion protein has improved editing activity in comparison to a parental base editor comprising said deaminase fused to the amino terminus of said RGN.
174. The method of any one of embodiments 171-173, wherein said at least one heterologous polypeptide is inserted within said RGN comprising an amino acid sequence with at least 90% sequence identity to SEQ ID NO: 1 immediately after the amino acid position selected from the group consisting of:
a) amino acid position corresponding to position 642 of SEQ ID NO: 1;
b) amino acid position corresponding to position 670 of SEQ ID NO: 1;
c) amino acid position corresponding to position 737 of SEQ ID NO: 1;
d) amino acid position corresponding to position 772 of SEQ ID NO: 1;
e) amino acid position corresponding to position 775 of SEQ ID NO: 1; and
f) amino acid position corresponding to position 778 of SEQ ID NO: 1; and
wherein said fusion protein is a base editor fusion protein, and wherein said base editor fusion protein has an editing window that is shifted in comparison to a parental base editor comprising said deaminase fused to the amino terminus of said RGN.
175. The method of any one of embodiments 171-174, wherein said deaminase lacks the first and last amino acid residues in comparison to a parental deaminase from which said deaminase is derived.
176. The method of any one of embodiments 171-175, wherein said deaminase comprises an adenine deaminase and said fusion protein is an adenosine base editor (ABE) fusion protein.
177. The method of embodiment 176, wherein said adenine deaminase comprises an amino acid sequence with at least 90% sequence identity to any one of SEQ ID NOs: 5, 6, 248-414, 596, 597, and 720-723.
178. The method of embodiment 176, wherein said adenine deaminase comprises an amino acid sequence with at least 95% sequence identity to any one of SEQ ID NOs: 5, 6, 248-414, 596, 597, and 720-723.
179. The method of embodiment 176, wherein said adenine deaminase comprises the amino acid sequence of any one of SEQ ID NOs: 5, 6, 248-414, 596, 597, and 720-723.
180. The method of embodiment 176, wherein said adenine deaminase comprises an amino acid sequence with at least 90% sequence identity to SEQ ID NO: 5 or 6.
181. The method of embodiment 176, wherein said adenine deaminase comprises an amino acid sequence with at least 95% sequence identity to SEQ ID NO: 5 or 6.
182. The method of embodiment 176, wherein said adenine deaminase comprises the amino acid sequence of SEQ ID NO: 5 or 6.
183. The method of embodiment 176, wherein said ABE fusion protein comprises an amino acid sequence with at least 90% sequence identity to any one of SEQ ID NOs: 8-10, 13-21, 27-31, 36-38, 40-41, 43-48, 647, and 650.
184. The method of embodiment 176, wherein said ABE fusion protein comprises an amino acid sequence with at least 95% sequence identity to any one of SEQ ID NOs: 8-10, 13-21, 27-31, 36-38, 40-41, 43-48, 647, and 650.
185. The method of embodiment 176, wherein said ABE fusion protein comprises the amino acid sequence set forth in any one of SEQ ID NOs: 8-10, 13-21, 27-31, 36-38, 40-41, 43-48, 647, and 650.
186. The method of embodiment 176, wherein said ABE fusion protein comprises an amino acid sequence with at least 90% sequence identity to any one of SEQ ID NOs: 8-10 and 14-16, and wherein said ABE fusion protein has an editing window that is shifted in comparison to a parental ABE base editor comprises said adenine deaminase fused to the amino terminus of said RGN.
187. The method of embodiment 176, wherein said ABE fusion protein comprises an amino acid sequence with at least 95% sequence identity to any one of SEQ ID NOs: 8-10 and 14-16, and wherein said ABE fusion protein has an editing window that is shifted in comparison to a parental ABE base editor comprising said adenine deaminase fused to the amino terminus of said RGN.
188. The method of embodiment 176, wherein said ABE fusion protein comprises the amino acid sequence set forth in any one of SEQ ID NOs: 8-10 and 14-16.
189. The method of any one of embodiments 176-188, wherein said modified target DNA molecule comprises an A>N mutation of at least one nucleotide within the target DNA molecule, wherein N is C, G, or T.
190. The method of embodiment 189, wherein said modified target DNA molecule comprises an A>G mutation of at least one nucleotide within the target DNA molecule.
191. The method of any one of embodiments 157-190, wherein said RGN is a nickase or a nuclease-inactive RGN.
192. The method of embodiment 191, wherein said RGN nickase comprises an amino acid sequence with at least 95% sequence identity to any one of SEQ ID NOs: 49-56, 698, and 699 and retains nickase activity.
193. The method of embodiment 191, wherein said RGN nickase comprises the amino acid sequence of any one of SEQ ID NOs: 49-56, 698, and 699.
194. The method of any one of embodiments 157-193, wherein said RGN and said heterologous polypeptide are directly fused to one another with no linker sequences in between.
195. The method of any of embodiments 157-194, wherein the fusion protein further comprises at least one nuclear localization signal (NLS).
196. The method of any one of embodiments 157-195, wherein the fusion protein is codon optimized for expression in a eukaryotic cell.
197. The method of any one of embodiments 157-196, wherein at least one of said nucleotide sequences encoding the one or more guide RNAs and said nucleotide sequence encoding the fusion protein is operably linked to a promoter heterologous to said nucleotide sequence.
198. The method of any one of embodiments 157-197, wherein the target DNA sequence is a eukaryotic target DNA sequence.
199. The method of any one of embodiments 157-198, wherein the target DNA sequence is located adjacent to a protospacer adjacent motif (PAM) that is recognized by the RGN.
200. The method of any one of embodiments 157-199, wherein nucleotide sequences encoding the one or more guide RNAs and the nucleotide sequence encoding a fusion protein are located on one vector.
201. The method of any one of embodiments 157-200, wherein said target DNA sequence is a eukaryotic target DNA sequence.
202. The method of any of embodiments 157-201, wherein the target DNA molecule is within a cell.
203. The method of embodiment 202, wherein the cell is a eukaryotic cell.
204. The method of embodiment 203, wherein the eukaryotic cell is a plant cell.
205. The method of embodiment 203, wherein the eukaryotic cell is a mammalian cell.
206. The method of embodiment 205, wherein the mammalian cell is a human cell.
207. The method of embodiment 206, wherein the human cell is an immune cell.
208. The method of embodiment 207, wherein the immune cell is a stem cell.
209. The method of embodiment 208, wherein the stem cell is an induced pluripotent stem cell.
210. The method of embodiment 203, wherein the eukaryotic cell is an insect cell.
211. The method of embodiment 202, wherein the cell is a prokaryotic cell.
212. The method of any one of embodiments 202-211, further comprising selecting a cell comprising said modified target DNA molecule.
213. A cell comprising a modified target DNA molecule according to the method of embodiment 212.
214. The cell of embodiment 213, wherein the cell is a eukaryotic cell.
215. The cell of embodiment 214, wherein the eukaryotic cell is a plant cell.
216. A plant comprising the cell of embodiment 215.
217. A seed comprising the cell of embodiment 215.
218. The cell of embodiment 214, wherein the eukaryotic cell is a mammalian cell.
219. The cell of embodiment 218, wherein the mammalian cell is a human cell.
220. The cell of embodiment 219, wherein the human cell is an immune cell.
221. The cell of embodiment 220, wherein the immune cell is a stem cell.
222. The cell of embodiment 221, wherein the stem cell is an induced pluripotent stem cell.
223. The cell of embodiment 214, wherein the eukaryotic cell is an insect cell.
224. The cell of embodiment 213, wherein the cell is a prokaryotic cell.
225. A pharmaceutical composition comprising the cell of any one of embodiments 218-222, and a pharmaceutically acceptable carrier.
226. A method for treating a subject having or at risk of developing a disease, disorder, or condition, the method comprising:
administering to the subject the fusion protein of any of embodiments 1-35 and 232-243, the RNP complex of embodiment 36, the nucleic acid molecule of any one of embodiments 37-73 and 244-255, the vector of any one of embodiments 74-77, the cell of any one of embodiments 84-88, 142-146, and 218-222, the system of any one of embodiments 100-139 and 256-267, or the pharmaceutical composition of any one of embodiments 94, 152, and 225.
227. The method of embodiment 226, wherein said disease is associated with a causal mutation and said treating comprises correcting said causal mutation.
228. Use of the fusion protein of any one of embodiments 1-35 and 232-243, the RNP complex of embodiment 36, the nucleic acid molecule of any one of embodiments 37-73 and 244-255, the vector of any one of embodiments 74-77, the cell of any one of embodiments 84-88, 142-146, and 218-222, or the system of any one of embodiments 100-139 and 256-267 for the treatment of a disease, disorder, or condition in a subject having or at risk of developing said disease, disorder, or condition.
229. The use of embodiment 228, wherein said disease is associated with a causal mutation and said treating comprises correcting said causal mutation.
230. Use of the fusion protein of any one of embodiments 1-35 and 232-243, the RNP complex of embodiment 36, the nucleic acid molecule of any one of embodiments 37-73 and 244-255, the vector of any one of embodiments 74-77, the cell of any one of embodiments 84-88, 142-146, and 218-222, or the system of any one of embodiments 100-139 and 256-267 for the manufacture of a medicament useful for treating a disease, disorder, or condition.
231. The use of embodiment 230, wherein said disease is associated with a causal mutation and an effective amount of said medicament corrects said causal mutation.
232. The fusion protein of embodiment 18, wherein said adenine deaminase comprises an amino acid sequence having at least 85% sequence identity to SEQ ID NO: 5, and comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
233. The fusion protein of embodiment 232, wherein said adenine deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
234. The fusion protein of embodiment 232, wherein said adenine deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
235. The fusion protein of embodiment 232, wherein said adenine deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
236. The fusion protein of any one of embodiments 232, 234, and 235, wherein said adenine deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
237. The fusion protein of any one of embodiments 232-236, wherein said deaminase has an amino acid sequence having at least 90% sequence identity to any one of SEQ ID NOs: 300-414, 596-598, and 720-723.
238. The fusion protein of any one of embodiments 232-236, wherein said deaminase has an amino acid sequence having at least 95% sequence identity to any one of SEQ ID NOs: 300-414, 596-598, and 720-723.
239. The fusion protein of any one of embodiments 232-236, wherein said deaminase has the amino acid sequence set forth as any one of SEQ ID NOs: 300-414, 596-598, and 720-723.
240. The fusion protein of any one of embodiments 232-239, wherein said fusion protein is selected from the group consisting of:
a) a fusion protein wherein said deaminase has an amino acid sequence having at least 90% sequence identity to SEQ ID NO: 375 and is inserted within said RGN having an amino acid sequence having at least 90% sequence identity to SEQ ID NO: 50 after an amino acid position corresponding to position 922 of SEQ ID NO: 2;
b) a fusion protein wherein said deaminase has an amino acid sequence having at least 90% sequence identity to SEQ ID NO: 375 and is inserted within said RGN having an amino acid sequence having at least 90% sequence identity to SEQ ID NO: 52 after an amino acid position corresponding to position 910 of SEQ ID NO: 4;
c) a fusion protein wherein said deaminase has an amino acid sequence having at least 90% sequence identity to SEQ ID NO: 319 and is inserted within said RGN having an amino acid sequence having at least 90% sequence identity to SEQ ID NO: 50 after an amino acid position corresponding to position 922 of SEQ ID NO: 2;
d) a fusion protein wherein said deaminase has an amino acid sequence having at least 90% sequence identity to SEQ ID NO: 319 and is inserted within said RGN having an amino acid sequence having at least 90% sequence identity to SEQ ID NO: 52 after an amino acid position corresponding to position 910 of SEQ ID NO: 4;
e) a fusion protein wherein said deaminase has an amino acid sequence having at least 90% sequence identity to SEQ ID NO: 375 and is inserted within said RGN having an amino acid sequence having at least 90% sequence identity to SEQ ID NO: 49 after an amino acid position corresponding to position 772 of SEQ ID NO: 1;
f) a fusion protein wherein said deaminase has an amino acid sequence having at least 90% sequence identity to SEQ ID NO: 375 and is inserted within said RGN having an amino acid sequence having at least 90% sequence identity to SEQ ID NO: 50 after an amino acid position corresponding to position 678 of SEQ ID NO: 2;
g) a fusion protein wherein said deaminase has an amino acid sequence having at least 90% sequence identity to SEQ ID NO: 319 and is inserted within said RGN having an amino acid sequence having at least 90% sequence identity to SEQ ID NO: 49 after an amino acid position corresponding to position 772 of SEQ ID NO: 1;
h) a fusion protein wherein said deaminase has an amino acid sequence having at least 90% sequence identity to SEQ ID NO: 597 and is inserted within said RGN having an amino acid sequence having at least 90% sequence identity to SEQ ID NO: 50 after an amino acid position corresponding to position 922 of SEQ ID NO: 2;
i) a fusion protein wherein said deaminase has an amino acid sequence having at least 90% sequence identity to SEQ ID NO: 319 and is inserted within said RGN having an amino acid sequence having at least 90% sequence identity to SEQ ID NO: 50 after an amino acid position corresponding to position 678 of SEQ ID NO: 2;
j) a fusion protein wherein said deaminase has an amino acid sequence having at least 90% sequence identity to SEQ ID NO: 406 and is inserted within said RGN having an amino acid sequence having at least 90% sequence identity to SEQ ID NO: 50 after an amino acid position corresponding to position 678 of SEQ ID NO: 2;
k) a fusion protein wherein said deaminase has an amino acid sequence having at least 90% sequence identity to SEQ ID NO: 597 and is inserted within said RGN having an amino acid sequence having at least 90% sequence identity to SEQ ID NO: 50 after an amino acid position corresponding to position 678 of SEQ ID NO: 2;
l) a fusion protein wherein said deaminase has an amino acid sequence having at least 90% sequence identity to SEQ ID NO: 596 and is inserted within said RGN having an amino acid sequence having at least 90% sequence identity to SEQ ID NO: 50 after an amino acid position corresponding to position 678 of SEQ ID NO: 2;
m) a fusion protein wherein said deaminase has an amino acid sequence having at least 90% sequence identity to SEQ ID NO: 406 and is inserted within said RGN having an amino acid sequence having at least 90% sequence identity to SEQ ID NO: 52 after an amino acid position corresponding to position 910 of SEQ ID NO: 4;
n) a fusion protein wherein said deaminase has an amino acid sequence having at least 90% sequence identity to SEQ ID NO: 597 and is inserted within said RGN having an amino acid sequence having at least 90% sequence identity to SEQ ID NO: 52 after an amino acid position corresponding to position 910 of SEQ ID NO: 4;
o) a fusion protein wherein said deaminase has an amino acid sequence having at least 90% sequence identity to SEQ ID NO: 406 and is inserted within said RGN having an amino acid sequence having at least 90% sequence identity to SEQ ID NO: 50 after an amino acid position corresponding to position 922 of SEQ ID NO: 2;
p) a fusion protein wherein said deaminase has an amino acid sequence having at least 90% sequence identity to SEQ ID NO: 596 and is inserted within said RGN having an amino acid sequence having at least 90% sequence identity to SEQ ID NO: 52 after an amino acid position corresponding to position 910 of SEQ ID NO: 4;
q) a fusion protein wherein said deaminase has an amino acid sequence having at least 90% sequence identity to SEQ ID NO: 596 and is inserted within said RGN having an amino acid sequence having at least 90% sequence identity to SEQ ID NO: 50 after an amino acid position corresponding to position 922 of SEQ ID NO: 2;
r) a fusion protein wherein said deaminase has an amino acid sequence having at least 90% sequence identity to SEQ ID NO: 406 and is inserted within said RGN having an amino acid sequence having at least 90% sequence identity to SEQ ID NO: 49 after an amino acid position corresponding to position 772 of SEQ ID NO: 1;
s) a fusion protein wherein said deaminase has an amino acid sequence having at least 90% sequence identity to SEQ ID NO: 596 and is inserted within said RGN having an amino acid sequence having at least 90% sequence identity to SEQ ID NO: 49 after an amino acid position corresponding to position 772 of SEQ ID NO: 1; and
t) a fusion protein wherein said deaminase has an amino acid sequence having at least 90% sequence identity to SEQ ID NO: 597 and is inserted within said RGN having an amino acid sequence having at least 90% sequence identity to SEQ ID NO: 49 after an amino acid position corresponding to position 772 of SEQ ID NO: 1.
241. The fusion protein of any one of embodiments 232-239, wherein said fusion protein is selected from the group consisting of:
a) a fusion protein wherein said deaminase has an amino acid sequence having at least 95% sequence identity to SEQ ID NO: 375 and is inserted within said RGN having an amino acid sequence having at least 95% sequence identity to SEQ ID NO: 50 after an amino acid position corresponding to position 922 of SEQ ID NO: 2;
b) a fusion protein wherein said deaminase has an amino acid sequence having at least 95% sequence identity to SEQ ID NO: 375 and is inserted within said RGN having an amino acid sequence having at least 95% sequence identity to SEQ ID NO: 52 after an amino acid position corresponding to position 910 of SEQ ID NO: 4;
c) a fusion protein wherein said deaminase has an amino acid sequence having at least 95% sequence identity to SEQ ID NO: 319 and is inserted within said RGN having an amino acid sequence having at least 95% sequence identity to SEQ ID NO: 50 after an amino acid position corresponding to position 922 of SEQ ID NO: 2;
d) a fusion protein wherein said deaminase has an amino acid sequence having at least 95% sequence identity to SEQ ID NO: 319 and is inserted within said RGN having an amino acid sequence having at least 95% sequence identity to SEQ ID NO: 52 after an amino acid position corresponding to position 910 of SEQ ID NO: 4;
e) a fusion protein wherein said deaminase has an amino acid sequence having at least 95% sequence identity to SEQ ID NO: 375 and is inserted within said RGN having an amino acid sequence having at least 95% sequence identity to SEQ ID NO: 49 after an amino acid position corresponding to position 772 of SEQ ID NO: 1;
f) a fusion protein wherein said deaminase has an amino acid sequence having at least 95% sequence identity to SEQ ID NO: 375 and is inserted within said RGN having an amino acid sequence having at least 95% sequence identity to SEQ ID NO: 50 after an amino acid position corresponding to position 678 of SEQ ID NO: 2;
g) a fusion protein wherein said deaminase has an amino acid sequence having at least 95% sequence identity to SEQ ID NO: 319 and is inserted within said RGN having an amino acid sequence having at least 95% sequence identity to SEQ ID NO: 49 after an amino acid position corresponding to position 772 of SEQ ID NO: 1;
h) a fusion protein wherein said deaminase has an amino acid sequence having at least 95% sequence identity to SEQ ID NO: 597 and is inserted within said RGN having an amino acid sequence having at least 95% sequence identity to SEQ ID NO: 50 after an amino acid position corresponding to position 922 of SEQ ID NO: 2;
i) a fusion protein wherein said deaminase has an amino acid sequence having at least 95% sequence identity to SEQ ID NO: 319 and is inserted within said RGN having an amino acid sequence having at least 95% sequence identity to SEQ ID NO: 50 after an amino acid position corresponding to position 678 of SEQ ID NO: 2;
j) a fusion protein wherein said deaminase has an amino acid sequence having at least 95% sequence identity to SEQ ID NO: 406 and is inserted within said RGN having an amino acid sequence having at least 95% sequence identity to SEQ ID NO: 50 after an amino acid position corresponding to position 678 of SEQ ID NO: 2;
k) a fusion protein wherein said deaminase has an amino acid sequence having at least 95% sequence identity to SEQ ID NO: 597 and is inserted within said RGN having an amino acid sequence having at least 95% sequence identity to SEQ ID NO: 50 after an amino acid position corresponding to position 678 of SEQ ID NO: 2;
l) a fusion protein wherein said deaminase has an amino acid sequence having at least 95% sequence identity to SEQ ID NO: 596 and is inserted within said RGN having an amino acid sequence having at least 95% sequence identity to SEQ ID NO: 50 after an amino acid position corresponding to position 678 of SEQ ID NO: 2;
m) a fusion protein wherein said deaminase has an amino acid sequence having at least 95% sequence identity to SEQ ID NO: 406 and is inserted within said RGN having an amino acid sequence having at least 95% sequence identity to SEQ ID NO: 52 after an amino acid position corresponding to position 910 of SEQ ID NO: 4;
n) a fusion protein wherein said deaminase has an amino acid sequence having at least 95% sequence identity to SEQ ID NO: 597 and is inserted within said RGN having an amino acid sequence having at least 95% sequence identity to SEQ ID NO: 52 after an amino acid position corresponding to position 910 of SEQ ID NO: 4;
o) a fusion protein wherein said deaminase has an amino acid sequence having at least 95% sequence identity to SEQ ID NO: 406 and is inserted within said RGN having an amino acid sequence having at least 95% sequence identity to SEQ ID NO: 50 after an amino acid position corresponding to position 922 of SEQ ID NO: 2;
p) a fusion protein wherein said deaminase has an amino acid sequence having at least 95% sequence identity to SEQ ID NO: 596 and is inserted within said RGN having an amino acid sequence having at least 95% sequence identity to SEQ ID NO: 52 after an amino acid position corresponding to position 910 of SEQ ID NO: 4;
q) a fusion protein wherein said deaminase has an amino acid sequence having at least 95% sequence identity to SEQ ID NO: 596 and is inserted within said RGN having an amino acid sequence having at least 95% sequence identity to SEQ ID NO: 50 after an amino acid position corresponding to position 922 of SEQ ID NO: 2;
r) a fusion protein wherein said deaminase has an amino acid sequence having at least 95% sequence identity to SEQ ID NO: 406 and is inserted within said RGN having an amino acid sequence having at least 95% sequence identity to SEQ ID NO: 49 after an amino acid position corresponding to position 772 of SEQ ID NO: 1;
s) a fusion protein wherein said deaminase has an amino acid sequence having at least 95% sequence identity to SEQ ID NO: 596 and is inserted within said RGN having an amino acid sequence having at least 95% sequence identity to SEQ ID NO: 49 after an amino acid position corresponding to position 772 of SEQ ID NO: 1; and
t) a fusion protein wherein said deaminase has an amino acid sequence having at least 95% sequence identity to SEQ ID NO: 597 and is inserted within said RGN having an amino acid sequence having at least 95% sequence identity to SEQ ID NO: 49 after an amino acid position corresponding to position 772 of SEQ ID NO: 1.
242. The fusion protein of any one of embodiments 232-239, wherein said fusion protein is selected from the group consisting of:
a) a fusion protein wherein said deaminase has the amino acid sequence set forth as SEQ ID NO: 375 and is inserted within said RGN having the amino acid sequence set forth as SEQ ID NO: 50 after an amino acid position corresponding to position 922 of SEQ ID NO: 2;
b) a fusion protein wherein said deaminase has the amino acid sequence set forth as SEQ ID NO: 375 and is inserted within said RGN having the amino acid sequence set forth as SEQ ID NO: 52 after an amino acid position corresponding to position 910 of SEQ ID NO: 4;
c) a fusion protein wherein said deaminase has the amino acid sequence set forth as SEQ ID NO: 319 and is inserted within said RGN having the amino acid sequence set forth as SEQ ID NO: 50 after an amino acid position corresponding to position 922 of SEQ ID NO: 2;
d) a fusion protein wherein said deaminase has the amino acid sequence set forth as SEQ ID NO: 319 and is inserted within said RGN having the amino acid sequence set forth as SEQ ID NO: 52 after an amino acid position corresponding to position 910 of SEQ ID NO: 4;
e) a fusion protein wherein said deaminase has the amino acid sequence set forth as SEQ ID NO: 375 and is inserted within said RGN having the amino acid sequence set forth as SEQ ID NO: 49 after an amino acid position corresponding to position 772 of SEQ ID NO: 1;
f) a fusion protein wherein said deaminase has the amino acid sequence set forth as SEQ ID NO: 375 and is inserted within said RGN having the amino acid sequence set forth as SEQ ID NO: 50 after an amino acid position corresponding to position 678 of SEQ ID NO: 2;
g) a fusion protein wherein said deaminase has the amino acid sequence set forth as SEQ ID NO: 319 and is inserted within said RGN having the amino acid sequence set forth as SEQ ID NO: 49 after an amino acid position corresponding to position 772 of SEQ ID NO: 1;
h) a fusion protein wherein said deaminase has the amino acid sequence set forth as SEQ ID NO: 597 and is inserted within said RGN having the amino acid sequence set forth as SEQ ID NO: 50 after an amino acid position corresponding to position 922 of SEQ ID NO: 2;
i) a fusion protein wherein said deaminase has the amino acid sequence set forth as SEQ ID NO: 319 and is inserted within said RGN having the amino acid sequence set forth as SEQ ID NO: 50 after an amino acid position corresponding to position 678 of SEQ ID NO: 2;
j) a fusion protein wherein said deaminase has the amino acid sequence set forth as SEQ ID NO: 406 and is inserted within said RGN having the amino acid sequence set forth as SEQ ID NO: 50 after an amino acid position corresponding to position 678 of SEQ ID NO: 2;
k) a fusion protein wherein said deaminase has the amino acid sequence set forth as SEQ ID NO: 597 and is inserted within said RGN having the amino acid sequence set forth as SEQ ID NO: 50 after an amino acid position corresponding to position 678 of SEQ ID NO: 2;
l) a fusion protein wherein said deaminase has the amino acid sequence set forth as SEQ ID NO: 596 and is inserted within said RGN having the amino acid sequence set forth as SEQ ID NO: 50 after an amino acid position corresponding to position 678 of SEQ ID NO: 2;
m) a fusion protein wherein said deaminase has the amino acid sequence set forth as SEQ ID NO: 406 and is inserted within said RGN having the amino acid sequence set forth as SEQ ID NO: 52 after an amino acid position corresponding to position 910 of SEQ ID NO: 4;
n) a fusion protein wherein said deaminase has the amino acid sequence set forth as SEQ ID NO: 597 and is inserted within said RGN having the amino acid sequence set forth as SEQ ID NO: 52 after an amino acid position corresponding to position 910 of SEQ ID NO: 4;
o) a fusion protein wherein said deaminase has the amino acid sequence set forth as SEQ ID NO: 406 and is inserted within said RGN having the amino acid sequence set forth as SEQ ID NO: 50 after an amino acid position corresponding to position 922 of SEQ ID NO: 2;
p) a fusion protein wherein said deaminase has the amino acid sequence set forth as SEQ ID NO: 596 and is inserted within said RGN having the amino acid sequence set forth as SEQ ID NO: 52 after an amino acid position corresponding to position 910 of SEQ ID NO: 4;
q) a fusion protein wherein said deaminase has the amino acid sequence set forth as SEQ ID NO: 596 and is inserted within said RGN having the amino acid sequence set forth as SEQ ID NO: 50 after an amino acid position corresponding to position 922 of SEQ ID NO: 2;
r) a fusion protein wherein said deaminase has the amino acid sequence set forth as SEQ ID NO: 406 and is inserted within said RGN having the amino acid sequence set forth as SEQ ID NO: 49 after an amino acid position corresponding to position 772 of SEQ ID NO: 1;
s) a fusion protein wherein said deaminase has the amino acid sequence set forth as SEQ ID NO: 596 and is inserted within said RGN having the amino acid sequence set forth as SEQ ID NO: 49 after an amino acid position corresponding to position 772 of SEQ ID NO: 1; and
t) a fusion protein wherein said deaminase has the amino acid sequence set forth as SEQ ID NO: 597 and is inserted within said RGN having the amino acid sequence set forth as SEQ ID NO: 49 after an amino acid position corresponding to position 772 of SEQ ID NO: 1.
243. The fusion protein of embodiment 242, wherein said fusion protein has the sequence set forth as any one of SEQ ID NOs: 599, 600, 602, 603, 606, 610, 612, 614, 615, 617, 619-624, 626, and 630-632.
244. The nucleic acid molecule of embodiment 54, wherein said adenine deaminase comprises an amino acid sequence having at least 85% sequence identity to SEQ ID NO: 5, and comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
245. The nucleic acid molecule of embodiment 244, wherein said adenine deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
246. The nucleic acid molecule of embodiment 244, wherein said adenine deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
247. The nucleic acid molecule of embodiment 244, wherein said adenine deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
248. The nucleic acid molecule of any one of embodiments 244, 246, and 247, wherein said adenine deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
249. The nucleic acid molecule of any one of embodiments 244-248, wherein said deaminase has an amino acid sequence having at least 90% sequence identity to any one of SEQ ID NOs: 300-414, 596-598, and 720-723.
250. The nucleic acid molecule of any one of embodiments 244-248, wherein said deaminase has an amino acid sequence having at least 95% sequence identity to any one of SEQ ID NOs: 300-414, 596-598, and 720-723.
251. The nucleic acid molecule of any one of embodiments 244-248, wherein said deaminase has the amino acid sequence set forth as any one of SEQ ID NOs: 300-414, 596-598, and 720-723.
252. The nucleic acid molecule of any one of embodiments 244-248, wherein said fusion protein is selected from the group consisting of:
a) a fusion protein wherein said deaminase has an amino acid sequence having at least 90% sequence identity to SEQ ID NO: 375 and is inserted within said RGN having an amino acid sequence having at least 90% sequence identity to SEQ ID NO: 50 after an amino acid position corresponding to position 922 of SEQ ID NO: 2;
b) a fusion protein wherein said deaminase has an amino acid sequence having at least 90% sequence identity to SEQ ID NO: 375 and is inserted within said RGN having an amino acid sequence having at least 90% sequence identity to SEQ ID NO: 52 after an amino acid position corresponding to position 910 of SEQ ID NO: 4;
c) a fusion protein wherein said deaminase has an amino acid sequence having at least 90% sequence identity to SEQ ID NO: 319 and is inserted within said RGN having an amino acid sequence having at least 90% sequence identity to SEQ ID NO: 50 after an amino acid position corresponding to position 922 of SEQ ID NO: 2;
d) a fusion protein wherein said deaminase has an amino acid sequence having at least 90% sequence identity to SEQ ID NO: 319 and is inserted within said RGN having an amino acid sequence having at least 90% sequence identity to SEQ ID NO: 52 after an amino acid position corresponding to position 910 of SEQ ID NO: 4;
e) a fusion protein wherein said deaminase has an amino acid sequence having at least 90% sequence identity to SEQ ID NO: 375 and is inserted within said RGN having an amino acid sequence having at least 90% sequence identity to SEQ ID NO: 49 after an amino acid position corresponding to position 772 of SEQ ID NO: 1;
f) a fusion protein wherein said deaminase has an amino acid sequence having at least 90% sequence identity to SEQ ID NO: 375 and is inserted within said RGN having an amino acid sequence having at least 90% sequence identity to SEQ ID NO: 50 after an amino acid position corresponding to position 678 of SEQ ID NO: 2;
g) a fusion protein wherein said deaminase has an amino acid sequence having at least 90% sequence identity to SEQ ID NO: 319 and is inserted within said RGN having an amino acid sequence having at least 90% sequence identity to SEQ ID NO: 49 after an amino acid position corresponding to position 772 of SEQ ID NO: 1;
h) a fusion protein wherein said deaminase has an amino acid sequence having at least 90% sequence identity to SEQ ID NO: 597 and is inserted within said RGN having an amino acid sequence having at least 90% sequence identity to SEQ ID NO: 50 after an amino acid position corresponding to position 922 of SEQ ID NO: 2;
i) a fusion protein wherein said deaminase has an amino acid sequence having at least 90% sequence identity to SEQ ID NO: 319 and is inserted within said RGN having an amino acid sequence having at least 90% sequence identity to SEQ ID NO: 50 after an amino acid position corresponding to position 678 of SEQ ID NO: 2;
j) a fusion protein wherein said deaminase has an amino acid sequence having at least 90% sequence identity to SEQ ID NO: 406 and is inserted within said RGN having an amino acid sequence having at least 90% sequence identity to SEQ ID NO: 50 after an amino acid position corresponding to position 678 of SEQ ID NO: 2;
k) a fusion protein wherein said deaminase has an amino acid sequence having at least 90% sequence identity to SEQ ID NO: 597 and is inserted within said RGN having an amino acid sequence having at least 90% sequence identity to SEQ ID NO: 50 after an amino acid position corresponding to position 678 of SEQ ID NO: 2;
l) a fusion protein wherein said deaminase has an amino acid sequence having at least 90% sequence identity to SEQ ID NO: 596 and is inserted within said RGN having an amino acid sequence having at least 90% sequence identity to SEQ ID NO: 50 after an amino acid position corresponding to position 678 of SEQ ID NO: 2;
m) a fusion protein wherein said deaminase has an amino acid sequence having at least 90% sequence identity to SEQ ID NO: 406 and is inserted within said RGN having an amino acid sequence having at least 90% sequence identity to SEQ ID NO: 52 after an amino acid position corresponding to position 910 of SEQ ID NO: 4;
n) a fusion protein wherein said deaminase has an amino acid sequence having at least 90% sequence identity to SEQ ID NO: 597 and is inserted within said RGN having an amino acid sequence having at least 90% sequence identity to SEQ ID NO: 52 after an amino acid position corresponding to position 910 of SEQ ID NO: 4;
o) a fusion protein wherein said deaminase has an amino acid sequence having at least 90% sequence identity to SEQ ID NO: 406 and is inserted within said RGN having an amino acid sequence having at least 90% sequence identity to SEQ ID NO: 50 after an amino acid position corresponding to position 922 of SEQ ID NO: 2;
p) a fusion protein wherein said deaminase has an amino acid sequence having at least 90% sequence identity to SEQ ID NO: 596 and is inserted within said RGN having an amino acid sequence having at least 90% sequence identity to SEQ ID NO: 52 after an amino acid position corresponding to position 910 of SEQ ID NO: 4;
q) a fusion protein wherein said deaminase has an amino acid sequence having at least 90% sequence identity to SEQ ID NO: 596 and is inserted within said RGN having an amino acid sequence having at least 90% sequence identity to SEQ ID NO: 50 after an amino acid position corresponding to position 922 of SEQ ID NO: 2;
r) a fusion protein wherein said deaminase has an amino acid sequence having at least 90% sequence identity to SEQ ID NO: 406 and is inserted within said RGN having an amino acid sequence having at least 90% sequence identity to SEQ ID NO: 49 after an amino acid position corresponding to position 772 of SEQ ID NO: 1;
s) a fusion protein wherein said deaminase has an amino acid sequence having at least 90% sequence identity to SEQ ID NO: 596 and is inserted within said RGN having an amino acid sequence having at least 90% sequence identity to SEQ ID NO: 49 after an amino acid position corresponding to position 772 of SEQ ID NO: 1; and
t) a fusion protein wherein said deaminase has an amino acid sequence having at least 90% sequence identity to SEQ ID NO: 597 and is inserted within said RGN having an amino acid sequence having at least 90% sequence identity to SEQ ID NO: 49 after an amino acid position corresponding to position 772 of SEQ ID NO: 1.
253. The nucleic acid molecule of any one of embodiments 244-248, wherein said fusion protein is selected from the group consisting of:
a) a fusion protein wherein said deaminase has an amino acid sequence having at least 95% sequence identity to SEQ ID NO: 375 and is inserted within said RGN having an amino acid sequence having at least 95% sequence identity to SEQ ID NO: 50 after an amino acid position corresponding to position 922 of SEQ ID NO: 2;
b) a fusion protein wherein said deaminase has an amino acid sequence having at least 95% sequence identity to SEQ ID NO: 375 and is inserted within said RGN having an amino acid sequence having at least 95% sequence identity to SEQ ID NO: 52 after an amino acid position corresponding to position 910 of SEQ ID NO: 4;
c) a fusion protein wherein said deaminase has an amino acid sequence having at least 95% sequence identity to SEQ ID NO: 319 and is inserted within said RGN having an amino acid sequence having at least 95% sequence identity to SEQ ID NO: 50 after an amino acid position corresponding to position 922 of SEQ ID NO: 2;
d) a fusion protein wherein said deaminase has an amino acid sequence having at least 95% sequence identity to SEQ ID NO: 319 and is inserted within said RGN having an amino acid sequence having at least 95% sequence identity to SEQ ID NO: 52 after an amino acid position corresponding to position 910 of SEQ ID NO: 4;
e) a fusion protein wherein said deaminase has an amino acid sequence having at least 95% sequence identity to SEQ ID NO: 375 and is inserted within said RGN having an amino acid sequence having at least 95% sequence identity to SEQ ID NO: 49 after an amino acid position corresponding to position 772 of SEQ ID NO: 1;
f) a fusion protein wherein said deaminase has an amino acid sequence having at least 95% sequence identity to SEQ ID NO: 375 and is inserted within said RGN having an amino acid sequence having at least 95% sequence identity to SEQ ID NO: 50 after an amino acid position corresponding to position 678 of SEQ ID NO: 2;
g) a fusion protein wherein said deaminase has an amino acid sequence having at least 95% sequence identity to SEQ ID NO: 319 and is inserted within said RGN having an amino acid sequence having at least 95% sequence identity to SEQ ID NO: 49 after an amino acid position corresponding to position 772 of SEQ ID NO: 1;
h) a fusion protein wherein said deaminase has an amino acid sequence having at least 95% sequence identity to SEQ ID NO: 597 and is inserted within said RGN having an amino acid sequence having at least 95% sequence identity to SEQ ID NO: 50 after an amino acid position corresponding to position 922 of SEQ ID NO: 2;
i) a fusion protein wherein said deaminase has an amino acid sequence having at least 95% sequence identity to SEQ ID NO: 319 and is inserted within said RGN having an amino acid sequence having at least 95% sequence identity to SEQ ID NO: 50 after an amino acid position corresponding to position 678 of SEQ ID NO: 2;
j) a fusion protein wherein said deaminase has an amino acid sequence having at least 95% sequence identity to SEQ ID NO: 406 and is inserted within said RGN having an amino acid sequence having at least 95% sequence identity to SEQ ID NO: 50 after an amino acid position corresponding to position 678 of SEQ ID NO: 2;
k) a fusion protein wherein said deaminase has an amino acid sequence having at least 95% sequence identity to SEQ ID NO: 597 and is inserted within said RGN having an amino acid sequence having at least 95% sequence identity to SEQ ID NO: 50 after an amino acid position corresponding to position 678 of SEQ ID NO: 2;
l) a fusion protein wherein said deaminase has an amino acid sequence having at least 95% sequence identity to SEQ ID NO: 596 and is inserted within said RGN having an amino acid sequence having at least 95% sequence identity to SEQ ID NO: 50 after an amino acid position corresponding to position 678 of SEQ ID NO: 2;
m) a fusion protein wherein said deaminase has an amino acid sequence having at least 95% sequence identity to SEQ ID NO: 406 and is inserted within said RGN having an amino acid sequence having at least 95% sequence identity to SEQ ID NO: 52 after an amino acid position corresponding to position 910 of SEQ ID NO: 4;
n) a fusion protein wherein said deaminase has an amino acid sequence having at least 95% sequence identity to SEQ ID NO: 597 and is inserted within said RGN having an amino acid sequence having at least 95% sequence identity to SEQ ID NO: 52 after an amino acid position corresponding to position 910 of SEQ ID NO: 4;
o) a fusion protein wherein said deaminase has an amino acid sequence having at least 95% sequence identity to SEQ ID NO: 406 and is inserted within said RGN having an amino acid sequence having at least 95% sequence identity to SEQ ID NO: 50 after an amino acid position corresponding to position 922 of SEQ ID NO: 2;
p) a fusion protein wherein said deaminase has an amino acid sequence having at least 95% sequence identity to SEQ ID NO: 596 and is inserted within said RGN having an amino acid sequence having at least 95% sequence identity to SEQ ID NO: 52 after an amino acid position corresponding to position 910 of SEQ ID NO: 4;
q) a fusion protein wherein said deaminase has an amino acid sequence having at least 95% sequence identity to SEQ ID NO: 596 and is inserted within said RGN having an amino acid sequence having at least 95% sequence identity to SEQ ID NO: 50 after an amino acid position corresponding to position 922 of SEQ ID NO: 2;
r) a fusion protein wherein said deaminase has an amino acid sequence having at least 95% sequence identity to SEQ ID NO: 406 and is inserted within said RGN having an amino acid sequence having at least 95% sequence identity to SEQ ID NO: 49 after an amino acid position corresponding to position 772 of SEQ ID NO: 1;
s) a fusion protein wherein said deaminase has an amino acid sequence having at least 95% sequence identity to SEQ ID NO: 596 and is inserted within said RGN having an amino acid sequence having at least 95% sequence identity to SEQ ID NO: 49 after an amino acid position corresponding to position 772 of SEQ ID NO: 1; and
t) a fusion protein wherein said deaminase has an amino acid sequence having at least 95% sequence identity to SEQ ID NO: 597 and is inserted within said RGN having an amino acid sequence having at least 95% sequence identity to SEQ ID NO: 49 after an amino acid position corresponding to position 772 of SEQ ID NO: 1.
254. The nucleic acid molecule of any one of embodiments 244-248, wherein said fusion protein is selected from the group consisting of:
a) a fusion protein wherein said deaminase has the amino acid sequence set forth as SEQ ID NO: 375 and is inserted within said RGN having the amino acid sequence set forth as SEQ ID NO: 50 after an amino acid position corresponding to position 922 of SEQ ID NO: 2;
b) a fusion protein wherein said deaminase has the amino acid sequence set forth as SEQ ID NO: 375 and is inserted within said RGN having the amino acid sequence set forth as SEQ ID NO: 52 after an amino acid position corresponding to position 910 of SEQ ID NO: 4;
c) a fusion protein wherein said deaminase has the amino acid sequence set forth as SEQ ID NO: 319 and is inserted within said RGN having the amino acid sequence set forth as SEQ ID NO: 50 after an amino acid position corresponding to position 922 of SEQ ID NO: 2;
d) a fusion protein wherein said deaminase has the amino acid sequence set forth as SEQ ID NO: 319 and is inserted within said RGN having the amino acid sequence set forth as SEQ ID NO: 52 after an amino acid position corresponding to position 910 of SEQ ID NO: 4;
e) a fusion protein wherein said deaminase has the amino acid sequence set forth as SEQ ID NO: 375 and is inserted within said RGN having the amino acid sequence set forth as SEQ ID NO: 49 after an amino acid position corresponding to position 772 of SEQ ID NO: 1;
f) a fusion protein wherein said deaminase has the amino acid sequence set forth as SEQ ID NO: 375 and is inserted within said RGN having the amino acid sequence set forth as SEQ ID NO: 50 after an amino acid position corresponding to position 678 of SEQ ID NO: 2;
g) a fusion protein wherein said deaminase has the amino acid sequence set forth as SEQ ID NO: 319 and is inserted within said RGN having the amino acid sequence set forth as SEQ ID NO: 49 after an amino acid position corresponding to position 772 of SEQ ID NO: 1;
h) a fusion protein wherein said deaminase has the amino acid sequence set forth as SEQ ID NO: 597 and is inserted within said RGN having the amino acid sequence set forth as SEQ ID NO: 50 after an amino acid position corresponding to position 922 of SEQ ID NO: 2;
i) a fusion protein wherein said deaminase has the amino acid sequence set forth as SEQ ID NO: 319 and is inserted within said RGN having the amino acid sequence set forth as SEQ ID NO: 50 after an amino acid position corresponding to position 678 of SEQ ID NO: 2;
j) a fusion protein wherein said deaminase has the amino acid sequence set forth as SEQ ID NO: 406 and is inserted within said RGN having the amino acid sequence set forth as SEQ ID NO: 50 after an amino acid position corresponding to position 678 of SEQ ID NO: 2;
k) a fusion protein wherein said deaminase has the amino acid sequence set forth as SEQ ID NO: 597 and is inserted within said RGN having the amino acid sequence set forth as SEQ ID NO: 50 after an amino acid position corresponding to position 678 of SEQ ID NO: 2;
l) a fusion protein wherein said deaminase has the amino acid sequence set forth as SEQ ID NO: 596 and is inserted within said RGN having the amino acid sequence set forth as SEQ ID NO: 50 after an amino acid position corresponding to position 678 of SEQ ID NO: 2;
m) a fusion protein wherein said deaminase has the amino acid sequence set forth as SEQ ID NO: 406 and is inserted within said RGN having the amino acid sequence set forth as SEQ ID NO: 52 after an amino acid position corresponding to position 910 of SEQ ID NO: 4;
n) a fusion protein wherein said deaminase has the amino acid sequence set forth as SEQ ID NO: 597 and is inserted within said RGN having the amino acid sequence set forth as SEQ ID NO: 52 after an amino acid position corresponding to position 910 of SEQ ID NO: 4;
o) a fusion protein wherein said deaminase has the amino acid sequence set forth as SEQ ID NO: 406 and is inserted within said RGN having the amino acid sequence set forth as SEQ ID NO: 50 after an amino acid position corresponding to position 922 of SEQ ID NO: 2;
p) a fusion protein wherein said deaminase has the amino acid sequence set forth as SEQ ID NO: 596 and is inserted within said RGN having the amino acid sequence set forth as SEQ ID NO: 52 after an amino acid position corresponding to position 910 of SEQ ID NO: 4;
q) a fusion protein wherein said deaminase has the amino acid sequence set forth as SEQ ID NO: 596 and is inserted within said RGN having the amino acid sequence set forth as SEQ ID NO: 50 after an amino acid position corresponding to position 922 of SEQ ID NO: 2;
r) a fusion protein wherein said deaminase has the amino acid sequence set forth as SEQ ID NO: 406 and is inserted within said RGN having the amino acid sequence set forth as SEQ ID NO: 49 after an amino acid position corresponding to position 772 of SEQ ID NO: 1;
s) a fusion protein wherein said deaminase has the amino acid sequence set forth as SEQ ID NO: 596 and is inserted within said RGN having the amino acid sequence set forth as SEQ ID NO: 49 after an amino acid position corresponding to position 772 of SEQ ID NO: 1; and
t) a fusion protein wherein said deaminase has the amino acid sequence set forth as SEQ ID NO: 597 and is inserted within said RGN having the amino acid sequence set forth as SEQ ID NO: 49 after an amino acid position corresponding to position 772 of SEQ ID NO: 1.
255. The nucleic acid molecule of embodiment 254, wherein said fusion protein has the sequence set forth as any one of SEQ ID NOs: 599, 600, 602, 603, 606, 610, 612, 614, 615, 617, 619-624, 626, and 630-632.
256. The system of embodiment 117, wherein said adenine deaminase comprises an amino acid sequence having at least 85% sequence identity to SEQ ID NO: 5, and comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
257. The system of embodiment 256, wherein said adenine deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
258. The system of embodiment 256, wherein said adenine deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
259. The system of embodiment 256, wherein said adenine deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
260. The system of any one of embodiments 256, 258, and 259, wherein said adenine deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
261. The system of any one of embodiments 256-260, wherein said deaminase has an amino acid sequence having at least 90% sequence identity to any one of SEQ ID NOs: 300-414, 596-598, and 720-723.
262. The system of any one of embodiments 256-260, wherein said deaminase has an amino acid sequence having at least 95% sequence identity to any one of SEQ ID NOs: 300-414, 596-598, and 720-723.
263. The system of any one of embodiments 256-260, wherein said deaminase has the amino acid sequence set forth as any one of SEQ ID NOs: 300-414, 596-598, and 720-723.
264. The system of any one of embodiments 256-263, wherein said fusion protein is selected from the group consisting of:
a) a fusion protein wherein said deaminase has an amino acid sequence having at least 90% sequence identity to SEQ ID NO: 375 and is inserted within said RGN having an amino acid sequence having at least 90% sequence identity to SEQ ID NO: 50 after an amino acid position corresponding to position 922 of SEQ ID NO: 2;
b) a fusion protein wherein said deaminase has an amino acid sequence having at least 90% sequence identity to SEQ ID NO: 375 and is inserted within said RGN having an amino acid sequence having at least 90% sequence identity to SEQ ID NO: 52 after an amino acid position corresponding to position 910 of SEQ ID NO: 4;
c) a fusion protein wherein said deaminase has an amino acid sequence having at least 90% sequence identity to SEQ ID NO: 319 and is inserted within said RGN having an amino acid sequence having at least 90% sequence identity to SEQ ID NO: 50 after an amino acid position corresponding to position 922 of SEQ ID NO: 2;
d) a fusion protein wherein said deaminase has an amino acid sequence having at least 90% sequence identity to SEQ ID NO: 319 and is inserted within said RGN having an amino acid sequence having at least 90% sequence identity to SEQ ID NO: 52 after an amino acid position corresponding to position 910 of SEQ ID NO: 4;
e) a fusion protein wherein said deaminase has an amino acid sequence having at least 90% sequence identity to SEQ ID NO: 375 and is inserted within said RGN having an amino acid sequence having at least 90% sequence identity to SEQ ID NO: 49 after an amino acid position corresponding to position 772 of SEQ ID NO: 1;
f) a fusion protein wherein said deaminase has an amino acid sequence having at least 90% sequence identity to SEQ ID NO: 375 and is inserted within said RGN having an amino acid sequence having at least 90% sequence identity to SEQ ID NO: 50 after an amino acid position corresponding to position 678 of SEQ ID NO: 2;
g) a fusion protein wherein said deaminase has an amino acid sequence having at least 90% sequence identity to SEQ ID NO: 319 and is inserted within said RGN having an amino acid sequence having at least 90% sequence identity to SEQ ID NO: 49 after an amino acid position corresponding to position 772 of SEQ ID NO: 1;
h) a fusion protein wherein said deaminase has an amino acid sequence having at least 90% sequence identity to SEQ ID NO: 597 and is inserted within said RGN having an amino acid sequence having at least 90% sequence identity to SEQ ID NO: 50 after an amino acid position corresponding to position 922 of SEQ ID NO: 2;
i) a fusion protein wherein said deaminase has an amino acid sequence having at least 90% sequence identity to SEQ ID NO: 319 and is inserted within said RGN having an amino acid sequence having at least 90% sequence identity to SEQ ID NO: 50 after an amino acid position corresponding to position 678 of SEQ ID NO: 2;
j) a fusion protein wherein said deaminase has an amino acid sequence having at least 90% sequence identity to SEQ ID NO: 406 and is inserted within said RGN having an amino acid sequence having at least 90% sequence identity to SEQ ID NO: 50 after an amino acid position corresponding to position 678 of SEQ ID NO: 2;
k) a fusion protein wherein said deaminase has an amino acid sequence having at least 90% sequence identity to SEQ ID NO: 597 and is inserted within said RGN having an amino acid sequence having at least 90% sequence identity to SEQ ID NO: 50 after an amino acid position corresponding to position 678 of SEQ ID NO: 2;
l) a fusion protein wherein said deaminase has an amino acid sequence having at least 90% sequence identity to SEQ ID NO: 596 and is inserted within said RGN having an amino acid sequence having at least 90% sequence identity to SEQ ID NO: 50 after an amino acid position corresponding to position 678 of SEQ ID NO: 2;
m) a fusion protein wherein said deaminase has an amino acid sequence having at least 90% sequence identity to SEQ ID NO: 406 and is inserted within said RGN having an amino acid sequence having at least 90% sequence identity to SEQ ID NO: 52 after an amino acid position corresponding to position 910 of SEQ ID NO: 4;
n) a fusion protein wherein said deaminase has an amino acid sequence having at least 90% sequence identity to SEQ ID NO: 597 and is inserted within said RGN having an amino acid sequence having at least 90% sequence identity to SEQ ID NO: 52 after an amino acid position corresponding to position 910 of SEQ ID NO: 4;
o) a fusion protein wherein said deaminase has an amino acid sequence having at least 90% sequence identity to SEQ ID NO: 406 and is inserted within said RGN having an amino acid sequence having at least 90% sequence identity to SEQ ID NO: 50 after an amino acid position corresponding to position 922 of SEQ ID NO: 2;
p) a fusion protein wherein said deaminase has an amino acid sequence having at least 90% sequence identity to SEQ ID NO: 596 and is inserted within said RGN having an amino acid sequence having at least 90% sequence identity to SEQ ID NO: 52 after an amino acid position corresponding to position 910 of SEQ ID NO: 4;
q) a fusion protein wherein said deaminase has an amino acid sequence having at least 90% sequence identity to SEQ ID NO: 596 and is inserted within said RGN having an amino acid sequence having at least 90% sequence identity to SEQ ID NO: 50 after an amino acid position corresponding to position 922 of SEQ ID NO: 2;
r) a fusion protein wherein said deaminase has an amino acid sequence having at least 90% sequence identity to SEQ ID NO: 406 and is inserted within said RGN having an amino acid sequence having at least 90% sequence identity to SEQ ID NO: 49 after an amino acid position corresponding to position 772 of SEQ ID NO: 1;
s) a fusion protein wherein said deaminase has an amino acid sequence having at least 90% sequence identity to SEQ ID NO: 596 and is inserted within said RGN having an amino acid sequence having at least 90% sequence identity to SEQ ID NO: 49 after an amino acid position corresponding to position 772 of SEQ ID NO: 1; and
t) a fusion protein wherein said deaminase has an amino acid sequence having at least 90% sequence identity to SEQ ID NO: 597 and is inserted within said RGN having an amino acid sequence having at least 90% sequence identity to SEQ ID NO: 49 after an amino acid position corresponding to position 772 of SEQ ID NO: 1.
265. The system of any one of embodiments 256-263, wherein said fusion protein is selected from the group consisting of:
a) a fusion protein wherein said deaminase has an amino acid sequence having at least 95% sequence identity to SEQ ID NO: 375 and is inserted within said RGN having an amino acid sequence having at least 95% sequence identity to SEQ ID NO: 50 after an amino acid position corresponding to position 922 of SEQ ID NO: 2;
b) a fusion protein wherein said deaminase has an amino acid sequence having at least 95% sequence identity to SEQ ID NO: 375 and is inserted within said RGN having an amino acid sequence having at least 95% sequence identity to SEQ ID NO: 52 after an amino acid position corresponding to position 910 of SEQ ID NO: 4;
c) a fusion protein wherein said deaminase has an amino acid sequence having at least 95% sequence identity to SEQ ID NO: 319 and is inserted within said RGN having an amino acid sequence having at least 95% sequence identity to SEQ ID NO: 50 after an amino acid position corresponding to position 922 of SEQ ID NO: 2;
d) a fusion protein wherein said deaminase has an amino acid sequence having at least 95% sequence identity to SEQ ID NO: 319 and is inserted within said RGN having an amino acid sequence having at least 95% sequence identity to SEQ ID NO: 52 after an amino acid position corresponding to position 910 of SEQ ID NO: 4;
e) a fusion protein wherein said deaminase has an amino acid sequence having at least 95% sequence identity to SEQ ID NO: 375 and is inserted within said RGN having an amino acid sequence having at least 95% sequence identity to SEQ ID NO: 49 after an amino acid position corresponding to position 772 of SEQ ID NO: 1;
f) a fusion protein wherein said deaminase has an amino acid sequence having at least 95% sequence identity to SEQ ID NO: 375 and is inserted within said RGN having an amino acid sequence having at least 95% sequence identity to SEQ ID NO: 50 after an amino acid position corresponding to position 678 of SEQ ID NO: 2;
g) a fusion protein wherein said deaminase has an amino acid sequence having at least 95% sequence identity to SEQ ID NO: 319 and is inserted within said RGN having an amino acid sequence having at least 95% sequence identity to SEQ ID NO: 49 after an amino acid position corresponding to position 772 of SEQ ID NO: 1;
h) a fusion protein wherein said deaminase has an amino acid sequence having at least 95% sequence identity to SEQ ID NO: 597 and is inserted within said RGN having an amino acid sequence having at least 95% sequence identity to SEQ ID NO: 50 after an amino acid position corresponding to position 922 of SEQ ID NO: 2;
i) a fusion protein wherein said deaminase has an amino acid sequence having at least 95% sequence identity to SEQ ID NO: 319 and is inserted within said RGN having an amino acid sequence having at least 95% sequence identity to SEQ ID NO: 50 after an amino acid position corresponding to position 678 of SEQ ID NO: 2;
j) a fusion protein wherein said deaminase has an amino acid sequence having at least 95% sequence identity to SEQ ID NO: 406 and is inserted within said RGN having an amino acid sequence having at least 95% sequence identity to SEQ ID NO: 50 after an amino acid position corresponding to position 678 of SEQ ID NO: 2;
k) a fusion protein wherein said deaminase has an amino acid sequence having at least 95% sequence identity to SEQ ID NO: 597 and is inserted within said RGN having an amino acid sequence having at least 95% sequence identity to SEQ ID NO: 50 after an amino acid position corresponding to position 678 of SEQ ID NO: 2;
l) a fusion protein wherein said deaminase has an amino acid sequence having at least 95% sequence identity to SEQ ID NO: 596 and is inserted within said RGN having an amino acid sequence having at least 95% sequence identity to SEQ ID NO: 50 after an amino acid position corresponding to position 678 of SEQ ID NO: 2;
m) a fusion protein wherein said deaminase has an amino acid sequence having at least 95% sequence identity to SEQ ID NO: 406 and is inserted within said RGN having an amino acid sequence having at least 95% sequence identity to SEQ ID NO: 52 after an amino acid position corresponding to position 910 of SEQ ID NO: 4;
n) a fusion protein wherein said deaminase has an amino acid sequence having at least 95% sequence identity to SEQ ID NO: 597 and is inserted within said RGN having an amino acid sequence having at least 95% sequence identity to SEQ ID NO: 52 after an amino acid position corresponding to position 910 of SEQ ID NO: 4;
o) a fusion protein wherein said deaminase has an amino acid sequence having at least 95% sequence identity to SEQ ID NO: 406 and is inserted within said RGN having an amino acid sequence having at least 95% sequence identity to SEQ ID NO: 50 after an amino acid position corresponding to position 922 of SEQ ID NO: 2;
p) a fusion protein wherein said deaminase has an amino acid sequence having at least 95% sequence identity to SEQ ID NO: 596 and is inserted within said RGN having an amino acid sequence having at least 95% sequence identity to SEQ ID NO: 52 after an amino acid position corresponding to position 910 of SEQ ID NO: 4;
q) a fusion protein wherein said deaminase has an amino acid sequence having at least 95% sequence identity to SEQ ID NO: 596 and is inserted within said RGN having an amino acid sequence having at least 95% sequence identity to SEQ ID NO: 50 after an amino acid position corresponding to position 922 of SEQ ID NO: 2;
r) a fusion protein wherein said deaminase has an amino acid sequence having at least 95% sequence identity to SEQ ID NO: 406 and is inserted within said RGN having an amino acid sequence having at least 95% sequence identity to SEQ ID NO: 49 after an amino acid position corresponding to position 772 of SEQ ID NO: 1;
s) a fusion protein wherein said deaminase has an amino acid sequence having at least 95% sequence identity to SEQ ID NO: 596 and is inserted within said RGN having an amino acid sequence having at least 95% sequence identity to SEQ ID NO: 49 after an amino acid position corresponding to position 772 of SEQ ID NO: 1; and
t) a fusion protein wherein said deaminase has an amino acid sequence having at least 95% sequence identity to SEQ ID NO: 597 and is inserted within said RGN having an amino acid sequence having at least 95% sequence identity to SEQ ID NO: 49 after an amino acid position corresponding to position 772 of SEQ ID NO: 1.
266. The system of any one of embodiments 256-263, wherein said fusion protein is selected from the group consisting of:
a) a fusion protein wherein said deaminase has the amino acid sequence set forth as SEQ ID NO: 375 and is inserted within said RGN having the amino acid sequence set forth as SEQ ID NO: 50 after an amino acid position corresponding to position 922 of SEQ ID NO: 2;
b) a fusion protein wherein said deaminase has the amino acid sequence set forth as SEQ ID NO: 375 and is inserted within said RGN having the amino acid sequence set forth as SEQ ID NO: 52 after an amino acid position corresponding to position 910 of SEQ ID NO: 4;
c) a fusion protein wherein said deaminase has the amino acid sequence set forth as SEQ ID NO: 319 and is inserted within said RGN having the amino acid sequence set forth as SEQ ID NO: 50 after an amino acid position corresponding to position 922 of SEQ ID NO: 2;
d) a fusion protein wherein said deaminase has the amino acid sequence set forth as SEQ ID NO: 319 and is inserted within said RGN having the amino acid sequence set forth as SEQ ID NO: 52 after an amino acid position corresponding to position 910 of SEQ ID NO: 4;
e) a fusion protein wherein said deaminase has the amino acid sequence set forth as SEQ ID NO: 375 and is inserted within said RGN having the amino acid sequence set forth as SEQ ID NO: 49 after an amino acid position corresponding to position 772 of SEQ ID NO: 1;
f) a fusion protein wherein said deaminase has the amino acid sequence set forth as SEQ ID NO: 375 and is inserted within said RGN having the amino acid sequence set forth as SEQ ID NO: 50 after an amino acid position corresponding to position 678 of SEQ ID NO: 2;
g) a fusion protein wherein said deaminase has the amino acid sequence set forth as SEQ ID NO: 319 and is inserted within said RGN having the amino acid sequence set forth as SEQ ID NO: 49 after an amino acid position corresponding to position 772 of SEQ ID NO: 1;
h) a fusion protein wherein said deaminase has the amino acid sequence set forth as SEQ ID NO: 597 and is inserted within said RGN having the amino acid sequence set forth as SEQ ID NO: 50 after an amino acid position corresponding to position 922 of SEQ ID NO: 2;
i) a fusion protein wherein said deaminase has the amino acid sequence set forth as SEQ ID NO: 319 and is inserted within said RGN having the amino acid sequence set forth as SEQ ID NO: 50 after an amino acid position corresponding to position 678 of SEQ ID NO: 2;
j) a fusion protein wherein said deaminase has the amino acid sequence set forth as SEQ ID NO: 406 and is inserted within said RGN having the amino acid sequence set forth as SEQ ID NO: 50 after an amino acid position corresponding to position 678 of SEQ ID NO: 2;
k) a fusion protein wherein said deaminase has the amino acid sequence set forth as SEQ ID NO: 597 and is inserted within said RGN having the amino acid sequence set forth as SEQ ID NO: 50 after an amino acid position corresponding to position 678 of SEQ ID NO: 2;
l) a fusion protein wherein said deaminase has the amino acid sequence set forth as SEQ ID NO: 596 and is inserted within said RGN having the amino acid sequence set forth as SEQ ID NO: 50 after an amino acid position corresponding to position 678 of SEQ ID NO: 2;
m) a fusion protein wherein said deaminase has the amino acid sequence set forth as SEQ ID NO: 406 and is inserted within said RGN having the amino acid sequence set forth as SEQ ID NO: 52 after an amino acid position corresponding to position 910 of SEQ ID NO: 4;
n) a fusion protein wherein said deaminase has the amino acid sequence set forth as SEQ ID NO: 597 and is inserted within said RGN having the amino acid sequence set forth as SEQ ID NO: 52 after an amino acid position corresponding to position 910 of SEQ ID NO: 4;
o) a fusion protein wherein said deaminase has the amino acid sequence set forth as SEQ ID NO: 406 and is inserted within said RGN having the amino acid sequence set forth as SEQ ID NO: 50 after an amino acid position corresponding to position 922 of SEQ ID NO: 2;
p) a fusion protein wherein said deaminase has the amino acid sequence set forth as SEQ ID NO: 596 and is inserted within said RGN having the amino acid sequence set forth as SEQ ID NO: 52 after an amino acid position corresponding to position 910 of SEQ ID NO: 4;
q) a fusion protein wherein said deaminase has the amino acid sequence set forth as SEQ ID NO: 596 and is inserted within said RGN having the amino acid sequence set forth as SEQ ID NO: 50 after an amino acid position corresponding to position 922 of SEQ ID NO: 2;
r) a fusion protein wherein said deaminase has the amino acid sequence set forth as SEQ ID NO: 406 and is inserted within said RGN having the amino acid sequence set forth as SEQ ID NO: 49 after an amino acid position corresponding to position 772 of SEQ ID NO: 1;
s) a fusion protein wherein said deaminase has the amino acid sequence set forth as SEQ ID NO: 596 and is inserted within said RGN having the amino acid sequence set forth as SEQ ID NO: 49 after an amino acid position corresponding to position 772 of SEQ ID NO: 1; and
t) a fusion protein wherein said deaminase has the amino acid sequence set forth as SEQ ID NO: 597 and is inserted within said RGN having the amino acid sequence set forth as SEQ ID NO: 49 after an amino acid position corresponding to position 772 of SEQ ID NO: 1.
267. The system of embodiment 266, wherein said fusion protein has the sequence set forth as any one of SEQ ID NOs: 599, 600, 602, 603, 606, 610, 612, 614, 615, 617, 619-624, 626, and 630-632.
268. The method of embodiment 176, wherein said adenine deaminase comprises an amino acid sequence having at least 85% sequence identity to SEQ ID NO: 5, and comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
269. The method of embodiment 268, wherein said adenine deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
270. The method of embodiment 268, wherein said adenine deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
271. The method of embodiment 268, wherein said adenine deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
272. The method of any one of embodiments 268, 270, and 271, wherein said adenine deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
273. The method of any one of embodiments 268-272, wherein said deaminase has an amino acid sequence having at least 90% sequence identity to any one of SEQ ID NOs: 300-414, 596-598, and 720-723.
274. The method of any one of embodiments 268-272, wherein said deaminase has an amino acid sequence having at least 95% sequence identity to any one of SEQ ID NOs: 300-414, 596-598, and 720-723.
275. The method of any one of embodiments 268-272, wherein said deaminase has the amino acid sequence set forth as any one of SEQ ID NOs: 300-414, 596-598, and 720-723.
276. The method of any one of embodiments 268-272, wherein said fusion protein is selected from the group consisting of:
a) a fusion protein wherein said deaminase has an amino acid sequence having at least 90% sequence identity to SEQ ID NO: 375 and is inserted within said RGN having an amino acid sequence having at least 90% sequence identity to SEQ ID NO: 50 after an amino acid position corresponding to position 922 of SEQ ID NO: 2;
b) a fusion protein wherein said deaminase has an amino acid sequence having at least 90% sequence identity to SEQ ID NO: 375 and is inserted within said RGN having an amino acid sequence having at least 90% sequence identity to SEQ ID NO: 52 after an amino acid position corresponding to position 910 of SEQ ID NO: 4;
c) a fusion protein wherein said deaminase has an amino acid sequence having at least 90% sequence identity to SEQ ID NO: 319 and is inserted within said RGN having an amino acid sequence having at least 90% sequence identity to SEQ ID NO: 50 after an amino acid position corresponding to position 922 of SEQ ID NO: 2;
d) a fusion protein wherein said deaminase has an amino acid sequence having at least 90% sequence identity to SEQ ID NO: 319 and is inserted within said RGN having an amino acid sequence having at least 90% sequence identity to SEQ ID NO: 52 after an amino acid position corresponding to position 910 of SEQ ID NO: 4;
e) a fusion protein wherein said deaminase has an amino acid sequence having at least 90% sequence identity to SEQ ID NO: 375 and is inserted within said RGN having an amino acid sequence having at least 90% sequence identity to SEQ ID NO: 49 after an amino acid position corresponding to position 772 of SEQ ID NO: 1;
f) a fusion protein wherein said deaminase has an amino acid sequence having at least 90% sequence identity to SEQ ID NO: 375 and is inserted within said RGN having an amino acid sequence having at least 90% sequence identity to SEQ ID NO: 50 after an amino acid position corresponding to position 678 of SEQ ID NO: 2;
g) a fusion protein wherein said deaminase has an amino acid sequence having at least 90% sequence identity to SEQ ID NO: 319 and is inserted within said RGN having an amino acid sequence having at least 90% sequence identity to SEQ ID NO: 49 after an amino acid position corresponding to position 772 of SEQ ID NO: 1;
h) a fusion protein wherein said deaminase has an amino acid sequence having at least 90% sequence identity to SEQ ID NO: 597 and is inserted within said RGN having an amino acid sequence having at least 90% sequence identity to SEQ ID NO: 50 after an amino acid position corresponding to position 922 of SEQ ID NO: 2;
i) a fusion protein wherein said deaminase has an amino acid sequence having at least 90% sequence identity to SEQ ID NO: 319 and is inserted within said RGN having an amino acid sequence having at least 90% sequence identity to SEQ ID NO: 50 after an amino acid position corresponding to position 678 of SEQ ID NO: 2;
j) a fusion protein wherein said deaminase has an amino acid sequence having at least 90% sequence identity to SEQ ID NO: 406 and is inserted within said RGN having an amino acid sequence having at least 90% sequence identity to SEQ ID NO: 50 after an amino acid position corresponding to position 678 of SEQ ID NO: 2;
k) a fusion protein wherein said deaminase has an amino acid sequence having at least 90% sequence identity to SEQ ID NO: 597 and is inserted within said RGN having an amino acid sequence having at least 90% sequence identity to SEQ ID NO: 50 after an amino acid position corresponding to position 678 of SEQ ID NO: 2;
l) a fusion protein wherein said deaminase has an amino acid sequence having at least 90% sequence identity to SEQ ID NO: 596 and is inserted within said RGN having an amino acid sequence having at least 90% sequence identity to SEQ ID NO: 50 after an amino acid position corresponding to position 678 of SEQ ID NO: 2;
m) a fusion protein wherein said deaminase has an amino acid sequence having at least 90% sequence identity to SEQ ID NO: 406 and is inserted within said RGN having an amino acid sequence having at least 90% sequence identity to SEQ ID NO: 52 after an amino acid position corresponding to position 910 of SEQ ID NO: 4;
n) a fusion protein wherein said deaminase has an amino acid sequence having at least 90% sequence identity to SEQ ID NO: 597 and is inserted within said RGN having an amino acid sequence having at least 90% sequence identity to SEQ ID NO: 52 after an amino acid position corresponding to position 910 of SEQ ID NO: 4;
o) a fusion protein wherein said deaminase has an amino acid sequence having at least 90% sequence identity to SEQ ID NO: 406 and is inserted within said RGN having an amino acid sequence having at least 90% sequence identity to SEQ ID NO: 50 after an amino acid position corresponding to position 922 of SEQ ID NO: 2;
p) a fusion protein wherein said deaminase has an amino acid sequence having at least 90% sequence identity to SEQ ID NO: 596 and is inserted within said RGN having an amino acid sequence having at least 90% sequence identity to SEQ ID NO: 52 after an amino acid position corresponding to position 910 of SEQ ID NO: 4;
q) a fusion protein wherein said deaminase has an amino acid sequence having at least 90% sequence identity to SEQ ID NO: 596 and is inserted within said RGN having an amino acid sequence having at least 90% sequence identity to SEQ ID NO: 50 after an amino acid position corresponding to position 922 of SEQ ID NO: 2;
r) a fusion protein wherein said deaminase has an amino acid sequence having at least 90% sequence identity to SEQ ID NO: 406 and is inserted within said RGN having an amino acid sequence having at least 90% sequence identity to SEQ ID NO: 49 after an amino acid position corresponding to position 772 of SEQ ID NO: 1;
s) a fusion protein wherein said deaminase has an amino acid sequence having at least 90% sequence identity to SEQ ID NO: 596 and is inserted within said RGN having an amino acid sequence having at least 90% sequence identity to SEQ ID NO: 49 after an amino acid position corresponding to position 772 of SEQ ID NO: 1; and
t) a fusion protein wherein said deaminase has an amino acid sequence having at least 90% sequence identity to SEQ ID NO: 597 and is inserted within said RGN having an amino acid sequence having at least 90% sequence identity to SEQ ID NO: 49 after an amino acid position corresponding to position 772 of SEQ ID NO: 1.
277. The method of any one of embodiments 268-272, wherein said fusion protein is selected from the group consisting of:
a) a fusion protein wherein said deaminase has an amino acid sequence having at least 95% sequence identity to SEQ ID NO: 375 and is inserted within said RGN having an amino acid sequence having at least 95% sequence identity to SEQ ID NO: 50 after an amino acid position corresponding to position 922 of SEQ ID NO: 2;
b) a fusion protein wherein said deaminase has an amino acid sequence having at least 95% sequence identity to SEQ ID NO: 375 and is inserted within said RGN having an amino acid sequence having at least 95% sequence identity to SEQ ID NO: 52 after an amino acid position corresponding to position 910 of SEQ ID NO: 4;
c) a fusion protein wherein said deaminase has an amino acid sequence having at least 95% sequence identity to SEQ ID NO: 319 and is inserted within said RGN having an amino acid sequence having at least 95% sequence identity to SEQ ID NO: 50 after an amino acid position corresponding to position 922 of SEQ ID NO: 2;
d) a fusion protein wherein said deaminase has an amino acid sequence having at least 95% sequence identity to SEQ ID NO: 319 and is inserted within said RGN having an amino acid sequence having at least 95% sequence identity to SEQ ID NO: 52 after an amino acid position corresponding to position 910 of SEQ ID NO: 4;
e) a fusion protein wherein said deaminase has an amino acid sequence having at least 95% sequence identity to SEQ ID NO: 375 and is inserted within said RGN having an amino acid sequence having at least 95% sequence identity to SEQ ID NO: 49 after an amino acid position corresponding to position 772 of SEQ ID NO: 1;
f) a fusion protein wherein said deaminase has an amino acid sequence having at least 95% sequence identity to SEQ ID NO: 375 and is inserted within said RGN having an amino acid sequence having at least 95% sequence identity to SEQ ID NO: 50 after an amino acid position corresponding to position 678 of SEQ ID NO: 2;
g) a fusion protein wherein said deaminase has an amino acid sequence having at least 95% sequence identity to SEQ ID NO: 319 and is inserted within said RGN having an amino acid sequence having at least 95% sequence identity to SEQ ID NO: 49 after an amino acid position corresponding to position 772 of SEQ ID NO: 1;
h) a fusion protein wherein said deaminase has an amino acid sequence having at least 95% sequence identity to SEQ ID NO: 597 and is inserted within said RGN having an amino acid sequence having at least 95% sequence identity to SEQ ID NO: 50 after an amino acid position corresponding to position 922 of SEQ ID NO: 2;
i) a fusion protein wherein said deaminase has an amino acid sequence having at least 95% sequence identity to SEQ ID NO: 319 and is inserted within said RGN having an amino acid sequence having at least 95% sequence identity to SEQ ID NO: 50 after an amino acid position corresponding to position 678 of SEQ ID NO: 2;
j) a fusion protein wherein said deaminase has an amino acid sequence having at least 95% sequence identity to SEQ ID NO: 406 and is inserted within said RGN having an amino acid sequence having at least 95% sequence identity to SEQ ID NO: 50 after an amino acid position corresponding to position 678 of SEQ ID NO: 2;
k) a fusion protein wherein said deaminase has an amino acid sequence having at least 95% sequence identity to SEQ ID NO: 597 and is inserted within said RGN having an amino acid sequence having at least 95% sequence identity to SEQ ID NO: 50 after an amino acid position corresponding to position 678 of SEQ ID NO: 2;
l) a fusion protein wherein said deaminase has an amino acid sequence having at least 95% sequence identity to SEQ ID NO: 596 and is inserted within said RGN having an amino acid sequence having at least 95% sequence identity to SEQ ID NO: 50 after an amino acid position corresponding to position 678 of SEQ ID NO: 2;
m) a fusion protein wherein said deaminase has an amino acid sequence having at least 95% sequence identity to SEQ ID NO: 406 and is inserted within said RGN having an amino acid sequence having at least 95% sequence identity to SEQ ID NO: 52 after an amino acid position corresponding to position 910 of SEQ ID NO: 4;
n) a fusion protein wherein said deaminase has an amino acid sequence having at least 95% sequence identity to SEQ ID NO: 597 and is inserted within said RGN having an amino acid sequence having at least 95% sequence identity to SEQ ID NO: 52 after an amino acid position corresponding to position 910 of SEQ ID NO: 4;
o) a fusion protein wherein said deaminase has an amino acid sequence having at least 95% sequence identity to SEQ ID NO: 406 and is inserted within said RGN having an amino acid sequence having at least 95% sequence identity to SEQ ID NO: 50 after an amino acid position corresponding to position 922 of SEQ ID NO: 2;
p) a fusion protein wherein said deaminase has an amino acid sequence having at least 95% sequence identity to SEQ ID NO: 596 and is inserted within said RGN having an amino acid sequence having at least 95% sequence identity to SEQ ID NO: 52 after an amino acid position corresponding to position 910 of SEQ ID NO: 4;
q) a fusion protein wherein said deaminase has an amino acid sequence having at least 95% sequence identity to SEQ ID NO: 596 and is inserted within said RGN having an amino acid sequence having at least 95% sequence identity to SEQ ID NO: 50 after an amino acid position corresponding to position 922 of SEQ ID NO: 2;
r) a fusion protein wherein said deaminase has an amino acid sequence having at least 95% sequence identity to SEQ ID NO: 406 and is inserted within said RGN having an amino acid sequence having at least 95% sequence identity to SEQ ID NO: 49 after an amino acid position corresponding to position 772 of SEQ ID NO: 1;
s) a fusion protein wherein said deaminase has an amino acid sequence having at least 95% sequence identity to SEQ ID NO: 596 and is inserted within said RGN having an amino acid sequence having at least 95% sequence identity to SEQ ID NO: 49 after an amino acid position corresponding to position 772 of SEQ ID NO: 1; and
t) a fusion protein wherein said deaminase has an amino acid sequence having at least 95% sequence identity to SEQ ID NO: 597 and is inserted within said RGN having an amino acid sequence having at least 95% sequence identity to SEQ ID NO: 49 after an amino acid position corresponding to position 772 of SEQ ID NO: 1.
278. The method of any one of embodiments 268-272, wherein said fusion protein is selected from the group consisting of:
a) a fusion protein wherein said deaminase has the amino acid sequence set forth as SEQ ID NO: 375 and is inserted within said RGN having the amino acid sequence set forth as SEQ ID NO: 50 after an amino acid position corresponding to position 922 of SEQ ID NO: 2;
b) a fusion protein wherein said deaminase has the amino acid sequence set forth as SEQ ID NO: 375 and is inserted within said RGN having the amino acid sequence set forth as SEQ ID NO: 52 after an amino acid position corresponding to position 910 of SEQ ID NO: 4;
c) a fusion protein wherein said deaminase has the amino acid sequence set forth as SEQ ID NO: 319 and is inserted within said RGN having the amino acid sequence set forth as SEQ ID NO: 50 after an amino acid position corresponding to position 922 of SEQ ID NO: 2;
d) a fusion protein wherein said deaminase has the amino acid sequence set forth as SEQ ID NO: 319 and is inserted within said RGN having the amino acid sequence set forth as SEQ ID NO: 52 after an amino acid position corresponding to position 910 of SEQ ID NO: 4;
e) a fusion protein wherein said deaminase has the amino acid sequence set forth as SEQ ID NO: 375 and is inserted within said RGN having the amino acid sequence set forth as SEQ ID NO: 49 after an amino acid position corresponding to position 772 of SEQ ID NO: 1;
f) a fusion protein wherein said deaminase has the amino acid sequence set forth as SEQ ID NO: 375 and is inserted within said RGN having the amino acid sequence set forth as SEQ ID NO: 50 after an amino acid position corresponding to position 678 of SEQ ID NO: 2;
g) a fusion protein wherein said deaminase has the amino acid sequence set forth as SEQ ID NO: 319 and is inserted within said RGN having the amino acid sequence set forth as SEQ ID NO: 49 after an amino acid position corresponding to position 772 of SEQ ID NO: 1;
h) a fusion protein wherein said deaminase has the amino acid sequence set forth as SEQ ID NO: 597 and is inserted within said RGN having the amino acid sequence set forth as SEQ ID NO: 50 after an amino acid position corresponding to position 922 of SEQ ID NO: 2;
i) a fusion protein wherein said deaminase has the amino acid sequence set forth as SEQ ID NO: 319 and is inserted within said RGN having the amino acid sequence set forth as SEQ ID NO: 50 after an amino acid position corresponding to position 678 of SEQ ID NO: 2;
j) a fusion protein wherein said deaminase has the amino acid sequence set forth as SEQ ID NO: 406 and is inserted within said RGN having the amino acid sequence set forth as SEQ ID NO: 50 after an amino acid position corresponding to position 678 of SEQ ID NO: 2;
k) a fusion protein wherein said deaminase has the amino acid sequence set forth as SEQ ID NO: 597 and is inserted within said RGN having the amino acid sequence set forth as SEQ ID NO: 50 after an amino acid position corresponding to position 678 of SEQ ID NO: 2;
l) a fusion protein wherein said deaminase has the amino acid sequence set forth as SEQ ID NO: 596 and is inserted within said RGN having the amino acid sequence set forth as SEQ ID NO: 50 after an amino acid position corresponding to position 678 of SEQ ID NO: 2;
m) a fusion protein wherein said deaminase has the amino acid sequence set forth as SEQ ID NO: 406 and is inserted within said RGN having the amino acid sequence set forth as SEQ ID NO: 52 after an amino acid position corresponding to position 910 of SEQ ID NO: 4;
n) a fusion protein wherein said deaminase has the amino acid sequence set forth as SEQ ID NO: 597 and is inserted within said RGN having the amino acid sequence set forth as SEQ ID NO: 52 after an amino acid position corresponding to position 910 of SEQ ID NO: 4;
o) a fusion protein wherein said deaminase has the amino acid sequence set forth as SEQ ID NO: 406 and is inserted within said RGN having the amino acid sequence set forth as SEQ ID NO: 50 after an amino acid position corresponding to position 922 of SEQ ID NO: 2;
p) a fusion protein wherein said deaminase has the amino acid sequence set forth as SEQ ID NO: 596 and is inserted within said RGN having the amino acid sequence set forth as SEQ ID NO: 52 after an amino acid position corresponding to position 910 of SEQ ID NO: 4;
q) a fusion protein wherein said deaminase has the amino acid sequence set forth as SEQ ID NO: 596 and is inserted within said RGN having the amino acid sequence set forth as SEQ ID NO: 50 after an amino acid position corresponding to position 922 of SEQ ID NO: 2;
r) a fusion protein wherein said deaminase has the amino acid sequence set forth as SEQ ID NO: 406 and is inserted within said RGN having the amino acid sequence set forth as SEQ ID NO: 49 after an amino acid position corresponding to position 772 of SEQ ID NO: 1;
s) a fusion protein wherein said deaminase has the amino acid sequence set forth as SEQ ID NO: 596 and is inserted within said RGN having the amino acid sequence set forth as SEQ ID NO: 49 after an amino acid position corresponding to position 772 of SEQ ID NO: 1; and
t) a fusion protein wherein said deaminase has the amino acid sequence set forth as SEQ ID NO: 597 and is inserted within said RGN having the amino acid sequence set forth as SEQ ID NO: 49 after an amino acid position corresponding to position 772 of SEQ ID NO: 1.
279. The method of embodiment 268, wherein said fusion protein has the sequence set forth as any one of SEQ ID NOs: 599, 600, 602, 603, 606, 610, 612, 614, 615, 617, 619-624, 626, and 630-632.

### AND

1. A deaminase comprising an amino acid sequence having at least 85% sequence identity to SEQ ID NO: 5, and comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
2. The deaminase of embodiment 1, wherein the deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
3. The deaminase of embodiment 1, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
4. The deaminase of embodiment 1 or 3, wherein said deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
5. The deaminase of any one of embodiments 1, 3, and 4, wherein said deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
6. The deaminase of any one of embodiments 1-5, wherein said deaminase comprises an amino acid sequence having at least 90% sequence identity to any one of SEQ ID NOs: 300-414, 596-598, and 720-723.
7. The deaminase of any one of embodiments 1-5, wherein said deaminase comprises an amino acid sequence having at least 95% sequence identity to any one of SEQ ID NOs: 300-414, 596-598, and 720-723.
8. The deaminase of any one of embodiments 1-5, wherein said deaminase comprises an amino acid sequence of any one of SEQ ID NOs: 300-414, 596-598, and 720-723.
9. The deaminase of any one of embodiments 1-8, wherein said deaminase has improved deaminase activity when compared to SEQ ID NO: 5.
10. The deaminase of any one of embodiments 1-9, wherein the deaminase is an adenine deaminase.
11. A nucleic acid molecule comprising a polynucleotide encoding a deaminase having at least 85% sequence identity to SEQ ID NO: 5, wherein the deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
12. The nucleic acid molecule of embodiment 11, wherein the deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
13. The nucleic acid molecule of embodiment 11, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
14. The nucleic acid molecule of embodiment 11 or 13, wherein said deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
15. The nucleic acid molecule of any one of embodiments 11, 13, and 14, wherein said deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
16. The nucleic acid molecule of any one of embodiments 11-15, wherein said deaminase comprises an amino acid sequence having at least 90% sequence identity to any one of SEQ ID NOs: 300-414, 596-598, and 720-723.
17. The nucleic acid molecule of any one of embodiments 11-15, wherein said deaminase comprises an amino acid sequence having at least 95% sequence identity to any one of SEQ ID NOs: 300-414, 596-598, and 720-723.
18. The nucleic acid molecule of any one of embodiments 11-15, wherein said deaminase comprises an amino acid sequence of any one of SEQ ID NOs: 300-414, 596-598, and 720-723.
19. The nucleic acid molecule any one of embodiments 11-18, wherein said deaminase has improved deaminase activity when compared to SEQ ID NO: 5.
20. The nucleic acid molecule of any one of embodiments 11-19, wherein the deaminase is an adenine deaminase.
21. The nucleic acid molecule of any one of embodiments 11-20, further comprising a heterologous promoter operably linked to said polynucleotide.
22. A vector comprising the nucleic acid molecule of any one of embodiments 11-21.
23. A cell comprising the deaminase of any one of embodiments 1-10, the nucleic acid molecule of any one of embodiments 11-21, or the vector of embodiment 22.
24. A pharmaceutical composition comprising a pharmaceutically acceptable carrier and the deaminase of any one of embodiments 1-10, the nucleic acid molecule of any one of embodiments 11-21, or the vector of embodiment 22.
25. A method for making a deaminase comprising introducing into a cell the nucleic acid molecule of any one of embodiments 11-21 or the vector of embodiment 22 and culturing the cell under conditions in which the deaminase is expressed.
26. The method of embodiment 25, further comprising purifying said deaminase.
27. A fusion protein comprising a DNA-binding polypeptide and a deaminase having at least 85% sequence identity to SEQ ID NO: 5, wherein the deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
28. The fusion protein of embodiment 27, wherein the deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
29. The fusion protein of embodiment 27, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or www) a S at a position corresponding to position 81 of SEQ ID NO: 5.
30. The fusion protein of embodiment 27 or 29, wherein said deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
31. The fusion protein of any one of embodiments 27, 29, and 30, wherein said deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
32. The fusion protein of any one of embodiments 27-31, wherein said deaminase comprises an amino acid sequence having at least 90% sequence identity to any one of SEQ ID NOs: 300-414, 596-598, and 720-723.
33. The fusion protein of any one of embodiments 27-31, wherein said deaminase comprises an amino acid sequence having at least 95% sequence identity to any one of SEQ ID NOs: 300-414, 596-598, and 720-723.
34. The fusion protein of any one of embodiments 27-31, wherein said deaminase comprises an amino acid sequence of any one of SEQ ID NOs: 300-414, 596-598, and 720-723.
35. The fusion protein of any one of embodiments 27-34, wherein said deaminase has improved deaminase activity when compared to SEQ ID NO: 5.
36. The fusion protein of any one of embodiments 27-35, wherein the deaminase is an adenine deaminase.
37. The fusion protein of any one of embodiments 27-36, wherein the DNA-binding polypeptide is a meganuclease, zinc finger fusion protein, or a TALEN.
38. The fusion protein of any one of embodiments 27-36, wherein the DNA-binding polypeptide is an RNA-guided, DNA-binding polypeptide.
39. The fusion protein of embodiment 38, wherein the RNA-guided, DNA-binding polypeptide is an RNA-guided nuclease (RGN) polypeptide.
40. The fusion protein of embodiment 39, wherein the RGN is a Type II CRISPR-Cas polypeptide.
41. The fusion protein of embodiment 39, wherein the RGN is a Type V CRISPR-Cas polypeptide.
42. The fusion protein of any one of embodiments 39-41, wherein the RGN is an RGN nickase.
43. The fusion protein of embodiment 39, wherein the RGN is a Cas9, a CasX, a CasY, a Cpf1, a C2cl, a C2c2, a C2c3, a GeoCas9, a CjCas9, a Casl2a, a Casl2b, a Casl2g, a Casl2h, a Casl2i, a Casl3b, a Casl3c, a Casl3d, a Casl4, a Csn2, an xCas9, an SpCas9-NG, an LbCasl2a, an AsCasl2a, a Cas9-KKH, a circularly permuted Cas9, an Argonaute (Ago), a SmacCas9, or a Spy-macCas9 domain.
44. The fusion protein of embodiment 39, wherein the RGN has an amino acid sequence having at least 95% sequence identity to any one of SEQ ID NOs: 1-4, 49-162, 435, 575, 576, 698, and 699.
45. The fusion protein of embodiment 42, wherein the RGN nickase has the amino acid sequence set forth as any one of SEQ ID NOs: 49-52 and 698.
46. The fusion protein of any of embodiments 27-45, wherein the fusion protein further comprises at least one nuclear localization signal (NLS).
47. The fusion protein of any one of embodiments 27-31, wherein said fusion protein is selected from the group consisting of:
a) a fusion protein comprising said deaminase, wherein said deaminase comprises an amino acid sequence having at least 90% sequence identity to SEQ ID NO: 319 and is fused to the N-terminus of an RGN comprising an amino acid sequence having at least 90% sequence identity to SEQ ID NO: 49;
b) a fusion protein comprising said deaminase, wherein said deaminase comprises an amino acid sequence having at least 90% sequence identity to SEQ ID NO: 375 and is fused to the N-terminus of an RGN comprising an amino acid sequence having at least 90% sequence identity to SEQ ID NO: 49;
c) a fusion protein comprising said deaminase, wherein said deaminase comprises an amino acid sequence having at least 90% sequence identity to SEQ ID NO: 406 and is fused to the N-terminus of an RGN comprising an amino acid sequence having at least 90% sequence identity to SEQ ID NO: 49;
d) a fusion protein comprising said deaminase, wherein said deaminase comprises an amino acid sequence having at least 90% sequence identity to SEQ ID NO: 596 and is fused to the N-terminus of an RGN comprising an amino acid sequence having at least 90% sequence identity to SEQ ID NO: 49;
e) a fusion protein comprising said deaminase, wherein said deaminase comprises an amino acid sequence having at least 90% sequence identity to SEQ ID NO: 597 and is fused to the N-terminus of an RGN comprising an amino acid sequence having at least 90% sequence identity to SEQ ID NO: 49;
f) a fusion protein comprising said deaminase, wherein said deaminase comprises an amino acid sequence having at least 90% sequence identity to SEQ ID NO: 303 and is fused to the N-terminus of an RGN comprising an amino acid sequence having at least 90% sequence identity to SEQ ID NO: 49;
g) a fusion protein comprising said deaminase, wherein said deaminase comprises an amino acid sequence having at least 90% sequence identity to SEQ ID NO: 366 and is fused to the N-terminus of an RGN comprising an amino acid sequence having at least 90% sequence identity to SEQ ID NO: 49; and
h) a fusion protein comprising said deaminase, wherein said deaminase comprises an amino acid sequence having at least 90% sequence identity to SEQ ID NO: 720 and is fused to the N-terminus of an RGN comprising an amino acid sequence having at least 90% sequence identity to SEQ ID NO: 49.
48. The fusion protein of any one of embodiments 27-31, wherein said fusion protein is selected from the group consisting of:
a) a fusion protein comprising said deaminase, wherein said deaminase comprises an amino acid sequence having at least 95% sequence identity to SEQ ID NO: 319 and is fused to the N-terminus of an RGN comprising an amino acid sequence having at least 95% sequence identity to SEQ ID NO: 49;
b) a fusion protein comprising said deaminase, wherein said deaminase comprises an amino acid sequence having at least 95% sequence identity to SEQ ID NO: 375 and is fused to the N-terminus of an RGN comprising an amino acid sequence having at least 95% sequence identity to SEQ ID NO: 49;
c) a fusion protein comprising said deaminase, wherein said deaminase comprises an amino acid sequence having at least 95% sequence identity to SEQ ID NO: 406 and is fused to the N-terminus of an RGN comprising an amino acid sequence having at least 95% sequence identity to SEQ ID NO: 49;
d) a fusion protein comprising said deaminase, wherein said deaminase comprises an amino acid sequence having at least 95% sequence identity to SEQ ID NO: 596 and is fused to the N-terminus of an RGN comprising an amino acid sequence having at least 95% sequence identity to SEQ ID NO: 49;
e) a fusion protein comprising said deaminase, wherein said deaminase comprises an amino acid sequence having at least 95% sequence identity to SEQ ID NO: 597 and is fused to the N-terminus of an RGN comprising an amino acid sequence having at least 95% sequence identity to SEQ ID NO: 49;
f) a fusion protein comprising said deaminase, wherein said deaminase comprises an amino acid sequence having at least 95% sequence identity to SEQ ID NO: 303 and is fused to the N-terminus of an RGN comprising an amino acid sequence having at least 95% sequence identity to SEQ ID NO: 49;
g) a fusion protein comprising said deaminase, wherein said deaminase comprises an amino acid sequence having at least 95% sequence identity to SEQ ID NO: 366 and is fused to the N-terminus of an RGN comprising an amino acid sequence having at least 95% sequence identity to SEQ ID NO: 49; and
h) a fusion protein comprising said deaminase, wherein said deaminase comprises an amino acid sequence having at least 95% sequence identity to SEQ ID NO: 720 and is fused to the N-terminus of an RGN comprising an amino acid sequence having at least 95% sequence identity to SEQ ID NO: 49.
49. The fusion protein of any one of embodiments 27-31, wherein said fusion protein is selected from the group consisting of:
a) a fusion protein comprising said deaminase, wherein said deaminase comprises the amino acid sequence set forth as SEQ ID NO: 319 and is fused to the N-terminus of an RGN comprising the amino acid sequence set forth as SEQ ID NO: 49;
b) a fusion protein comprising said deaminase, wherein said deaminase comprises the amino acid sequence set forth as SEQ ID NO: 375 and is fused to the N-terminus of an RGN comprising the amino acid sequence set forth as SEQ ID NO: 49;
c) a fusion protein comprising said deaminase, wherein said deaminase comprises the amino acid sequence set forth as SEQ ID NO: 406 and is fused to the N-terminus of an RGN comprising the amino acid sequence set forth as SEQ ID NO: 49;
d) a fusion protein comprising said deaminase, wherein said deaminase comprises the amino acid sequence set forth as SEQ ID NO: 596 and is fused to the N-terminus of an RGN comprising the amino acid sequence set forth as SEQ ID NO: 49;
e) a fusion protein comprising said deaminase, wherein said deaminase comprises the amino acid sequence set forth as SEQ ID NO: 597 and is fused to the N-terminus of an RGN comprising the amino acid sequence set forth as SEQ ID NO: 49;
f) a fusion protein comprising said deaminase, wherein said deaminase comprises the amino acid sequence set forth as SEQ ID NO: 303 and is fused to the N-terminus of an RGN comprising the amino acid sequence set forth as SEQ ID NO: 49;
g) a fusion protein comprising said deaminase, wherein said deaminase comprises the amino acid sequence set forth as SEQ ID NO: 366 and is fused to the N-terminus of an RGN comprising the amino acid sequence set forth as SEQ ID NO: 49; and
h) a fusion protein comprising said deaminase, wherein said deaminase comprises the amino acid sequence set forth as SEQ ID NO: 720 and is fused to the N-terminus of an RGN comprising the amino acid sequence set forth as SEQ ID NO: 49.
50. The fusion protein of any one of embodiments 47-49, wherein said fusion protein has the sequence set forth as any one of SEQ ID NOs: 577, 578, 582, 584, 588, 593, 594, 604, 605, 616, and 729.
51. A ribonucleoprotein (RNP) complex comprising the fusion protein of any one of embodiments 38-50 and a guide RNA bound to the fusion protein.
52. A nucleic acid molecule comprising a polynucleotide encoding a fusion protein comprising a DNA-binding polypeptide and a deaminase, wherein the deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
53. The nucleic acid molecule of embodiment 52, wherein the deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
1) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
54. The nucleic acid molecule of embodiment 52, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
55. The nucleic acid molecule of embodiment 52 or 54, wherein said deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
56. The nucleic acid molecule of any one of embodiments 52, 54, and 55, wherein said deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
57. The nucleic acid molecule of any one of embodiments 52-56, wherein said deaminase comprises an amino acid sequence having at least 90% sequence identity to any one of SEQ ID NOs: 300-414, 596-598, and 720-723.
58. The nucleic acid molecule of any one of embodiments 52-56, wherein said deaminase comprises an amino acid sequence having at least 95% sequence identity to any one of SEQ ID NOs: 300-414, 596-598, and 720-723.
59. The nucleic acid molecule of any one of embodiments 52-56, wherein said deaminase comprises an amino acid sequence of any one of SEQ ID NOs: 300-414, 596-598, and 720-723.
60. The nucleic acid molecule of any one of embodiments 52-59, wherein said deaminase has improved deaminase activity when compared to SEQ ID NO: 5.
61. The nucleic acid molecule of any one of embodiments 52-60, wherein the deaminase is an adenine deaminase.
62. The nucleic acid molecule of any one of embodiments 52-61, wherein the DNA-binding polypeptide is a meganuclease, zinc finger fusion protein, or a TALEN.
63. The nucleic acid molecule of any one of embodiments 52-61, wherein the DNA-binding polypeptide is an RNA-guided, DNA-binding polypeptide.
64. The nucleic acid molecule of embodiment 63, wherein the RNA-guided, DNA-binding polypeptide is an RNA-guided nuclease (RGN) polypeptide.
65. The nucleic acid molecule of embodiment 64, wherein the RGN is a Type II CRISPR-Cas polypeptide.
66. The nucleic acid molecule of embodiment 64, wherein the RGN is a Type V CRISPR-Cas polypeptide.
67. The nucleic acid molecule of any one of embodiments 64-66, wherein the RGN is an RGN nickase.
68. The nucleic acid molecule of embodiment 64, wherein the RGN polypeptide is a Cas9, a CasX, a CasY, a Cpf1, a C2cl, a C2c2, a C2c3, a GeoCas9, a CjCas9, a Casl2a, a Casl2b, a Casl2g, a Casl2h, a Casl2i, a Casl3b, a Casl3c, a Casl3d, a Cas14, a Csn2, an xCas9, an SpCas9-NG, an LbCasl2a, an AsCasl2a, a Cas9-KKH, a circularly permuted Cas9, an Argonaute (Ago), a SmacCas9, or a Spy-macCas9 domain.
69. The nucleic acid molecule of embodiment 64, wherein the RGN has an amino acid sequence having at least 95% sequence identity to any one of SEQ ID NOs: 1-4, 49-162, 435, 575, 576, 698, and 699.
70. The nucleic acid molecule of embodiment 67, wherein the RGN nickase has the amino acid sequence set forth as any one of SEQ ID NOs: 49-52 and 698.
71. The nucleic acid molecule of any of embodiments 52-70, wherein the fusion protein further comprises at least one nuclear localization signal (NLS).
72. The nucleic acid molecule of any one of embodiments 52-56, wherein said fusion protein is selected from the group consisting of:
a) a fusion protein comprising said deaminase, wherein said deaminase comprises an amino acid sequence having at least 90% sequence identity to SEQ ID NO: 319 and is fused to the N-terminus of an RGN comprising an amino acid sequence having at least 90% sequence identity to SEQ ID NO: 49;
b) a fusion protein comprising said deaminase, wherein said deaminase comprises an amino acid sequence having at least 90% sequence identity to SEQ ID NO: 375 and is fused to the N-terminus of an RGN comprising an amino acid sequence having at least 90% sequence identity to SEQ ID NO: 49;
c) a fusion protein comprising said deaminase, wherein said deaminase comprises an amino acid sequence having at least 90% sequence identity to SEQ ID NO: 406 and is fused to the N-terminus of an RGN comprising an amino acid sequence having at least 90% sequence identity to SEQ ID NO: 49;
d) a fusion protein comprising said deaminase, wherein said deaminase comprises an amino acid sequence having at least 90% sequence identity to SEQ ID NO: 596 and is fused to the N-terminus of an RGN comprising an amino acid sequence having at least 90% sequence identity to SEQ ID NO: 49;
e) a fusion protein comprising said deaminase, wherein said deaminase comprises an amino acid sequence having at least 90% sequence identity to SEQ ID NO: 597 and is fused to the N-terminus of an RGN comprising an amino acid sequence having at least 90% sequence identity to SEQ ID NO: 49;
f) a fusion protein comprising said deaminase, wherein said deaminase comprises an amino acid sequence having at least 90% sequence identity to SEQ ID NO: 303 and is fused to the N-terminus of an RGN comprising an amino acid sequence having at least 90% sequence identity to SEQ ID NO: 49;
g) a fusion protein comprising said deaminase, wherein said deaminase comprises an amino acid sequence having at least 90% sequence identity to SEQ ID NO: 366 and is fused to the N-terminus of an RGN comprising an amino acid sequence having at least 90% sequence identity to SEQ ID NO: 49; and
h) a fusion protein comprising said deaminase, wherein said deaminase comprises an amino acid sequence having at least 90% sequence identity to SEQ ID NO: 720 and is fused to the N-terminus of an RGN comprising an amino acid sequence having at least 90% sequence identity to SEQ ID NO: 49.
73. The nucleic acid molecule of any one of embodiments 52-56, wherein said fusion protein is selected from the group consisting of:
a) a fusion protein comprising said deaminase, wherein said deaminase comprises an amino acid sequence having at least 95% sequence identity to SEQ ID NO: 319 and is fused to the N-terminus of an RGN comprising an amino acid sequence having at least 95% sequence identity to SEQ ID NO: 49;
b) a fusion protein comprising said deaminase, wherein said deaminase comprises an amino acid sequence having at least 95% sequence identity to SEQ ID NO: 375 and is fused to the N-terminus of an RGN comprising an amino acid sequence having at least 95% sequence identity to SEQ ID NO: 49;
c) a fusion protein comprising said deaminase, wherein said deaminase comprises an amino acid sequence having at least 95% sequence identity to SEQ ID NO: 406 and is fused to the N-terminus of an RGN comprising an amino acid sequence having at least 95% sequence identity to SEQ ID NO: 49;
d) a fusion protein comprising said deaminase, wherein said deaminase comprises an amino acid sequence having at least 95% sequence identity to SEQ ID NO: 596 and is fused to the N-terminus of an RGN comprising an amino acid sequence having at least 95% sequence identity to SEQ ID NO: 49;
e) a fusion protein comprising said deaminase, wherein said deaminase comprises an amino acid sequence having at least 95% sequence identity to SEQ ID NO: 597 and is fused to the N-terminus of an RGN comprising an amino acid sequence having at least 95% sequence identity to SEQ ID NO: 49;
f) a fusion protein comprising said deaminase, wherein said deaminase comprises an amino acid sequence having at least 95% sequence identity to SEQ ID NO: 303 and is fused to the N-terminus of an RGN comprising an amino acid sequence having at least 95% sequence identity to SEQ ID NO: 49;
g) a fusion protein comprising said deaminase, wherein said deaminase comprises an amino acid sequence having at least 95% sequence identity to SEQ ID NO: 366 and is fused to the N-terminus of an RGN comprising an amino acid sequence having at least 95% sequence identity to SEQ ID NO: 49; and
h) a fusion protein comprising said deaminase, wherein said deaminase comprises an amino acid sequence having at least 95% sequence identity to SEQ ID NO: 720 and is fused to the N-terminus of an RGN comprising an amino acid sequence having at least 95% sequence identity to SEQ ID NO: 49.
74. The nucleic acid molecule of any one of embodiments 52-56, wherein said fusion protein is selected from the group consisting of:
a) a fusion protein comprising said deaminase, wherein said deaminase comprises the amino acid sequence set forth as SEQ ID NO: 319 and is fused to the N-terminus of an RGN comprising the amino acid sequence set forth as SEQ ID NO: 49;
b) a fusion protein comprising said deaminase, wherein said deaminase comprises the amino acid sequence set forth as SEQ ID NO: 375 and is fused to the N-terminus of an RGN comprising the amino acid sequence set forth as SEQ ID NO: 49;
c) a fusion protein comprising said deaminase, wherein said deaminase comprises the amino acid sequence set forth as SEQ ID NO: 406 and is fused to the N-terminus of an RGN comprising the amino acid sequence set forth as SEQ ID NO: 49;
d) a fusion protein comprising said deaminase, wherein said deaminase comprises the amino acid sequence set forth as SEQ ID NO: 596 and is fused to the N-terminus of an RGN comprising the amino acid sequence set forth as SEQ ID NO: 49;
e) a fusion protein comprising said deaminase, wherein said deaminase comprises the amino acid sequence set forth as SEQ ID NO: 597 and is fused to the N-terminus of an RGN comprising the amino acid sequence set forth as SEQ ID NO: 49;
f) a fusion protein comprising said deaminase, wherein said deaminase comprises the amino acid sequence set forth as SEQ ID NO: 303 and is fused to the N-terminus of an RGN comprising the amino acid sequence set forth as SEQ ID NO: 49;
g) a fusion protein comprising said deaminase, wherein said deaminase comprises the amino acid sequence set forth as SEQ ID NO: 366 and is fused to the N-terminus of an RGN comprising the amino acid sequence set forth as SEQ ID NO: 49; and
h) a fusion protein comprising said deaminase, wherein said deaminase comprises the amino acid sequence set forth as SEQ ID NO: 720 and is fused to the N-terminus of an RGN comprising the amino acid sequence set forth as SEQ ID NO: 49.
75. The nucleic acid molecule of any one of embodiments 72-74, wherein said fusion protein has the sequence set forth as any one of SEQ ID NOs: 577, 578, 582, 584, 588, 593, 594, 604, 605, 616, and 729.
76. The nucleic acid molecule of any one of embodiments 52-75, wherein the fusion protein is codon optimized for expression in a eukaryotic cell.
77. The nucleic acid molecule of any one of embodiments 52-75, wherein the fusion protein is codon optimized for expression in a prokaryotic cell.
78. A vector comprising the nucleic acid molecule of any one of embodiments 52-77.
79. A vector comprising the nucleic acid molecule of any one of embodiments 64-75, further comprising at least one nucleotide sequence encoding a guide RNA capable of hybridizing to a non-target strand of a target sequence.
80. The vector of embodiment 79, wherein the guide RNA is a single guide RNA (sgRNA).
81. The vector of embodiment 79, wherein the guide RNA is a dual guide RNA.
82. A cell comprising the fusion protein of any of embodiments 27-50 or the RNP complex of embodiment 51.
83. A cell comprising the fusion protein of any one of embodiments 27-50, wherein the cell further comprises a guide RNA.
84. A cell comprising the nucleic acid molecule of any one of embodiments 52-77.
85. A cell comprising the vector of any one of embodiments 78-81.
86. The cell of any one of embodiments 82-85, wherein the cell is a prokaryotic cell.
87. The cell of any one of embodiments 82-85, wherein the cell is a eukaryotic cell.
88. The cell of embodiment 87, wherein the eukaryotic cell is a mammalian cell.
89. The cell of embodiment 88, wherein the mammalian cell is a human cell.
90. The cell of embodiment 89, wherein the human cell is an immune cell.
91. The cell of embodiment 90, wherein the immune cell is a stem cell.
92. The cell of embodiment 91, wherein the stem cell is an induced pluripotent stem cell.
93. The cell of embodiment 87, wherein the eukaryotic cell is an insect or avian cell.
94. The cell of embodiment 87, wherein the eukaryotic cell is a fungal cell.
95. The cell of embodiment 87, wherein the eukaryotic cell is a plant cell.
96. A plant comprising the cell of embodiment 95.
97. A seed comprising the cell of embodiment 95.
98. A pharmaceutical composition comprising a pharmaceutically acceptable carrier and the fusion protein of any one of embodiments 27-50, the RNP complex of embodiment 51, the nucleic acid molecule of any one of embodiments 52-77, the vector of any one of embodiments 78-81, or the cell of any one of embodiments 88-92.
99. A method for making a fusion protein comprising culturing the cell of any one of embodiments 82-95 under conditions in which the fusion protein is expressed.
100. A method for making a fusion protein comprising introducing into a cell the nucleic acid molecule of any of embodiments 52-77 or a vector of any one of embodiments 78-81 and culturing the cell under conditions in which the fusion protein is expressed.
101. The method of embodiment 99 or 100, further comprising purifying said fusion protein.
102. A method for making an RGN fusion ribonucleoprotein complex, comprising introducing into a cell the nucleic acid molecule of any one of embodiments 63-70 and 72-75 and a nucleic acid molecule comprising an expression cassette encoding for a guide RNA, or the vector of any of embodiments 79-81, and culturing the cell under conditions in which the fusion protein and the gRNA are expressed and form an RGN fusion ribonucleoprotein complex.
103. The method of embodiment 102, further comprising purifying said RGN fusion ribonucleoprotein complex.
104. A system for modifying a target DNA molecule comprising a target DNA sequence, said system comprising:
a) a fusion protein comprising an RNA-guided nuclease polypeptide and a deaminase, wherein the deaminase has an amino acid sequence having at least 85% sequence identity to SEQ ID NO: 5, or a nucleotide sequence encoding said fusion protein, wherein said deaminase comprises at least one of the following amino acid residues:
   i) a C at a position corresponding to position 2 of SEQ ID NO: 5;
   ii) a F or C at a position corresponding to position 22 of SEQ ID NO: 5;
   iii) a Q at a position corresponding to position 23 of SEQ ID NO: 5;
   iv) a N at a position corresponding to position 35 of SEQ ID NO: 5;
   v) a L at a position corresponding to position 40 of SEQ ID NO: 5;
   vi) a Q at a position corresponding to position 46 of SEQ ID NO: 5;
   vii) a A or M at a position corresponding to position 68 of SEQ ID NO: 5;
   viii) a H at a position corresponding to position 72 of SEQ ID NO: 5;
   ix) a W, A, Q, Y, or D at a position corresponding to position 75 of SEQ ID NO: 5;
   x) a G at a position corresponding to position 76 of SEQ ID NO: 5;
   xi) a S at a position corresponding to position 81 of SEQ ID NO: 5;
   xii) a M at a position corresponding to position 105 of SEQ ID NO: 5;
   xiii) a H at a position corresponding to position 108 of SEQ ID NO: 5;
   xiv) a L at a position corresponding to position 109 of SEQ ID NO: 5;
   xv) a I or L at a position corresponding to position 117 of SEQ ID NO: 5;
   xvi) a F at a position corresponding to position 120 of SEQ ID NO: 5;
   xvii) a E, T, A, G, or V at a position corresponding to position 121 of SEQ ID NO: 5;
   xviii) a I or H at a position corresponding to position 122 of SEQ ID NO: 5;
   xix) a K at a position corresponding to position 125 of SEQ ID NO: 5;
   xx) a A at a position corresponding to position 126 of SEQ ID NO: 5;
   xxi) a H at a position corresponding to position 135 of SEQ ID NO: 5;
   xxii) a V at a position corresponding to position 137 of SEQ ID NO: 5;
   xxiii) a Y at a position corresponding to position 138 of SEQ ID NO: 5;
   xxiv) a L at a position corresponding to position 139 of SEQ ID NO: 5;
   xxv) a K or A at a position corresponding to position 142 of SEQ ID NO: 5;
   xxvi) a A, Q, L, or M at a position corresponding to position 145 of SEQ ID NO: 5;
   xxvii) a K at a position corresponding to position 148 of SEQ ID NO: 5;
   xxviii) a Q at a position corresponding to position 151 of SEQ ID NO: 5;
   xxix) a E or R at a position corresponding to position 153 of SEQ ID NO: 5;
   xxx) a W at a position corresponding to position 155 of SEQ ID NO: 5;
   xxxi) a R or V at a position corresponding to position 156 of SEQ ID NO: 5;
   xxxii) a F at a position corresponding to position 157 of SEQ ID NO: 5;
   xxxiii) a R at a position corresponding to position 158 of SEQ ID NO: 5;
   xxxiv) a Q at a position corresponding to position 159 of SEQ ID NO: 5;
   xxxv) a D or W at a position corresponding to position 160 of SEQ ID NO: 5;
   xxxvi) a W, H, V, or A at a position corresponding to position 162 of SEQ ID NO: 5;
   xxxvii) a R at a position corresponding to position 165 of SEQ ID NO: 5; and
   xxxviii) a H at a position corresponding to position 166 of SEQ ID NO: 5; and
b) one or more guide RNAs capable of hybridizing to a non-target strand of said target DNA sequence or one or more nucleotide sequences encoding the one or more guide RNAs; and
wherein the one or more guide RNAs are capable of forming a complex with the fusion protein in order to direct said fusion protein to bind to said target DNA sequence and modify the target DNA molecule.
105. The system of embodiment 104, wherein said deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
106. The system of embodiment 104, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
107. The system of embodiment 104 or 106, wherein said deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
108. The system of any one of embodiments 104, 106, and 107, wherein said deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
109. The system of any one of embodiments 104-108, wherein said deaminase comprises an amino acid sequence having at least 90% sequence identity to any one of SEQ ID NOs: 300-414, 596-598, and 720-723.
110. The system of any one of embodiments 104-108, wherein said deaminase comprises an amino acid sequence having at least 95% sequence identity to any one of SEQ ID NOs: 300-414, 596-598, and 720-723.
111. The system of any one of embodiments 104-108, wherein said deaminase comprises an amino acid sequence of any one of SEQ ID NOs: 300-414, 596-598, and 720-723.
112. The system of any one of embodiments 104-111, wherein said deaminase has improved deaminase activity when compared to SEQ ID NO: 5.
113. The system of any one of embodiments 104-111, wherein the deaminase is an adenine deaminase.
114. The system of any one of embodiments 104-113, wherein at least one of said nucleotide sequences encoding the one or more guide RNAs and said nucleotide sequence encoding the fusion protein is operably linked to a promoter heterologous to said nucleotide sequence.
115. The system of any one of embodiments 104-114, wherein the target DNA sequence is a eukaryotic target DNA sequence.
116. The system of any one of embodiments 104-115, wherein the target DNA sequence is located adjacent to a protospacer adjacent motif (PAM) that is recognized by the RGN.
117. The system of any one of embodiments 104-116, wherein the RGN of the fusion protein is a Type II CRISPR-Cas polypeptide.
118. The system of any one of embodiments 104-116, wherein the RGN of the fusion protein is a Type V CRISPR-Cas polypeptide.
119. The system of any one of embodiments 104-116, wherein the RGN is a Cas9, a CasX, a CasY, a Cpf1, a C2cl, a C2c2, a C2c3, a GeoCas9, a CjCas9, a Casl2a, a Casl2b, a Casl2g, a Casl2h, a Casl2i, a Casl3b, a Casl3c, a Casl3d, a Casl4, a Csn2, an xCas9, an SpCas9-NG, an LbCasl2a, an AsCasl2a, a Cas9-KKH, a circularly permuted Cas9, an Argonaute (Ago), a SmacCas9, or a Spy-macCas9 domain.
120. The system of any one of embodiments 104-116, wherein the RGN of the fusion protein has an amino acid sequence having at least 95% sequence identity to any one of SEQ ID NOs: 1-4, 49-162, 435, 575, 576, 698, and 699.
121. The system of any one of embodiments 104-116, wherein the RGN of the fusion protein is an RGN nickase.
122. The system of embodiment 121, wherein the RGN nickase has the amino acid sequence set forth as any one of SEQ ID NOs: 49-52 and 698.
123. The system of any one of embodiments 104-122, wherein the fusion protein comprises one or more nuclear localization signals.
124. The system of any one of embodiments 104-108, wherein said fusion protein is selected from the group consisting of:
a) a fusion protein comprising said deaminase, wherein said deaminase comprises an amino acid sequence having at least 90% sequence identity to SEQ ID NO: 319 and is fused to the N-terminus of an RGN comprising an amino acid sequence having at least 90% sequence identity to SEQ ID NO: 49;
b) a fusion protein comprising said deaminase, wherein said deaminase comprises an amino acid sequence having at least 90% sequence identity to SEQ ID NO: 375 and is fused to the N-terminus of an RGN comprising an amino acid sequence having at least 90% sequence identity to SEQ ID NO: 49;
c) a fusion protein comprising said deaminase, wherein said deaminase comprises an amino acid sequence having at least 90% sequence identity to SEQ ID NO: 406 and is fused to the N-terminus of an RGN comprising an amino acid sequence having at least 90% sequence identity to SEQ ID NO: 49;
d) a fusion protein comprising said deaminase, wherein said deaminase comprises an amino acid sequence having at least 90% sequence identity to SEQ ID NO: 596 and is fused to the N-terminus of an RGN comprising an amino acid sequence having at least 90% sequence identity to SEQ ID NO: 49;
e) a fusion protein comprising said deaminase, wherein said deaminase comprises an amino acid sequence having at least 90% sequence identity to SEQ ID NO: 597 and is fused to the N-terminus of an RGN comprising an amino acid sequence having at least 90% sequence identity to SEQ ID NO: 49;
f) a fusion protein comprising said deaminase, wherein said deaminase comprises an amino acid sequence having at least 90% sequence identity to SEQ ID NO: 303 and is fused to the N-terminus of an RGN comprising an amino acid sequence having at least 90% sequence identity to SEQ ID NO: 49;
g) a fusion protein comprising said deaminase, wherein said deaminase comprises an amino acid sequence having at least 90% sequence identity to SEQ ID NO: 366 and is fused to the N-terminus of an RGN comprising an amino acid sequence having at least 90% sequence identity to SEQ ID NO: 49; and
h) a fusion protein comprising said deaminase, wherein said deaminase comprises an amino acid sequence having at least 90% sequence identity to SEQ ID NO: 720 and is fused to the N-terminus of an RGN comprising an amino acid sequence having at least 90% sequence identity to SEQ ID NO: 49.
125. The system of any one of embodiments 104-108, wherein said fusion protein is selected from the group consisting of:
a) a fusion protein comprising said deaminase, wherein said deaminase comprises an amino acid sequence having at least 95% sequence identity to SEQ ID NO: 319 and is fused to the N-terminus of an RGN comprising an amino acid sequence having at least 95% sequence identity to SEQ ID NO: 49;
b) a fusion protein comprising said deaminase, wherein said deaminase comprises an amino acid sequence having at least 95% sequence identity to SEQ ID NO: 375 and is fused to the N-terminus of an RGN comprising an amino acid sequence having at least 95% sequence identity to SEQ ID NO: 49;
c) a fusion protein comprising said deaminase, wherein said deaminase comprises an amino acid sequence having at least 95% sequence identity to SEQ ID NO: 406 and is fused to the N-terminus of an RGN comprising an amino acid sequence having at least 95% sequence identity to SEQ ID NO: 49;
d) a fusion protein comprising said deaminase, wherein said deaminase comprises an amino acid sequence having at least 95% sequence identity to SEQ ID NO: 596 and is fused to the N-terminus of an RGN comprising an amino acid sequence having at least 95% sequence identity to SEQ ID NO: 49;
e) a fusion protein comprising said deaminase, wherein said deaminase comprises an amino acid sequence having at least 95% sequence identity to SEQ ID NO: 597 and is fused to the N-terminus of an RGN comprising an amino acid sequence having at least 95% sequence identity to SEQ ID NO: 49;
f) a fusion protein comprising said deaminase, wherein said deaminase comprises an amino acid sequence having at least 95% sequence identity to SEQ ID NO: 303 and is fused to the N-terminus of an RGN comprising an amino acid sequence having at least 95% sequence identity to SEQ ID NO: 49;
g) a fusion protein comprising said deaminase, wherein said deaminase comprises an amino acid sequence having at least 95% sequence identity to SEQ ID NO: 366 and is fused to the N-terminus of an RGN comprising an amino acid sequence having at least 95% sequence identity to SEQ ID NO: 49; and
h) a fusion protein comprising said deaminase, wherein said deaminase comprises an amino acid sequence having at least 95% sequence identity to SEQ ID NO: 720 and is fused to the N-terminus of an RGN comprising an amino acid sequence having at least 95% sequence identity to SEQ ID NO: 49.
126. The system of any one of embodiments 104-108, wherein said fusion protein is selected from the group consisting of:
a) a fusion protein comprising said deaminase, wherein said deaminase comprises the amino acid sequence set forth as SEQ ID NO: 319 and is fused to the N-terminus of an RGN comprising the amino acid sequence set forth as SEQ ID NO: 49;
b) a fusion protein comprising said deaminase, wherein said deaminase comprises the amino acid sequence set forth as SEQ ID NO: 375 and is fused to the N-terminus of an RGN comprising the amino acid sequence set forth as SEQ ID NO: 49;
c) a fusion protein comprising said deaminase, wherein said deaminase comprises the amino acid sequence set forth as SEQ ID NO: 406 and is fused to the N-terminus of an RGN comprising the amino acid sequence set forth as SEQ ID NO: 49;
d) a fusion protein comprising said deaminase, wherein said deaminase comprises the amino acid sequence set forth as SEQ ID NO: 596 and is fused to the N-terminus of an RGN comprising the amino acid sequence set forth as SEQ ID NO: 49;
e) a fusion protein comprising said deaminase, wherein said deaminase comprises the amino acid sequence set forth as SEQ ID NO: 597 and is fused to the N-terminus of an RGN comprising the amino acid sequence set forth as SEQ ID NO: 49;
f) a fusion protein comprising said deaminase, wherein said deaminase comprises the amino acid sequence set forth as SEQ ID NO: 303 and is fused to the N-terminus of an RGN comprising the amino acid sequence set forth as SEQ ID NO: 49;
g) a fusion protein comprising said deaminase, wherein said deaminase comprises the amino acid sequence set forth as SEQ ID NO: 366 and is fused to the N-terminus of an RGN comprising the amino acid sequence set forth as SEQ ID NO: 49; and
h) a fusion protein comprising said deaminase, wherein said deaminase comprises the amino acid sequence set forth as SEQ ID NO: 720 and is fused to the N-terminus of an RGN comprising the amino acid sequence set forth as SEQ ID NO: 49.
127. The system of any one of embodiments 124-126, wherein said fusion protein has the sequence set forth as any one of SEQ ID NOs: 577, 578, 582, 584, 588, 593, 594, 604, 605, 616, and 729.
128. The system of any one of embodiments 104-127, wherein the fusion protein is codon optimized for expression in a eukaryotic cell.
129. The system of any one of embodiments 104-128, wherein nucleotide sequences encoding the one or more guide RNAs and the nucleotide sequence encoding a fusion protein are located on one vector.
130. A cell comprising the system of any one of embodiments 104-129.
131. The cell of embodiment 130, wherein the cell is a eukaryotic cell.
132. The cell of embodiment 131, wherein the eukaryotic cell is a mammalian cell.
133. The cell of embodiment 132, wherein the mammalian cell is a human cell.
134. The cell of embodiment 133, wherein the human cell is an immune cell.
135. The cell of embodiment 134, wherein the immune cell is a stem cell.
136. The cell of embodiment 135, wherein the stem cell is an induced pluripotent stem cell.
137. The cell of embodiment 131, wherein the eukaryotic cell is an insect cell.
138. The cell of embodiment 131, wherein the eukaryotic cell is a plant cell.
139. A plant comprising the cell of embodiment 138.
140. A seed comprising the cell of embodiment 138.
141. The cell of embodiment 130, wherein the cell is a prokaryotic cell.
142. A pharmaceutical composition comprising a pharmaceutically acceptable carrier and the system of any one of embodiments 104-129 or the cell of any one of embodiments 132-136.
143. A method for modifying a target DNA molecule comprising a target DNA sequence, said method comprising delivering a system according to any one of embodiments 104-129 to said target DNA molecule or a cell comprising the target DNA molecule.
144. The method of embodiment 143, wherein said modified target DNA molecule comprises an A>N mutation of at least one nucleotide within the target DNA molecule, wherein N is C, G, or T.
145. The method of embodiment 144, wherein said modified target DNA molecule comprises an A>G mutation of at least one nucleotide within the target DNA molecule.
146. A method for modifying a target DNA molecule comprising a target sequence comprising:
a) assembling an RGN-deaminase ribonucleotide complex in vitro by combining:
   i) one or more guide RNAs capable of hybridizing to a non-target strand of the target DNA sequence; and
   ii) a fusion protein comprising an RNA-guided nuclease polypeptide (RGN), and at least one deaminase, wherein the deaminase has an amino acid sequence having at least 85% sequence identity to SEQ ID NO: 5;
under conditions suitable for formation of the RGN-deaminase ribonucleotide complex; wherein said deaminase has at least one of the following amino acid residues:
   A) a C at a position corresponding to position 2 of SEQ ID NO: 5;
   B) a F or C at a position corresponding to position 22 of SEQ ID NO: 5;
   C) a Q at a position corresponding to position 23 of SEQ ID NO: 5;
   D) a N at a position corresponding to position 35 of SEQ ID NO: 5;
   E) a L at a position corresponding to position 40 of SEQ ID NO: 5;
   F) a Q at a position corresponding to position 46 of SEQ ID NO: 5;
   G) a A or M at a position corresponding to position 68 of SEQ ID NO: 5;
   H) a H at a position corresponding to position 72 of SEQ ID NO: 5;
   I) a W, A, Q, Y, or D at a position corresponding to position 75 of SEQ ID NO: 5;
   J) a G at a position corresponding to position 76 of SEQ ID NO: 5;
   K) a S at a position corresponding to position 81 of SEQ ID NO: 5;
   L) a M at a position corresponding to position 105 of SEQ ID NO: 5;
   M) a H at a position corresponding to position 108 of SEQ ID NO: 5;
   N) a L at a position corresponding to position 109 of SEQ ID NO: 5;
   O) a I or L at a position corresponding to position 117 of SEQ ID NO: 5;
   P) a F at a position corresponding to position 120 of SEQ ID NO: 5;
   Q) a E, T, A, G, or V at a position corresponding to position 121 of SEQ ID NO: 5;
   R) a I or H at a position corresponding to position 122 of SEQ ID NO: 5;
   S) a K at a position corresponding to position 125 of SEQ ID NO: 5;
   T) a A at a position corresponding to position 126 of SEQ ID NO: 5;
   U) a H at a position corresponding to position 135 of SEQ ID NO: 5;
   V) a V at a position corresponding to position 137 of SEQ ID NO: 5;
   W) a Y at a position corresponding to position 138 of SEQ ID NO: 5;
   X) a L at a position corresponding to position 139 of SEQ ID NO: 5;
   Y) a K or A at a position corresponding to position 142 of SEQ ID NO: 5;
   Z) a A, Q, L, or M at a position corresponding to position 145 of SEQ ID NO: 5;
   AA) a K at a position corresponding to position 148 of SEQ ID NO: 5;
   BB) a Q at a position corresponding to position 151 of SEQ ID NO: 5;
   CC) a E or R at a position corresponding to position 153 of SEQ ID NO: 5;
   DD) a W at a position corresponding to position 155 of SEQ ID NO: 5;
   EE) a R or V at a position corresponding to position 156 of SEQ ID NO: 5;
   FF) a F at a position corresponding to position 157 of SEQ ID NO: 5;
   GG) a R at a position corresponding to position 158 of SEQ ID NO: 5;
   HH) a Q at a position corresponding to position 159 of SEQ ID NO: 5;
   II) a D or W at a position corresponding to position 160 of SEQ ID NO: 5;
   JJ) a W, H, V, or A at a position corresponding to position 162 of SEQ ID NO: 5;
   KK) a R at a position corresponding to position 165 of SEQ ID NO: 5; and
   LL) a H at a position corresponding to position 166 of SEQ ID NO: 5; and
b) contacting said target DNA molecule or a cell comprising said target DNA molecule with the in vitro-assembled RGN-deaminase ribonucleotide complex;
wherein the one or more guide RNAs hybridize to a non-target strand of the target DNA sequence, thereby directing said fusion protein to bind to said target DNA sequence and modification of the target DNA molecule occurs.
147. The method of embodiment 146, wherein said deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
148. The method of embodiment 146, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
149. The method of embodiment 146 or 148, wherein said deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
150. The method of any one of embodiments 146, 148, and 149, wherein said deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
151. The method of any one of embodiments 146-150, wherein said deaminase has an amino acid sequence having at least 90% sequence identity to any one of SEQ ID NOs: 300-414, 596-598, and 720-723.
152. The method of any one of embodiments 146-150, wherein said deaminase has an amino acid sequence having at least 95% sequence identity to any one of SEQ ID NOs: 300-414, 596-598, and 720-723.
153. The method of any one of embodiments 146-150, wherein said deaminase has the amino acid sequence of any one of SEQ ID NOs: 300-414, 596-598, and 720-723.
154. The method of any one of embodiments 146-153, wherein said deaminase has improved deaminase activity when compared to SEQ ID NO: 5.
155. The method of any one of embodiments 146-154, wherein the deaminase is an adenine deaminase.
156. The method of any one of embodiments 146-155, wherein the RGN of the fusion protein is a Type II CRISPR-Cas polypeptide.
157. The method of any of embodiments 146-155, wherein the RGN of the fusion protein is a Type V CRISPR-Cas polypeptide.
158. The method of any one of embodiments 146-155, wherein the RGN is a Cas9, a CasX, a CasY, a Cpf1, a C2cl, a C2c2, a C2c3, a GeoCas9, a CjCas9, a Cas12a, a Cas12b, a Casl2g, a Cas12h, a Cas12i, a Cas13b, a Cas13c, a Cas13d, a Cas14, a Csn2, an xCas9, an SpCas9-NG, an LbCasl2a, an AsCasl2a, a Cas9-KKH, a circularly permuted Cas9, an Argonaute (Ago), a SmacCas9, or a Spy-macCas9 domain.
159. The method of any of embodiments 146-155, wherein the RGN of the fusion protein has an amino acid sequence having at least 95% sequence identity to any one of SEQ ID NOs: 1-4, 49-162, 435, 575, 576, 698, and 699.
160. The method of any of embodiments 146-155, wherein the RGN of the fusion protein is an RGN nickase.
161. The method of embodiment 160, wherein the RGN nickase has the amino acid sequence set forth as any one of SEQ ID NOs: 49-52 and 698.
162. The method of any of embodiments 146-161, wherein the fusion protein comprises one or more nuclear localization signals.
163. The method of any one of embodiments 146-162, wherein said fusion protein is selected from the group consisting of:
a) a fusion protein comprising said deaminase, wherein said deaminase comprises an amino acid sequence having at least 90% sequence identity to SEQ ID NO: 319 and is fused to the N-terminus of an RGN comprising an amino acid sequence having at least 90% sequence identity to SEQ ID NO: 49;
b) a fusion protein comprising said deaminase, wherein said deaminase comprises an amino acid sequence having at least 90% sequence identity to SEQ ID NO: 375 and is fused to the N-terminus of an RGN comprising an amino acid sequence having at least 90% sequence identity to SEQ ID NO: 49;
c) a fusion protein comprising said deaminase, wherein said deaminase comprises an amino acid sequence having at least 90% sequence identity to SEQ ID NO: 406 and is fused to the N-terminus of an RGN comprising an amino acid sequence having at least 90% sequence identity to SEQ ID NO: 49;
d) a fusion protein comprising said deaminase, wherein said deaminase comprises an amino acid sequence having at least 90% sequence identity to SEQ ID NO: 596 and is fused to the N-terminus of an RGN comprising an amino acid sequence having at least 90% sequence identity to SEQ ID NO: 49;
e) a fusion protein comprising said deaminase, wherein said deaminase comprises an amino acid sequence having at least 90% sequence identity to SEQ ID NO: 597 and is fused to the N-terminus of an RGN comprising an amino acid sequence having at least 90% sequence identity to SEQ ID NO: 49;
f) a fusion protein comprising said deaminase, wherein said deaminase comprises an amino acid sequence having at least 90% sequence identity to SEQ ID NO: 303 and is fused to the N-terminus of an RGN comprising an amino acid sequence having at least 90% sequence identity to SEQ ID NO: 49;
g) a fusion protein comprising said deaminase, wherein said deaminase comprises an amino acid sequence having at least 90% sequence identity to SEQ ID NO: 366 and is fused to the N-terminus of an RGN comprising an amino acid sequence having at least 90% sequence identity to SEQ ID NO: 49; and
h) a fusion protein comprising said deaminase, wherein said deaminase comprises an amino acid sequence having at least 90% sequence identity to SEQ ID NO: 720 and is fused to the N-terminus of an RGN comprising an amino acid sequence having at least 90% sequence identity to SEQ ID NO: 49.
164. The method of any one of embodiments 146-162, wherein said fusion protein is selected from the group consisting of:
a) a fusion protein comprising said deaminase, wherein said deaminase comprises an amino acid sequence having at least 95% sequence identity to SEQ ID NO: 319 and is fused to the N-terminus of an RGN comprising an amino acid sequence having at least 95% sequence identity to SEQ ID NO: 49;
b) a fusion protein comprising said deaminase, wherein said deaminase comprises an amino acid sequence having at least 95% sequence identity to SEQ ID NO: 375 and is fused to the N-terminus of an RGN comprising an amino acid sequence having at least 95% sequence identity to SEQ ID NO: 49;
c) a fusion protein comprising said deaminase, wherein said deaminase comprises an amino acid sequence having at least 95% sequence identity to SEQ ID NO: 406 and is fused to the N-terminus of an RGN comprising an amino acid sequence having at least 95% sequence identity to SEQ ID NO: 49;
d) a fusion protein comprising said deaminase, wherein said deaminase comprises an amino acid sequence having at least 95% sequence identity to SEQ ID NO: 596 and is fused to the N-terminus of an RGN comprising an amino acid sequence having at least 95% sequence identity to SEQ ID NO: 49;
e) a fusion protein comprising said deaminase, wherein said deaminase comprises an amino acid sequence having at least 95% sequence identity to SEQ ID NO: 597 and is fused to the N-terminus of an RGN comprising an amino acid sequence having at least 95% sequence identity to SEQ ID NO: 49;
f) a fusion protein comprising said deaminase, wherein said deaminase comprises an amino acid sequence having at least 95% sequence identity to SEQ ID NO: 303 and is fused to the N-terminus of an RGN comprising an amino acid sequence having at least 95% sequence identity to SEQ ID NO: 49;
g) a fusion protein comprising said deaminase, wherein said deaminase comprises an amino acid sequence having at least 95% sequence identity to SEQ ID NO: 366 and is fused to the N-terminus of an RGN comprising an amino acid sequence having at least 95% sequence identity to SEQ ID NO: 49; and
h) a fusion protein comprising said deaminase, wherein said deaminase comprises an amino acid sequence having at least 95% sequence identity to SEQ ID NO: 720 and is fused to the N-terminus of an RGN comprising an amino acid sequence having at least 95% sequence identity to SEQ ID NO: 49.
165. The method of any one of embodiments 146-162, wherein said fusion protein is selected from the group consisting of:
a) a fusion protein comprising said deaminase, wherein said deaminase comprises the amino acid sequence set forth as SEQ ID NO: 319 and is fused to the N-terminus of an RGN comprising the amino acid sequence set forth as SEQ ID NO: 49;
b) a fusion protein comprising said deaminase, wherein said deaminase comprises the amino acid sequence set forth as SEQ ID NO: 375 and is fused to the N-terminus of an RGN comprising the amino acid sequence set forth as SEQ ID NO: 49;
c) a fusion protein comprising said deaminase, wherein said deaminase comprises the amino acid sequence set forth as SEQ ID NO: 406 and is fused to the N-terminus of an RGN comprising the amino acid sequence set forth as SEQ ID NO: 49;
d) a fusion protein comprising said deaminase, wherein said deaminase comprises the amino acid sequence set forth as SEQ ID NO: 596 and is fused to the N-terminus of an RGN comprising the amino acid sequence set forth as SEQ ID NO: 49;
e) a fusion protein comprising said deaminase, wherein said deaminase comprises the amino acid sequence set forth as SEQ ID NO: 597 and is fused to the N-terminus of an RGN comprising the amino acid sequence set forth as SEQ ID NO: 49;
f) a fusion protein comprising said deaminase, wherein said deaminase comprises the amino acid sequence set forth as SEQ ID NO: 303 and is fused to the N-terminus of an RGN comprising the amino acid set forth as SEQ ID NO: 49;
g) a fusion protein comprising said deaminase, wherein said deaminase comprises the amino acid sequence set forth as SEQ ID NO: 366 and is fused to the N-terminus of an RGN comprising the amino acid sequence set forth as SEQ ID NO: 49; and
h) a fusion protein comprising said deaminase, wherein said deaminase comprises the amino acid sequence set forth as SEQ ID NO: 720 and is fused to the N-terminus of an RGN comprising the amino acid sequence set forth as SEQ ID NO: 49.
166. The method of any one of embodiments 163-165, wherein said fusion protein has the sequence set forth as any one of SEQ ID NOs: 577, 578, 582, 584, 588, 593, 594, 604, 605, 616, and 729.
167. The method of any of embodiments 146-166, wherein the fusion protein is codon optimized for expression in a eukaryotic cell.
168. The method of any one of embodiments 146-167, wherein said modified target DNA molecule comprises an A>N mutation of at least one nucleotide within the target DNA molecule, wherein N is C, G, or T.
169. The method of embodiment 168, wherein said modified target DNA molecule comprises an A>G mutation of at least one nucleotide within the target DNA molecule.
170. The method of any one of embodiments 146-169, wherein said target DNA sequence is a eukaryotic target DNA sequence.
171. The method of any of embodiments 146-170, wherein said target DNA sequence is located adjacent to a protospacer adjacent motif (PAM).
172. The method of any one of embodiments 146-171, wherein the target DNA molecule comprises a causal mutation for a disease or disorder and wherein modification of the target DNA molecule corrects said causal mutation.
173. The method of embodiment 172, wherein the correction of the causal mutation comprises correcting a nonsense mutation.
174. The method of any of embodiments 146-173, wherein the target DNA molecule is within a cell.
175. The method of embodiment 174, wherein the cell is a eukaryotic cell.
176. The method of embodiment 175, wherein the eukaryotic cell is a plant cell.
177. The method of embodiment 175, wherein the eukaryotic cell is a mammalian cell.
178. The method of embodiment 177, wherein the mammalian cell is a human cell.
179. The method of embodiment 178, wherein the human cell is an immune cell.
180. The method of embodiment 179, wherein the immune cell is a stem cell.
181. The method of embodiment 180, wherein the stem cell is an induced pluripotent stem cell.
182. The method of embodiment 175, wherein the eukaryotic cell is an insect cell.
183. The method of embodiment 174, wherein the cell is a prokaryotic cell.
184. The method of any one of embodiments 174-183, further comprising selecting a cell comprising said modified DNA molecule.
185. A cell comprising a modified target DNA sequence according to the method of embodiment 184.
186. The cell of embodiment 185, wherein the cell is a eukaryotic cell.
187. The cell of embodiment 186, wherein the eukaryotic cell is a plant cell.
188. A plant comprising the cell of embodiment 187.
189. A seed comprising the cell of embodiment 187.
190. The cell of embodiment 187, wherein the eukaryotic cell is a mammalian cell.
191. The cell of embodiment 190, wherein the mammalian cell is a human cell.
192. The cell of embodiment 191, wherein the human cell is an immune cell.
193. The cell of embodiment 192, wherein the immune cell is a stem cell.
194. The cell of embodiment 193, wherein the stem cell is an induced pluripotent stem cell.
195. The cell of embodiment 186, wherein the eukaryotic cell is an insect cell.
196. The cell of embodiment 185, wherein the cell is a prokaryotic cell.
197. A pharmaceutical composition comprising the cell of any one of embodiments 190-194, and a pharmaceutically acceptable carrier.
198. A composition comprising:
a) a fusion protein comprising a DNA-binding polypeptide and a first adenine deaminase, or a nucleic acid molecule encoding the fusion protein; and
b) a second adenine deaminase having at least 85% sequence identity to SEQ ID NO: 5; or a nucleic acid molecule encoding the deaminase, wherein the second deaminase comprises at least one of the following amino acid residues:
   i) a C at a position corresponding to position 2 of SEQ ID NO: 5;
   ii) a F or C at a position corresponding to position 22 of SEQ ID NO: 5;
   iii) a Q at a position corresponding to position 23 of SEQ ID NO: 5;
   iv) a N at a position corresponding to position 35 of SEQ ID NO: 5;
   v) a L at a position corresponding to position 40 of SEQ ID NO: 5;
   vi) a Q at a position corresponding to position 46 of SEQ ID NO: 5;
   vii) a A or M at a position corresponding to position 68 of SEQ ID NO: 5;
   viii) a H at a position corresponding to position 72 of SEQ ID NO: 5;
   ix) a W, A, Q, Y, or D at a position corresponding to position 75 of SEQ ID NO: 5;
   x) a G at a position corresponding to position 76 of SEQ ID NO: 5;
   xi) a S at a position corresponding to position 81 of SEQ ID NO: 5;
   xii) a M at a position corresponding to position 105 of SEQ ID NO: 5;
   xiii) a H at a position corresponding to position 108 of SEQ ID NO: 5;
   xiv) a L at a position corresponding to position 109 of SEQ ID NO: 5;
   xv) a I or L at a position corresponding to position 117 of SEQ ID NO: 5;
   xvi) a F at a position corresponding to position 120 of SEQ ID NO: 5;
   xvii) a E, T, A, G, or V at a position corresponding to position 121 of SEQ ID NO: 5;
   xviii) a I or H at a position corresponding to position 122 of SEQ ID NO: 5;
   xix) a K at a position corresponding to position 125 of SEQ ID NO: 5;
   xx) a A at a position corresponding to position 126 of SEQ ID NO: 5;
   xxi) a H at a position corresponding to position 135 of SEQ ID NO: 5;
   xxii) a V at a position corresponding to position 137 of SEQ ID NO: 5;
   xxiii) a Y at a position corresponding to position 138 of SEQ ID NO: 5;
   xxiv) a L at a position corresponding to position 139 of SEQ ID NO: 5;
   xxv) a K or A at a position corresponding to position 142 of SEQ ID NO: 5;
   xxvi) a A, Q, L, or M at a position corresponding to position 145 of SEQ ID NO: 5;
   xxvii) a K at a position corresponding to position 148 of SEQ ID NO: 5;
   xxviii) a Q at a position corresponding to position 151 of SEQ ID NO: 5;
   xxix) a E or R at a position corresponding to position 153 of SEQ ID NO: 5;
   xxx) a W at a position corresponding to position 155 of SEQ ID NO: 5;
   xxxi) a R or V at a position corresponding to position 156 of SEQ ID NO: 5;
   xxxii) a F at a position corresponding to position 157 of SEQ ID NO: 5;
   xxxiii) a R at a position corresponding to position 158 of SEQ ID NO: 5;
   xxxiv) a Q at a position corresponding to position 159 of SEQ ID NO: 5;
   xxxv) a D or W at a position corresponding to position 160 of SEQ ID NO: 5;
   xxxvi) a W, H, V, or A at a position corresponding to position 162 of SEQ ID NO: 5;
   xxxvii) a R at a position corresponding to position 165 of SEQ ID NO: 5; and
   xxxviii) a H at a position corresponding to position 166 of SEQ ID NO: 5.
199. The composition of embodiment 198, wherein said second deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
200. The composition of embodiment 198, wherein said second deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
201. The composition of embodiment 198 or 200, wherein said second deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
202. The composition of any one of embodiments 198, 200, and 201, wherein said second deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
203. The composition of any one of embodiments 198-202, wherein said second adenine deaminase has at least 90% sequence identity to any one of SEQ ID NOs: 300-414, 596-598, and 720-723.
204. The composition of any one of embodiments 198-202, wherein said second adenine deaminase has 95% sequence identity to any one of SEQ ID NOs: 300-414, 596-598, and 720-723.
205. The composition of any one of embodiments 198-202, wherein said second adenine deaminase has the amino acid sequence of any one of SEQ ID NOs: 300-414, 596-598, and 720-723.
206. The composition of any one of embodiments 198-205, wherein the first adenine deaminase has at least 85% sequence identity to SEQ ID NO: 5 and comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
207. The composition of embodiment 206, wherein said first deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
208. The composition of embodiment 206, wherein said first deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
209. The composition of embodiment 206 or 208, wherein said first deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
210. The composition of any one of embodiments 206, 208, or 209, wherein said first deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
211. The composition of any one of embodiments 206-210, wherein the first adenine deaminase has at least 90% sequence identity to any one of SEQ ID NOs: 300-414, 596, 597, and 598, 720-723.
212. The composition of any one of embodiments 206-210, wherein the first adenine deaminase has 95% sequence identity to any one of SEQ ID NOs: 300-414, 596-598, and 720-723.
213. The composition of any one of embodiments 206-210, wherein the first adenine deaminase has the amino acid sequence of any one of SEQ ID NOs: 300-414, 596-598, and 720-723.
214. The composition of any one of embodiments 198-213, wherein the DNA-binding polypeptide is a meganuclease, zinc finger fusion protein, or a TALEN.
215. The composition of any one of embodiments 198-213, wherein the DNA-binding polypeptide is an RNA-guided, DNA-binding polypeptide.
216. The composition of embodiment 215, wherein the RNA-guided, DNA-binding polypeptide is an RNA-guided nuclease (RGN) polypeptide.
217. The composition of embodiment 216, wherein the RGN is an RGN nickase.
218. A vector comprising a first nucleic acid molecule encoding a fusion protein and a second nucleic acid molecule encoding a second deaminase, wherein said fusion protein comprises a DNA-binding polypeptide and a first adenine deaminase, and wherein said second adenine deaminase has at least 85% sequence identity to SEQ ID NO: 5, and wherein said second adenine deaminase has at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
219. The vector of embodiment 218, wherein said second deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
220. The vector of embodiment 218, wherein said second deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
221. The vector of embodiment 218 or 220, wherein said second deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position corresponding to position 145 of SEQ ID NO: 5;
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
222. The vector of any one of embodiments 218, 220, and 221, wherein said second deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
223. The vector of any one of embodiments 218-222, wherein said second adenine deaminase has at least 90% sequence identity to SEQ ID NO: 5.
224. The vector of any one of embodiments 218-222, wherein said second adenine deaminase has at least 95% sequence identity to SEQ ID NO: 5.
225. The vector of any one of embodiments 218-222, wherein said second adenine deaminase has an amino acid sequence of any one of SEQ ID NOs: 300-414, 596-598, and 720-723.
226. The vector of any one of embodiments 218-225, wherein the first adenine deaminase has at least 85% sequence identity to SEQ ID NO: 5, and comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.
227. The vector of embodiment 226, wherein said first deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a A at a position corresponding to position 68 of SEQ ID NO: 5;
g) a W, A, Q, or Y at a position corresponding to position 75 of SEQ ID NO: 5;
h) a G at a position corresponding to position 76 of SEQ ID NO: 5;
i) a S at a position corresponding to position 81 of SEQ ID NO: 5;
j) a M at a position corresponding to position 105 of SEQ ID NO: 5;
k) a H at a position corresponding to position 108 of SEQ ID NO: 5;
l) a L at a position corresponding to position 109 of SEQ ID NO: 5;
m) a I or L at a position corresponding to position 117 of SEQ ID NO: 5;
n) a A or G at a position corresponding to position 121 of SEQ ID NO: 5;
o) a H at a position corresponding to position 122 of SEQ ID NO: 5;
p) a A at a position corresponding to position 126 of SEQ ID NO: 5;
q) a H at a position corresponding to position 135 of SEQ ID NO: 5;
r) a V at a position corresponding to position 137 of SEQ ID NO: 5;
s) a Y at a position corresponding to position 138 of SEQ ID NO: 5;
t) a L at a position corresponding to position 139 of SEQ ID NO: 5;
u) a K or A at a position corresponding to position 142 of SEQ ID NO: 5;
v) a A, Q, L, or M at a position corresponding to position 145 of SEQ ID NO: 5;
w) a E or R at a position corresponding to position 153 of SEQ ID NO: 5;
x) a W at a position corresponding to position 155 of SEQ ID NO: 5;
y) a R at a position corresponding to position 156 of SEQ ID NO: 5;
z) a F at a position corresponding to position 157 of SEQ ID NO: 5;
aa) a R at a position corresponding to position 158 of SEQ ID NO: 5;
bb) a Q at a position corresponding to position 159 of SEQ ID NO: 5;
cc) a D or W at a position corresponding to position 160 of SEQ ID NO: 5;
dd) a A or W at a position corresponding to position 162 of SEQ ID NO: 5; and
ee) a R at a position corresponding to position 165 of SEQ ID NO: 5.
228. The vector of embodiment 226, wherein said first deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.
229. The vector of embodiment 226 or 228, wherein said first deaminase comprises:
a) a N at a position corresponding to position 35 of SEQ ID NO: 5 and a Q at a position corresponding to position 46 of SEQ ID NO: 5;
b) a S at a position corresponding to position 81 of SEQ ID NO: 5 and a R at a position corresponding to position 156 of SEQ ID NO: 5;
c) a Q at a position corresponding to position 46 of SEQ ID NO: 5 and a R at a position corresponding to position 156 of SEQ ID NO: 5;
d) a N at a position corresponding to position 35 of SEQ ID NO: 5, a Q at a position corresponding to position 46 of SEQ ID NO: 5, and a W at a position corresponding to position 162 of SEQ ID NO: 5;
e) a W at a position corresponding to position 155 of SEQ ID NO: 5, a R at a position corresponding to position 156 of SEQ ID NO: 5, and a W at a position corresponding to position 162 of SEQ ID NO: 5;
f) a N at a position corresponding to position 35 of SEQ ID NO: 5, a R at a position corresponding to position 156 of SEQ ID NO: 5, and a W at a position corresponding to position 162 of SEQ ID NO: 5;
g) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
h) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
i) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
j) a S at a position corresponding to position 81 of SEQ ID NO: 5, and a W at a position corresponding to position 155 of SEQ ID NO: 5;
k) a W at a position corresponding to position 75 of SEQ ID NO: 5, and a S at a position corresponding to position 81 of SEQ ID NO: 5;
l) a S at a position corresponding to position 81 of SEQ ID NO: 5, and a D at a position corresponding to position 160 of SEQ ID NO: 5;
m) a S at a position corresponding to position 81 of SEQ ID NO: 5, and a A at a position corresponding to position 145 of SEQ ID NO: 5;
n) a W at a position corresponding to position 75 of SEQ ID NO: 5, and a W at a position corresponding to position 155 of SEQ ID NO: 5;
o) a W at a position corresponding to position 75 of SEQ ID NO: 5, and a A at a position
p) a A at a position corresponding to position 145 of SEQ ID NO: 5, and a D at a position corresponding to position 160 of SEQ ID NO: 5;
q) a W at a position corresponding to position 75 of SEQ ID NO: 5, a S at a position corresponding to position 81 of SEQ ID NO: 5, and a W at a position corresponding to position 155 of SEQ ID NO: 5;
r) a S at a position corresponding to position 81 of SEQ ID NO: 5, a W at a position corresponding to position 155 of SEQ ID NO: 5, and a D at a position corresponding to position 160 of SEQ ID NO: 5;
s) a S at a position corresponding to position 81 of SEQ ID NO: 5, a A at a position corresponding to position 145 of SEQ ID NO: 5, and a W at a position corresponding to position 155 of SEQ ID NO: 5;
t) a L at a position corresponding to position 40 of SEQ ID NO: 5, and a M at a position corresponding to position 105 of SEQ ID NO: 5;
u) a L at a position corresponding to position 40 of SEQ ID NO: 5, and a M at a position corresponding to position 145 of SEQ ID NO: 5;
v) a L at a position corresponding to position 40 of SEQ ID NO: 5, and a Q at a position corresponding to position 159 of SEQ ID NO: 5;
w) a L at a position corresponding to position 40 of SEQ ID NO: 5, and a W at a position corresponding to position 160 of SEQ ID NO: 5;
x) a M at a position corresponding to position 105 of SEQ ID NO: 5, and a M at a position corresponding to position 145 of SEQ ID NO: 5;
y) a M at a position corresponding to position 105 of SEQ ID NO: 5, and a W at a position corresponding to position 160 of SEQ ID NO: 5;
z) a M at a position corresponding to position 145 of SEQ ID NO: 5, and a Q at a position corresponding to position 159 of SEQ ID NO: 5;
aa) a M at a position corresponding to position 145 of SEQ ID NO: 5, and a W at a position corresponding to position 160 of SEQ ID NO: 5;
bb) a Q at a position corresponding to position 159 of SEQ ID NO: 5, and a W at a position corresponding to position 160 of SEQ ID NO: 5;
cc) a S at a position corresponding to position 81 of SEQ ID NO: 5, a M at a position corresponding to position 145 of SEQ ID NO: 5, and a W at a position corresponding to position 160 of SEQ ID NO: 5;
dd) a A at a position corresponding to position 68 of SEQ ID NO: 5, and a S at a position corresponding to position 81 of SEQ ID NO: 5;
ee) a G at a position corresponding to position 76 of SEQ ID NO: 5, and a S at a position corresponding to position 81 of SEQ ID NO: 5;
ff) a S at a position corresponding to position 81 of SEQ ID NO: 5, and a L at a position corresponding to position 117 of SEQ ID NO: 5;
gg) a A at a position corresponding to position 68 of SEQ ID NO: 5, and a G at a position corresponding to position 76 of SEQ ID NO: 5;
hh) a A at a position corresponding to position 68 of SEQ ID NO: 5, and a L at a position corresponding to position 117 of SEQ ID NO: 5;
ii) a G at a position corresponding to position 76 of SEQ ID NO: 5, and a L at a position corresponding to position 117 of SEQ ID NO: 5;
jj) a A at a position corresponding to position 68 of SEQ ID NO: 5, a G at a position corresponding to position 76 of SEQ ID NO: 5, and a S at a position corresponding to position 81 of SEQ ID NO: 5;
kk) a A at a position corresponding to position 68 of SEQ ID NO: 5, a S at a position corresponding to position 81 of SEQ ID NO: 5, and a L at a position corresponding to position 117 of SEQ ID NO: 5;
ll) a G at a position corresponding to position 76 of SEQ ID NO: 5, a S at a position corresponding to position 81 of SEQ ID NO: 5, and a L at a position corresponding to position 117 of SEQ ID NO: 5;
mm) a A at a position corresponding to position 68 of SEQ ID NO: 5, a G at a position corresponding to position 76 of SEQ ID NO: 5, and a L at a position corresponding to position 117 of SEQ ID NO: 5;
nn) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
oo) a A at a position corresponding to position 68 of SEQ ID NO: 5 and a S at a position corresponding to position 81 of SEQ ID NO: 5;
pp) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
qq) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
rr) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
ss) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position
tt) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
uu) a S at a position corresponding to position 81 of SEQ ID NO: 5; or
vv) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
230. The vector of any one of embodiments 226, 228, and 229, wherein said first deaminase comprises:
a) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
b) a W at a position corresponding to position 75 of SEQ ID NO: 5, and a W at a position corresponding to position 155 of SEQ ID NO: 5;
c) a S at a position corresponding to position 81 of SEQ ID NO: 5, a W at a position corresponding to position 155 of SEQ ID NO: 5, and a D at a position corresponding to position 160 of SEQ ID NO: 5;
d) a S at a position corresponding to position 81 of SEQ ID NO: 5, a A at a position corresponding to position 145 of SEQ ID NO: 5, and a W at a position corresponding to position 155 of SEQ ID NO: 5;
e) a S at a position corresponding to position 81 of SEQ ID NO: 5, a M at a position corresponding to position 145 of SEQ ID NO: 5, and a W at a position corresponding to position 160 of SEQ ID NO: 5;
f) a G at a position corresponding to position 76 of SEQ ID NO: 5, and a S at a position corresponding to position 81 of SEQ ID NO: 5;
g) a S at a position corresponding to position 81 of SEQ ID NO: 5, and a L at a position corresponding to position 117 of SEQ ID NO: 5;
h) a A at a position corresponding to position 68 of SEQ ID NO: 5 and a S at a position corresponding to position 81 of SEQ ID NO: 5;
i) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
j) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
k) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
l) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
m) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5;
n) a S at a position corresponding to position 81 of SEQ ID NO: 5; or
o) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5.
231. The vector of any one of embodiments 226-230, wherein the first adenine deaminase has at least 90% sequence identity to any one of SEQ ID NOs: 300-414, 596-598, and 720-723.
232. The vector of any one of embodiments 226-230, wherein the first adenine deaminase has at least 95% sequence identity to any one of SEQ ID NOs: 300-414, 596-598, and 720-723.
233. The vector of any one of embodiments 226-230, wherein the first adenine deaminase has an amino acid sequence of any one of SEQ ID NOs: 300-414, 596-598, and 720-723.
234. The vector of any one of embodiments 218-233, wherein the DNA-binding polypeptide is a meganuclease, zinc finger fusion protein, or a TALEN.
235. The vector of any one of embodiments 218-233, wherein the DNA-binding polypeptide is an RNA-guided, DNA-binding polypeptide.
236. The vector of embodiment 235, wherein the RNA-guided, DNA-binding polypeptide is an RNA-guided nuclease (RGN) polypeptide.
237. The vector of embodiment 236, wherein the RGN is an RGN nickase.
238. A cell comprising the composition of any one of embodiments 186-205 or the vector of any one of embodiments 218-237.
239. A pharmaceutical composition comprising a pharmaceutically acceptable carrier and the composition of any one of embodiments 198-217, the vector of any one of embodiments 218-237, or the cell of embodiment 238.
240. A method for treating a subject having or at risk of developing a disease, disorder, or condition, the method comprising:
administering to the subject the fusion protein of any of embodiments 27-50, the RNP complex of embodiment 51, the nucleic acid molecule of any one of embodiments 52-77, the vector of any one of embodiments 78-81 and 218-237, the cell of any one of embodiments 88-92, 132-136, 190-194, and 238, the system of any one of embodiments 104-129, the composition of any one of embodiments 196-217, or the pharmaceutical composition of any one of embodiments 98, 142, 197, and 239.
241. The method of embodiment 240, wherein said disease is associated with a causal mutation and said treating comprises correcting said causal mutation.
242. Use of the fusion protein of any one of embodiments 27-50, the RNP complex of embodiment 51, the nucleic acid molecule of any one of embodiments 52-77, the vector of any one of embodiments 78-81 and 218-237, the cell of any one of embodiments 88-92, 132-136, 190-194, and 238, the system of any one of embodiments 104-129, or the composition of any one of embodiments 196-217 for the treatment of a disease, disorder, or condition in a subject having or at risk of developing said disease, disorder, or condition.
243. The use of embodiment 242, wherein said disease is associated with a causal mutation and said treating comprises correcting said causal mutation.
244. Use of the fusion protein of any one of embodiments 27-50, the RNP complex of embodiment 51, the nucleic acid molecule of any one of embodiments 52-77, the vector of any one of embodiments 78-81 and 218-237, the cell of any one of embodiments 88-92, 132-136, 190-194, and 238, the system of any one of embodiments 104-129, or the composition of any one of embodiments 196-217 for the manufacture of a medicament useful for treating a disease, disorder, or condition.
245. The use of embodiment 244, wherein said disease is associated with a causal mutation and an effective amount of said medicament corrects said causal mutation.

The following examples are offered by way of illustration and not by way of limitation.

### EXPERIMENTAL

### Example 1: Inlaid Base Editors are Functional and Some Exhibit a Shift in Editing Window

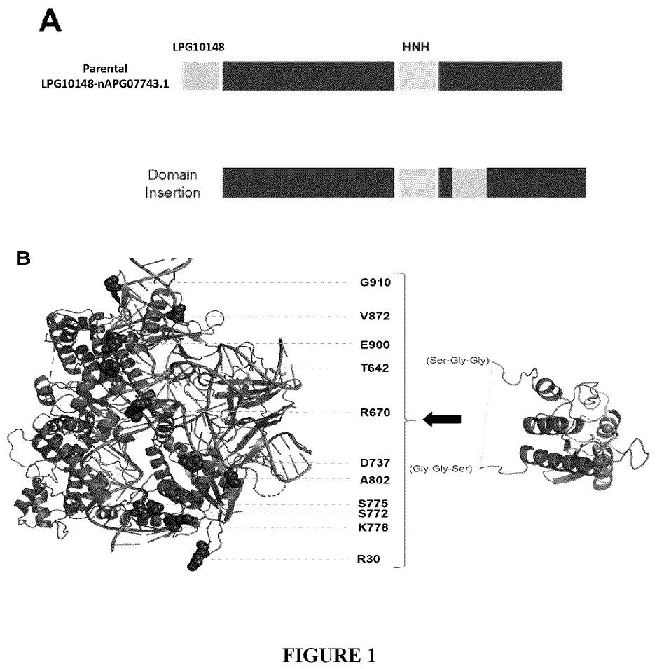

The parental adenine base editor (ABE; LPG50148-nAPG07433.1) was created by the end-to-end fusion between LPG50148 (set forth as SEQ ID NO: 5 and was originally disclosed in WO 2022/056254, which is incorporated in its entirety by reference herein) and a nickase version of APG07433.1 with a D11A mutation (set forth as SEQ ID NO: 49 and was originally disclosed in WO 2019/236566, which is incorporated in its entirety by reference herein) with a linker between (L32 linker sequence set forth as SEQ ID NO: 705). Constructs with LPG50148 inserted into nAPG07433.1 are depicted in FIG. 1A. The insertion positions were selected based on a structural analysis to position the deaminase active site to bases outside the canonical editing window.

LPG50148 (minus the starting methionine, set forth as SEQ ID NO: 5, and flanked with a short GGS linker at the N-terminus and the SGG linker at the C-terminus) was inserted immediately after the following amino acid residues in nAPG07433.1 (numbering according to SEQ ID NO: 1): S772 (referred to as LPG20002, the sequence of which is set forth as SEQ ID NO: 8), S775 (referred to as LPG20003, the sequence of which is set forth as SEQ ID NO: 9), K778 (referred to as LPG20004, the sequence of which is set forth as SEQ ID NO: 10), E900 (referred to as LPG20005, the sequence of which is set forth as SEQ ID NO: 11), G910 (referred to as LPG20006, the sequence of which is set forth as SEQ ID NO: 12), R30 (referred to as LPG20007, the sequence of which is set forth as SEQ ID NO: 13), T642 (referred to as LPG20008, the sequence of which is set forth as SEQ ID NO: 14), R670 (referred to as LPG20009, the sequence of which is set forth as SEQ ID NO: 15), D737 (referred to as LPG20010, the sequence of which is set forth as SEQ ID NO: 16), and A802 (referred to as LPG20011, the sequence of which is set forth as SEQ ID NO: 17).

A domain replacement strategy was also performed in which the LPG50148 deaminase replaced the nAPG07433.1 HNH domain. This fusion protein is referred to herein as LPG20001 and is set forth as SEQ ID NO: 428. The base editors were fused with NLS sequences at the N- and C-termini.

For each inlaid base editor (IBE) and the HNH replacement protein, the editing efficiency in human cells was measured. IBEs, the HNH replacement protein, and guide RNAs were delivered as plasmids encoding the same into HEK293T cells.

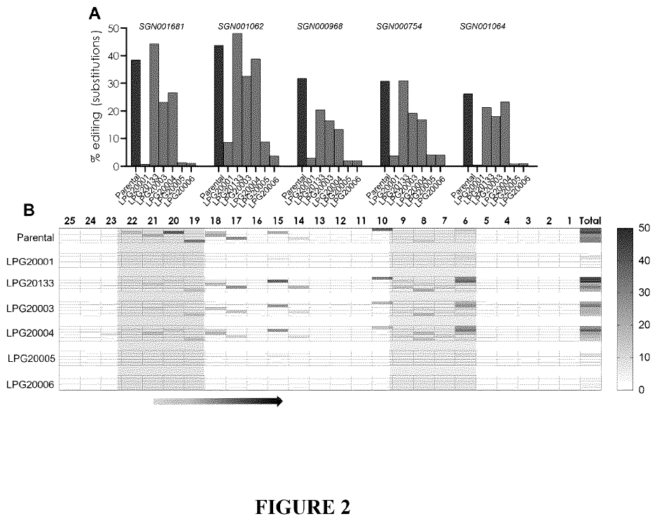

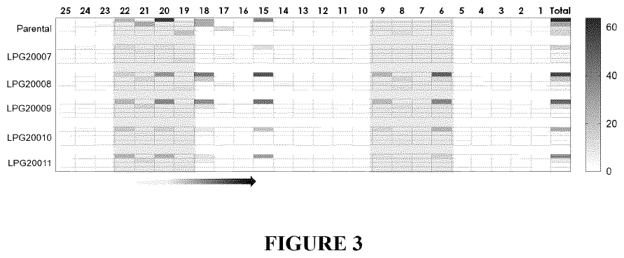

The results presented in FIG. 2B show a significant decrease in editing efficiency between positions 19-22 of the editing window and an increase between positions 6-9 for LPG20002-LPG20004. LPG20008-LPG20010 also show a decrease in editing efficiency between positions 19-22 and an increase between positions 6-9 (FIG. 3). Therefore, these new engineered base editors exhibit a particular shift in the editing window proximal to the PAM. For the domain replacement strategy, LPG20001 showed almost no editing (data not shown). The deaminase was also inserted into the nAPG07433.1 protein in which the HNH domain was deleted and these proteins also showed very low editing (data not shown). Additionally, the deaminase was inserted into a nuclease-inactive APG07433.1 at several positions. Upon testing in cells, these IBEs (LPG20134-LPG20138) showed almost no editing as well (data not shown).

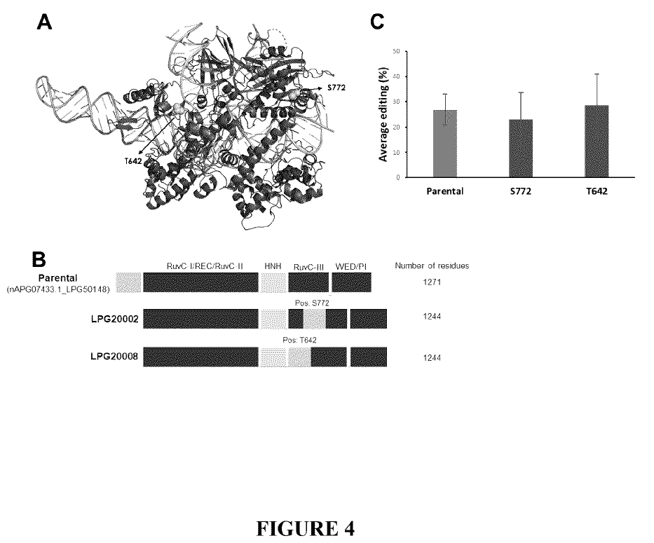

Two constructs, LPG20002 and LPG20008, with altered editing windows and the highest editing efficiency, were selected for additional studies. These constructs show higher editing for positions A6-A9, are smaller than the end-to-end fusion, and total editing efficiency is comparable with the parental ABE (FIG. 4).

### Example 2: Deletion of Linkers within Inlaid Base Editors

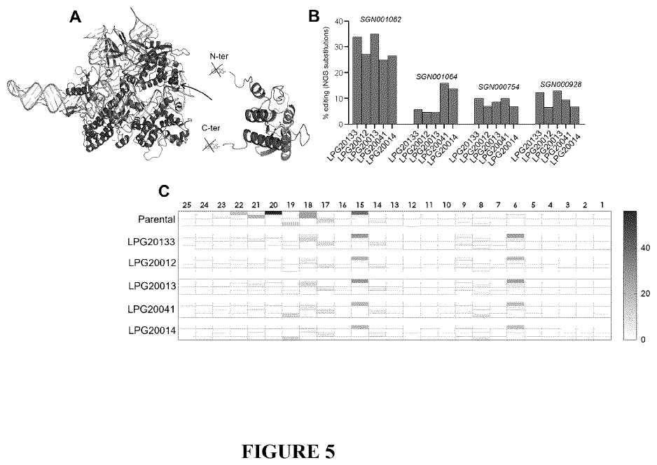

Inlaid base editors LPG20002 and LPG20008 exhibited a broad editing window (FIGS. 2B and 3), an important feature for gene knockout targeting splice sites. However, a more focused window would be desirable to fix a specific mutation. In order to reduce the width of the editing window, it was hypothesized that deleting the GGS linkers between LPG50148 and nAPG07433.1 could reduce conformational flexibility, generating more focused deamination. The N-terminal, C-terminal, or both linkers from LPG20002 were deleted (FIG. 5A). LPG20002 with the amino terminal linker deleted is set forth as SEQ ID NO: 18 and referred to herein as LPG20012. LPG20002 with the carboxy terminal linker deleted is set forth as SEQ ID NO: 19 and is referred to herein as LPG20013. In addition to both linkers being deleted, LPG50148 was also truncated by a single amino acid at each terminal in the construct set forth as SEQ ID NO: 20 (LPG20014). IBEs and guide RNAs were delivered as plasmids encoding the same into HEK293T cells.

As expected, LPG20002 is still active after deletion of the linkers (FIG. 5B), demonstrating the GGS linkers are not essential. However, although the linker deletions did not reduce the editing window width for LPG20002 (FIG. 5C), these deletions decrease protein size, which is helpful for AAV delivery.

### Example 3: Therapeutic Potential of Inlaid Base Editors

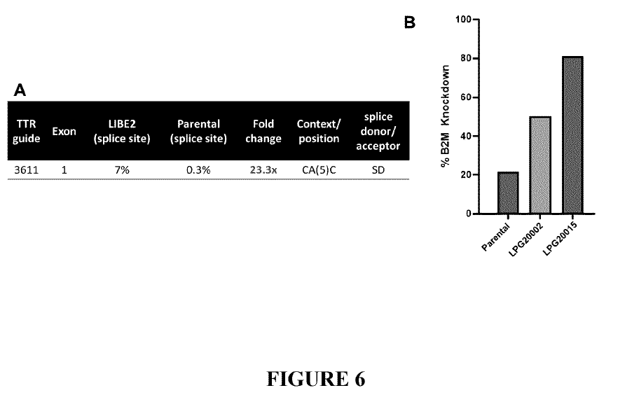

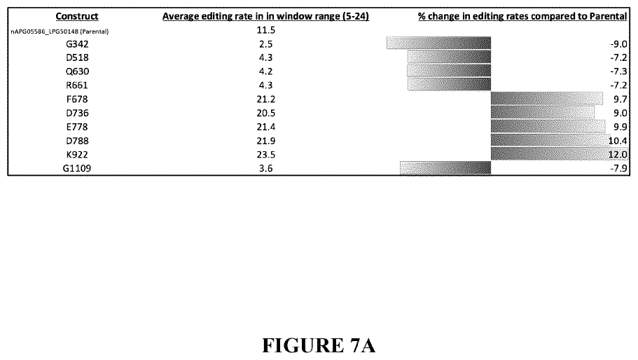

The therapeutic potential of the inlaid base editors was demonstrated by editing Hereditary ATTR Amyloidosis splice sites and by knocking out the P2 microglobulin (B2M) gene (FIG. 6). Hereditary ATTR amyloidosis is caused by a mutation within the transthyretin (TTR) gene, which results in an abnormal TTR protein that is unstable and readily misfolds, forming aggregates that deposit as amyloids in various organs and tissues in the body. ABE splice site targeting is a strategy to knockout TTR production in the liver. In these studies, LPG20002 or parental ABE and guide RNAs were delivered as plasmids encoding the same via nucleofection into HEK cells. LPG20002 showed 23.3-fold higher editing than the parental ABE for editing a consensus splice donor sequence in exon 1 of the TTR gene, showing the therapeutic potential for this application (FIG. 6A).

Allogeneic stem cells are potent sources for correcting cell malfunctions. However, the risk of immune rejection restrains the clinical use of these cells. B2M knockout is a strategy to bypass this limitation. Thus, the efficiency of IBEs to knockout the B2M gene was evaluated. IBEs and the parental ABE were delivered as mRNA and guide RNAs were delivered as RNA molecules via nucleofection into T cells. As shown in FIG. 6B, LPG20002 showed ~2.5-fold higher efficiency than the parental ABE. In addition, a second version of LPG20002 (LPG20015 set forth as SEQ ID NO: 21) was generated by replacing the wild-type LPG50148 with a V81S variant (generating LPG50208, set forth as SEQ ID NO: 6). This LPG20015 showed A4-fold higher efficiency to knockout B2M than parental (FIG. 6B).

### Example 4: NAPG05586 Inlaid Base Editors

The parental adenine base editor (ABE; LPG50208-nAPG05586) was created by the end-to-end fusion between LPG50208 (LPG50148 with a V81S mutation; set forth as SEQ ID NO: 6) and a nickase version of APG05586 with a D7A mutation (set forth as SEQ ID NO: 50 and was originally disclosed in WO 2021/217002, which is incorporated in its entirety by reference herein) with a linker between (L32 linker sequence, set forth as SEQ ID NO: 705). Insertion positions for IBEs were selected based on a structural analysis.

LPG50208 was inserted immediately after the following amino acid residues in nAPG05586 (numbering according to SEQ ID NO: 2): G342 (within the Rec-2 domain); D518 (border residue between RuvC-2 and L1); Q630 (HNH domain); R661 (HNH domain); F678 (L-2 linker); D736 (RuvC-3 domain); E778 (RuvC-3 domain); D788 (RuvC-3 domain); K922 (WED domain); and G1109 (PI domain). The sequences of the nAPG05586 G342 IBE (referred to herein as LPG20016), D518 IBE (referred to herein as LPG20017), Q630 IBE (referred to herein as LPG20018), R661 IBE (referred to herein as LPG20019), F678 IBE (referred to herein as LPG20020), D736 IBE (referred to herein as LPG20021), E778 IBE (referred to herein as LPG20022), D788 IBE (referred to herein as LPG20023), K922 IBE (referred to herein as LPG20024), and G1109 IBE (referred to herein as LPG20025) are set forth as SEQ ID NOs: 23-32, respectively. The base editors were fused with NLS sequences at the N- and C-termini.

For each nAPG05586 IBE, the editing efficiency in human cells was measured with four guides (3 targeting B2M and 1 targeting TRAC) known to allow for APG05586 editing. IBEs and guide RNAs were delivered as plasmids encoding the same into HEK293T cells.

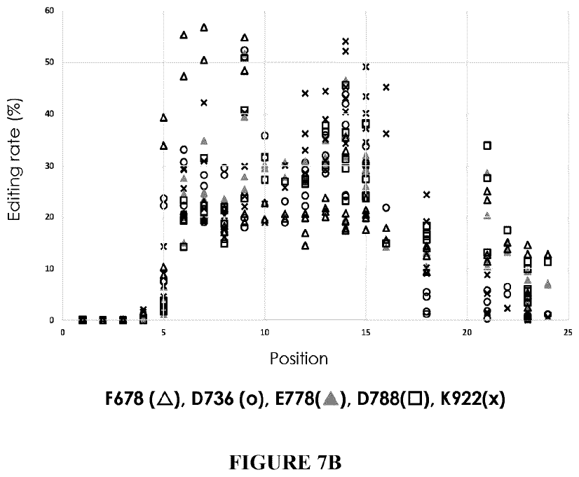

nAPG05586 IBEs with insertions immediately following the F678, D736, E778, D788, and K922 residues had editing rates greater than the parental ABE, with the K922 IBE having the greatest activity (see FIG. 7A). The remaining IBEs had lower activity than the parental ABE.

FIG. 7B provides the editing rates at every adenine position within the editing window (5-24) for each of the nAPG05586 IBEs with positions 5-7, 9 (also edited highly by nAPG05586 E778 IBE) and 24 being edited the greatest by the nAPG05586 F678 IBE and the highest maximal editing at position 23 by nAPG05586 F678 IBE. Positions 8 and 10 were edited the most by the nAPG05586 G736 IBE. Positions 21 and 22 were edited the most by the nAPG05586 D788 IBE and the highest average editing activity at positions 18 and 23 was by nAPG05586 D788 IBE. Positions 12-16 were edited the greatest by the nAPG05586 K922 IBE and the highest average editing rate at position 11 and the highest maximal activity at position 18 were by nAPG05586 K922 IBE.

### Example 5: NAPG01604 Inlaid Base Editors

The parental adenine base editor (ABE; LPG50208-nAPG01604) was created by the end-to-end fusion between LPG50208 (LPG50148 with a V81S mutation; set forth as SEQ ID NO: 6) and a nickase version of APGO1604 with a D10A mutation (set forth as SEQ ID NO: 51 and was originally disclosed in WO 2021/217002, which is incorporated in its entirety by reference herein) with a linker between (L32 linker sequence set forth as SEQ ID NO: 705). Insertion positions for IBEs were selected based on a structural analysis.

LPG50318 (set forth as SEQ ID NO: 598), similar to LPG50208 but with an additional A137V mutation, was inserted, flanked with a GSSG linker on both the N- and C-termini, immediately after the following amino acid residues in nAPG01604 (numbering according to SEQ ID NO: 3): G597 (referred to as LPG20027 or inlaid1.1), S631 (referred to as LPG20028 or inlaid2.1), K725 (referred to as LPG20029 or inlaid3.1), K739 (referred to as LPG20030 or inlaid4.1), and V744 (referred to as LPG20031 or inlaid5.1).

The sequences of the LPG20027, LPG20028, LPG20029, LPG20030, and LPG20031 are set forth as SEQ ID NOs: 34-38, respectively. The base editors were fused with NLS sequences at the N- and C-termini.

For each nAPG01604 inlaid base editor, the editing efficiency in human cells was measured with four guides (3 targeting B2M and 1 targeting TRAC) known to allow for APGO1604 editing. IBEs and guide RNAs were delivered as plasmids encoding the same into HEK293T cells.

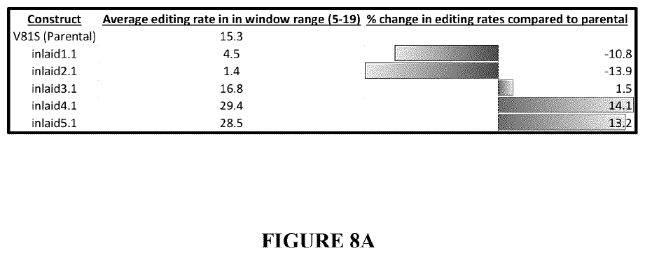

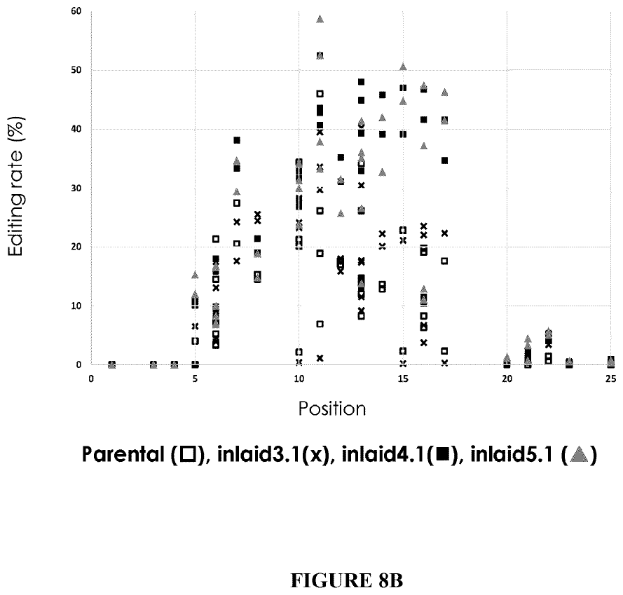

LPG20029, LPG20030, and LPG20031 had greater editing activity than the end-to-end ABE, with the LPG20027 and LPG20028 exhibiting lower editing activity than the end-to-end ABE (see FIG. 8A).

FIG. 8B provides the editing rates at adenines at positions 5-17 within the editing window for each of the nAPG01604 IBEs, with the exception of position 9 which was not edited by any of the nAPG01604 IBEs within these experiments. Position 8 was edited the most by LPG20029 (inlaid3.1; K725). Positions 6, 7, and 12-14 were edited the most by LPG20030 (inlaid4.1; K739), and the highest average activity at position 16 was by LPG20030 (inlaid4.1; K739). Positions 5, 10, 11, 15, and 17 were edited the most by LPG20031 (inlaid5.1; V744), and the highest maximal activity at position 16 was by LPG20031 (inlaid5.1; V744).

### Example 6: NLPG10145 Inlaid Base Editors

The parental adenine base editor (ABE; LPG50208-nLPG10145) was created by the end-to-end fusion between LPG50208 (LPG50148 with a V81S mutation; set forth as SEQ ID NO: 6) and a nickase version of LPG10145 with a D16A mutation (set forth as SEQ ID NO: 52 and was originally disclosed in WO 2023/139557, which is incorporated in its entirety by reference herein). Insertion positions for IBEs were selected based on a structural analysis.

LPG50208 (SEQ ID NO: 6, flanked with a short SGG linker at the N-terminus and GGS at the C-terminus) was inserted immediately after the following amino acid residues in nLPG10145 (numbering according to SEQ ID NO: 4): G347 (within the Rec-2 domain), D524 (border residue between RuvC-2 and L1), Q640 (HNH domain), R666 (HNH domain), D680 (L-2 linker), N740 (RuvC-3 domain), S785 (RuvC-3 domain), P910 (WED domain), and G1077 (PI domain). The sequences of the LPG10145 G347 IBE (referred to herein as LPG20032), D524 IBE (referred to herein as LPG20033), Q640 IBE (referred to herein as LPG20034), R666 IBE (referred to herein as LPG20035), D680 IBE (referred to herein as LPG20036), N740 IBE (referred to herein as LPG20037), S785 IBE (referred to herein as LPG20038), P910 IBE (referred to herein as LPG20039), and G1077 IBE (referred to herein as LPG20040) are set forth as SEQ ID NOs: 40-48, respectively.

For each nLPG10145 IBE, the editing efficiency in human cells was measured with four guides (3 targeting B2M and 1 targeting TRAC) known to allow for LPG10145 editing. IBEs and guide RNAs were delivered as plasmids encoding the same into HEK293T cells.

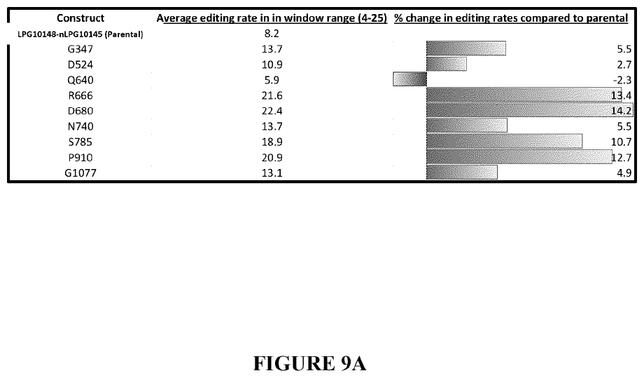

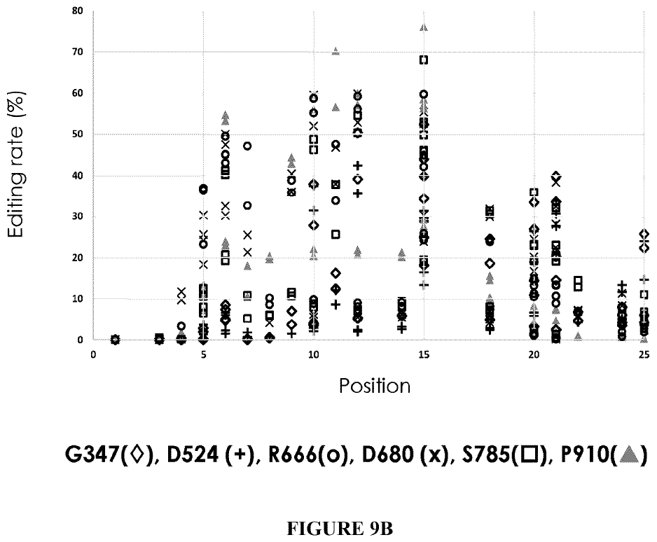

Most of the nLPG10145 IBEs had greater editing activity than the parental ABE, with the P910 IBE demonstrating the highest levels of ABE editing (see FIG. 9A). The Q640 IBE was the only tested LPG10145 IBE with lower activity than the parental ABE.

FIG. 9B provides the editing rates at every adenine position within the editing window (4-25) for each of the nLPG10145 IBEs, with the exception of positions 13, 16, 17, 19, and 23 which were not edited by any of the nLPG10145 IBEs within these experiments. Position 4 was edited the most by nLPG10145 D680 IBE, and the highest maximal activity at position 18 and highest average activity at position 21 was by nLPG10145 D680 IBE. Position 5 was edited the most by nLPG10145 R666 IBE and the highest average activity at position 6 was by nLPG10145 R666 IBE. Positions 20 and 22 were edited the most by nLPG10145 S785 IBE and the highest average editing activity at position 18 was by nLPG10145 S785 IBE. Position 24 was edited the greatest by nLPG10145 D524 IBE and position 25 by nLPG10145 G347 IBE. The highest maximal activity at position 21 was by nLPG10145 G347 IBE. Positions 8-12, 14, and 15 were edited the most by the nLPG10145 P910 IBE, and the highest maximal activity at position 6 was by nLPG10145 P910 IBE.

### Example 7: Directed Evolution of an Adenosine Base Editor (ABE) to Identify ABEs with Improved Editing Efficiency

The parental ABE (LPG50148-dAPG07433.1) was created by the end-to-end fusion between LPG50148 (set forth as SEQ ID NO: 5, without the starting methionine, was originally disclosed in WO 2022/056254, which is incorporated in its entirety by reference herein) and a nuclease inactive version of APG07433.1 that bears D1 1A and H604A mutations to inactivate both RuvC and HNH nuclease domains (disclosed in WO 2019/236566, which is incorporated in its entirety by reference herein). The parental LPG50148-dAPG07433.1 ABE was used in bacterial assays, whereas a LPG50148-nAPG07433.1 ABE was used as the parental ABE in human cell assays described below (LPG50148 fused to the D1 1A nickase version of APG07433.1).

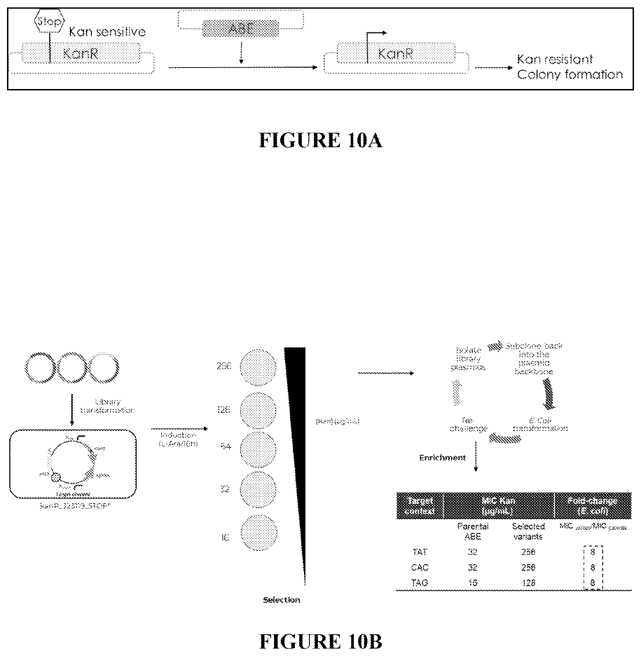

To engineer second generation ABEs with improved activity, a kanamycin selection system was established in E. coli that enables iterative rounds of directed evolution (FIG. 10A). This system is based on introducing a stop codon within the kanamycin resistance gene that makes it inactive and renders E. coli cells sensitive to kanamycin treatment. A library of ABE variants can be targeted to edit the A base(s) within the stop codon, thus converting it to a sense codon, where the kanamycin resistance gene is reactivated to permit survival of any E. coli cells expressing active ABEs. These variants are then sequenced to identify beneficial mutations which are promoted to testing in human cells.

To understand sequence-function relationships that can guide further rounds of directed evolution, a deep mutational scanning library was designed, wherein each residue of LPG50148 was mutated to all possible 20 amino acids. Using the kanamycin selection system, selection steps were performed using different sequence contexts (TAT, CAC, and TAG) and increased stringency (FIG. 10B). After the enrichment steps, we identified variants showing up to 8-fold activity improvement measured by increased antibiotic resistance (MICkan). These variants were sequenced, and the mutations were identified. Variants selected from the single mutation variant library were then promoted to human cell evaluation. Next, lead mutations with the highest human cell editing activity were combined and re-evaluated in human cells.

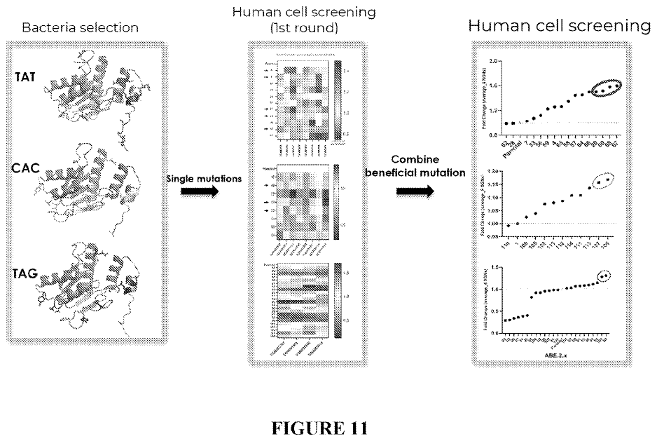

When the selection in bacteria was performed against TAT sequence contexts, 32 single mutations were identified after the enrichment steps (Table 2). The single mutations were incorporated into a human codon-optimized LPG50148-nAPG07433.1 fusion construct. The performance of these evolved deaminases in ABEs was evaluated by editing HEK293T cells at eight endogenous genomic sites. Single mutations showing the highest average fold-change were combined (Table 3) and tested to identify the top hits: variants LPG50148.2.20 (variant 20), LPG50148.2.60 (variant 60), LPG50148.2.67 (variant 67), and LPG50148.2.68 (variant 68), which showed about 1.6 times higher editing at canonical positions (A5-A7) in the protospacer and about four times higher editing at non-canonical positions (A3-A4 and A8-A10) as compared with the parental ABE (FIG. 11).

TABLE 2
TAT single mutations

| | | Mutations in relation |
|---|---|---|
| LPG50148 ABE | SEQ | to SEQ ID NO: 5 |
| Variant | ID NO | (LPG50148) |
| LPG50148.2.1 | 300 | L35N |
| LPG50148.2.2 | 301 | R46Q |
| LPG50148.2.3 | 302 | V68M |
| LPG50148.2.4 | 303 | V81S |
| LPG50148.2.5 | 304 | S108H |
| LPG50148.2.6 | 305 | N121T |
| LPG50148.2.7 | 306 | F155W |
| LPG50148.2.8 | 307 | N156R |
| LPG50148.2.9 | 308 | L162W |
| LPG50148.2.10 | 309 | L162H |
| LPG50148.2.25 | 324 | M117I |
| LPG50148.2.26 | 325 | P138Y |
| LPG50148.2.27 | 326 | I75W |
| LPG50148.2.28 | 327 | A160D |
| LPG50148.2.29 | 328 | M125K |
| LPG50148.2.30 | 329 | N126A |
| LPG50148.2.31 | 330 | Q158R |
| LPG50148.2.32 | 331 | E139L |
| LPG50148.2.33 | 332 | C145A |
| LPG50148.2.34 | 333 | L162V |
| LPG50148.2.35 | 334 | N121E |
| LPG50148.2.36 | 335 | L120F |
| LPG50148.2.37 | 336 | P151Q |
| LPG50148.2.38 | 337 | Y148K |
| LPG50148.2.39 | 338 | G142K |
| LPG50148.2.40 | 339 | I165R |
| LPG50148.2.41 | 340 | N166H |
| LPG50148.2.42 | 341 | N2C |
| LPG50148.2.43 | 342 | D23Q |
| LPG50148.2.44 | 343 | G135H |
| LPG50148.2.45 | 344 | L137V |
| LPG50148.2.46 | 345 | L162A |

TABLE 3
TAT combined mutations

| | | Mutations in relation |
|---|---|---|
| LPG50148 ABE | SEQ | to SEQ ID NO: 5 |
| Variant | ID NO | (LPG50148) |
| LPG50148.2.11 | 310 | L35N, L162W |
| LPG50148.2.12 | 311 | L35N, R46Q |
| LPG50148.2.13 | 312 | V81S, N156R, |
| LPG50148.2.14 | 313 | R46Q, N156R |
| LPG50148.2.15 | 314 | N156R, L162W |
| LPG50148.2.16 | 315 | L35N, V81S, L162W |
| LPG50148.2.17 | 316 | L35N, R46Q, L162W |
| LPG50148.2.18 | 317 | F155W, N156R, L162W |
| LPG50148.2.19 | 318 | L35N, N156R, L162W |
| LPG50148.2.20 | 319 | L35N, V81S, N156R, |
| | | L162W |
| LPG50148.2.21 | 320 | V68M, S108H, N156R, |
| | | L162W |
| LPG50148.2.22 | 321 | R46Q, V68M, S108H, |
| | | N156R, L162W |
| LPG50148.2.23 | 322 | V68M, V81S, S108H, |
| | | N156R, L162W |
| LPG50148.2.24 | 323 | L35N, R46Q, V68M, |
| | | V81S, S108H, F155W, |
| | | N156R, L162W |
| LPG50148.2.56 | 355 | V81S, F155W |
| LPG50148.2.57 | 356 | I75W, V81S |
| LPG50148.2.58 | 357 | V81S, A160D |
| LPG50148.2.59 | 358 | V81S, C145A |
| LPG50148.2.60 | 359 | I75W, F155W |
| LPG50148.2.61 | 360 | F155W, A60D |
| LPG50148.2.62 | 361 | C145A, F155W |
| LPG50148.2.63 | 362 | I75W, A160D |
| LPG50148.2.64 | 363 | I75W, C145A |
| LPG50148.2.65 | 364 | C145A, A160D |
| LPG50148.2.66 | 365 | I75W, V81S, F155W |
| LPG50148.2.67 | 366 | V81S, F155W, A160D |
| LPG50148.2.68 | 367 | V81S, C145A, F155W |
| LPG50148.2.69 | 368 | I75W, V81S, A160D |
| LPG50148.2.70 | 369 | I75W, V81S, C145A |
| LPG50148.2.71 | 370 | V81S, C145A, A160D |
| LPG50148.2.72 | 371 | I75W, F155W, A160D |
| LPG50148.2.73 | 372 | I75W, C145A, F155W |
| LPG50148.2.74 | 373 | C145A, F155W, A160D |
| LPG50148.2.75 | 374 | I75W, C145A, A160D |
| LPG50148.2.76 | 375 | I75W, V81S, F155W, |
| | | A160D |
| LPG50148.2.77 | 376 | I75W, V81S, C145A, |
| | | F155W |
| LPG50148.2.78 | 377 | V81S, C145A, F155W, |
| | | A160D |
| LPG50148.2.79 | 378 | I75W, V81S, C145A, |
| | | A160D |
| LPG50148.2.80 | 379 | I75W, C145A, F155W, |
| | | A160D |
| LPG50148.2.81 | 380 | I75W, V81S, C145A, |
| | | F155W, A160D |

When the selection in bacteria was performed against CAC sequence contexts, 9 single mutations were identified after the enrichment steps (Table 4). The single mutations were incorporated into a human codon-optimized LPG50148-nAPG07433.1 fusion construct. The performance of these evolved deaminases in ABEs was evaluated by editing HEK293T cells at eight endogenous genomic sites. Single mutations showing the highest average fold-change were combined (Table 5) and tested to identify the top hits: variants LPG50148.2.106 (variant 106) and LPG50148.2.107 (variant 107) (FIG. 11).

TABLE 4
CAC single mutations

| | | Mutations in relation |
|---|---|---|
| LPG50148 ABE | SEQ | to SEQ ID NO: 5 |
| Variant | ID NO | (LPG50148) |
| LPG50148.2.47 | 346 | R22F |
| LPG50148.2.48 | 347 | V68A |
| LPG50148.2.49 | 348 | Y72H |
| LPG50148.2.50 | 349 | I75D |
| LPG50148.2.51 | 350 | D76G |
| LPG50148.2.52 | 351 | M117L |
| LPG50148.2.53 | 352 | Y122I |
| LPG50148.2.54 | 353 | N156V |
| LPG50148.2.55 | 354 | Q157F |

TABLE 5
CAC combined mutations

| | | Mutations in relation |
|---|---|---|
| LPG50148 ABE | SEQ | to SEQ ID NO: 5 |
| Variant | ID NO | (LPG50148) |
| LPG50148.2.105 | 404 | V68A, V81S |
| LPG50148.2.106 | 405 | D76G, V81S |
| LPG50148.2.107 | 406 | V81S, M117L |
| LPG50148.2.108 | 407 | V68A, D76G |
| LPG50148.2.109 | 408 | V68A, M117L |
| LPG50148.2.110 | 409 | D76G, M117L |
| LPG50148.2.111 | 410 | V68A, D76G, V81S |
| LPG50148.2.112 | 411 | V68A, V81S, M117L |
| LPG50148.2.113 | 412 | D76G, V81S, M117L |
| LPG50148.2.114 | 413 | V68A, D76G, M117L |
| LPG50148.2.115 | 414 | V68A, D76G, V815, |
| | | M117L |

When the selection in bacteria was performed against TAG sequence contexts, 6 single mutations were identified after the enrichment steps (Table 6). The single mutations were incorporated into a human codon-optimized LPG50148-nAPG07433.1 fusion construct. The performance of these evolved deaminases in ABEs was evaluated by editing HEK293T cells at three endogenous genomic sites. Single mutations showing the highest average fold-change were combined (Table 7) and tested to identify the top hits: variants LPG50148.2.88 (variant 88) and LPG50148.2.103 (variant 103) (FIG. 11).

TABLE 6
TAG single mutations

| | | Mutations in relation |
|---|---|---|
| LPG50148 ABE | SEQ | to SEQ ID NO: 5 |
| Variant | ID NO | (LPG50148) |
| LPG50148.2.82 | 381 | I40L |
| LPG50148.2.83 | 382 | V105M |
| LPG50148.2.84 | 383 | N121V |
| LPG50148.2.85 | 384 | C145M |
| LPG50148.2.86 | 385 | K159Q |
| LPG50148.2.87 | 386 | A160W |

TABLE 7
TAG combined mutations

| | | Mutations in relation |
|---|---|---|
| LPG50148 ABE | SEQ | to SEQ ID NO: 5 |
| Variant | ID NO | (LPG50148) |
| LPG50148.2.88 | 387 | I40L, V105M |
| LPG50148.2.89 | 388 | I40L, N121V |
| LPG50148.2.90 | 389 | I40L, C145M |
| LPG50148.2.91 | 390 | I40L, K159Q |
| LPG50148.2.92 | 391 | I40L, A160W |
| LPG50148.2.93 | 392 | V105M, N121V, |
| LPG50148.2.94 | 393 | V105M, C145M |
| LPG50148.2.95 | 394 | V105M, K159Q |
| LPG50148.2.96 | 395 | V105M, A160W |
| LPG50148.2.97 | 396 | N121V, C145M |
| LPG50148.2.98 | 397 | N121V, K159Q |
| LPG50148.2.99 | 398 | N121V, A160W |
| LPG50148.2.100 | 399 | C145M, K159Q |
| LPG50148.2.101 | 400 | C145M, A160W |
| LPG50148.2.102 | 401 | K159Q, A160W |
| LPG50148.2.103 | 402 | V81S, C145M, A160W |
| LPG50148.2.104 | 403 | V81S, N121V, K159Q |

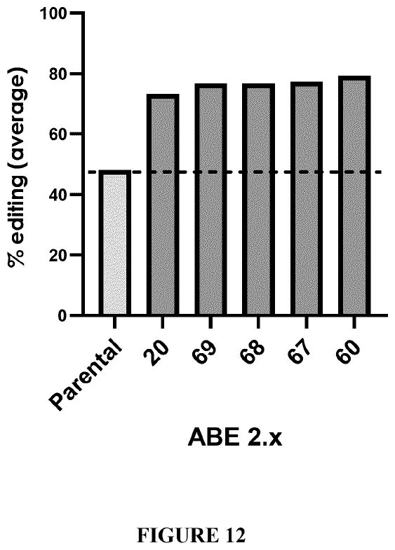

In addition, when delivered as an mRNA construct, the evolved LPG50148 ABE variant LPG50148 numbers 20, 60, 67, 68, and 69 enable editing in human cells, which showed editing up to ~80% and an average improvement by ~1.6 fold compared to the parental ABE (FIG. 12).

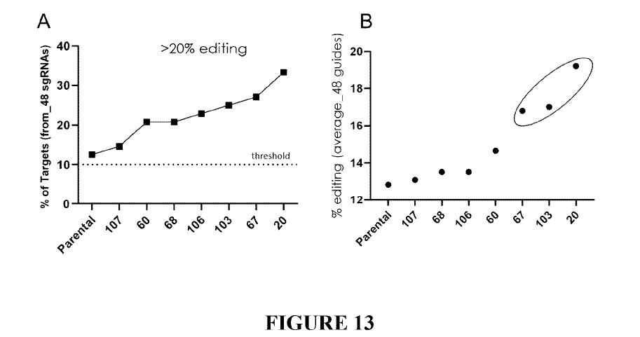

Next, evolved LPG50148 ABE variant numbers 20, 60, 67, 68, 103, 106, and 107 were characterized by a comprehensive set of targets in order to identify the second generation ABEs with the most robust activity and highest specificity (Table 8). Robust improvements in base editing efficiency was observed for all variants (FIG. 13A). Mean indel frequency and A to T/C transversion was barely above background (0-1%) for all variants tested. The ABEs comprising LPG50148 variants LPG50148.20, LPG50148.67, and LPG50148.103 significantly outperformed the parental ABE in editing efficiency among the forty eight genomic sites (FIG. 13B). Variant 20 showed improvement (up to 5-fold) for 94% of the tested guides (Table 8).

TABLE 8
Total A to G substitution (%) of all ABE constructs in HEK293T cells after plasmid delivery
across 48 genomic sites.

| Guide ID | Parental | 20 | 60 | 67 | 68 | 103 | 106 | 107 |
|---|---|---|---|---|---|---|---|---|
| SGN002364 | 18.96 | 26.55 | 19.71 | 25.45 | 15.33 | 18.73 | 19.97 | 15.36 |
| SGN004631 | 4.29 | 10.71 | 4.81 | 5.11 | 2.67 | 5.03 | 5.45 | 2.97 |
| SGN004632 | 10.95 | 12.64 | 10.68 | 10.74 | 5.37 | 10.41 | 10.84 | 6.54 |
| SGN004633 | 3.91 | 7.15 | 5.47 | 6.97 | 2.95 | 7.02 | 4.82 | 3.79 |
| SGN004634 | 0.75 | 0.45 | 1.12 | 0.84 | 1.18 | 0.58 | 0.47 | 0 |
| SGN005417 | 0.74 | 0.65 | 0.59 | 0.8 | 0.63 | 0.49 | 0.48 | 0.46 |
| SGN003707 | 48.59 | 56.23 | 56.65 | 54.45 | 30.56 | 57.16 | 40.49 | 36.34 |
| SGN001256 | 34.11 | 48.71 | 52.09 | 62.21 | 34.22 | 54.99 | 48.21 | 28.22 |
| SGN004997 | 17.33 | 30.23 | 31.78 | 30.38 | 11.88 | 36.91 | 31.45 | 16.17 |
| SGN001947 | 27.28 | 45.95 | 30.18 | 55.32 | 32.29 | 51.72 | 37.66 | 37.93 |
| SGN001950 | 33.57 | 26.4 | 38.19 | 39.14 | 27.78 | 41.84 | 26.98 | 16.49 |
| SGN001270 | 15.03 | 18.7 | 13.02 | 16.51 | 20.79 | 19.26 | 21.59 | 19.48 |
| SGN001960 | 28.95 | 26.96 | 26.1 | 21.11 | 29.33 | 25.9 | 20.95 | 18.53 |
| SGN001963 | 14.55 | 30.74 | 25.46 | 27.57 | 30.86 | 25.89 | 21.13 | 23.28 |
| SGN001966 | 6.71 | 6.93 | 7.03 | 6.57 | 3.05 | 2.06 | 1.84 | 1.76 |
| SGN005418 | 38.94 | 49.17 | 43.14 | 43.96 | 78 | 74.36 | 75.6 | 75 |
| SGN005419 | 24.21 | 27.02 | 24.22 | 25.81 | 75.73 | 73.55 | 70.73 | 70.63 |
| SGN005434 | 6.24 | 14.4 | 7.33 | 3.78 | 9.92 | 7.33 | 6.98 | 10.12 |
| SGN005111 | 8.07 | 14.1 | 8 | 9.3 | 8.98 | 4.25 | 7.35 | 9.23 |
| SGN004143 | 13.01 | 41 | 19.6 | 27.5 | 21 | 24 | 17.3 | 26 |
| SGN005436 | 2.41 | 3.57 | 2.5 | 3.34 | 5.84 | 5.44 | 5.22 | 5.7 |
| SGN002385 | 6.75 | 15.23 | 7.8 | 7.5 | 9.67 | 8.88 | 5.93 | 9.88 |
| SGN000414 | 9.37 | 13.53 | 10.48 | 11.96 | 11.9 | 9.59 | 6.89 | 11.66 |
| SGN001259 | 18.92 | 22.15 | 18.91 | 16.54 | 14.06 | 15.96 | 15.57 | 16.79 |
| SGN001274 | 15.53 | 20.21 | 16.02 | 19.1 | 14.18 | 15.42 | 14.24 | 15.73 |
| SGN001275 | 13.57 | 20.24 | 14.61 | 15.31 | 11.17 | 11.64 | 12.09 | 12.24 |
| SGN003003 | 7.39 | 17.49 | 10.72 | 12.33 | 10.99 | 11.47 | 12.51 | 7.03 |
| SGN003004 | 9.95 | 12.24 | 9.36 | 10.51 | 7.7 | 10.72 | 7.84 | 6.46 |
| SGN005430 | 17.67 | 25.56 | 20.23 | 24.38 | 21.85 | 24.95 | 16.88 | 21.71 |
| SGN005431 | 15.28 | 29.52 | 20.72 | 23.16 | 19.09 | 25.12 | 20.77 | 18.58 |
| SGN005432 | 9.56 | 16.86 | 11.81 | 12.07 | 6.01 | 14.59 | 11.12 | 12.19 |
| SGN005420 | 4.45 | 10.24 | 5.47 | 8.63 | 5.22 | 7.99 | 5.05 | 5.73 |
| SGN001880 | 8.94 | 13.76 | 8.33 | 12.54 | 8.5 | 10.84 | 8.43 | 8.55 |
| SGN005433 | 9.14 | 16 | 10.93 | 12.39 | 10.66 | 11.9 | 10.75 | 9.37 |
| SGN005425 | 8.63 | 15.6 | 7.66 | 14.41 | 9.8 | 12.41 | 9.59 | 9.95 |
| SGN005423 | 12.17 | 18.58 | 10.63 | 17.08 | 10.94 | 16.39 | 16.58 | 13.89 |
| SGN005426 | 10.99 | 13.95 | 9.79 | 9.21 | 4.61 | 7.06 | 3.15 | 4.48 |
| SGN005424 | 13.88 | 11.88 | 10.4 | 8.43 | 5.88 | 12.07 | 2.14 | 0.83 |
| SGN004995 | 20.36 | 28.61 | 20.32 | 24.6 | 18.19 | 20.18 | 18.75 | 17.88 |
| SGN001974 | 2.9 | 8.01 | 6.2 | 3 | 7.5 | 7.5 | 9 | 8 |
| SGN004032 | 17.52 | 19.11 | 18.07 | 19.44 | 7.19 | 10.79 | 7.26 | 7.37 |
| SGN004387 | 14.08 | 16.9 | 15.14 | 12.34 | 7.25 | 8.06 | 7.34 | 7.17 |
| SGN003610 | 12.26 | 17.18 | 8.39 | 10.54 | 8.31 | 12.02 | 3.53 | 9.39 |
| SGN003613 | 13.74 | 22.17 | 12.25 | 13.88 | 10.76 | 16.41 | 10.07 | 11.83 |
| SGN003614 | 6.38 | 14.51 | 7.5 | 8.87 | 8.85 | 10.18 | 3.16 | 10.43 |
| SGN003616 | 6.51 | 17.15 | 10.41 | 14.52 | 13.36 | 13.38 | 4.2 | 11.82 |
| SGN003618 | 7.1 | 12.45 | 8.32 | 20.78 | 8.35 | 13.29 | 8.18 | 7.56 |
| SGN003621 | 6.76 | 17.29 | 8.66 | 9.92 | 9.37 | 13.2 | 4.99 | 9.73 |

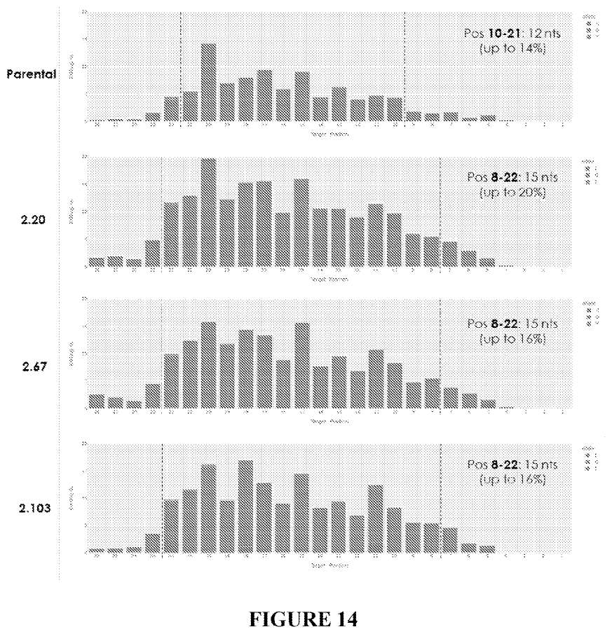

The ABE variants 20, 67, and 103 showed a larger optimal window (8-22 nucleotides 5′ of the PAM sequence on the non-target strand) compared with the parental ABE (10-21), broadening the targeting scope for efficient A base editing (FIG. 14).

Exhaustive analysis of this dataset provided details relating to the preferred 5′-NA-3′ and 5′-AN-3′ editing context. For 5′NA, TA>CA>>AA>GA for variant 60, and TA>CAGA>AA for all other constructs. For all variants, 5′-AN-3′ showed the following preference AC>ATAG>AA.

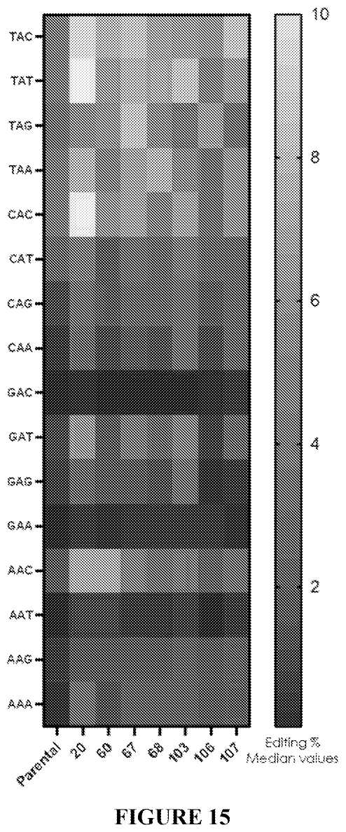

Variant 67 showed higher editing for TAG context, while variant 20 performed better across all the other sequence contexts (FIG. 15).

Taken together, these results indicate that more efficient ABEs have been developed through directed evolution of the deaminase of a parental ABE (LPG50148-nAPG07433.1).

### Example 8: Demonstration of Gene Editing Activity in Plant Cells

Base-editing activity of an RGN-deaminase fusion protein of the invention is demonstrated in plant cells using protocols adapted from Li, et al., 2013 (Nat. Biotech. 31:688-691). Briefly, an expression vector comprising an expression cassette capable of expressing in plant cells an RGN-deaminase fusion protein operably linked to a SV40 nuclear localization signal (SEQ ID NO: 430) and a second expression cassette encoding a guide RNA targeting one or more sites in the plant PDS gene that flank an appropriate PAM sequence are introduced into Nicotiana benthamiana mesophyll protoplasts using PEG-mediated transformation. The transformed protoplasts are incubated in the dark for up to 36 hr. Genomic DNA is isolated from the protoplasts using a DNeasy Plant Mini Kit (Qiagen). The genomic region flanking the RGN target site is PCR amplified, products are purified, and the purified PCR products are analyzed using Next Generation Sequencing on Illumina MiSeq. Typically, 100,000 of 250 bp paired-end reads (2×100,000 reads) are generated per amplicon. The reads are analyzed using CRISPResso (Pinello, et al. 2016 Nature Biotech, 34:695-697) to calculate the rates of editing. Output alignments are analyzed for INDEL formation or introduction of specific adenine mutations.

### Example 9: Inlaid Base Editors with Improved Editing Work by mRNA Delivery to Primary Mouse Hepatocytes

Example 9.1: A vial of primary mouse hepatocytes was thawed in a 37° C. heat bath, and the cells were plated in plating media (Williams E media+fetal bovine serum+cocktail A+dexamethasone; Thermo Fisher) at a density of 10k cells per well. After 4-6 hr in the incubator (37° C., 5% CO2), the cells were lipofected with mRNA and sgRNA (1:3 ng ratio) using Lipofectamine MessengerMAX and 2% fetal bovine serum. Messenger RNAs encoding LPG20046 (LPG50148.2.76 fused to the N-terminus of nAPG07433.1; set forth as SEQ ID NO: 577), LPG20047 (LPG50148.2.20 fused to the N-terminus of nAPG07433.1; set forth as SEQ ID NO: 578), LPG20123 (LPG50148.2.76 inserted after S772 of nAPG07433.1; set forth as SEQ ID NO: 580), or LPG20124 (LPG50148.2.20 inserted after S772 of nAPG07433.1; set forth as SEQ ID NO: 579) were delivered along with guide RNAs. The following day, the media was exchanged to maintenance media (Williams E media+cocktail B+dexamethasone Thermo Fisher), and the cells were allowed to incubate for 3 more days after which the genomic DNA was extracted for next-generation sequence (NGS) analysis. The base editing rate for each position in the target HAO1 gene is calculated by taking the conversion rate of A to G at each position throughout the target sequence. Results are shown in Tables 9-13.

TABLE 9
LPG50148.2.20 N-terminal fusion to nAPG07433.1
(LPG20047; SEQ ID NO: 578) base editing with
shortened and full length guide RNAs

| | Position in | Average BE |
|---|---|---|
| SGN | Target | Rate |
| SGN001432 | A10 | 41.1 |
| SGN001432 | A3 | 0.15 |
| SGN001432 | A1 | 0.05 |
| SGN001433 | A16 | 54.05 |
| SGN001433 | A9 | 51.25 |
| SGN001433 | A7 | 4.5 |
| SGN001434 | A18 | 36.35 |
| SGN001434 | A11 | 39 |
| SGN001434 | A9 | 24.85 |
| SGN001434 | A2 | 0 |
| SGN001435 | A22 | 30.95 |
| SGN001435 | A15 | 47.5 |
| SGN001435 | A13 | 44.45 |
| SGN001435 | A6 | 6 |
| SGN008382 | A10 | 43.35 |
| SGN008382 | A3 | 0.15 |
| SGN008382 | A1 | 0 |
| SGN008383 | A16 | 60 |
| SGN008383 | A9 | 65.6 |
| SGN008383 | A7 | 3.85 |
| SGN008384 | A18 | 28.05 |
| SGN008384 | A11 | 32.3 |
| SGN008384 | A9 | 22.1 |
| SGN008384 | A2 | 0 |
| SGN008385 | A22 | 32.95 |
| SGN008385 | A15 | 49.15 |
| SGN008385 | A13 | 43.95 |
| SGN008385 | A6 | 2.7 |

SGN001432, SGN001433, SGN001434, and SGN001435 contain the full-length backbone for the APG07433.1 nuclease (set forth as SEQ ID NO: 693). SGN008382, SGN008383, SGN008384, and SGN008385 contain a shortened APG07433.1 backbone (set forth as SEQ ID NO: 694). The base editor containing an N-terminal fusion of LPG50148.2.20 to nAPG07433.1 retains high editing at similar positions across several targets when SGNs with the shortened backbones are used.

TABLE 10
LPG50148.2.76 N-terminal fusion to nAPG07433.1
(LPG20046; SEQ ID NO: 577) base editing with
shortened and full length guide RNAs

| | | Average BE |
|---|---|---|
| SGN | Position | Rate |
| SGN001432 | A10 | 59.6 |
| SGN001432 | A3 | 0.5 |
| SGN001432 | A1 | 0.05 |
| SGN001433 | A16 | 56.05 |
| SGN001433 | A9 | 53.9 |
| SGN001433 | A7 | 13.9 |
| SGN001434 | A18 | 35.05 |
| SGN001434 | A11 | 39.7 |
| SGN001434 | A9 | 37.05 |
| SGN001434 | A2 | 0 |
| SGN001435 | A22 | 36 |
| SGN001435 | A15 | 40.7 |
| SGN001435 | A13 | 41.85 |
| SGN001435 | A6 | 10.2 |
| SGN008382 | A10 | 67.35 |
| SGN008382 | A3 | 0.35 |
| SGN008382 | A1 | 0.1 |
| SGN008383 | A16 | 62.75 |
| SGN008383 | A9 | 61.85 |
| SGN008383 | A7 | 16.4 |
| SGN008384 | A18 | 38.6 |
| SGN008384 | A11 | 44.65 |
| SGN008384 | A9 | 42.8 |
| SGN008384 | A2 | 0 |
| SGN008385 | A22 | 42.2 |
| SGN008385 | A15 | 48.9 |
| SGN008385 | A13 | 49.4 |
| SGN008385 | A6 | 14 |

SGN001432, SGN001433, SGN001434, and SGN001435 contain the full-length backbone for the APG07433.1 nuclease (set forth as SEQ ID NO: 693). SGN008382, SGN008383, SGN008384, and SGN008385 contain a shortened APG07433.1 backbone (set forth as SEQ ID NO: 694). The base editor containing an N-terminal fusion of LPG50148.2.76 to nAPG07433.1 retains high editing at similar positions across several targets when SGNs with shortened backbones are used.

TABLE 11
LPG50148.2.20 inlaid fusion in nAPG07433.1 (LPG20124; SEQ ID NO:
579) base editing with shortened and full length guide RNAs

| | | Average BE |
|---|---|---|
| SGN | Position | Rate |
| SGN001432 | A10 | 33.8 |
| SGN001432 | A3 | 0.05 |
| SGN001432 | A1 | 0 |
| SGN001433 | A16 | 29.05 |
| SGN001433 | A9 | 42 |
| SGN001433 | A7 | 12 |
| SGN001434 | A18 | 16.25 |
| SGN001434 | A11 | 22.85 |
| SGN001434 | A9 | 16.1 |
| SGN001434 | A2 | 0 |
| SGN001435 | A22 | 12.1 |
| SGN001435 | A15 | 42.35 |
| SGN001435 | A13 | 36.75 |
| SGN001435 | A6 | 18.05 |
| SGN008382 | A10 | 38.55 |
| SGN008382 | A3 | 0 |
| SGN008382 | A1 | 0 |
| SGN008383 | A16 | 11.5 |
| SGN008383 | A9 | 18.3 |
| SGN008383 | A7 | 5.8 |
| SGN008384 | A18 | 16.1 |
| SGN008384 | A11 | 22.55 |
| SGN008384 | A9 | 14.2 |
| SGN008384 | A2 | 0 |
| SGN008385 | A22 | 8.85 |
| SGN008385 | A15 | 40.8 |
| SGN008385 | A13 | 34.25 |
| SGN008385 | A6 | 16.8 |

TABLE 12
LPG50148.2.76 inlaid fusion in nAPG07433.1 (LPG20123; SEQ ID NO:
580) base editing with shortened and full length guide RNAs

| | | Average BE |
|---|---|---|
| SGN | Position | Rate |
| SGN001432 | A10 | 31.1 |
| SGN001432 | A3 | 0.25 |
| SGN001432 | A1 | 0 |
| SGN001433 | A16 | 19.1 |
| SGN001433 | A9 | 21.95 |
| SGN001433 | A7 | 16.6 |
| SGN001434 | A18 | 19.95 |
| SGN001434 | A11 | 23.4 |
| SGN001434 | A9 | 18.25 |
| SGN001434 | A2 | 0 |
| SGN001435 | A22 | 38.1 |
| SGN001435 | A15 | 43.55 |
| SGN001435 | A13 | 46.2 |
| SGN001435 | A6 | 33 |
| SGN008382 | A10 | 46.55 |
| SGN008382 | A3 | 0.3 |
| SGN008382 | A1 | 0 |
| SGN008383 | A16 | 25.85 |
| SGN008383 | A9 | 28.9 |
| SGN008383 | A7 | 20.85 |
| SGN008384 | A18 | 28.1 |
| SGN008384 | A11 | 32.45 |
| SGN008384 | A9 | 26.55 |
| SGN008384 | A2 | 0 |
| SGN008385 | A22 | 44.35 |
| SGN008385 | A15 | 50.6 |
| SGN008385 | A13 | 52.7 |
| SGN008385 | A6 | 37.6 |

Effective editing in mouse primary hepatocytes was observed by mRNA delivery of the base editors. Both inlaid, N-terminal and C-terminal fusions (data not shown) of the improved deaminases are effective. For some targets, N- or C-terminal fusions of the deaminase have higher editing rates than the inlaid base editor designs. Additionally, the editors tested work with both shortened (M) and full-length (F) guide RNA backbones.

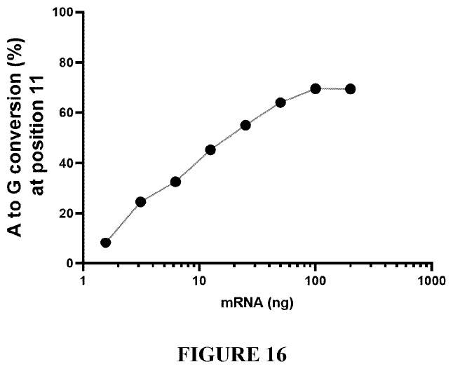

Example 9.2: DNA templates used to make mRNA were amplified by PCR reactions. Then the IVT (in vitro transcription) reactions were carried out, and followed by DNase I and ProK treatment to remove DNA templates and enzymes. The mRNAs were then purified by plate-based silica membrane. A dose response was performed with mRNA delivery. Effective dose-dependent editing was observed through delivery of ABE mRNA and a synthetic guide RNA via lipofection in mouse primary hepatocytes. 80 ng of guide RNA SGN008393 was co-transfected with varying amounts of mRNA. Results of base editing at position 11 with a base editor comprising the evolved deaminase LPG50148.2.20 fused to the N-terminus of nAPG07433.1 (referred to as LPG20047 set forth as SEQ ID NO: 578) are shown in FIG. 16. The LPG20047 base editor is active at lower doses of mRNA in a base editor construct.

### Example 10: Generation of Deaminases LPG50319 and LPG50320 and Site Saturation Mutagenesis Engineering to Generate Improved Deaminase LPG50324

Deaminases LPG50319 (also referred to herein as LPG50148.2.107 and set forth as SEQ ID NO: 406) and LPG50320 (set forth as SEQ ID NO: 596) were created by combining two frequently beneficial mutations observed in the E. coli screen previously described in Example 7. Both contain the V81S mutation as well as a second mutation that was identified in a parallel selection in E. coli against a CAC context. Specifically, LPG50319 has M117L (along with V81S) and LPG50320 has V68A (along with V81S) in relation to the parental LPG50148 deaminase.

During a subsequent evolution round, LPG50265 (also known as LPG50148.2.67, set forth as SEQ ID NO: 366) was used as the new parental deaminase. A combinatorial library was created on top of LPG50265 via site saturation mutagenesis, changing four hotspot positions identified in the first round. The library was then screened in E. coli, resulting in the selection of the deaminase LPG50324 (set forth as SEQ ID NO: 597) under high stringency conditions. LPG50324 has seven mutations (175Y, V81S, M117L, N121G, C145L, F155W, and A160D) in relation to the parental LPG50148 deaminase set forth as SEQ ID NO: 5.

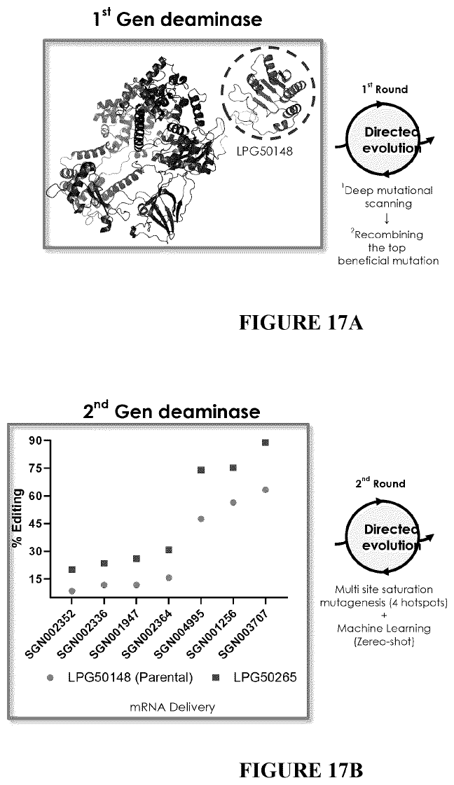

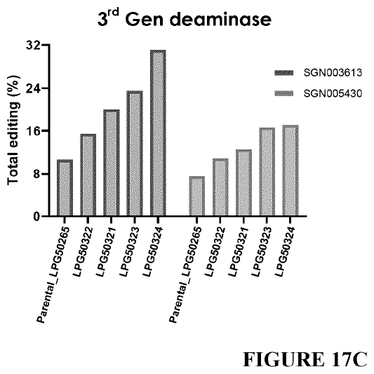

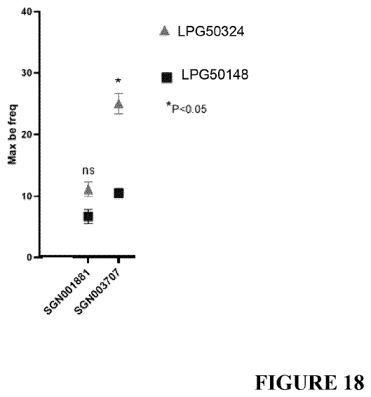

The directed evolution scheme can be found in FIG. 17. FIG. 18 shows the improved activity of LPG50324 over the parental LPG50148. The experiments in FIG. 17C were performed in HEK293T cells (10K cells/reaction) transfected with base editor plasmid (160 ng) and guide plasmid (40 ng) at the ratio of 4:1 using Lipofectamine P3000 transfection reagent. The sequences for LPG50148, LPG50265, LPG50321, LPG50322, LPG50323, and LPG50324 are set forth as SEQ ID NOs: 5, 366, 720, 721, 722, and 597, respectively, and the base editor constructs are named LPG50148_nAPG07433.1, LPG20130, LPG20126, LPG20127, LPG20128, and LPG20129, respectively, which comprise the deaminase fused to the N-terminus of the nAPG07433.1 nickase. The cells were harvested 48 h post-transfection and subjected to genomic DNA extraction using QuickExtract DNA extract solution. The genomic region targeted by the sgRNA was amplified by PCR, and the PCR amplicons were subsequently analyzed by NGS to evaluate the editing efficiency. The experiment in FIG. 18 was performed in primary human hepatocytes as described in Example 11.

An analysis of base-editing efficiencies was performed for several nAPG07433.1-ABE variants in human primary T-cells using mRNA delivery. Briefly, six separate vials of T-cells (donor-008) were thawed, activated, and cultured in three separate flasks by two different scientists. Three days post-activation, 30K cells per condition were nucleofected with 2 gg mRNA+4 gg sgRNA in P3 solution using program EO-115. A total of ten guide RNAs, targeting five different genes, were tested with each ABE variant. Three replicates per condition were performed to assess reproducibility. Four days post-nucleofection, cells were harvested and genomic DNA was extracted using QuickExtract. On-target, as well as predicted off-target, editing was determined by PCR amplification of the relevant genomic loci and sequenced by Illumina short-read sequencing.

The max base editing rate (Max BE Rate) is calculated by taking the conversion rate of A to G at each position throughout the guide and returning the maximum editing rate at a single position. Total adenine substitution rate (Total A Substitution Rate) calculates the percent of reads that contain at least one adenine to guanine edit.

Note that LPG50148.2.4, LPG50148.2.20, LPG50148.2.24, LPG5148.2.27, LPG50148.2.60, LPG50148.2.67, LPG50148.2.68, LPG50148.2.76, LPG50148.2.103, LPG50148.2.106, and LPG50148.2.107 are referred to by alternative names in Tables 14-18 and in this example and Examples 11-14. The alternative names are outlined in Table 13.

TABLE 13
Alternative nomenclature for various
LPG50148 variants nomenclature

| | Alternative |
|---|---|
| LPG5014.2._ nomenclature | nomenclature |
| LPG50148.2.4 | LPG50208 |
| LPG50148.2.20 | LPG50221 |
| LPG50148.2.24 | LPG50225 |
| LPG50148.2.27 | LPG50228 |
| LPG50148.2.60 | LPG50258 |
| LPG50148.2.67 | LPG50265 |
| LPG50148.2.68 | LPG50266 |
| LPG50148.2.76 | LPG50274 |
| LPG50148.2.103 | LPG50432 |
| LPG50148.2.106 | LPG50298 |
| LPG50148.2.107 | LPG50319 |

TABLE 14
Performance of evolved deaminases and different fusion protein architectures on nAPG07433.1

| | | | | | | | Average |
|---|---|---|---|---|---|---|---|
| | | | | | | Average | Total A |
| | | Fusion | Type | | Target | of Max | Substitution |
| Deaminase | Nuclease | Protein | of Fusion | SGN | gene | BE Rate | Rate |
| LPG50148 | nAPG07433.1 | LPG20048 | N-term | SGN001256 | B2M | 41.53 | 58.85 |
| LPG50148 | nAPG07433.1 | LPG20048 | N-term | SGN001947 | B2M | 6.77 | 13.51 |
| LPG50148 | nAPG07433.1 | LPG20048 | N-term | SGN001960 | B2M | 68.56 | 84.98 |
| LPG50148 | nAPG07433.1 | LPG20048 | N-term | SGN002259 | Gene A | 3.37 | 9.73 |
| LPG50148 | nAPG07433.1 | LPG20048 | N-term | SGN002336 | Gene B | 7.96 | 12.88 |
| LPG50148 | nAPG07433.1 | LPG20048 | N-term | SGN002352 | Gene C | 3.87 | 10.99 |
| LPG50148 | nAPG07433.1 | LPG20048 | N-term | SGN002364 | Gene C | 8.08 | 20.16 |
| LPG50148 | nAPG07433.1 | LPG20048 | N-term | SGN002372 | Gene D | 94.13 | 94.05 |
| LPG50148 | nAPG07433.1 | LPG20048 | N-term | SGN003707 | B2M | 28.60 | 55.64 |
| LPG50148 | nAPG07433.1 | LPG20048 | N-term | SGN004995 | TRAC | 37.61 | 54.65 |
| LPG50432 | nAPG07433.1 | LPG20058 | N-term | SGN001256 | B2M | 65.06 | 78.95 |
| LPG50432 | nAPG07433.1 | LPG20058 | N-term | SGN001947 | B2M | 12.13 | 17.59 |
| LPG50432 | nAPG07433.1 | LPG20058 | N-term | SGN001960 | B2M | 54.25 | 85.90 |
| LPG50432 | nAPG07433.1 | LPG20058 | N-term | SGN002259 | Gene A | 7.44 | 15.53 |
| LPG50432 | nAPG07433.1 | LPG20058 | N-term | SGN002336 | Gene B | 20.23 | 26.37 |
| LPG50432 | nAPG07433.1 | LPG20058 | N-term | SGN002352 | Gene C | 4.89 | 13.29 |
| LPG50432 | nAPG07433.1 | LPG20058 | N-term | SGN002364 | Gene C | 8.85 | 21.56 |
| LPG50432 | nAPG07433.1 | LPG20058 | N-term | SGN002372 | Gene D | 91.12 | 91.81 |
| LPG50432 | nAPG07433.1 | LPG20058 | N-term | SGN003707 | B2M | 62.14 | 87.65 |
| LPG50432 | nAPG07433.1 | LPG20058 | N-term | SGN004995 | TRAC | 45.02 | 59.64 |
| LPG50298 | nAPG07433.1 | LPG20059 | N-term | SGN001256 | B2M | 65.94 | 80.93 |
| LPG50298 | nAPG07433.1 | LPG20059 | N-term | SGN001947 | B2M | 19.13 | 31.00 |
| LPG50298 | nAPG07433.1 | LPG20059 | N-term | SGN001960 | B2M | 80.61 | 91.38 |
| LPG50298 | nAPG07433.1 | LPG20059 | N-term | SGN002259 | Gene A | 10.24 | 20.95 |
| LPG50298 | nAPG07433.1 | LPG20059 | N-term | SGN002336 | Gene B | 26.98 | 36.55 |
| LPG50298 | nAPG07433.1 | LPG20059 | N-term | SGN002352 | Gene C | 10.86 | 22.54 |
| LPG50298 | nAPG07433.1 | LPG20059 | N-term | SGN002364 | Gene C | 19.37 | 30.34 |
| LPG50298 | nAPG07433.1 | LPG20059 | N-term | SGN002372 | Gene D | 92.60 | 92.98 |
| LPG50298 | nAPG07433.1 | LPG20059 | N-term | SGN003707 | B2M | 72.58 | 93.19 |
| LPG50298 | nAPG07433.1 | LPG20059 | N-term | SGN004995 | TRAC | 69.22 | 83.82 |
| LPG50319 | nAPG07433.1 | LPG20060 | N-term | SGN001256 | B2M | 72.94 | 82.63 |
| LPG50319 | nAPG07433.1 | LPG20060 | N-term | SGN001947 | B2M | 23.38 | 37.24 |
| LPG50319 | nAPG07433.1 | LPG20060 | N-term | SGN001960 | B2M | 85.69 | 93.36 |
| LPG50319 | nAPG07433.1 | LPG20060 | N-term | SGN002259 | Gene A | 10.98 | 20.71 |
| LPG50319 | nAPG07433.1 | LPG20060 | N-term | SGN002336 | Gene B | 32.18 | 41.65 |
| LPG50319 | nAPG07433.1 | LPG20060 | N-term | SGN002352 | Gene C | 12.59 | 24.75 |
| LPG50319 | nAPG07433.1 | LPG20060 | N-term | SGN002364 | Gene C | 27.38 | 40.40 |
| LPG50319 | nAPG07433.1 | LPG20060 | N-term | SGN002372 | Gene D | 93.60 | 93.53 |
| LPG50319 | nAPG07433.1 | LPG20060 | N-term | SGN003707 | B2M | 76.23 | 93.10 |
| LPG50319 | nAPG07433.1 | LPG20060 | N-term | SGN004995 | TRAC | 73.46 | 88.29 |
| LPG50221 | nAPG07433.1 | LPG20051 | N-term | SGN001256 | B2M | 46.76 | 63.20 |
| LPG50221 | nAPG07433.1 | LPG20051 | N-term | SGN001947 | B2M | 13.37 | 23.72 |
| LPG50221 | nAPG07433.1 | LPG20051 | N-term | SGN001960 | B2M | 63.89 | 81.81 |
| LPG50221 | nAPG07433.1 | LPG20051 | N-term | SGN002259 | Gene A | 5.21 | 12.35 |
| LPG50221 | nAPG07433.1 | LPG20051 | N-term | SGN002336 | Gene B | 10.64 | 18.70 |
| LPG50221 | nAPG07433.1 | LPG20051 | N-term | SGN002352 | Gene C | 4.90 | 14.17 |
| LPG50221 | nAPG07433.1 | LPG20051 | N-term | SGN002364 | Gene C | 8.34 | 22.25 |
| LPG50221 | nAPG07433.1 | LPG20051 | N-term | SGN002372 | Gene D | 91.86 | 92.07 |
| LPG50221 | nAPG07433.1 | LPG20051 | N-term | SGN003707 | B2M | 39.59 | 73.82 |
| LPG50221 | nAPG07433.1 | LPG20051 | N-term | SGN004995 | TRAC | 36.09 | 51.74 |
| LPG50225 | nAPG07433.1 | LPG20053 | N-term | SGN001256 | B2M | 35.71 | 54.14 |
| LPG50225 | nAPG07433.1 | LPG20053 | N-term | SGN001947 | B2M | 9.06 | 15.49 |
| LPG50225 | nAPG07433.1 | LPG20053 | N-term | SGN001960 | B2M | 74.18 | 85.35 |
| LPG50225 | nAPG07433.1 | LPG20053 | N-term | SGN002259 | Gene A | 2.62 | 8.30 |
| LPG50225 | nAPG07433.1 | LPG20053 | N-term | SGN002336 | Gene B | 6.13 | 11.70 |
| LPG50225 | nAPG07433.1 | LPG20053 | N-term | SGN002352 | Gene C | 5.59 | 12.99 |
| LPG50225 | nAPG07433.1 | LPG20053 | N-term | SGN002364 | Gene C | 10.09 | 19.22 |
| LPG50225 | nAPG07433.1 | LPG20053 | N-term | SGN002372 | Gene D | 91.90 | 92.28 |
| LPG50225 | nAPG07433.1 | LPG20053 | N-term | SGN003707 | B2M | 59.48 | 77.06 |
| LPG50225 | nAPG07433.1 | LPG20053 | N-term | SGN004995 | TRAC | 41.49 | 58.96 |
| LPG50228 | nAPG07433.1 | LPG20054 | N-term | SGN001256 | B2M | 52.81 | 68.30 |
| LPG50228 | nAPG07433.1 | LPG20054 | N-term | SGN001947 | B2M | 17.71 | 27.18 |
| LPG50228 | nAPG07433.1 | LPG20054 | N-term | SGN001960 | B2M | 73.23 | 88.86 |
| LPG50228 | nAPG07433.1 | LPG20054 | N-term | SGN002259 | Gene A | 11.57 | 22.04 |
| LPG50228 | nAPG07433.1 | LPG20054 | N-term | SGN002336 | Gene B | 20.69 | 29.16 |
| LPG50228 | nAPG07433.1 | LPG20054 | N-term | SGN002352 | Gene C | 5.93 | 16.10 |
| LPG50228 | nAPG07433.1 | LPG20054 | N-term | SGN002364 | Gene C | 11.30 | 22.60 |
| LPG50228 | nAPG07433.1 | LPG20054 | N-term | SGN002372 | Gene D | 92.93 | 92.90 |
| LPG50228 | nAPG07433.1 | LPG20054 | N-term | SGN003707 | B2M | 59.07 | 85.55 |
| LPG50228 | nAPG07433.1 | LPG20054 | N-term | SGN004995 | TRAC | 54.77 | 69.97 |
| LPG50208 | nAPG07433.1 | LPG20050 | N-term | SGN001256 | B2M | 63.43 | 76.31 |
| LPG50208 | nAPG07433.1 | LPG20050 | N-term | SGN001947 | B2M | 17.82 | 29.15 |
| LPG50208 | nAPG07433.1 | LPG20050 | N-term | SGN001960 | B2M | 82.36 | 91.53 |
| LPG50208 | nAPG07433.1 | LPG20050 | N-term | SGN002259 | Gene A | 9.05 | 18.30 |
| LPG50208 | nAPG07433.1 | LPG20050 | N-term | SGN002336 | Gene B | 20.69 | 29.30 |
| LPG50208 | nAPG07433.1 | LPG20050 | N-term | SGN002352 | Gene C | 10.16 | 20.44 |
| LPG50208 | nAPG07433.1 | LPG20050 | N-term | SGN002364 | Gene C | 17.88 | 30.49 |
| LPG50208 | nAPG07433.1 | LPG20050 | N-term | SGN002372 | Gene D | 91.23 | 91.35 |
| LPG50208 | nAPG07433.1 | LPG20050 | N-term | SGN003707 | B2M | 66.88 | 89.93 |
| LPG50208 | nAPG07433.1 | LPG20050 | N-term | SGN004995 | TRAC | 63.01 | 79.26 |
| LPG50320 | nAPG07433.1 | LPG20052 | N-term | SGN001256 | B2M | 68.24 | 77.48 |
| LPG50320 | nAPG07433.1 | LPG20052 | N-term | SGN001947 | B2M | 25.92 | 41.71 |
| LPG50320 | nAPG07433.1 | LPG20052 | N-term | SGN001960 | B2M | 80.60 | 89.41 |
| LPG50320 | nAPG07433.1 | LPG20052 | N-term | SGN002259 | Gene A | 10.87 | 21.96 |
| LPG50320 | nAPG07433.1 | LPG20052 | N-term | SGN002336 | Gene B | 25.74 | 35.68 |
| LPG50320 | nAPG07433.1 | LPG20052 | N-term | SGN002352 | Gene C | 14.94 | 29.13 |
| LPG50320 | nAPG07433.1 | LPG20052 | N-term | SGN002364 | Gene C | 30.27 | 45.68 |
| LPG50320 | nAPG07433.1 | LPG20052 | N-term | SGN002372 | Gene D | 92.44 | 92.48 |
| LPG50320 | nAPG07433.1 | LPG20052 | N-term | SGN003707 | B2M | 66.50 | 87.30 |
| LPG50320 | nAPG07433.1 | LPG20052 | N-term | SGN004995 | TRAC | 71.76 | 87.15 |
| LPG50208 | nAPG07433.1 | LPG20044 | Inlaid642 | SGN001256 | B2M | 30.63 | 41.77 |
| LPG50208 | nAPG07433.1 | LPG20044 | Inlaid642 | SGN001947 | B2M | 8.61 | 14.77 |
| LPG50208 | nAPG07433.1 | LPG20044 | Inlaid642 | SGN001960 | B2M | 47.26 | 56.96 |
| LPG50208 | nAPG07433.1 | LPG20044 | Inlaid642 | SGN002259 | Gene A | 2.17 | 5.66 |
| LPG50208 | nAPG07433.1 | LPG20044 | Inlaid642 | SGN002336 | Gene B | 13.70 | 15.70 |
| LPG50208 | nAPG07433.1 | LPG20044 | Inlaid642 | SGN002352 | Gene C | 1.92 | 7.26 |
| LPG50208 | nAPG07433.1 | LPG20044 | Inlaid642 | SGN002364 | Gene C | 6.74 | 10.08 |
| LPG50208 | nAPG07433.1 | LPG20044 | Inlaid642 | SGN002372 | Gene D | 65.57 | 68.73 |
| LPG50208 | nAPG07433.1 | LPG20044 | Inlaid642 | SGN003707 | B2M | 65.74 | 77.06 |
| LPG50208 | nAPG07433.1 | LPG20044 | Inlaid642 | SGN004995 | TRAC | 35.32 | 42.38 |
| LPG50208 | nAPG07433.1 | LPG20045 | Inlaid772 | SGN001256 | B2M | 51.13 | 67.01 |
| LPG50208 | nAPG07433.1 | LPG20045 | Inlaid772 | SGN001947 | B2M | 26.41 | 38.61 |
| LPG50208 | nAPG07433.1 | LPG20045 | Inlaid772 | SGN001960 | B2M | 62.45 | 76.35 |
| LPG50208 | nAPG07433.1 | LPG20045 | Inlaid772 | SGN002259 | Gene A | 8.83 | 13.31 |
| LPG50208 | nAPG07433.1 | LPG20045 | Inlaid772 | SGN002336 | Gene B | 41.31 | 42.73 |
| LPG50208 | nAPG07433.1 | LPG20045 | Inlaid772 | SGN002352 | Gene C | 10.55 | 20.54 |
| LPG50208 | nAPG07433.1 | LPG20045 | Inlaid772 | SGN002364 | Gene C | 13.06 | 19.86 |
| LPG50208 | nAPG07433.1 | LPG20045 | Inlaid772 | SGN002372 | Gene D | 59.43 | 69.72 |
| LPG50208 | nAPG07433.1 | LPG20045 | Inlaid772 | SGN003707 | B2M | 54.69 | 78.24 |
| LPG50208 | nAPG07433.1 | LPG20045 | Inlaid772 | SGN004995 | TRAC | 23.91 | 36.30 |
| LPG50258 | nAPG07433.1 | LPG20055 | N-term | SGN001256 | B2M | 49.72 | 69.98 |
| LPG50258 | nAPG07433.1 | LPG20055 | N-term | SGN001947 | B2M | 16.43 | 27.75 |
| LPG50258 | nAPG07433.1 | LPG20055 | N-term | SGN001960 | B2M | 79.19 | 90.04 |
| LPG50258 | nAPG07433.1 | LPG20055 | N-term | SGN002259 | Gene A | 8.02 | 16.08 |
| LPG50258 | nAPG07433.1 | LPG20055 | N-term | SGN002336 | Gene B | 17.98 | 24.01 |
| LPG50258 | nAPG07433.1 | LPG20055 | N-term | SGN002352 | Gene C | 8.59 | 17.19 |
| LPG50258 | nAPG07433.1 | LPG20055 | N-term | SGN002364 | Gene C | 12.70 | 21.45 |
| LPG50258 | nAPG07433.1 | LPG20055 | N-term | SGN002372 | Gene D | 93.64 | 94.06 |
| LPG50258 | nAPG07433.1 | LPG20055 | N-term | SGN003707 | B2M | 72.08 | 87.28 |
| LPG50258 | InAPG07433.1 | LPG20055 | N-term | SGN004995 | TRAC | 58.38 | 73.39 |
| LPG50265 | nAPG07433.1 | LPG20056 | N-term | SGN001256 | B2M | 57.15 | 75.17 |
| LPG50265 | nAPG07433.1 | LPG20056 | N-term | SGN001947 | B2M | 18.53 | 26.03 |
| LPG50265 | nAPG07433.1 | LPG20056 | N-term | SGN001960 | B2M | 84.92 | 91.62 |
| LPG50265 | nAPG07433.1 | LPG20056 | N-term | SGN002259 | Gene A | 4.78 | 11.81 |
| LPG50265 | nAPG07433.1 | LPG20056 | N-term | SGN002336 | Gene B | 17.47 | 23.35 |
| LPG50265 | nAPG07433.1 | LPG20056 | N-term | SGN002352 | Gene C | 12.52 | 20.04 |
| LPG50265 | nAPG07433.1 | LPG20056 | N-term | SGN002364 | Gene C | 20.84 | 30.75 |
| LPG50265 | nAPG07433.1 | LPG20056 | N-term | SGN002372 | Gene D | 93.50 | 93.87 |
| LPG50265 | nAPG07433.1 | LPG20056 | N-term | SGN003707 | B2M | 78.08 | 88.84 |
| LPG50265 | nAPG07433.1 | LPG20056 | N-term | SGN004995 | TRAC | 60.61 | 74.03 |
| LPG50266 | nAPG07433.1 | LPG20057 | N-term | SGN001256 | B2M | 60.11 | 76.46 |
| LPG50266 | nAPG07433.1 | LPG20057 | N-term | SGN001947 | B2M | 21.46 | 30.74 |
| LPG50266 | nAPG07433.1 | LPG20057 | N-term | SGN001960 | B2M | 85.45 | 91.38 |
| LPG50266 | nAPG07433.1 | LPG20057 | N-term | SGN002259 | Gene A | 6.64 | 14.43 |
| LPG50266 | nAPG07433.1 | LPG20057 | N-term | SGN002336 | Gene B | 16.18 | 24.13 |
| LPG50266 | nAPG07433.1 | LPG20057 | N-term | SGN002352 | Gene C | 13.47 | 21.35 |
| LPG50266 | nAPG07433.1 | LPG20057 | N-term | SGN002364 | Gene C | 19.03 | 27.85 |
| LPG50266 | nAPG07433.1 | LPG20057 | N-term | SGN002372 | Gene D | 92.69 | 92.98 |
| LPG50266 | nAPG07433.1 | LPG20057 | N-term | SGN003707 | B2M | 76.91 | 89.70 |
| LPG50266 | nAPG07433.1 | LPG20057 | N-term | SGN004995 | TRAC | 63.19 | 77.50 |
| LPG50274 | nAPG07433.1 | LPG20049 | N-term | SGN001256 | B2M | 79.31 | 85.78 |
| LPG50274 | nAPG07433.1 | LPG20049 | N-term | SGN001947 | B2M | 45.61 | 64.55 |
| LPG50274 | nAPG07433.1 | LPG20049 | N-term | SGN001960 | B2M | 84.21 | 87.08 |
| LPG50274 | nAPG07433.1 | LPG20049 | N-term | SGN002259 | Gene A | 20.63 | 32.57 |
| LPG50274 | nAPG07433.1 | LPG20049 | N-term | SGN002336 | Gene B | 41.81 | 49.88 |
| LPG50274 | nAPG07433.1 | LPG20049 | N-term | SGN002352 | Gene C | 26.46 | 38.98 |
| LPG50274 | nAPG07433.1 | LPG20049 | N-term | SGN002364 | Gene C | 35.49 | 42.45 |
| LPG50274 | nAPG07433.1 | LPG20049 | N-term | SGN002372 | Gene D | 88.66 | 89.07 |
| LPG50274 | nAPG07433.1 | LPG20049 | N-term | SGN003707 | B2M | 86.65 | 89.38 |
| LPG50274 | nAPG07433.1 | LPG20049 | N-term | SGN004995 | TRAC | 76.68 | 82.52 |

Evolved variants demonstrated up to a 50% increase in activity on average by mRNA delivery in primary T cells. Some architectures have guide-specific effects suggesting a relationship between specific target sequences and base editor architecture exists and was not apparent before empirical testing. The results in Table 14 demonstrate up to a 50% increase in A-to-G editing activity by evolved ABE variants by mRNA delivery in T-cells, with the LPG20049 fusion protein, comprising LPG50274/LPG50148.2.76 fused to the N-terminus of nAPG07433.1, ranking the highest on average.

### Example 11: Evaluation of mRNA Delivery to Primary Human Hepatocytes

### Methods:

Using the previously tested inlaid and terminal fusion architectures, evolved deaminases were combined with RNA-guided nucleases to generate different adenine base editors. DNA templates used to make mRNA were amplified by PCR reactions. Then the IVT (in vitro transcription) reactions were carried out, and followed by DNase I and ProK treatment to remove DNA templates and enzymes. The mRNAs were then purified by plate-based silica membrane. Primary human hepatocytes were transfected with ABE mRNAs and guide RNA (ABE mRNA: 112 ng; sgRNA: 38 ng) using Lipofectamine MessengerMax Transfection Reagent. The cells were harvested at 96 hours post-transfection and subjected to genomic DNA extraction by using QuickExtract DNA extract solution. The genomic region targeted by the sgRNA was amplified by PCR and the PCR amplicons were subsequently analyzed by NGS to evaluate the editing efficiency. The max base editing rate (Max Editing) is calculated by taking the conversion rate of adenine to guanine at each position throughout the guide and returning the maximum editing rate at a single position.

TABLE 15
Base editing rates of evolved deaminases in multiple RNA-guided nucleases with different
fusion architectures in primary human hepatocytes

| | | | Fusion | | | | |
|---|---|---|---|---|---|---|---|
| | | Fusion | SEQ ID | Type of | | | Max |
| Deaminase | Nuclease | construct | NO | Fusion | Gene | SGN | Editing |
| LPG50221 | nAPG07433.1 | LPG20047 | 578 | N-term | B2M | SGN003707 | 21 |
| LPG50221 | nAPG07433.1 | LPG20073 | 612 | Inlaid772 | B2M | SGN003707 | 26.3 |
| LPG50221 | nLPG10145 | LPG20068 | 607 | N-term | B2M | SGN003018 | 21.9 |
| LPG50221 | nLPG10145 | LPG20064 | 603 | Inlaid910 | B2M | SGN003018 | 24.5 |
| LPG50221 | nAPG05586 | LPG20072 | 611 | N-term | B2M | SGN003540 | 16.1 |
| LPG50221 | nAPG05586 | LPG20076 | 615 | Inlaid678 | B2M | SGN003540 | 13.7 |
| LPG50221 | nAPG05586 | LPG20063 | 602 | Inlaid922 | B2M | SGN003540 | 19.8 |
| LPG50274 | nAPG07433.1 | LPG20046 | 577 | N-term | B2M | SGN003707 | 20.8 |
| LPG50274 | nAPG07433.1 | LPG20067 | 606 | Inlaid772 | B2M | SGN003707 | 19.6 |
| LPG50274 | nLPG10145 | LPG20074 | 613 | N-term | B2M | SGN003018 | 19.2 |
| LPG50274 | nLPG10145 | LPG20062 | 600 | Inlaid910 | B2M | SGN003018 | 22.7 |
| LPG50274 | nAPG05586 | LPG20069 | 608 | N-term | B2M | SGN003540 | 16.2 |
| LPG50274 | nAPG05586 | LPG20071 | 610 | Inlaid678 | B2M | SGN003540 | 11.3 |
| LPG50274 | nAPG05586 | LPG20061 | 599 | Inlaid922 | B2M | SGN003540 | 16.4 |
| LPG50319 | nAPG07433.1 | LPG20077 | 616 | N-term | B2M | SGN003707 | 28.3 |
| LPG50319 | nAPG07433.1 | LPG20096 | 630 | Inlaid772 | B2M | SGN003707 | 25.5 |
| LPG50319 | nLPG10145 | LPG20094 | 628 | N-term | B2M | SGN003018 | 26.6 |
| LPG50319 | nLPG10145 | LPG20087 | 621 | Inlaid910 | B2M | SGN003018 | 42.8 |
| LPG50319 | nAPG05586 | LPG20091 | 625 | N-term | B2M | SGN003540 | 21.6 |
| LPG50319 | nAPG05586 | LPG20083 | 617 | Inlaid678 | B2M | SGN003540 | 12.6 |
| LPG50319 | nAPG05586 | LPG20089 | 623 | Inlaid922 | B2M | SGN003540 | 19 |
| LPG50320 | nAPG07433.1 | LPG20065 | 604 | N-term | B2M | SGN003707 | 18.3 |
| LPG50320 | nAPG07433.1 | LPG20097 | 631 | Inlaid772 | B2M | SGN003707 | 22.4 |
| LPG50320 | nLPG10145 | LPG20084 | 618 | N-term | B2M | SGN003018 | 31.9 |
| LPG50320 | nLPG10145 | LPG20090 | 624 | Inlaid910 | B2M | SGN003018 | 34.9 |
| LPG50320 | nAPG05586 | LPG20093 | 627 | N-term | B2M | SGN003540 | 21.7 |
| LPG50320 | nAPG05586 | LPG20086 | 620 | Inlaid678 | B2M | SGN003540 | 13.8 |
| LPG50320 | nAPG05586 | LPG20092 | 626 | Inlaid922 | B2M | SGN003540 | 21.6 |
| LPG50324 | nAPG07433.1 | LPG20066 | 605 | N-term | B2M | SGN003707 | 22.1 |
| LPG50324 | nAPG07433.1 | LPG20098 | 632 | Inlaid772 | B2M | SGN003707 | 19.5 |
| LPG50324 | nLPG10145 | LPG20095 | 629 | N-term | B2M | SGN003018 | 20.3 |
| LPG50324 | nLPG10145 | LPG20088 | 622 | Inlaid910 | B2M | SGN003018 | 38.9 |
| LPG50324 | nAPG05586 | LPG20070 | 609 | N-term | B2M | SGN003540 | 16.9 |
| LPG50324 | nAPG05586 | LPG20085 | 619 | Inlaid678 | B2M | SGN003540 | 17.4 |
| LPG50324 | nAPG05586 | LPG20075 | 614 | Inlaid922 | B2M | SGN003540 | 28 |

Effective editing in human primary hepatocytes was observed by mRNA delivery of the base editors. Both inlaid and N-terminal fusions of the improved deaminases are effective with 3 unique nucleases (nAPG07433.1, nAPG05586 and nLPG10145).

### Example 12: Editing in HepG2 Cells

HepG2 cells were transfected with ABE mRNA and guide RNA at the ratio of 3:1 (ABE mRNA: 150 ng and sgRNA: 50 ng) using Lipofectamine MessengerMax Transfection Reagent. The cells were harvested 48 h post-transfection and subjected to genomic DNA extraction by using QuickExtract DNA extract solution. The genomic region targeted by the sgRNA was amplified by PCR and the PCR amplicons were subsequently analyzed by NGS to evaluate the editing efficiency. The max base editing rate (Max Editing) is calculated by taking the conversion rate of adenine to guanine at each position throughout the guide and returning the maximum editing rate at a single position.

TABLE 16
Base editing rates of evolved deaminases fused to
the amino terminus of nAPG07433.1 in HepG2 cells

| | | | | | Average |
|---|---|---|---|---|---|
| | Fusion | Type of | | Target | Max BE |
| Deaminase | Construct | Fusion | SGN | gene | Rate |
| LPG50148 | LPG20048 | N-term | SGN003707 | B2M | 36.625 |
| LPG50148 | LPG20048 | N-term | SGN00AAAA | Gene E | 46.4125 |
| LPG50148 | LPG20048 | N-term | SGN008971 | TRAC | 15.975 |
| LPG50221 | LPG20051 | N-term | SGN003707 | B2M | 40.9 |
| LPG50221 | LPG20051 | N-term | SGN00AAAA | Gene E | 32.925 |
| LPG50221 | LPG20051 | N-term | SGN008971 | TRAC | 9.65 |
| LPG50208 | LPG20050 | N-term | SGN003707 | B2M | 54.65 |
| LPG50208 | LPG20050 | N-term | SGN00AAAA | Gene E | 53.675 |
| LPG50208 | LPG20050 | N-term | SGN008971 | TRAC | 18.05 |
| LPG50320 | LPG20065 | N-term | SGN003707 | B2M | 57.9 |
| LPG50320 | LPG20065 | N-term | SGN00AAAA | Gene E | 59 |
| LPG50320 | LPG20065 | N-term | SGN008971 | TRAC | 17.55 |
| LPG50324 | LPG20066 | N-term | SGN003707 | B2M | 59.9 |
| LPG50324 | LPG20066 | N-term | SGN00AAAA | Gene E | 54.775 |
| LPG50324 | LPG20066 | N-term | SGN008971 | TRAC | 11.45 |
| LPG50274 | LPG20046 | N-term | SGN003707 | B2M | 73.3 |
| LPG50274 | LPG20046 | N-term | SGN00AAAA | Gene E | 56.85 |
| LPG50274 | LPG20046 | N-term | SGN008971 | TRAC | 16.95 |

TABLE 17
Base editing rates of evolved deaminases in nAPG05586
with different fusion architectures in HepG2 cells

| | Fusion | Type of | | Target | Average BE |
|---|---|---|---|---|---|
| Deaminase | Protein | Fusion | SGN | gene | Max |
| LPG50221 | LPG20076 | Inlaid678 | SGN003486 | TRAC | 17.6 |
| LPG50221 | LPG20076 | Inlaid678 | SGN003540 | B2M | 20.65 |
| LPG50274 | LPG20063 | Inlaid922 | SGN003486 | TRAC | 37.3666667 |
| LPG50274 | LPG20063 | Inlaid922 | SGN003540 | B2M | 44.2 |
| LPG50221 | LPG20071 | Inlaid678 | SGN003486 | TRAC | 30.25 |
| LPG50221 | LPG20071 | Inlaid678 | SGN003540 | B2M | 31.15 |
| LPG50274 | LPG20061 | Inlaid922 | SGN003486 | TRAC | 54.0333333 |
| LPG50274 | LPG20061 | Inlaid922 | SGN003540 | B2M | 67.6 |

Inlaid configurations for nAPG05586 and nAPG07433.1 with lead deaminases are capable of editing in HepG2 cells with mRNA delivery.

### Example 13: NLPG10196 N-Terminal Fusion and Inlaid Base Editors Function with Evolved Deaminases

Based on sequence and structural analysis, four inlaid constructs for nLPG10196 were prepared in addition to an N-terminal fusion. Messenger RNAs were expressed and purified for the LPG10196 base editor constructs. Briefly, DNA templates used to make mRNA were amplified by PCR reactions. Then the IVT (in vitro transcription) reactions were carried out, and followed by DNase I and ProK treatment to remove DNA templates and enzymes. The mRNAs were then purified by plate-based silica membrane. For base editing, 150 ng mRNA of mRNA and 50 ng of sgRNA (synthetic guide) were lipofected with MessengerMax into HEK293T cells seeded at 10,000 cells/well. Cells were incubated for three days and then genomic DNA was extracted. PCR amplification was used to amplify the edited regions of interest and sent for NGS sequencing. The max base editing rate (Max BE Rate) is calculated by taking the conversion rate of adenine to guanine at each position throughout the guide and returning the maximum editing rate at a single position.

TABLE 18
nLPG10196 base editors function in HEK293T cells by mRNA delivery

| | Fusion | Fusion | | Target | Average of |
|---|---|---|---|---|---|
| Deaminase | Location | protein | SGN | gene | Max BE Rate |
| LPG50221 | N-terminus | LPG20099 | SGN005326 | TRAC | 5.750% |
| LPG50221 | N-terminus | LPG20099 | SGN005329 | B2M | 0.000% |
| LPG50274 | N-terminus | LPG20100 | SGN005326 | TRAC | 3.034% |
| LPG50274 | N-terminus | LPG20100 | SGN005329 | B2M | 0.124% |
| LPG50324 | N-terminus | LPG20101 | SGN005326 | TRAC | 8.132% |
| LPG50324 | N-terminus | LPG20101 | SGN005329 | B2M | 0.080% |
| LPG50319 | N-terminus | LPG20102 | SGN005326 | TRAC | 5.996% |
| LPG50319 | N-terminus | LPG20102 | SGN005329 | B2M | 0.208% |
| LPG50274 | inlaid T806 | LPG20111 | SGN005326 | TRAC | 3.018% |
| LPG50274 | inlaid T806 | LPG20111 | SGN005329 | B2M | 0.000% |
| LPG50274 | Inlaid D700 | LPG20120 | SGN005326 | TRAC | 0.000% |
| LPG50274 | Inlaid D700 | LPG20120 | SGN005329 | B2M | 0.000% |
| LPG50274 | inlaid 1797 | LPG20121 | SGN005326 | TRAC | 0.057% |
| LPG50274 | inlaid I797 | LPG20121 | SGN005329 | B2M | 0.000% |
| LPG50274 | inlaid G766 | LPG20122 | SGN005326 | TRAC | 4.263% |

Several deaminases functioned as N-terminal fusions to nLPG10196. Several in-laid positions were tested but only two inlaid positions showed editing above background (LPG20122 and LPG20111) suggesting LPG10196 has site-specific requirements for in-laid base editor constructs. The N-terminal fusions showed higher editing at SGN005329 compared to SGN005326 with all deaminases tested. LPG10196 is capable of base editing activity with the evolved deaminases but there are target-specific and deaminase-specific factors contributing to base editing rates.

### Example 14: Discovery and Evaluation of LPG50310 Variant

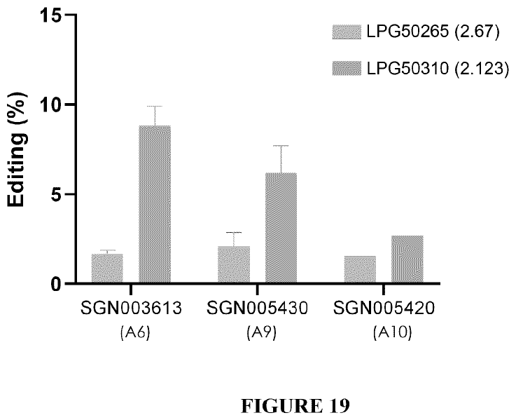

38 beneficial mutations dispersed among 28 positions from the first round of selection were identified. These mutations were combined to form a library featuring 1-28 mutations per variant (average of 19 mutations). Subsequently, this library was subjected to higher stringency selection conditions, utilizing kanamycin concentrations of 256-512 μg/ml and two stop codons, in order to identify the most promising variants. Among these, a particularly enriched variant, LPG50310 (set forth as SEQ ID NO: 723), was discovered, which was then evaluated in mammalian cells. The evaluation was performed in HEK293T cells (10K cells/reaction) transfected with base editor plasmid (160 ng) and guide plasmid (40 ng) at the ratio of 4:1 using Lipofectamine P3000 transfection reagent. The base editor sequence is set forth as SEQ ID NO: 727 and named LPG20125, which comprises the LPG50310 deaminase fused to the N-terminus of the nAPG07433.1 nickase. The cells were harvested 48 h post-transfection and subjected to genomic DNA extraction using QuickExtract DNA extract solution. The genomic region targeted by the sgRNA was amplified by PCR, and the PCR amplicons were subsequently analyzed by NGS to evaluate the editing efficiency. Results are shown in FIG. 19, which compares base editor LPG20125 (comprising the LPG50310 fused to the N-terminus of nAPG07433.1) to LPG20130 (comprising the LPG50265 deaminase fused to the N-terminus of nAPG07433.1) editing with the sgRNAs SGN00CCCC, GN005430, and SGN005420, each of which target a different gene at positions A6, A9, and A10, respectively.

TABLE 19
Additional mutations within presently disclosed evolved deaminases
in relation to the parental LPG50148 deaminase (SEQ ID NO: 5).

| | | |
|---|---|---|
| R22C | I75A | I75Q |
| I75Y | K109L | N121A |
| N121G | Y122H | G142A |
| C145Q | C145L | Q153E |
| Q153R | | |

TABLE 20
Sequences of the application

| Sequence name | SEQ ID NO |
|---|---|
| APG07433.1 | 1 |
| APG05586 | 2 |
| APG01604 | 3 |
| LPG10145 | 4 |
| LPG50148 (minus starting methionine) | 5 |
| LPG50148 V81S (minus starting methionine) | 6 |
| LPG50148 nAPG07433.1_parental | 7 |
| nAPG07433.1 inlaidS772_LPG50148 (LPG20002) | 8 |
| nAPG07433.1 inlaidS775 LPG50148 (LPG20003) | 9 |
| nAPG07433.1 inlaidK778 LPG50148 (LPG20004) | 10 |
| nAPG07433.1 inlaidE900 LPG50148 (LPG20005) | 11 |
| nAPG07433.1 inlaidG910 LPG50148 (LPG20006) | 12 |
| nAPG07433.1 inlaidR30 LPG50148 (LPG20007) | 13 |
| nAPG07433.1 inlaidT642 LPG50148 (LPG20008) | 14 |
| nAPG07433.1 inlaidR670 LPG50148 (LPG20009) | 15 |
| nAPG07433.1 inlaidD737_LPG50148 (LPG20010) | 16 |
| nAPG07433.1 inlaidA802_LPG50148 (LPG20011) | 17 |
| nAPG07433.1inlaidS772_LPG50148 (delNterlinker/ | 18 |
| LPG20012) | |
| nAPG07433.1_inlaidS772LPG50148 (delCterlinker/ | 19 |
| LPG20013) | |
| nAPG07433.1inlaidS772LPG50148_dellaaNandC | 20 |
| (nolinker/laaNandC/ LPG20014) | |
| nAPG07433.1_inlaidS772_LPG50148 + V81Smutant | 21 |
| (LPG20015) | |
| LPG50148 + V81S nAPG05586_parental | 22 |
| nAPG05586 inlaidG342_LPG50148 + V81S (LPG20016) | 23 |
| nAPG05586 inlaidD518 LPG50148 + V81S (LPG20017) | 24 |
| nAPG05586 inlaidQ630_LPG50148 + V81S (LPG20018) | 25 |
| nAPG05586_inlaidR661_LPG50148 + V81S (LPG20019) | 26 |
| nAPG05586 inlaidF678 LPG50148 + V81S (LPG20020) | 27 |
| nAPG05586 inlaidD736_LPG50148 + V81S (LPG20021) | 28 |
| nAPG05586_inlaidE778_LPG50148 + V81S (LPG20022) | 29 |
| nAPG05586 inlaidD788 LPG50148 + V81S (LPG20023) | 30 |
| nAPG05586 inlaidK922 LPG50148 + V81S (LPG20024) | 31 |
| nAPG05586 inlaidG1109_LPG50148 + V81S (LPG20025) | 32 |
| LPG50148 + V81S nAPG01604_parental | 33 |
| nAPG01604 inlaidG597_LPG50148 + V81S + A137V | 34 |
| (LPG20027) | |
| nAPG01604 inlaidS631_LPG50148 + V81S + A137V | 35 |
| (LPG20028) | |
| nAPG01604_inlaidK725_LPG50148 + V81S + A137V | 36 |
| (LPG20029) | |
| nAPG01604 inlaidK739_LPG50148 + V81S + A137V | 37 |
| (LPG20030) | |
| nAPG01604 inlaidV744 LPG50148 + V81S + A137V | 38 |
| (LPG20031) | |
| LPG50148 + V81S nLPG10145_parental | 39 |
| nLPG10145 inlaidG347_LPG50148 + V81S (LPG20032) | 40 |
| nLPG10145 inlaidD524 LPG50148 + V81S (LPG20033) | 41 |
| nLPG10145 inlaidQ640 LPG50148 + V81S (LPG20034) | 42 |
| nLPG10145 inlaidR666 LPG50148 + V81S (LPG20035) | 43 |
| nLPG10145 inlaidD680 LPG50148 + V81S (LPG20036) | 44 |
| nLPG10145 inlaidN740 LPG50148 + V81S (LPG20037) | 45 |
| nLPG10145 inlaidS785 LPG50148 + V81S (LPG20038) | 46 |
| nLPG10145 inlaidP910_LPG50148 + V81S (LPG20039) | 47 |
| nLPG10145 inlaidG1077 LPG50148 + V81S (LPG20040) | 48 |
| nAPG07433.1 (D11A) | 49 |
| nAPG05586 (D11A) | 50 |
| nAPG01604 (D11A) | 51 |
| nLPG10145 (D11A) | 52 |
| nAPG05586 (H840A) | 53 |
| nAPG01604 (H840A) | 54 |
| nLPG10145 (H840A) | 55 |
| nAPG07433.1 (H840A) | 56 |
| APG08290.1 | 57 |
| APG05459.1 | 58 |
| APG007513.1 | 59 |
| APG05083.1 | 60 |
| APG04583.1 | 61 |
| APG01688.1 | 62 |
| APG00969 | 63 |
| APG03128 | 64 |
| APG00771 | 65 |
| APG05733 | 66 |
| APG06207 | 67 |
| APG01647 | 68 |
| APG08032 | 69 |
| APG05712 | 70 |
| APG01658 | 71 |
| APG06498 | 72 |
| APG09882 | 73 |
| APG02675 | 74 |
| APG01405 | 75 |
| APG06250 | 76 |
| APG06877 | 77 |
| APG09053 | 78 |
| APG04293 | 79 |
| APG01308 | 80 |
| APG06646 | 81 |
| APG06622 | 82 |
| APG02787 | 83 |
| APG06248 | 84 |
| APG06007 | 85 |
| APG02874 | 86 |
| APG03850 | 87 |
| APG07553 | 88 |
| APG03031 | 89 |
| APG09208 | 90 |
| APG08770 | 91 |
| APG08167 | 92 |
| APG03021 | 93 |
| APG06015 | 94 |
| APG09344 | 95 |
| APG07991 | 96 |
| APG01868 | 97 |
| APG02998 | 98 |
| APG09298 | 99 |
| APG06251 | 100 |
| APG03066 | 101 |
| APG01560 | 102 |
| APG02777 | 103 |
| APG05761 | 104 |
| APG02479 | 105 |
| APG08385 | 106 |
| APG09217 | 107 |
| APG06657 | 108 |
| LPG10134 | 109 |
| LPG10136 | 110 |
| LPG10138 | 111 |
| LPG10139 | 112 |
| LPG10141 | 113 |
| LPG10146 | 114 |
| LPG10147 | 115 |
| LPG10150 | 116 |
| LPG10155 | 117 |
| LPG10159 | 118 |
| LPG10160 | 119 |
| LPG10165 | 120 |
| LPG10166 | 121 |
| LPG10167 | 122 |
| LPG10168 | 123 |
| LPG10169 | 124 |
| LPG10171 | 125 |
| LPG10186 | 126 |
| LPG10190 | 127 |
| LPG10191 | 128 |
| LPG10194 | 129 |
| LPG10195 | 130 |
| LPG10196 | 131 |
| LPG10197 | 132 |
| LPG10198 | 133 |
| LPG10200 | 134 |
| LPG10203 | 135 |
| LPG10204 | 136 |
| LPG10205 | 137 |
| LPG10207 | 138 |
| LPG10208 | 139 |
| APG09748 | 140 |
| APG02789 | 141 |
| APG09106 | 142 |
| APG07339 | 143 |
| APG09624 | 144 |
| APG03003 | 145 |
| APG05405 | 146 |
| APG09777 | 147 |
| APG05680 | 148 |
| APG02119 | 149 |
| APG03285 | 150 |
| APG04998 | 151 |
| APG07078 | 152 |
| APG06369 | 153 |
| APG03847 | 154 |
| APG05625 | 155 |
| APG03759 | 156 |
| APG05123 | 157 |
| APG03524 | 158 |
| APG07446 | 159 |
| APG07641 | 160 |
| APG01250 | 161 |
| APG02261 | 162 |
| LPG10134 crRNA repeat | 163 |
| LPG10136 crRNA repeat | 164 |
| LPG10138 crRNA repeat | 165 |
| LPG10139 crRNA repeat | 166 |
| LPG10141 crRNA repeat | 167 |
| LPG10145 crRNA repeat | 168 |
| LPG10146 crRNA repeat | 169 |
| LPG10147 crRNA repeat | 170 |
| LPG10150 crRNA repeat | 171 |
| LPG10155 crRNA repeat | 172 |
| LPG10159 crRNA repeat | 173 |
| LPG10160 crRNA repeat | 174 |
| LPG10134 tracrRNA | 175 |
| LPG10136 tracrRNA | 176 |
| LPG10138 tracrRNA | 177 |
| LPG10139 tracrRNA | 178 |
| LPG10141 tracrRNA | 179 |
| LPG10145 tracrRNA | 180 |
| LPG10146 tracrRNA | 181 |
| LPG10147 tracrRNA | 182 |
| LPG10150 tracrRNA | 183 |
| LPG10155 tracrRNA | 184 |
| LPG10159 tracrRNA | 185 |
| LPG10160 tracrRNA | 186 |
| LPG10165 crRNA repeat | 187 |
| LPG10166 crRNA repeat | 188 |
| LPG10167 crRNA repeat | 189 |
| LPG10168 crRNA repeat | 190 |
| LPG10169 crRNA repeat | 191 |
| LPG10171 crRNA repeat | 192 |
| LPG10186 crRNA repeat | 193 |
| LPG10190 crRNA repeat | 194 |
| LPG10191 crRNA repeat | 195 |
| LPG10194 crRNA repeat | 196 |
| LPG10195 crRNA repeat | 197 |
| LPG10196 crRNA repeat | 198 |
| LPG10197 crRNA repeat | 199 |
| LPG10198 crRNA repeat | 200 |
| LPG10200 crRNA repeat | 201 |
| LPG10203 crRNA repeat | 202 |
| LPG10204 crRNA repeat | 203 |
| LPG10205 crRNA repeat | 204 |
| LPG10205 crRNA repeat 2 | 205 |
| LPG10207 crRNA repeat | 206 |
| LPG10208 crRNA repeat | 207 |
| LPG10165 tracrRNA | 208 |
| LPG10166 tracrRNA | 209 |
| LPG10167 tracrRNA | 210 |
| LPG10168 tracrRNA | 211 |
| LPG10169 tracrRNA | 212 |
| LPG10171 tracrRNA | 213 |
| LPG10186 tracrRNA | 214 |
| LPG10190 tracrRNA | 215 |
| LPG10191 tracrRNA | 216 |
| LPG10194 tracrRNA | 217 |
| LPG10195 tracrRNA | 218 |
| LPG10196 tracrRNA | 219 |
| LPG10197 tracrRNA | 220 |
| LPG10198 tracrRNA | 221 |
| LPG10200 tracrRNA | 222 |
| LPG10203 tracrRNA | 223 |
| LPG10204 tracrRNA | 224 |
| LPG10205 tracrRNA | 225 |
| LPG10205 tracrRNA2 | 226 |
| LPG10207 tracrRNA | 227 |
| LPG10208 tracrRNA | 228 |
| APG05241 | 229 |
| APG07280 | 230 |
| APG09260 | 231 |
| APG08360 | 232 |
| APG09980 | 233 |
| APG07386 | 234 |
| APG09688 | 235 |
| APG05840 | 236 |
| APG02316 | 237 |
| APG00868 | 238 |
| APG30125 | 239 |
| APG30126 | 240 |
| APG30127 | 241 |
| APG30128 | 242 |
| APG30129 | 243 |
| APG30130 | 244 |
| APG09980.1 | 245 |
| APG05840.1 | 246 |
| APG00868.1 | 247 |
| APG09982 | 248 |
| APG03724 | 249 |
| APG09949 | 250 |
| APG08196 | 251 |
| APG06333 | 252 |
| APG06489 | 253 |
| APG08449 | 254 |
| APG05174 | 255 |
| APG09102 | 256 |
| APG05723 | 257 |
| LPG50140 | 258 |
| LPG50141 | 259 |
| LPG50142 | 260 |
| LPG50143 | 261 |
| LPG50144 | 262 |
| LPG50145 | 263 |
| LPG50146 | 264 |
| LPG50147 | 265 |
| LPG50149 | 266 |
| LPG50150 | 267 |
| LPG50151 | 268 |
| LPG50152 | 269 |
| LPG50153 | 270 |
| LPG50154 | 271 |
| LPG50155 | 272 |
| LPG50156 | 273 |
| LPG50157 | 274 |
| LPG50158 | 275 |
| LPG50159 | 276 |
| LPG50160 | 277 |
| LPG50161 | 278 |
| LPG50162 | 279 |
| LPG50163 | 280 |
| LPG50164 | 281 |
| LPG50165 | 282 |
| LPG50166 | 283 |
| LPG50167 | 284 |
| LPG50168 | 285 |
| LPG50169 | 286 |
| LPG50170 | 287 |
| LPG50171 | 288 |
| LPG50172 | 289 |
| LPG50173 | 290 |
| LPG50174 | 291 |
| LPG50175 | 292 |
| LPG50176 | 293 |
| LPG50177 | 294 |
| LPG50178 | 295 |
| LPG50179 | 296 |
| LPG50180 | 297 |
| LPG50181 | 298 |
| LPG50182 | 299 |
| LPG50148.2.1 | 300 |
| LPG50148.2.2 | 301 |
| LPG50148.2.3 | 302 |
| LPG50148.2.4 | 303 |
| LPG50148.2.5 | 304 |
| LPG50148.2.6 | 305 |
| LPG50148.2.7 | 306 |
| LPG50148.2.8 | 307 |
| LPG50148.2.9 | 308 |
| LPG50148.2.10 | 309 |
| LPG50148.2.11 | 310 |
| LPG50148.2.12 | 311 |
| LPG50148.2.13 | 312 |
| LPG50148.2.14 | 313 |
| LPG50148.2.15 | 314 |
| LPG50148.2.16 | 315 |
| LPG50148.2.17 | 316 |
| LPG50148.2.18 | 317 |
| LPG50148.2.19 | 318 |
| LPG50148.2.20 | 319 |
| LPG50148.2.21 | 320 |
| LPG50148.2.22 | 321 |
| LPG50148.2.23 | 322 |
| LPG50148.2.24 | 323 |
| LPG50148.2.25 | 324 |
| LPG50148.2.26 | 325 |
| LPG50148.2.27 | 326 |
| LPG50148.2.28 | 327 |
| LPG50148.2.29 | 328 |
| LPG50148.2.30 | 329 |
| LPG50148.2.31 | 330 |
| LPG50148.2.32 | 331 |
| LPG50148.2.33 | 332 |
| LPG50148.2.34 | 333 |
| LPG50148.2.35 | 334 |
| LPG50148.2.36 | 335 |
| LPG50148.2.37 | 336 |
| LPG50148.2.38 | 337 |
| LPG50148.2.39 | 338 |
| LPG50148.2.40 | 339 |
| LPG50148.2.41 | 340 |
| LPG50148.2.42 | 341 |
| LPG50148.2.43 | 342 |
| LPG50148.2.44 | 343 |
| LPG50148.2.45 | 344 |
| LPG50148.2.46 | 345 |
| LPG50148.2.47 | 346 |
| LPG50148.2.48 | 347 |
| LPG50148.2.49 | 348 |
| LPG50148.2.50 | 349 |
| LPG50148.2.51 | 350 |
| LPG50148.2.52 | 351 |
| LPG50148.2.53 | 352 |
| LPG50148.2.54 | 353 |
| LPG50148.2.55 | 354 |
| LPG50148.2.56 | 355 |
| LPG50148.2.57 | 356 |
| LPG50148.2.58 | 357 |
| LPG50148.2.59 | 358 |
| LPG50148.2.60 | 359 |
| LPG50148.2.61 | 360 |
| LPG50148.2.62 | 361 |
| LPG50148.2.63 | 362 |
| LPG50148.2.64 | 363 |
| LPG50148.2.65 | 364 |
| LPG50148.2.66 | 365 |
| LPG50148.2.67 | 366 |
| LPG50148.2.68 | 367 |
| LPG50148.2.69 | 368 |
| LPG50148.2.70 | 369 |
| LPG50148.2.71 | 370 |
| LPG50148.2.72 | 371 |
| LPG50148.2.73 | 372 |
| LPG50148.2.74 | 373 |
| LPG50148.2.75 | 374 |
| LPG50148.2.76 | 375 |
| LPG50148.2.77 | 376 |
| LPG50148.2.78 | 377 |
| LPG50148.2.79 | 378 |
| LPG50148.2.80 | 379 |
| LPG50148.2.81 | 380 |
| LPG50148.2.82 | 381 |
| LPG50148.2.83 | 382 |
| LPG50148.2.84 | 383 |
| LPG50148.2.85 | 384 |
| LPG50148.2.86 | 385 |
| LPG50148.2.87 | 386 |
| LPG50148.2.88 | 387 |
| LPG50148.2.89 | 388 |
| LPG50148.2.90 | 389 |
| LPG50148.2.91 | 390 |
| LPG50148.2.92 | 391 |
| LPG50148.2.93 | 392 |
| LPG50148.2.94 | 393 |
| LPG50148.2.95 | 394 |
| LPG50148.2.96 | 395 |
| LPG50148.2.97 | 396 |
| LPG50148.2.98 | 397 |
| LPG50148.2.99 | 398 |
| LPG50148.2.100 | 399 |
| LPG50148.2.101 | 400 |
| LPG50148.2.102 | 401 |
| LPG50148.2.103 | 402 |
| LPG50148.2.104 | 403 |
| LPG50148.2.105 | 404 |
| LPG50148.2.106 | 405 |
| LPG50148.2.107 (also known as LPG50319) | 406 |
| LPG50148.2.108 | 407 |
| LPG50148.2.109 | 408 |
| LPG50148.2.110 | 409 |
| LPG50148.2.111 | 410 |
| LPG50148.2.112 | 411 |
| LPG50148.2.113 | 412 |
| LPG50148.2.114 | 413 |
| LPG50148.2.115 | 414 |
| Streptococcus pyogenes Cas9 | 415 |
| APG07433.1 crRNA repeat | 416 |
| APG07433.1 tracrRNA | 417 |
| APG07433.1 PAM | 418 (NNNNCC) |
| APG05586 crRNA repeat | 419 |
| APG05586 tracrRNA | 420 |
| APG05586 PAM | 421 (NNRYA) |
| APG01604 crRNA repeat | 422 |
| APG01604 tracrRNA | 423 |
| APG01604 PAM | 424 (NNGRR) |
| LPG10145 crRNA repeat | 425 |
| LPG10145 tracrRNA | 426 |
| LPG10145 PAM | 427 (NNGG) |
| nAPG07433.1_HNHreplacement_LPG50148 (LPG20001) | 428 |
| nAPG07433.1 inlaidS772LPG50148 (Nolinker/ LPG20041) | 429 |
| SV40 NLS | 430 |
| Nucleoplasmin NLS | 431 |
| APG05083.1 crRNA repeat | 432 |
| APG05083.1 tracrRNA | 433 |
| APG05083.1 PAM | 434 (NNNNCC) |
| APG07513.1 | 435 |
| APG07513.1 crRNA repeat | 436 |
| APG07513.1 tracrRNA | 437 |
| APG07513.1 PAM | 438 (NNNNCC) |
| APG08290.1 crRNA repeat | 439 |
| APG08290.1 tracrRNA | 440 |
| APG08290.1 PAM | 441 (NNRNCC) |
| APG05459.1 crRNA repeat | 442 |
| APG05459.1 tracrRNA | 443 |
| APG05459.1 PAM | 444 (NGG) |
| APG04583.1 crRNA repeat | 445 |
| APG04583.1 tracrRNA | 446 |
| APG04583.1 PAM | 447 (NNRAATY) |
| APG01688.1 crRNA repeat | 448 |
| APG01688.1 tracrRNA | 449 |
| APG01688.1 PAM | 450 (NNRNNC) |
| APG00969 crRNA repeat | 451 |
| APG00969 tracrRNA | 452 |
| APG00969 PAM | 453 (NNARV) |
| APG03128 crRNA repeat | 454 |
| APG03128 tracrRNA | 455 |
| APG03128 PAM | 456 (NNGGNNC) |
| APG09748 crRNA repeat | 457 |
| APG09748 tracrRNA | 458 |
| APG09748 PAM | 459 (DTTN) |
| APG00771 crRNA repeat | 460 |
| APG00771 tracrRNA | 461 |
| APG00771 PAM | 462 (NNRAAT) |
| APG02789 crRNA repeat | 463 |
| APG02789 tracrRNA | 464 |
| APG02789 PAM | 465 (ATTN) |
| APG09106 crRNA repeat | 466 |
| APG09106 tracrRNA | 467 |
| APG09106 PAM | 468 (DTTN) |
| APG05733.1 crRNA repeat | 469 |
| APG05733.1 tracrRNA | 470 |
| APG05733.1 PAM | 471 (NNNNCC) |
| APG06207.1 crRNA repeat | 472 |
| APG06207.1 tracrRNA | 473 |
| APG06207.1 PAM | 474 (NRRTTT) |
| APG01647.1 crRNA repeat | 475 |
| APG01647.1 tracrRNA | 476 |
| APG01647.1 PAM | 477 (NNRRNGG) |
| APG08032.1 crRNA repeat | 478 |
| APG08032.1 tracrRNA | 479 |
| APG08032.1 PAM | 480 (NNRAAR) |
| APG05712.1 crRNA repeat | 481 |
| APG05712.1 tracrRNA | 482 |
| APG05712.1 PAM | 483 (NNRNAT) |
| APG01658.1 crRNA repeat | 484 |
| APG01658.1 tracrRNA | 485 |
| APG01658.1 PAM | 486 (NNGG) |
| APG06498.1 crRNA repeat | 487 |
| APG06498.1 tracrRNA | 488 |
| APG06498.1 PAM | 489 (NNNNCC) |
| APG09106.1 crRNA repeat | 490 |
| APG09106.1 tracrRNA | 491 |
| APG09106.1 PAM | 492 (DTTN) |
| APG09882.1 crRNA repeat | 493 |
| APG09882.1 tracrRNA | 494 |
| APG09882.1 PAM | 495 (NNNNA) |
| APG02675.1 crRNA repeat | 496 |
| APG02675.1 tracrRNA | 497 |
| APG02675.1 PAM | 498 (NRRTTT) |
| APG01405.1 crRNA repeat | 499 |
| APG01405.1 tracrRNA | 500 |
| APG01405.1 PAM | 501 (NNRAAT) |
| APG06250.1 crRNA repeat | 502 |
| APG06250.1 tracrRNA | 503 |
| APG06250.1 PAM | 504 (NNRRNG) |
| APG06877.1 crRNA repeat | 505 |
| APG06877.1 tracrRNA | 506 |
| APG06877.1 PAM | 507 (NNNNCC) |
| APG09053.1 crRNA repeat | 508 |
| APG09053.1 tracrRNA | 509 |
| APG09053.1 PAM | 510 (NGG) |
| APG04293.1 crRNA repeat | 511 |
| APG04293.1 tracrRNA | 512 |
| APG04293.1 PAM | 513 (NNNNC) |
| APG01308.1 crRNA repeat | 514 |
| APG01308.1 tracrRNA | 515 |
| APG01308.1 PAM | 516 (NNNNNC) |
| APG06646.1 crRNA repeat | 517 |
| APG06646.1 tracrRNA | 518 |
| APG06646.1 PAM | 519 (NNNNC) |
| APG09624 crRNA repeat | 520 |
| APG09624 tracrRNA | 521 |
| APG05405 crRNA repeat | 522 |
| APG05405 tracrRNA | 523 |
| APG06622 crRNA repeat | 524 |
| APG06622 tracrRNA | 525 |
| APG06622 PAM | 526 (NNAARG) |
| APG02787 crRNA repeat | 527 |
| APG02787 tracrRNA | 528 |
| APG02787 PAM | 529 (NNRRNA) |
| APG06248 crRNA repeat | 530 |
| APG06248 tracrRNA | 531 |
| APG06248 PAM | 532 (NNRAA) |
| APG06007 crRNA repeat | 533 |
| APG06007 tracrRNA | 534 |
| APG06007 PAM | 535 (NNNNTC) |
| APG02874 crRNA repeat | 536 |
| APG02874 tracrRNA | 537 |
| APG02874 PAM | 538 (NNNNCC) |
| APG03850 crRNA repeat | 539 |
| APG03850 tracrRNA | 540 |
| APG03850 PAM | 541 (NNNNG) |
| APG07553 crRNA repeat | 542 |
| APG07553 tracrRNA | 543 |
| APG07553 PAM | 544 (NNNNGNNA) |
| APG03031 crRNA repeat | 545 |
| APG03031 tracrRNA | 546 |
| APG03031 PAM | 547 (NNNNCC) |
| APG09208 crRNA repeat | 548 |
| APG09208 tracrRNA | 549 |
| APG09208 PAM | 550 (NNNNC) |
| APG08770 crRNA repeat | 551 |
| APG08770 tracrRNA | 552 |
| APG08770 PAM | 553 (NNRYA) |
| APG08167 crRNA repeat | 554 |
| APG08167 tracrRNA | 555 |
| APG08167 PAM | 556 (NNGRR) |
| APG03021 crRNA repeat | 557 |
| APG03021 tracrRNA | 558 |
| APG03021 PAM | 559 (NNNAAA) |
| APG06015 crRNA repeat | 560 |
| APG06015 tracrRNA | 561 |
| APG06015 PAM | 562(NNAAA) |
| APG09344 crRNA repeat | 563 |
| APG09344 tracrRNA | 564 |
| APG09344 PAM | 565 (NAG) |
| APG07991 crRNA repeat | 566 |
| APG07991 tracrRNA | 567 |
| APG07991 PAM | 568 (NGG) |
| APG01868 crRNA repeat | 569 |
| APG01868 tracrRNA | 570 |
| APG01868 PAM | 571 (NGG) |
| APG02998 crRNA repeat | 572 |
| APG02998 tracrRNA | 573 |
| APG02998 PAM | 574 (NGG) |
| APG07433.1 deletion variant | 575 |
| APG08290.1 deletion variant | 576 |
| LPG20046 | 577 |
| LPG20047 | 578 |
| LPG20124 | 579 |
| LPG20123 | 580 |
| LPG20048 | 581 |
| LPG20051 | 582 |
| LPG20055 | 583 |
| LPG20056 | 584 |
| LPG20057 | 585 |
| LPG20058 | 586 |
| LPG20059 | 587 |
| LPG20060 | 588 |
| LPG20050 | 589 |
| LPG20053 | 590 |
| LPG20054 | 591 |
| LPG20044 | 592 |
| LPG20052 | 593 |
| LPG20049 | 594 |
| LPG20045 | 595 |
| LPG50320 | 596 |
| LPG50324 | 597 |
| LPG50318 | 598 |
| LPG20061 | 599 |
| LPG20062 | 600 |
| SGN008971 target sequence | 601 |
| LPG20063 | 602 |
| LPG20064 | 603 |
| LPG20065 | 604 |
| LPG20066 | 605 |
| LPG20067 | 606 |
| LPG20068 | 607 |
| LPG20069 | 608 |
| LPG20070 | 609 |
| LPG20071 | 610 |
| LPG20072 | 611 |
| LPG20073 | 612 |
| LPG20074 | 613 |
| LPG20075 | 614 |
| LPG20076 | 615 |
| LPG20077 | 616 |
| LPG20083 | 617 |
| LPG20084 | 618 |
| LPG20085 | 619 |
| LPG20086 | 620 |
| LPG20087 | 621 |
| LPG20088 | 622 |
| LPG20089 | 623 |
| LPG20090 | 624 |
| LPG20091 | 625 |
| LPG20092 | 626 |
| LPG20093 | 627 |
| LPG20094 | 628 |
| LPG20095 | 629 |
| LPG20096 | 630 |
| LPG20097 | 631 |
| LPG20098 | 632 |
| LPG50318 | 633 |
| deadAPG07433.1_inlaidS772_LPG50148 (LPG20134) | 634 |
| deadAPG07433.1_inlaidS775_LPG50148 (LPG20135) | 635 |
| deadAPG07433.1_inlaidK778_LPG50148 (LPG20136) | 636 |
| deadAPG07433.1 inlaidE900_LPG50148 (LPG20137) | 637 |
| deadAPG07433.1_inlaidG910_LPG50148 (LPG20138) | 638 |
| LPG20130 | 639 |
| SGN002336 | 640 |
| SGN002364 | 641 |
| Peptide linker | 642 |
| LPG20099 | 643 |
| LPG20100 | 644 |
| LPG20101 | 645 |
| LPG20102 | 646 |
| LPG20111 | 647 |
| LPG20120 | 648 |
| LPG20121 | 649 |
| LPG20122 | 650 |
| SGN001432 | 651 |
| SGN001433 | 652 |
| SGN001434 | 653 |
| SGN001435 | 654 |
| SGN008382 | 655 |
| SGN008383 | 656 |
| SGN008384 | 657 |
| SGN008385 | 658 |
| SGN008393 | 659 |
| SGN001432 target seg | 660 |
| SGN001433 target seq | 661 |
| SGN001434 target seq | 662 |
| SGN001435 target seq | 663 |
| SGN008382 target seq | 664 |
| SGN008383 target seq | 665 |
| SGN008384 target seg | 666 |
| SGN008385 target seq | 667 |
| SGN008393 target seq | 668 |
| SGN001256 | 669 |
| SGN001947 | 670 |
| SGN001960 | 671 |
| SGN003707 | 672 |
| SGN004995 | 673 |
| SGN001256 target sequence | 674 |
| SGN001947 target sequence | 675 |
| SGN001960 target sequence | 676 |
| SGN003707 target sequence | 677 |
| SGN004995 target sequence | 678 |
| SGN003018 | 679 |
| SGN003540 | 680 |
| SGN003018 target sequence | 681 |
| SGN003540 target sequence | 682 |
| SGN003486 | 683 |
| SGN002352 | 684 |
| SGN001881 | 685 |
| SGN003486 target sequence | 686 |
| SGN005430 | 687 |
| SGN005420 | 688 |
| SGN005326 | 689 |
| SGN005329 | 690 |
| SGN005326 target sequence | 691 |
| SGN005329 target sequence | 692 |
| Full-length APG07433.1 backbone (F) | 693 |
| Shortened APG07433.1 backbone (M) | 694 |
| APG05586 Backbone | 695 |
| Shortened LPG10145 backbone | 696 |
| Shortened LPG10196 backbone | 697 |
| nLPG10196 (D11A) | 698 |
| nLPG10196 (H840A) | 699 |
| LAGLIDADG | 700 |
| Streptococcus thermophilus Cas9 | 701 |
| Streptococcus aureus Cas9 | 702 |
| Francisella novicida Cas12a | 703 |
| L16 Linker sequence | 704 |
| L32 Linker sequence | 705 |
| NLS linker | 706 |
| P(AP)3 linker | 707 |
| (EAAAK)2 linker | 708 |
| 24A linker | 709 |
| 24B linker | 710 |
| GS16 linker | 711 |
| 18x linker | 712 |
| POLDI NLS | 713 |
| ADAR2 NLS | 714 |
| Iwr1 NLS | 715 |
| OpT NLS | 716 |
| SV40 Bipartite NLS | 717 |
| c-Myc NLS | 718 |
| NLS linker | 719 |
| LPG50321 | 720 |
| LPG50322 | 721 |
| LPG50323 | 722 |
| LPG50310 | 723 |
| LPG20126 | 724 |
| LPG20127 | 725 |
| LPG20128 | 726 |
| LPG20125 | 727 |
| NLS linker | 728 |
| LPG20129 | 729 |
| SGN008971 | 730 |

1.-140. (canceled)

141. A deaminase comprising an amino acid sequence having at least 85% sequence identity to SEQ ID NO: 5, and comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.

142. The deaminase of claim 141, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.

143. The deaminase of claim 141, wherein said deaminase comprises an amino acid sequence having at least 90% sequence identity to or comprises the amino acid sequence set forth as any one of SEQ ID NOs: 300-414, 596-598, and 720-723.

144. (canceled)

145. The deaminase of claim 141, wherein said deaminase has improved deaminase activity when compared to SEQ ID NO: 5 and/or wherein the deaminase is an adenine deaminase.

146. (canceled)

147. A nucleic acid molecule comprising a polynucleotide encoding a deaminase having at least 85% sequence identity to SEQ ID NO: 5, wherein the deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.

148. The nucleic acid molecule of claim 147, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.

149. The nucleic acid molecule of claim 147, wherein said deaminase comprises an amino acid sequence having at least 90% sequence identity to or comprises the amino acid sequence set forth as any one of SEQ ID NOs: 300-414, 596-598, and 720-723.

150. (canceled)

151. The nucleic acid molecule of claim 147, wherein said deaminase has improved deaminase activity when compared to SEQ ID NO: 5 and/or wherein the deaminase is an adenine deaminase.

152. (canceled)

153. The nucleic acid molecule of claim 147, further comprising a heterologous promoter operably linked to said polynucleotide.

154. A vector comprising the nucleic acid molecule of claim 147.

155.-157. (canceled)

158. A fusion protein comprising a DNA-binding polypeptide and a deaminase having at least 85% sequence identity to SEQ ID NO: 5, wherein the deaminase comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.

159. The fusion protein of claim 158, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.

160. The fusion protein of claim 158, wherein said deaminase comprises an amino acid sequence having at least 90% sequence identity to or comprises the amino acid sequence set forth as any one of SEQ ID NOs: 300-414, 596-598, and 720-723.

161.-163. (canceled)

164. The fusion protein of claim 158, wherein the DNA-binding polypeptide is an RNA-guided nuclease (RGN) polypeptide.

165. The fusion protein of claim 164, wherein the RGN is an RGN nickase.

166. The fusion protein of claim 164, wherein the RGN has an amino acid sequence having at least 95% sequence identity to any one of SEQ ID NOs: 1-4, 49-162, 435, 575, 576, 698, and 699.

167. The fusion protein of claim 165, wherein the RGN nickase has the amino acid sequence set forth as any one of SEQ ID NOs: 49-52 and 698.

168. The fusion protein of claim 158, wherein said fusion protein is selected from the group consisting of:
a) a fusion protein comprising said deaminase, wherein said deaminase comprises an amino acid sequence having at least 90% sequence identity to or comprises the amino acid sequence set forth as SEQ ID NO: 319 and is fused to the N-terminus of an RGN comprising an amino acid sequence having at least 90% sequence identity to or comprises the amino acid sequence set forth as SEQ ID NO: 49;
b) a fusion protein comprising said deaminase, wherein said deaminase comprises an amino acid sequence having at least 90% sequence identity to or comprises the amino acid sequence set forth as SEQ ID NO: 375 and is fused to the N-terminus of an RGN comprising an amino acid sequence having at least 90% sequence identity to or comprises the amino acid sequence set forth as SEQ ID NO: 49;
c) a fusion protein comprising said deaminase, wherein said deaminase comprises an amino acid sequence having at least 90% sequence identity to or comprises the amino acid sequence set forth as SEQ ID NO: 406 and is fused to the N-terminus of an RGN comprising an amino acid sequence having at least 90% sequence identity to or comprises the amino acid sequence set forth as SEQ ID NO: 49;
d) a fusion protein comprising said deaminase, wherein said deaminase comprises an amino acid sequence having at least 90% sequence identity to or comprises the amino acid sequence set forth as SEQ ID NO: 596 and is fused to the N-terminus of an RGN comprising an amino acid sequence having at least 90% sequence identity to or comprises the amino acid sequence set forth as SEQ ID NO: 49;
e) a fusion protein comprising said deaminase, wherein said deaminase comprises an amino acid sequence having at least 90% sequence identity to or comprises the amino acid sequence set forth as SEQ ID NO: 597 and is fused to the N-terminus of an RGN comprising an amino acid sequence having at least 90% sequence identity to or comprises the amino acid sequence set forth as SEQ ID NO: 49;
f) a fusion protein comprising said deaminase, wherein said deaminase comprises an amino acid sequence having at least 90% sequence identity to or comprises the amino acid sequence set forth as SEQ ID NO: 303 and is fused to the N-terminus of an RGN comprising an amino acid sequence having at least 90% sequence identity to or comprises the amino acid sequence set forth as SEQ ID NO: 49;
g) a fusion protein comprising said deaminase, wherein said deaminase comprises an amino acid sequence having at least 90% sequence identity to or comprises the amino acid sequence set forth as SEQ ID NO: 366 and is fused to the N-terminus of an RGN comprising an amino acid sequence having at least 90% sequence identity to or comprises the amino acid sequence set forth as SEQ ID NO: 49; and
h) a fusion protein comprising said deaminase, wherein said deaminase comprises an amino acid sequence having at least 90% sequence identity to or comprises the amino acid sequence set forth as SEQ ID NO: 720 and is fused to the N-terminus of an RGN comprising an amino acid sequence having at least 90% sequence identity to or comprises the amino acid sequence set forth as SEQ ID NO: 49.

169. (canceled)

170. The fusion protein of claim 158, wherein said fusion protein has the sequence set forth as any one of SEQ ID NOs: 577, 578, 582, 584, 588, 593, 594, 604, 605, 616, and 729.

171. A ribonucleoprotein (RNP) complex comprising the fusion protein of claim 158 and a guide RNA bound to the fusion protein.

172. A nucleic acid molecule comprising a polynucleotide encoding a fusion protein comprising a DNA-binding polypeptide and a deaminase, wherein the deaminase comprises an amino acid sequence having at least 85% sequence identity to SEQ ID NO: 5 and comprises at least one of the following amino acid residues:
a) a C at a position corresponding to position 2 of SEQ ID NO: 5;
b) a F or C at a position corresponding to position 22 of SEQ ID NO: 5;
c) a Q at a position corresponding to position 23 of SEQ ID NO: 5;
d) a N at a position corresponding to position 35 of SEQ ID NO: 5;
e) a L at a position corresponding to position 40 of SEQ ID NO: 5;
f) a Q at a position corresponding to position 46 of SEQ ID NO: 5;
g) a A or M at a position corresponding to position 68 of SEQ ID NO: 5;
h) a H at a position corresponding to position 72 of SEQ ID NO: 5;
i) a W, A, Q, Y, or D at a position corresponding to position 75 of SEQ ID NO: 5;
j) a G at a position corresponding to position 76 of SEQ ID NO: 5;
k) a S at a position corresponding to position 81 of SEQ ID NO: 5;
l) a M at a position corresponding to position 105 of SEQ ID NO: 5;
m) a H at a position corresponding to position 108 of SEQ ID NO: 5;
n) a L at a position corresponding to position 109 of SEQ ID NO: 5;
o) a I or L at a position corresponding to position 117 of SEQ ID NO: 5;
p) a F at a position corresponding to position 120 of SEQ ID NO: 5;
q) a E, T, A, G, or V at a position corresponding to position 121 of SEQ ID NO: 5;
r) a I or H at a position corresponding to position 122 of SEQ ID NO: 5;
s) a K at a position corresponding to position 125 of SEQ ID NO: 5;
t) a A at a position corresponding to position 126 of SEQ ID NO: 5;
u) a H at a position corresponding to position 135 of SEQ ID NO: 5;
v) a V at a position corresponding to position 137 of SEQ ID NO: 5;
w) a Y at a position corresponding to position 138 of SEQ ID NO: 5;
x) a L at a position corresponding to position 139 of SEQ ID NO: 5;
y) a K or A at a position corresponding to position 142 of SEQ ID NO: 5;
z) a A, Q, L, or M at a position corresponding to position 145 of SEQ ID NO: 5;
aa) a K at a position corresponding to position 148 of SEQ ID NO: 5;
bb) a Q at a position corresponding to position 151 of SEQ ID NO: 5;
cc) a E or R at a position corresponding to position 153 of SEQ ID NO: 5;
dd) a W at a position corresponding to position 155 of SEQ ID NO: 5;
ee) a R or V at a position corresponding to position 156 of SEQ ID NO: 5;
ff) a F at a position corresponding to position 157 of SEQ ID NO: 5;
gg) a R at a position corresponding to position 158 of SEQ ID NO: 5;
hh) a Q at a position corresponding to position 159 of SEQ ID NO: 5;
ii) a D or W at a position corresponding to position 160 of SEQ ID NO: 5;
jj) a W, H, V, or A at a position corresponding to position 162 of SEQ ID NO: 5;
kk) a R at a position corresponding to position 165 of SEQ ID NO: 5; and
ll) a H at a position corresponding to position 166 of SEQ ID NO: 5.

173. The nucleic acid molecule of claim 172, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.

174.-177. (canceled)

178. The nucleic acid molecule of claim 172, wherein the DNA-binding polypeptide is an RNA-guided nuclease (RGN) polypeptide.

179. The nucleic acid molecule of claim 178, wherein the RGN is an RGN nickase.

180.-184. (canceled)

185. A vector comprising the nucleic acid molecule of claim 172.

186. (canceled)

187. A cell comprising the fusion protein of claim 158.

188.-193. (canceled)

194. A pharmaceutical composition comprising a pharmaceutically acceptable carrier and the fusion protein of claim 158.

195. (canceled)

196. A system for modifying a target DNA molecule comprising a target DNA sequence, said system comprising:
a) a fusion protein comprising an RNA-guided nuclease polypeptide and a deaminase, wherein the deaminase has an amino acid sequence having at least 85% sequence identity to SEQ ID NO: 5, or a nucleotide sequence encoding said fusion protein, wherein said deaminase comprises at least one of the following amino acid residues:
i) a C at a position corresponding to position 2 of SEQ ID NO: 5;
ii) a F or C at a position corresponding to position 22 of SEQ ID NO: 5;
iii) a Q at a position corresponding to position 23 of SEQ ID NO: 5;
iv) a N at a position corresponding to position 35 of SEQ ID NO: 5;
v) a L at a position corresponding to position 40 of SEQ ID NO: 5;
vi) a Q at a position corresponding to position 46 of SEQ ID NO: 5;
vii) a A or M at a position corresponding to position 68 of SEQ ID NO: 5;
viii) a H at a position corresponding to position 72 of SEQ ID NO: 5;
ix) a W, A, Q, Y, or D at a position corresponding to position 75 of SEQ ID NO: 5;
x) a G at a position corresponding to position 76 of SEQ ID NO: 5;
xi) a S at a position corresponding to position 81 of SEQ ID NO: 5;
xii) a M at a position corresponding to position 105 of SEQ ID NO: 5;
xiii) a H at a position corresponding to position 108 of SEQ ID NO: 5;
xiv) a L at a position corresponding to position 109 of SEQ ID NO: 5;
xv) a I or L at a position corresponding to position 117 of SEQ ID NO: 5;
xvi) a F at a position corresponding to position 120 of SEQ ID NO: 5;
xvii) a E, T, A, G, or V at a position corresponding to position 121 of SEQ ID NO: 5;
xviii) a I or H at a position corresponding to position 122 of SEQ ID NO: 5;
xix) a K at a position corresponding to position 125 of SEQ ID NO: 5;
xx) a A at a position corresponding to position 126 of SEQ ID NO: 5;
xxi) a H at a position corresponding to position 135 of SEQ ID NO: 5;
xxii) a V at a position corresponding to position 137 of SEQ ID NO: 5;
xxiii) a Y at a position corresponding to position 138 of SEQ ID NO: 5;
xxiv) a L at a position corresponding to position 139 of SEQ ID NO: 5;
xxv) a K or A at a position corresponding to position 142 of SEQ ID NO: 5;
xxvi) a A, Q, L, or M at a position corresponding to position 145 of SEQ ID NO: 5;
xxvii) a K at a position corresponding to position 148 of SEQ ID NO: 5;
xxviii) a Q at a position corresponding to position 151 of SEQ ID NO: 5;
xxix) a E or R at a position corresponding to position 153 of SEQ ID NO: 5;
xxx) a W at a position corresponding to position 155 of SEQ ID NO: 5;
xxxi) a R or V at a position corresponding to position 156 of SEQ ID NO: 5;
xxxii) a F at a position corresponding to position 157 of SEQ ID NO: 5;
xxxiii) a R at a position corresponding to position 158 of SEQ ID NO: 5;
xxxiv) a Q at a position corresponding to position 159 of SEQ ID NO: 5;
xxxv) a D or W at a position corresponding to position 160 of SEQ ID NO: 5;
xxxvi) a W, H, V, or A at a position corresponding to position 162 of SEQ ID NO: 5;
xxxvii) a R at a position corresponding to position 165 of SEQ ID NO: 5; and
xxxviii) a H at a position corresponding to position 166 of SEQ ID NO: 5; and
b) one or more guide RNAs capable of hybridizing to a non-target strand of said target DNA sequence or one or more nucleotide sequences encoding the one or more guide RNAs.

197. The system of claim 196, wherein said deaminase comprises:
a) a N at a position corresponding to position 35 of SEQ ID NO: 5 and a W at a position corresponding to position 162 of SEQ ID NO: 5;
b) a N at a position corresponding to position 35 of SEQ ID NO: 5 and a Q at a position corresponding to position 46 of SEQ ID NO: 5;
c) a S at a position corresponding to position 81 of SEQ ID NO: 5 and a R at a position corresponding to position 156 of SEQ ID NO: 5;
d) a Q at a position corresponding to position 46 of SEQ ID NO: 5 and a R at a position corresponding to position 156 of SEQ ID NO: 5;
e) a R at a position corresponding to position 156 of SEQ ID NO: 5 and a W at a position corresponding to position 162 of SEQ ID NO: 5;
f) a A at a position corresponding to position 68 of SEQ ID NO: 5 and a S at a position corresponding to position 81 of SEQ ID NO: 5;
g) a N at a position corresponding to position 35 of SEQ ID NO: 5, a S at a position corresponding to position 81 of SEQ ID NO: 5, and a W at position 162 of SEQ ID NO: 5;
h) a N at a position corresponding to position 35 of SEQ ID NO: 5, a Q at a position corresponding to position 46 of SEQ ID NO: 5, and a W at a position corresponding to position 162 of SEQ ID NO: 5;
i) a W at a position corresponding to position 155 of SEQ ID NO: 5, a R at a position corresponding to position 156 of SEQ ID NO: 5, and a W at a position corresponding to position 162 of SEQ ID NO: 5;
j) a N at a position corresponding to position 35 of SEQ ID NO: 5, a R at a position corresponding to position 156 of SEQ ID NO: 5, and a W at a position corresponding to position 162 of SEQ ID NO: 5;
k) a N at a position corresponding to position 35 of SEQ ID NO: 5, a S at a position corresponding to position 81 of SEQ ID NO: 5, a R at a position corresponding to position 156 of SEQ ID NO: 5, and a W at a position corresponding to position 162 of SEQ ID NO: 5;
l) a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
m) a Q at a position corresponding to position 46 of SEQ ID NO: 5, a M at a position corresponding to position 68 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
n) a M at a position corresponding to position 68 of SEQ ID NO: 5, a S at a position corresponding to position 81 of SEQ ID NO: 5, a H at a position corresponding to position 108 of SEQ ID NO: 5, a R at a position corresponding to position 156 of SEQ ID NO: 5, and a W at a position corresponding to position 162 of SEQ ID NO: 5;
o) a N at a position corresponding to position 35, a Q at a position corresponding to position 46 of SEQ ID NO: 5, a M at a position corresponding to position 68 of SEQ ID NO: 5, a S at a position corresponding to position 81 of SEQ ID NO: 5, a H at a position corresponding to position 108 of SEQ ID NO: 5, a W at a position corresponding to position 155 of SEQ ID NO: 5, a R at a position corresponding to position 156 of SEQ ID NO: 5, and a W at a position corresponding to position 162 of SEQ ID NO: 5;
p) a S at a position corresponding to position 81 of SEQ ID NO: 5, and a W at a position corresponding to position 155 of SEQ ID NO: 5;
q) a W at a position corresponding to position 75 of SEQ ID NO: 5, and a S at a position corresponding to position 81 of SEQ ID NO: 5;
r) a S at a position corresponding to position 81 of SEQ ID NO: 5, and a D at a position corresponding to position 160 of SEQ ID NO: 5;
s) a S at a position corresponding to position 81 of SEQ ID NO: 5, and a A at a position corresponding to position 145 of SEQ ID NO: 5;
t) a W at a position corresponding to position 75 of SEQ ID NO: 5, and a W at a position corresponding to position 155 of SEQ ID NO: 5;
u) a W at a position corresponding to position 155 of SEQ ID NO: 5, and a D at a position corresponding to position 160 of SEQ ID NO: 5;
v) a A at a position corresponding to position 145 of SEQ ID NO: 5, and a W at a position corresponding to position 155 of SEQ ID NO: 5;
w) a W at a position corresponding to position 75 of SEQ ID NO: 5, and a D at a position corresponding to position 160 of SEQ ID NO: 5;
x) a W at a position corresponding to position 75 of SEQ ID NO: 5, and a A at a position corresponding to position 145 of SEQ ID NO: 5;
y) a A at a position corresponding to position 145 of SEQ ID NO: 5, and a D at a position corresponding to position 160 of SEQ ID NO: 5;
z) a W at a position corresponding to position 75 of SEQ ID NO: 5, a S at a position corresponding to position 81 of SEQ ID NO: 5, and a W at a position corresponding to position 155 of SEQ ID NO: 5;
aa) a S at a position corresponding to position 81 of SEQ ID NO: 5, a W at a position corresponding to position 155 of SEQ ID NO: 5, and a D at a position corresponding to position 160 of SEQ ID NO: 5;
bb) a S at a position corresponding to position 81 of SEQ ID NO: 5, a A at a position corresponding to position 145 of SEQ ID NO: 5, and a W at a position corresponding to position 155 of SEQ ID NO: 5;
cc) a W at a position corresponding to position 75 of SEQ ID NO: 5, a S at a position corresponding to position 81 of SEQ ID NO: 5, and a D at a position corresponding to position 160 of SEQ ID NO: 5;
dd) a W at a position corresponding to position 75 of SEQ ID NO: 5, a S at a position corresponding to position 81 of SEQ ID NO: 5, and a A at a position corresponding to position 145 of SEQ ID NO: 5;
ee) a S at a position corresponding to position 81 of SEQ ID NO: 5, a A at a position corresponding to position 145 of SEQ ID NO: 5, and a D at a position corresponding to position 160 of SEQ ID NO: 5;
ff) a W at a position corresponding to position 75 of SEQ ID NO: 5, a W at a position corresponding to position 155 of SEQ ID NO: 5, and a D at a position corresponding to position 160 of SEQ ID NO: 5;
gg) a W at a position corresponding to position 75 of SEQ ID NO: 5, a A at a position corresponding to position 145 of SEQ ID NO: 5, and a W at a position corresponding to position 155 of SEQ ID NO: 5;
hh) a A at a position corresponding to position 145 of SEQ ID NO: 5, a W at a position corresponding to position 155 of SEQ ID NO: 5, and a D at a position corresponding to position 160 of SEQ ID NO: 5;
ii) a W at a position corresponding to position 75 of SEQ ID NO: 5, a A at a position corresponding to position 145 of SEQ ID NO: 5, and a D at a position corresponding to position 160 of SEQ ID NO: 5;
jj) a W at a position corresponding to position 75 of SEQ ID NO: 5, a S at a position corresponding to position 81 of SEQ ID NO: 5, a W at a position corresponding to position 155 of SEQ ID NO: 5, and a D at a position corresponding to position 160 of SEQ ID NO: 5;
kk) a W at a position corresponding to position 75 of SEQ ID NO: 5, a S at a position corresponding to position 81 of SEQ ID NO: 5, a A at a position corresponding to position 145 of SEQ ID NO: 5, and a W at a position corresponding to position 155 of SEQ ID NO: 5;
ll) a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
mm) a W at a position corresponding to position 75 of SEQ ID NO: 5, a S at a position corresponding to position 81 of SEQ ID NO: 5, a A at a position corresponding to position 145 of SEQ ID NO: 5, and a D at a position corresponding to position 160 of SEQ ID NO: 5;
nn) a W at a position corresponding to position 75 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
oo) a W at a position corresponding to position 75 of SEQ ID NO: 5, a S at a position corresponding to position 81 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
pp) a L at a position corresponding to position 40 of SEQ ID NO: 5, and a M at a position corresponding to position 105 of SEQ ID NO: 5;
qq) a L at a position corresponding to position 40 of SEQ ID NO: 5, and a V at a position corresponding to position 121 of SEQ ID NO: 5;
rr) a L at a position corresponding to position 40 of SEQ ID NO: 5, and a M at a position corresponding to position 145 of SEQ ID NO: 5;
ss) a L at a position corresponding to position 40 of SEQ ID NO: 5, and a Q at a position corresponding to position 159 of SEQ ID NO: 5;
tt) a L at a position corresponding to position 40 of SEQ ID NO: 5, and a W at a position corresponding to position 160 of SEQ ID NO: 5;
uu) a M at a position corresponding to position 105 of SEQ ID NO: 5, and a V at a position corresponding to position 121 of SEQ ID NO: 5;
vv) a M at a position corresponding to position 105 of SEQ ID NO: 5, and a M at a position corresponding to position 145 of SEQ ID NO: 5;
ww) a M at a position corresponding to position 105 of SEQ ID NO: 5, and a Q at a position corresponding to position 159 of SEQ ID NO: 5;
xx) a M at a position corresponding to position 105 of SEQ ID NO: 5, and a W at a position corresponding to position 160 of SEQ ID NO: 5;
yy) a V at a position corresponding to position 121 of SEQ ID NO: 5, and a M at a position corresponding to position 145 of SEQ ID NO: 5;
zz) a V at a position corresponding to position 121 of SEQ ID NO: 5, and a Q at a position corresponding to position 159 of SEQ ID NO: 5;
aaa) a V at a position corresponding to position 121 of SEQ ID NO: 5, and a W at a position corresponding to position 160 of SEQ ID NO: 5;
bbb) a M at a position corresponding to position 145 of SEQ ID NO: 5, and a Q at a position corresponding to position 159 of SEQ ID NO: 5;
ccc) a M at a position corresponding to position 145 of SEQ ID NO: 5, and a W at a position corresponding to position 160 of SEQ ID NO: 5;
ddd) a Q at a position corresponding to position 159 of SEQ ID NO: 5, and a W at a position corresponding to position 160 of SEQ ID NO: 5;
eee) a S at a position corresponding to position 81 of SEQ ID NO: 5, a M at a position corresponding to position 145 of SEQ ID NO: 5, and a W at a position corresponding to position 160 of SEQ ID NO: 5;
fff) a S at a position corresponding to position 81 of SEQ ID NO: 5, a V at a position corresponding to position 121 of SEQ ID NO: 5, and a Q at a position corresponding to position 159 of SEQ ID NO: 5;
ggg) a A at a position corresponding to position 68 of SEQ ID NO: 5, and a S at a position corresponding to position 81 of SEQ ID NO: 5;
hhh) a G at a position corresponding to position 76 of SEQ ID NO: 5, and a S at a position corresponding to position 81 of SEQ ID NO: 5;
iii) a S at a position corresponding to position 81 of SEQ ID NO: 5, and a L at a position corresponding to position 117 of SEQ ID NO: 5;
jjj) a A at a position corresponding to position 68 of SEQ ID NO: 5, and a G at a position corresponding to position 76 of SEQ ID NO: 5;
kkk) a A at a position corresponding to position 68 of SEQ ID NO: 5, and a L at a position corresponding to position 117 of SEQ ID NO: 5;
lll) a G at a position corresponding to position 76 of SEQ ID NO: 5, and a L at a position corresponding to position 117 of SEQ ID NO: 5;
mmm) a A at a position corresponding to position 68 of SEQ ID NO: 5, a G at a position corresponding to position 76 of SEQ ID NO: 5, and a S at a position corresponding to position 81 of SEQ ID NO: 5;
nnn) a A at a position corresponding to position 68 of SEQ ID NO: 5, a S at a position corresponding to position 81 of SEQ ID NO: 5, and a L at a position corresponding to position 117 of SEQ ID NO: 5;
ooo) a G at a position corresponding to position 76 of SEQ ID NO: 5, a S at a position corresponding to position 81 of SEQ ID NO: 5, and a L at a position corresponding to position 117 of SEQ ID NO: 5;
ppp) a A at a position corresponding to position 68 of SEQ ID NO: 5, a G at a position corresponding to position 76 of SEQ ID NO: 5, and a L at a position corresponding to position 117 of SEQ ID NO: 5;
qqq) a A at a position corresponding to position 68 of SEQ ID NO: 5, a G at a position corresponding to position 76 of SEQ ID NO: 5, a S at a position corresponding to position 81 of SEQ ID NO: 5, and a L at a position corresponding to position 117 of SEQ ID NO: 5;
rrr) a S at a position corresponding to position 81 of SEQ ID NO: 5, a L at a position corresponding to position 109 of SEQ ID NO: 5, a E at a position corresponding to position 153 of SEQ ID NO: 5, a W at a position corresponding to position 155 of SEQ ID NO: 5, and a D at a position corresponding to position 160 of SEQ ID NO: 5;
sss) a A at a position corresponding to position 75 of SEQ ID NO: 5, a S at a position corresponding to position 81 of SEQ ID NO: 5, a L at a position corresponding to position 117 of SEQ ID NO: 5, a A at a position corresponding to position 121 of SEQ ID NO: 5, a A at a position corresponding to position 145 of SEQ ID NO: 5, a W at a position corresponding to position 155 of SEQ ID NO: 5, and a D at a position corresponding to position 160 of SEQ ID NO: 5;
ttt) a Q at a position corresponding to position 75 of SEQ ID NO: 5, a S at a position corresponding to position 81 of SEQ ID NO: 5, a I at a position corresponding to position 117 of SEQ ID NO: 5, a Q at a position corresponding to position 145 of SEQ ID NO: 5, a W at a position corresponding to position 155 of SEQ ID NO: 5, and a D at a position corresponding to position 160 of SEQ ID NO: 5;
uuu) a Y at a position corresponding to position 75 of SEQ ID NO: 5, a S at a position corresponding to position 81 of SEQ ID NO: 5, a L at a position corresponding to position 117 of SEQ ID NO: 5, a G at a position corresponding to position 121 of SEQ ID NO: 5, a L at a position corresponding to position 145 of SEQ ID NO: 5, a W at a position corresponding to position 155 of SEQ ID NO: 5, and a D at a position corresponding to position 160 of SEQ ID NO: 5;
vvv) a C at a position corresponding to position 22 of SEQ ID NO: 5, a A at a position corresponding to position 68 of SEQ ID NO: 5, a Y at a position corresponding to position 75 of SEQ ID NO: 5, a G at a position corresponding to position 76 of SEQ ID NO: 5, a S at a position corresponding to position 81 of SEQ ID NO: 5, a H at a position corresponding to position 108 of SEQ ID NO: 5, a L at a position corresponding to position 117 of SEQ ID NO: 5, a H at a position corresponding to position 122 of SEQ ID NO: 5, a Y at a position corresponding to position 138 of SEQ ID NO: 5, a L at a position corresponding to position 139 of SEQ ID NO: 5, a A at a position corresponding to position 142 of SEQ ID NO: 5, a A at a position corresponding to position 145 of SEQ ID NO: 5, a R at a position corresponding to position 153 of SEQ ID NO: 5, a W at a position corresponding to position 155 of SEQ ID NO: 5, a R at a position corresponding to position 156 of SEQ ID NO: 5, a D at a position corresponding to position 160 of SEQ ID NO: 5, and a H at a position corresponding to position 162 of SEQ ID NO: 5; or
www) a S at a position corresponding to position 81 of SEQ ID NO: 5.

198.-201. (canceled)

202. The system of claim 196, wherein at least one of said nucleotide sequences encoding the one or more guide RNAs and said nucleotide sequence encoding the fusion protein is operably linked to a promoter heterologous to said nucleotide sequence.

203.-215. (canceled)

216. A method for modifying a target DNA molecule comprising a target DNA sequence, said method comprising delivering a system according to claim 196 to said target DNA molecule or a cell comprising the target DNA molecule.

217. A method for modifying a target DNA molecule comprising a target sequence comprising:
a) assembling an RGN-deaminase ribonucleotide complex in vitro by combining:
i) one or more guide RNAs capable of hybridizing to a non-target strand of the target DNA sequence; and
ii) a fusion protein comprising an RNA-guided nuclease polypeptide (RGN), and at least one deaminase, wherein the deaminase has an amino acid sequence having at least 85% sequence identity to SEQ ID NO: 5;
under conditions suitable for formation of the RGN-deaminase ribonucleotide complex; wherein said deaminase has at least one of the following amino acid residues:
A) a C at a position corresponding to position 2 of SEQ ID NO: 5;
B) a F or C at a position corresponding to position 22 of SEQ ID NO: 5;
C) a Q at a position corresponding to position 23 of SEQ ID NO: 5;
D) a N at a position corresponding to position 35 of SEQ ID NO: 5;
E) a L at a position corresponding to position 40 of SEQ ID NO: 5;
F) a Q at a position corresponding to position 46 of SEQ ID NO: 5;
G) a A or M at a position corresponding to position 68 of SEQ ID NO: 5;
H) a H at a position corresponding to position 72 of SEQ ID NO: 5;
I) a W, A, Q, Y, or D at a position corresponding to position 75 of SEQ ID NO: 5;
J) a G at a position corresponding to position 76 of SEQ ID NO: 5;
K) a S at a position corresponding to position 81 of SEQ ID NO: 5;
L) a M at a position corresponding to position 105 of SEQ ID NO: 5;
M) a H at a position corresponding to position 108 of SEQ ID NO: 5;
N) a L at a position corresponding to position 109 of SEQ ID NO: 5;
O) a I or L at a position corresponding to position 117 of SEQ ID NO: 5;
P) a F at a position corresponding to position 120 of SEQ ID NO: 5;
Q) a E, T, A, G, or V at a position corresponding to position 121 of SEQ ID NO: 5;
R) a I or H at a position corresponding to position 122 of SEQ ID NO: 5;
S) a K at a position corresponding to position 125 of SEQ ID NO: 5;
T) a A at a position corresponding to position 126 of SEQ ID NO: 5;
U) a H at a position corresponding to position 135 of SEQ ID NO: 5;
V) a V at a position corresponding to position 137 of SEQ ID NO: 5;
W) a Y at a position corresponding to position 138 of SEQ ID NO: 5;
X) a L at a position corresponding to position 139 of SEQ ID NO: 5;
Y) a K or A at a position corresponding to position 142 of SEQ ID NO: 5;
Z) a A, Q, L, or M at a position corresponding to position 145 of SEQ ID NO: 5;
AA) a K at a position corresponding to position 148 of SEQ ID NO: 5;
BB) a Q at a position corresponding to position 151 of SEQ ID NO: 5;
CC) a E or R at a position corresponding to position 153 of SEQ ID NO: 5;
DD) a W at a position corresponding to position 155 of SEQ ID NO: 5;
EE) a R or V at a position corresponding to position 156 of SEQ ID NO: 5;
FF) a F at a position corresponding to position 157 of SEQ ID NO: 5;
GG) a R at a position corresponding to position 158 of SEQ ID NO: 5;
HH) a Q at a position corresponding to position 159 of SEQ ID NO: 5;
II) a D or W at a position corresponding to position 160 of SEQ ID NO: 5;
JJ) a W, H, V, or A at a position corresponding to position 162 of SEQ ID NO: 5;
KK) a R at a position corresponding to position 165 of SEQ ID NO: 5; and
LL) a H at a position corresponding to position 166 of SEQ ID NO: 5; and
b) contacting said target DNA molecule or a cell comprising said target DNA molecule with the in vitro-assembled RGN-deaminase ribonucleotide complex;
wherein the one or more guide RNAs hybridize to a non-target strand of the target DNA sequence, thereby directing said fusion protein to bind to said target DNA sequence and modification of the target DNA molecule occurs.

218.-239. (canceled)

240. A method for treating a subject having or at risk of developing a disease, disorder, or condition, the method comprising:
administering to the subject the fusion protein of claim 158.

241.-245. (canceled)
